SEQUENCE LISTING (267 sequences)

SEQ ID NO: 1 - protein, 30 aa, artificial sequence
  Covid-19 surface peptide position 22-51
TQLPPAYTNSFTRGVYYPDKVFRSSVLHST

SEQ ID NO: 2 - protein, 30 aa, artificial sequence
  Covid-19 surface peptide position 35-64
GVYYPDKVFRSSVLHSTQDLFLPFFSNVTW

SEQ ID NO: 3 - protein, 30 aa, artificial sequence
  Covid-19 surface peptide position 76-105
TKRFDNPVLPFNDGVYFASTEKSNIIRGWI

SEQ ID NO: 4 - protein, 30 aa, artificial sequence
  Covid-19 surface peptide position 98-127
SNIIRGWIFGTTLDSKTQSLLIVNNATNVV

SEQ ID NO: 5 - protein, 30 aa, artificial sequence
  Covid-19 surface peptide position 253-282
DSSSGWTAGAAAYYVGYLQPRTFLLKYNEN

SEQ ID NO: 6 - protein, 30 aa, artificial sequence
  Covid-19 surface peptide position 391-420
CFTNVYADSFVIRGDEVRQIAPGQTGKIAD

SEQ ID NO: 7 - protein, 30 aa, artificial sequence
  Covid-19 surface peptide position 683-712
RARSVASQSIIAYTMSLGAENSVAYSNNSI

SEQ ID NO: 8 - protein, 30 aa, artificial sequence
  Covid-19 surface peptide position 701-730
AENSVAYSNNSIAIPTNFTISVTTEILPVS

SEQ ID NO: 9 - protein, 30 aa, artificial sequence
  Covid-19 surface peptide position 893-922
ALQIPFAMQMAYRFNGIGVTQNVLYENQKL

SEQ ID NO: 10 - protein, 30 aa, artificial sequence
  Covid-19 surface peptide position 898-927
FAMQMAYRFNGIGVTQNVLYENQKLIANQF

SEQ ID NO: 11 - protein, 30 aa, artificial sequence
  Covid-19 surface peptide position 1091-1120
REGVFVSNGTHWFVTQRNFYEPQIITTDNT

SEQ ID NO: 12 - protein, 30 aa, artificial sequence
  Covid-19 nucleocapsid peptide position 36-65
RSKQRRPQGLPNNTASWFTALTQHGKEDLK

SEQ ID NO: 13 - protein, 30 aa, artificial sequence
  Covid-19 nucleocapsid peptide position 255-284
SKKPRQKRTATKAYNVTQAFGRRGPEQTQG

SEQ ID NO: 14 - protein, 30 aa, artificial sequence
  Covid-19 nucleocapsid peptide position 290-319
ELIRQGTDYKHWPQIAQFAPSASAFFGMSR

SEQ ID NO: 15 - protein, 30 aa, artificial sequence
  Covid-19 nucleocapsid peptide position 384-413
QRQKKQQTVTLLPAADLDDFSKQLQQSMSS

SEQ ID NO: 16 - protein, 30 aa, artificial sequence
  Covid-19 membrane peptide position 93-122
LSYFIASFRLFARTRSMWSFNPETNILLNV

SEQ ID NO: 17 - protein, 30 aa, artificial sequence
  Covid-19 envelope peptide position 45-74
NIVNVSLVKPSFYVYSRVKNLNSSRVPDLL

SEQ ID NO: 18 - protein, 8 aa, artificial sequence
  Coronavirus part surface 893-922 b cell epitopes
AMQMAYRF

SEQ ID NO: 19 - protein, 8 aa, artificial sequence
  Coronavirus part surface 898-927 B cell epitopes
AMQMAYRF

SEQ ID NO: 20 - protein, 15 aa, artificial sequence
  Coronavirus nucleocapsid 36-65 b cell epitopes
RRPQGLPNNTASWFT

SEQ ID NO: 21 - protein, 18 aa, artificial sequence
  Coronavirus nucleocapsid 36-65 b cell epitopes
GLPNNTASWFTALTQHGK

SEQ ID NO: 22 - protein, 15 aa, artificial sequence
  Coronavirus nucleocapsid 36-65 b cell epitopes
RRPQGLPNNTASWFT

SEQ ID NO: 23 - protein, 18 aa, artificial sequence
  Coronavirus nucleocapsid 36-65 b cell epitopes
GLPNNTASWFTALTQHGK

SEQ ID NO: 24 - protein, 17 aa, artificial sequence
  Coronavirus nucleocapsid 290-319 b cell epitopes
IRQGTDYKHWPQIAQFA

SEQ ID NO: 25 - protein, 17 aa, artificial sequence
  Coronavirus nucleocapsid 290-319 b cell epitopes
KHWPQIAQFAPSASAFF

SEQ ID NO: 26 - protein, 8 aa, artificial sequence
  Coronavirus nucleocapsid 290-319 b cell epitopes
QGTDYKHW

SEQ ID NO: 27 - protein, 6 aa, artificial sequence
  Coronavirus nucleocapsid 383-413 b cell epitopes
LLPAAD

SEQ ID NO: 28 - protein, 9 aa, artificial sequence
  Best HLAI PEPI
YTNSFTRGV

SEQ ID NO: 29 - protein, 9 aa, artificial sequence
  Best HLAI PEPI
STQDLFLPF

SEQ ID NO: 30 - protein, 9 aa, artificial sequence
  Best HLAI PEPI
RFDNPVLPF

SEQ ID NO: 31 - protein, 9 aa, artificial sequence
  Best HLAI PEPI
IVNNATNVV

SEQ ID NO: 32 - protein, 9 aa, artificial sequence
  Best HLAI PEPI
YLQPRTFLL

SEQ ID NO: 33 - protein, 9 aa, artificial sequence
  Best HLAI PEPI
NVYADSFVI

SEQ ID NO: 34 - protein, 9 aa, artificial sequence
  Best HLAI PEPI
SIIAYTMSL

SEQ ID NO: 35 - protein, 9 aa, artificial sequence
  Best HLAI PEPI
FTISVTTEI

SEQ ID NO: 36 - protein, 9 aa, artificial sequence
  Best HLAI PEPI
FAMQMAYRF

SEQ ID NO: 37 - protein, 9 aa, artificial sequence
  Best HLAI PEPI
FVSNGTHWF

SEQ ID NO: 38 - protein, 9 aa, artificial sequence
  Best HLAI PEPI
NTASWFTAL

SEQ ID NO: 39 - protein, 9 aa, artificial sequence
  Best HLAI PEPI
KAYNVTQAF

SEQ ID NO: 40 - protein, 9 aa, artificial sequence
  Best HLAI PEPI
FAPSASAFF

SEQ ID NO: 41 - protein, 9 aa, artificial sequence
  Best HLAI PEPI
FSKQLQQSM

SEQ ID NO: 42 - protein, 9 aa, artificial sequence
  Best HLAI PEPI
RLFARTRSM

SEQ ID NO: 43 - protein, 9 aa, artificial sequence
  Best HLAI PEPI
YVYSRVKNL

SEQ ID NO: 44 - protein, 15 aa, artificial sequence
  Best HLAII PEPI
YYPDKVFRSSVLHST

SEQ ID NO: 45 - protein, 15 aa, artificial sequence
  Best HLAII PEPI
STQDLFLPFFSNVTW

SEQ ID NO: 46 - protein, 15 aa, artificial sequence
  Best HLAII PEPI
DGVYFASTEKSNIIR

SEQ ID NO: 47 - protein, 15 aa, artificial sequence
  Best HLAII PEPI
KTQSLLIVNNATNVV

SEQ ID NO: 48 - protein, 15 aa, artificial sequence
  Best HLAII PEPI
AAAYYVGYLQPRTFL

SEQ ID NO: 49 - protein, 15 aa, artificial sequence
  Best HLAII PEPI
CFTNVYADSFVIRGD

SEQ ID NO: 50 - protein, 15 aa, artificial sequence
  Best HLAII PEPI
SQSIIAYTMSLGAEN

SEQ ID NO: 51 - protein, 15 aa, artificial sequence
  Best HLAII PEPI
TNFTISVTTEILPVS

SEQ ID NO: 52 - protein, 15 aa, artificial sequence
  Best HLAII PEPI
ALQIPFAMQMAYRFN

SEQ ID NO: 53 - protein, 15 aa, artificial sequence
  Best HLAII PEPI
FAMQMAYRFNGIGVT

SEQ ID NO: 54 - protein, 15 aa, artificial sequence
  Best HLAII PEPI
HWFVTQRNFYEPQII

SEQ ID NO: 55 - protein, 15 aa, artificial sequence
  Best HLAII PEPI
NNTASWFTALTQHGK

SEQ ID NO: 56 - protein, 15 aa, artificial sequence
  Best HLAII PEPI
TATKAYNVTQAFGRR

SEQ ID NO: 57 - protein, 15 aa, artificial sequence
  Best HLAII PEPI
QIAQFAPSASAFFGM

SEQ ID NO: 58 - protein, 15 aa, artificial sequence
  Best HLAII PEPI
KKQQTVTLLPAADLD

SEQ ID NO: 59 - protein, 15 aa, artificial sequence
  Best HLAII PEPI
LSYFIASFRLFARTR

SEQ ID NO: 60 - protein, 15 aa, artificial sequence
  Best HLAII PEPI
KPSFYVYSRVKNLNS

SEQ ID NO: 61 - protein, 1273 aa, artificial sequence
  SARS-CoV-2 Surface glycoprotein alias Spike
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 62 - protein, 419 aa, artificial sequence
  SARS-CoV-2 Nucleocapsid phosphoprotein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 63 - protein, 75 aa, artificial sequence
  SARS-CoV-2 Envelope protein
MYSFVSEETGTLIVNSVLLFLAFVVFLLVTLAILTALRLCAYCCNIVNVSLVKPSFYVYSRVKNLNSSRVPDLLV

SEQ ID NO: 64 - protein, 222 aa, artificial sequence
  SARS-CoV-2 Membrane glycoprotein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 65 - protein, 9 aa, artificial sequence
  HLA-A*0201 epitope
FIAGLIAIV

SEQ ID NO: 66 - protein, 10 aa, artificial sequence
  HLA-A*0201 epitope
GLIAIVMVTI

SEQ ID NO: 67 - protein, 9 aa, artificial sequence
  HLA-A*0201 epitope
IITTDNTFV

SEQ ID NO: 68 - protein, 9 aa, artificial sequence
  HLA-A*0201 epitope
ALNTLVKQL

SEQ ID NO: 69 - protein, 9 aa, artificial sequence
  HLA-A*0201 epitope
LITGRLQSL

SEQ ID NO: 70 - protein, 9 aa, artificial sequence
  HLA-A*0201 epitope
LLLQYGSFC

SEQ ID NO: 71 - protein, 8 aa, artificial sequence
  HLA-A*0201 epitope
LQYGSFCT

SEQ ID NO: 72 - protein, 9 aa, artificial sequence
  HLA-A*0201 epitope
NLNESLIDL

SEQ ID NO: 73 - protein, 9 aa, artificial sequence
  HLA-A*0201 epitope
RLDKVEAEV

SEQ ID NO: 74 - protein, 9 aa, artificial sequence
  HLA-A*0201 epitope
RLNEVAKNL

SEQ ID NO: 75 - protein, 9 aa, artificial sequence
  HLA-A*0201 epitope
RLQSLQTYV

SEQ ID NO: 76 - protein, 9 aa, artificial sequence
  HLA-A*0201 epitope
VLNDILSRL

SEQ ID NO: 77 - protein, 9 aa, artificial sequence
  HLA-A*0201 epitope
VVFLHVTYV

SEQ ID NO: 78 - protein, 8 aa, artificial sequence
  HLA-A*0201 epitope
ILLNKHID

SEQ ID NO: 79 - protein, 9 aa, artificial sequence
  HLA-A*0201 epitope
FPRGQGVPI

SEQ ID NO: 80 - protein, 9 aa, artificial sequence
  HLA-A*0201 epitope
LLLLDRLNQ

SEQ ID NO: 81 - protein, 9 aa, artificial sequence
  HLA-A*0201 epitope
GMSRIGMEV

SEQ ID NO: 82 - protein, 9 aa, artificial sequence
  HLA-A*0201 epitope
ILLNKHIDA

SEQ ID NO: 83 - protein, 9 aa, artificial sequence
  HLA-A*0201 epitope
ALNTPKDHI

SEQ ID NO: 84 - protein, 9 aa, artificial sequence
  HLA-A*0201 epitope
LALLLLDRL

SEQ ID NO: 85 - protein, 9 aa, artificial sequence
  HLA-A*0201 epitope
LLLDRLNQL

SEQ ID NO: 86 - protein, 10 aa, artificial sequence
  HLA-A*0201 epitope
LLLLDRLNQL

SEQ ID NO: 87 - protein, 9 aa, artificial sequence
  HLA-A*0201 epitope
LQLPQGTTL

SEQ ID NO: 88 - protein, 9 aa, artificial sequence
  HLA-A*0201 epitope
AQFAPSASA

SEQ ID NO: 89 - protein, 9 aa, artificial sequence
  HLA-A*0201 epitope
TTLPKGFYA

SEQ ID NO: 90 - protein, 10 aa, artificial sequence
  HLA-A*0201 epitope
VLQLPQGTTL

SEQ ID NO: 91 - protein, 10 aa, artificial sequence
  HLA-A*2402 epitope
GYQPYRVVVL

SEQ ID NO: 92 - protein, 9 aa, artificial sequence
  HLA-A*2402 epitope
PYRVVVLSF

SEQ ID NO: 93 - protein, 9 aa, artificial sequence
  HLA-A*2402 epitope
LSPRWYFYY

SEQ ID NO: 94 - protein, 10 aa, artificial sequence
  HLA-A*0101 epitope
DSFKEELDKY

SEQ ID NO: 95 - protein, 10 aa, artificial sequence
  HLA-A*0101 epitope
LIDLQELGKY

SEQ ID NO: 96 - protein, 9 aa, artificial sequence
  HLA-A*0101 epitope
PYRVVVLSF

SEQ ID NO: 97 - protein, 9 aa, artificial sequence
  HLA-A*0101 epitope
GTTLPKGFY

SEQ ID NO: 98 - protein, 10 aa, artificial sequence
  HLA-A*0101 epitope
VTPSGTWLTY

SEQ ID NO: 99 - protein, 9 aa, artificial sequence
  HLA-A*0301 epitope
GSFCTQLNR

SEQ ID NO: 100 - protein, 9 aa, artificial sequence
  HLA-A*0301 epitope
GVVFLHVTY

SEQ ID NO: 101 - protein, 9 aa, artificial sequence
  HLA-A*0301 epitope
AQALNTLVK

SEQ ID NO: 102 - protein, 9 aa, artificial sequence
  HLA-A*0301 epitope
MTSCCSCLK

SEQ ID NO: 103 - protein, 9 aa, artificial sequence
  HLA-A*0301 epitope
ASANLAATK

SEQ ID NO: 104 - protein, 10 aa, artificial sequence
  HLA-A*0301 epitope
SLIDLQELGK

SEQ ID NO: 105 - protein, 9 aa, artificial sequence
  HLA-A*0301 epitope
SVLNDILSR

SEQ ID NO: 106 - protein, 10 aa, artificial sequence
  HLA-A*0301 epitope
TQNVLYENQK

SEQ ID NO: 107 - protein, 10 aa, artificial sequence
  HLA-A*0301 epitope
CMTSCCSCLK

SEQ ID NO: 108 - protein, 10 aa, artificial sequence
  HLA-A*0301 epitope
VQIDRLITGR

SEQ ID NO: 109 - protein, 9 aa, artificial sequence
  HLA-A*0301 epitope
KTFPPTEPK

SEQ ID NO: 110 - protein, 10 aa, artificial sequence
  HLA-A*0301 epitope
KTFPPTEPKK

SEQ ID NO: 111 - protein, 9 aa, artificial sequence
  HLA-A*0301 epitope
LSPRWYFYY

SEQ ID NO: 112 - protein, 9 aa, artificial sequence
  HLA-A*0301 epitope
ASAFFGMSR

SEQ ID NO: 113 - protein, 10 aa, artificial sequence
  HLA-A*0301 epitope
ATEGALNTPK

SEQ ID NO: 114 - protein, 10 aa, artificial sequence
  HLA-A*0301 epitope
QLPQGTTLPK

SEQ ID NO: 115 - protein, 9 aa, artificial sequence
  HLA-A*0301 epitope
QQQGQTVTK

SEQ ID NO: 116 - protein, 10 aa, artificial sequence
  HLA-A*0301 epitope
QQQQGQTVTK

SEQ ID NO: 117 - protein, 10 aa, artificial sequence
  HLA-A*0301 epitope
SASAFFGMSR

SEQ ID NO: 118 - protein, 10 aa, artificial sequence
  HLA-A*0301 epitope
SQASSRSSSR

SEQ ID NO: 119 - protein, 9 aa, artificial sequence
  HLA-A*0301 epitope
TPSGTWLTY

SEQ ID NO: 120 - protein, 9 aa, artificial sequence
  HLA-A*1101 epitope
GSFCTQLNR

SEQ ID NO: 121 - protein, 9 aa, artificial sequence
  HLA-A*1101 epitope
GVVFLHVTY

SEQ ID NO: 122 - protein, 9 aa, artificial sequence
  HLA-A*1101 epitope
AQALNTLVK

SEQ ID NO: 123 - protein, 9 aa, artificial sequence
  HLA-A*1101 epitope
MTSCCSCLK

SEQ ID NO: 124 - protein, 9 aa, artificial sequence
  HLA-A*1101 epitope
ASANLAATK

SEQ ID NO: 125 - protein, 10 aa, artificial sequence
  HLA-A*1101 epitope
SLIDLQELGK

SEQ ID NO: 126 - protein, 9 aa, artificial sequence
  HLA-A*1101 epitope
SVLNDILSR

SEQ ID NO: 127 - protein, 10 aa, artificial sequence
  HLA-A*1101 epitope
TQNVLYENQK

SEQ ID NO: 128 - protein, 10 aa, artificial sequence
  HLA-A*1101 epitope
CMTSCCSCLK

SEQ ID NO: 129 - protein, 10 aa, artificial sequence
  HLA-A*1101 epitope
VQIDRLITGR

SEQ ID NO: 130 - protein, 9 aa, artificial sequence
  HLA-A*1101 epitope
KTFPPTEPK

SEQ ID NO: 131 - protein, 10 aa, artificial sequence
  HLA-A*1101 epitope
KTFPPTEPKK

SEQ ID NO: 132 - protein, 9 aa, artificial sequence
  HLA-A*1101 epitope
LSPRWYFYY

SEQ ID NO: 133 - protein, 9 aa, artificial sequence
  HLA-A*1101 epitope
ASAFFGMSR

SEQ ID NO: 134 - protein, 10 aa, artificial sequence
  HLA-A*1101 epitope
ATEGALNTPK

SEQ ID NO: 135 - protein, 10 aa, artificial sequence
  HLA-A*1101 epitope
QLPQGTTLPK

SEQ ID NO: 136 - protein, 9 aa, artificial sequence
  HLA-A*1101 epitope
QQQGQTVTK

SEQ ID NO: 137 - protein, 10 aa, artificial sequence
  HLA-A*1101 epitope
QQQQGQTVTK

SEQ ID NO: 138 - protein, 10 aa, artificial sequence
  HLA-A*1101 epitope
SASAFFGMSR

SEQ ID NO: 139 - protein, 10 aa, artificial sequence
  HLA-A*1101 epitope
SQASSRSSSR

SEQ ID NO: 140 - protein, 9 aa, artificial sequence
  HLA-A*1101 epitope
TPSGTWLTY

SEQ ID NO: 141 - protein, 9 aa, artificial sequence
  HLA-A*6801 epitope
GSFCTQLNR

SEQ ID NO: 142 - protein, 9 aa, artificial sequence
  HLA-A*6801 epitope
GVVFLHVTY

SEQ ID NO: 143 - protein, 9 aa, artificial sequence
  HLA-A*6801 epitope
AQALNTLVK

SEQ ID NO: 144 - protein, 9 aa, artificial sequence
  HLA-A*6801 epitope
MTSCCSCLK

SEQ ID NO: 145 - protein, 9 aa, artificial sequence
  HLA-A*6801 epitope
ASANLAATK

SEQ ID NO: 146 - protein, 10 aa, artificial sequence
  HLA-A*6801 epitope
SLIDLQELGK

SEQ ID NO: 147 - protein, 9 aa, artificial sequence
  HLA-A*6801 epitope
SVLNDILSR

SEQ ID NO: 148 - protein, 10 aa, artificial sequence
  HLA-A*6801 epitope
TQNVLYENQK

SEQ ID NO: 149 - protein, 10 aa, artificial sequence
  HLA-A*6801 epitope
CMTSCCSCLK

SEQ ID NO: 150 - protein, 10 aa, artificial sequence
  HLA-A*6801 epitope
VQIDRLITGR

SEQ ID NO: 151 - protein, 9 aa, artificial sequence
  HLA-A*6801 epitope
KTFPPTEPK

SEQ ID NO: 152 - protein, 10 aa, artificial sequence
  HLA-A*6801 epitope
KTFPPTEPKK

SEQ ID NO: 153 - protein, 9 aa, artificial sequence
  HLA-A*6801 epitope
LSPRWYFYY

SEQ ID NO: 154 - protein, 9 aa, artificial sequence
  HLA-A*6801 epitope
ASAFFGMSR

SEQ ID NO: 155 - protein, 10 aa, artificial sequence
  HLA-A*6801 epitope
ATEGALNTPK

SEQ ID NO: 156 - protein, 10 aa, artificial sequence
  HLA-A*6801 epitope
QLPQGTTLPK

SEQ ID NO: 157 - protein, 9 aa, artificial sequence
  HLA-A*6801 epitope
QQQGQTVTK

SEQ ID NO: 158 - protein, 10 aa, artificial sequence
  HLA-A*6801 epitope
QQQQGQTVTK

SEQ ID NO: 159 - protein, 10 aa, artificial sequence
  HLA-A*6801 epitope
SASAFFGMSR

SEQ ID NO: 160 - protein, 10 aa, artificial sequence
  HLA-A*6801 epitope
SQASSRSSSR

SEQ ID NO: 161 - protein, 9 aa, artificial sequence
  HLA-A*6801 epitope
TPSGTWLTY

SEQ ID NO: 162 - protein, 10 aa, artificial sequence
  HLA-A*2301 epitope
GYQPYRVVVL

SEQ ID NO: 163 - protein, 9 aa, artificial sequence
  HLA-A*2301 epitope
PYRVVVLSF

SEQ ID NO: 164 - protein, 9 aa, artificial sequence
  HLA-A*2301 epitope
LSPRWYFYY

SEQ ID NO: 165 - protein, 9 aa, artificial sequence
  HLA-A*3101 epitope
GSFCTQLNR

SEQ ID NO: 166 - protein, 9 aa, artificial sequence
  HLA-A*3101 epitope
GVVFLHVTY

SEQ ID NO: 167 - protein, 9 aa, artificial sequence
  HLA-A*3101 epitope
AQALNTLVK

SEQ ID NO: 168 - protein, 9 aa, artificial sequence
  HLA-A*3101 epitope
MTSCCSCLK

SEQ ID NO: 169 - protein, 9 aa, artificial sequence
  HLA-A*3101 epitope
ASANLAATK

SEQ ID NO: 170 - protein, 10 aa, artificial sequence
  HLA-A*3101 epitope
SLIDLQELGK

SEQ ID NO: 171 - protein, 9 aa, artificial sequence
  HLA-A*3101 epitope
SVLNDILSR

SEQ ID NO: 172 - protein, 10 aa, artificial sequence
  HLA-A*3101 epitope
TQNVLYENQK

SEQ ID NO: 173 - protein, 10 aa, artificial sequence
  HLA-A*3101 epitope
CMTSCCSCLK

SEQ ID NO: 174 - protein, 10 aa, artificial sequence
  HLA-A*3101 epitope
VQIDRLITGR

SEQ ID NO: 175 - protein, 9 aa, artificial sequence
  HLA-A*3101 epitope
KTFPPTEPK

SEQ ID NO: 176 - protein, 10 aa, artificial sequence
  HLA-A*3101 epitope
KTFPPTEPKK

SEQ ID NO: 177 - protein, 9 aa, artificial sequence
  HLA-A*3101 epitope
LSPRWYFYY

SEQ ID NO: 178 - protein, 9 aa, artificial sequence
  HLA-A*3101 epitope
ASAFFGMSR

SEQ ID NO: 179 - protein, 10 aa, artificial sequence
  HLA-A*3101 epitope
ATEGALNTPK

SEQ ID NO: 180 - protein, 10 aa, artificial sequence
  HLA-A*3101 epitope
QLPQGTTLPK

SEQ ID NO: 181 - protein, 9 aa, artificial sequence
  HLA-A*3101 epitope
QQQGQTVTK

SEQ ID NO: 182 - protein, 10 aa, artificial sequence
  HLA-A*3101 epitope
QQQQGQTVTK

SEQ ID NO: 183 - protein, 10 aa, artificial sequence
  HLA-A*3101 epitope
SASAFFGMSR

SEQ ID NO: 184 - protein, 10 aa, artificial sequence
  HLA-A*3101 epitope
SQASSRSSSR

SEQ ID NO: 185 - protein, 9 aa, artificial sequence
  HLA-A*3101 epitope
TPSGTWLTY

SEQ ID NO: 186 - protein, 10 aa, artificial sequence
  HLA-B*0702 epitope
FPNITNLCPF

SEQ ID NO: 187 - protein, 10 aa, artificial sequence
  HLA-B*0702 epitope
APHGVVFLHV

SEQ ID NO: 188 - protein, 9 aa, artificial sequence
  HLA-B*0702 epitope
FPRGQGVPI

SEQ ID NO: 189 - protein, 10 aa, artificial sequence
  HLA-B*0702 epitope
APSASAFFGM

SEQ ID NO: 190 - protein, 9 aa, artificial sequence
  HLA-B*0801 epitope
FPRGQGVPI

SEQ ID NO: 191 - protein, 10 aa, artificial sequence
  HLA-B*3501 epitope
FPNITNLCPF

SEQ ID NO: 192 - protein, 10 aa, artificial sequence
  HLA-B*3501 epitope
APHGVVFLHV

SEQ ID NO: 193 - protein, 9 aa, artificial sequence
  HLA-B*3501 epitope
FPRGQGVPI

SEQ ID NO: 194 - protein, 10 aa, artificial sequence
  HLA-B*3501 epitope
APSASAFFGM

SEQ ID NO: 195 - protein, 9 aa, artificial sequence
  HLA-B*1501 epitope
LQIPFAMQM

SEQ ID NO: 196 - protein, 9 aa, artificial sequence
  HLA-B*1501 epitope
RVDFCGKGY

SEQ ID NO: 197 - protein, 10 aa, artificial sequence
  HLA-B*5101 epitope
FPNITNLCPF

SEQ ID NO: 198 - protein, 10 aa, artificial sequence
  HLA-B*5101 epitope
APHGVVFLHV

SEQ ID NO: 199 - protein, 9 aa, artificial sequence
  HLA-B*5101 epitope
FPRGQGVPI

SEQ ID NO: 200 - protein, 10 aa, artificial sequence
  HLA-B*5101 epitope
APSASAFFGM

SEQ ID NO: 201 - protein, 10 aa, artificial sequence
  HLA-B*1801 epitope
YEQYIKWPWY

SEQ ID NO: 202 - protein, 9 aa, artificial sequence
  HLA-B*2705 epitope
GRLQSLQTY

SEQ ID NO: 203 - protein, 9 aa, artificial sequence
  HLA-B*2705 epitope
RVDFCGKGY

SEQ ID NO: 204 - protein, 9 aa, artificial sequence
  HLA-B*2705 epitope
VRFPNITNL

SEQ ID NO: 205 - protein, 9 aa, artificial sequence
  HLA-A*3301 epitope
MTSCCSCLK

SEQ ID NO: 206 - protein, 10 aa, artificial sequence
  HLA-A*3301 epitope
SLIDLQELGK

SEQ ID NO: 207 - protein, 10 aa, artificial sequence
  HLA-A*3301 epitope
CMTSCCSCLK

SEQ ID NO: 208 - protein, 10 aa, artificial sequence
  HLA-A*3301 epitope
VQIDRLITGR

SEQ ID NO: 209 - protein, 10 aa, artificial sequence
  HLA-A*3301 epitope
SASAFFGMSR

SEQ ID NO: 210 - protein, 10 aa, artificial sequence
  HLA-A*3301 epitope
SQASSRSSSR

SEQ ID NO: 211 - protein, 9 aa, artificial sequence
  HLA-B*5801 epitope
LQIPFAMQM

SEQ ID NO: 212 - protein, 9 aa, artificial sequence
  HLA-B*5801 epitope
RVDFCGKGY

SEQ ID NO: 213 - protein, 9 aa, artificial sequence
  HLA-C*1502 epitope
LQIPFAMQM

SEQ ID NO: 214 - protein, 9 aa, artificial sequence
  HLA-C*1502 epitope
RVDFCGKGY

SEQ ID NO: 215 - protein, 9 aa, artificial sequence
  HLA-C*1402 epitope
VRFPNITNL

SEQ ID NO: 216 - protein, 30 aa, artificial sequence; identical to SEQ ID NO: 2
  SARS-CoV-2 fragment S 35-64

SEQ ID NO: 217 - protein, 30 aa, artificial sequence; identical to SEQ ID NO: 5
  SARS-CoV-2 fragment S 253-282

SEQ ID NO: 218 - protein, 30 aa, artificial sequence; identical to SEQ ID NO: 9
  SARS-CoV-2 fragment S 893-922

SEQ ID NO: 219 - protein, 30 aa, artificial sequence; identical to SEQ ID NO: 12
  SARS-CoV-2 fragment N 36-65

SEQ ID NO: 220 - protein, 30 aa, artificial sequence; identical to SEQ ID NO: 13
  SARS-CoV-2 fragment N 255-284

SEQ ID NO: 221 - protein, 30 aa, artificial sequence; identical to SEQ ID NO: 14
  SARS-CoV-2 fragment N 290-319

SEQ ID NO: 222 - protein, 30 aa, artificial sequence; identical to SEQ ID NO: 15
  SARS-CoV-2 fragment N 384-413

SEQ ID NO: 223 - protein, 30 aa, artificial sequence; identical to SEQ ID NO: 16
  SARS-CoV-2 fragment M 93-122

SEQ ID NO: 224 - protein, 30 aa, artificial sequence; identical to SEQ ID NO: 17
  SARS-CoV-2 fragment E 45-74

SEQ ID NO: 225 - protein, 30 aa, artificial sequence; identical to SEQ ID NO: 2
  PolyPEPI-SCoV-2 vaccine peptide sequences S2

SEQ ID NO: 226 - protein, 30 aa, artificial sequence; identical to SEQ ID NO: 5
  PolyPEPI-SCoV-2 vaccine peptide sequences S5

SEQ ID NO: 227 - protein, 30 aa, artificial sequence; identical to SEQ ID NO: 9
  PolyPEPI-SCoV-2 vaccine peptide sequences S9

SEQ ID NO: 228 - protein, 30 aa, artificial sequence; identical to SEQ ID NO: 12
  PolyPEPI-SCoV-2 vaccine peptide sequences N1

SEQ ID NO: 229 - protein, 30 aa, artificial sequence; identical to SEQ ID NO: 13
  PolyPEPI-SCoV-2 vaccine peptide sequences N2

SEQ ID NO: 230 - protein, 30 aa, artificial sequence; identical to SEQ ID NO: 14
  PolyPEPI-SCoV-2 vaccine peptide sequences N3

SEQ ID NO: 231 - protein, 30 aa, artificial sequence; identical to SEQ ID NO: 15
  PolyPEPI-SCoV-2 vaccine peptide sequences N4

SEQ ID NO: 232 - protein, 30 aa, artificial sequence; identical to SEQ ID NO: 16
  PolyPEPI-SCoV-2 vaccine peptide sequences M1

SEQ ID NO: 233 - protein, 30 aa, artificial sequence; identical to SEQ ID NO: 17
  PolyPEPI-SCoV-2 vaccine peptide sequences E1

SEQ ID NO: 234 - RNA, 90 nt, artificial sequence
  RNA encoding Covid-19 surface peptide position 22-51
  misc_feature (3)..(3): n is a, c, g, or u
  misc_feature (6)..(6): R is A or G
  misc_feature (9)..(9): n is a, c, g, or u
  misc_feature (12)..(12): n is a, c, g, or u
  misc_feature (15)..(15): n is a, c, g, or u
  misc_feature (18)..(18): n is a, c, g, or u
  misc_feature (21)..(21): Y is C or T/U
  misc_feature (24)..(24): n is a, c, g, or u
  misc_feature (27)..(27): Y is C or T/U
  misc_feature (28)..(28): W is A or T/U,
  misc_feature (29)..(29): S is G or C
  misc_feature (30)..(30): n is a, c, g, or u
  misc_feature (33)..(33): Y is C or T/U
  misc_feature (36)..(36): n is a, c, g, or u
  misc_feature (37)..(37): M is A or C
  misc_feature (39)..(39): n is a, c, g, or u
  misc_feature (42)..(42): n is a, c, g, or u
  misc_feature (45)..(45): n is a, c, g, or u
  misc_feature (48)..(48): Y is C or T/U
  misc_feature (51)..(51): Y is C or T/U
  misc_feature (54)..(54): n is a, c, g, or u
  misc_feature (57)..(57): Y is C or T/U
  misc_feature (60)..(60): Y is C or T/U
  misc_feature (63)..(63): n is a, c, g, or u
  misc_feature (66)..(66): Y is C or T/U
  misc_feature (67)..(67): M is A or C
  misc_feature (69)..(69): n is a, c, g, or u
  misc_feature (70)..(70): W is A or T/U,
  misc_feature (71)..(71): S is G or C
  misc_feature (72)..(72): n is a, c, g, or u
  misc_feature (73)..(73): W is A or T/U,
  misc_feature (74)..(74): S is G or C
  misc_feature (75)..(75): n is a, c, g, or u
  misc_feature (78)..(78): n is a, c, g, or u
  misc_feature (79)..(79): Y is C or T/U
  misc_feature (81)..(81): n is a, c, g, or u
  misc_feature (84)..(84): Y is C or T/U
  misc_feature (85)..(85): W is A or T/U,
  misc_feature (86)..(86): S is G or C
  misc_feature (87)..(87): n is a, c, g, or u
  misc_feature (90)..(90): n is a, c, g, or u
acncaryunccnccngcnuayacnaaywsnuuyacnmgnggngunuayuayccngayaargunuuymgnwsnwsngunyuncaywsnacn

SEQ ID NO: 235 - RNA, 90 nt, artificial sequence
  RNA encoding Covid-19 surface peptide position 35-64
  misc_feature (3)..(3): n is a, c, g, or u
  misc_feature (6)..(6): n is a, c, g, or u
  misc_feature (9)..(9): Y is C or T/U
  misc_feature (12)..(12): Y is C or T/U
  misc_feature (15)..(15): n is a, c, g, or u
  misc_feature (18)..(18): Y is C or T/U
  misc_feature (21)..(21): R is A or G
  misc_feature (24)..(24): n is a, c, g, or u
  misc_feature (27)..(27): Y is C or T/U
  misc_feature (28)..(28): M is A or C
  misc_feature (30)..(30): n is a, c, g, or u
  misc_feature (31)..(31): W is A or T/U
  misc_feature (32)..(32): S is G or C
  misc_feature (33)..(33): n is a, c, g, or u
  misc_feature (34)..(34): W is A or T/U
  misc_feature (35)..(35): S is G or C
  misc_feature (36)..(36): n is a, c, g, or u
  misc_feature (39)..(39): n is a, c, g, or u
  misc_feature (40)..(40): Y is C or T/U
  misc_feature (42)..(42): n is a, c, g, or u
  misc_feature (45)..(45): Y is C or T/U
  misc_feature (46)..(46): W is A or T/U
  misc_feature (47)..(47): S is G or C
  misc_feature (48)..(48): n is a, c, g, or u
  misc_feature (51)..(51): n is a, c, g, or u
  misc_feature (54)..(54): R is A or G
  misc_feature (57)..(58): Y is C or T/U
  misc_feature (60)..(60): n is a, c, g, or u
  misc_feature (63)..(64): Y is C or T/U
  misc_feature (66)..(66): n is a, c, g, or u
  misc_feature (69)..(69): n is a, c, g, or u
  misc_feature (72)..(72): Y is C or T/U
  misc_feature (75)..(75): Y is C or T/U
  misc_feature (76)..(76): W is A or T/U
  misc_feature (77)..(77): S is G or C
  misc_feature (78)..(78): n is a, c, g, or u
  misc_feature (81)..(81): Y is C or T/U
  misc_feature (84)..(84): n is a, c, g, or u
  misc_feature (87)..(87): n is a, c, g, or u
ggngunuayuayccngayaargunuuymgnwsnwsngunyuncaywsnacncargayyunuuyyunccnuuyuuywsnaaygunacnugg

SEQ ID NO: 236 - RNA, 90 nt, artificial sequence
  RNA encoding Covid-19 surface peptide position 76-105
  misc_feature (3)..(3): n is a, c, g, or u
  misc_feature (6)..(6): R is A or G
  misc_feature (7)..(7): M is A or C
  misc_feature (9)..(9): n is a, c, g, or u
  misc_feature (12)..(12): Y is C or T/U
  misc_feature (15)..(15): Y is C or T/U
  misc_feature (18)..(18): Y is C or T/U
  misc_feature (21)..(21): n is a, c, g, or u
  misc_feature (24)..(24): n is a, c, g, or u
  misc_feature (25)..(25): Y is C or T/U
  misc_feature (27)..(27): n is a, c, g, or u
  misc_feature (30)..(30): n is a, c, g, or u
  misc_feature (33)..(33): Y is C or T/U
  misc_feature (36)..(36): Y is C or T/U
  misc_feature (39)..(39): Y is C or T/U
  misc_feature (42)..(42): n is a, c, g, or u
  misc_feature (45)..(45): n is a, c, g, or u
  misc_feature (48)..(48): Y is C or T/U
  misc_feature (51)..(51): Y is C or T/U
  misc_feature (54)..(54): n is a, c, g, or u
  misc_feature (55)..(55): W is A or T/U
  misc_feature (56)..(56): S is G or C
  misc_feature (57)..(57): n is a, c, g, or u
  misc_feature (60)..(60): n is a, c, g, or u
  misc_feature (63)..(63): R is A or G
  misc_feature (66)..(66): R is A or G
  misc_feature (67)..(67): W is A or T/U
  misc_feature (68)..(68): S is G or C
  misc_feature (69)..(69): n is a, c, g, or u
  misc_feature (72)..(72): Y is C or T/U
  misc_feature (75)..(75): H is A or C or T/U
  misc_feature (78)..(78): H is A or C or T/U
  misc_feature (79)..(79): M is A or C
  misc_feature (81)..(81): n is a, c, g, or u
  misc_feature (84)..(84): n is a, c, g, or u
  misc_feature (90)..(90): H is A or C or T/U
acnaarmgnuuygayaayccngunyunccnuuyaaygayggngunuayuuygcnwsnacngaraarwsnaayauhauhmgnggnuggauh

SEQ ID NO: 237 - RNA, 90 nt, artificial sequence
  RNA encoding Covid-19 surface peptide position 98-127
  misc_feature (1)..(1): W is A or T/U
  misc_feature (2)..(2): S is G or C
  misc_feature (3)..(3): n is a, c, g, or u
  misc_feature (6)..(6): Y is C or T/U
  misc_feature (9)..(9): H is A or C or T/U
  misc_feature (12)..(12): H is A or C or T/U
  misc_feature (13)..(13): M is A or C
  misc_feature (15)..(15): n is a, c, g, or u
  misc_feature (18)..(18): n is a, c, g, or u
  misc_feature (24)..(24): H is A or C or T/U
  misc_feature (27)..(27): Y is C or T/U
  misc_feature (30)..(30): n is a, c, g, or u
  misc_feature (33)..(33): n is a, c, g, or u
  misc_feature (36)..(36): n is a, c, g, or u
  misc_feature (37)..(37): Y is C or T/U
  misc_feature (39)..(39): n is a, c, g, or u
  misc_feature (42)..(42): Y is C or T/U
  misc_feature (43)..(43): W is A or T/U
  misc_feature (44)..(44): S is G or C
  misc_feature (45)..(45): n is a, c, g, or u
  misc_feature (48)..(48): R is A or G
  misc_feature (51)..(51): n is a, c, g, or u
  misc_feature (54)..(54): R is A or G
  misc_feature (55)..(55): W is A or T/U
  misc_feature (56)..(56): S is G or C
  misc_feature (57)..(57): n is a, c, g, or u
  misc_feature (58)..(58): Y is C or T/U
  misc_feature (60)..(60): n is a, c, g, or u
  misc_feature (61)..(61): Y is C or T/U
  misc_feature (63)..(63): n is a, c, g, or u
  misc_feature (65)..(65): H is A or C or T/U
  misc_feature (69)..(69): n is a, c, g, or u
  misc_feature (72)..(72): Y is C or T/U
  misc_feature (75)..(75): Y is C or T/U
  misc_feature (78)..(78): n is a, c, g, or u
  misc_feature (81)..(81): Y is C or T/U
  misc_feature (87)..(87): n is a, c, g, or u
  misc_feature (90)..(90): n is a, c, g, or u
wsnaayauhauhmgnggnuggauhuuyggnacnacnyungaywsnaaracncarwsnyunyunauhgunaayaaygcnacnaaygungun

SEQ ID NO: 238 - RNA, 90 nt, artificial sequence
  RNA encoding Covid-19 surface peptide position 253-282
  misc_feature (3)..(3): Y is C or T/U
  misc_feature (4)..(4): W is A or T/U
  misc_feature (5)..(5): S is G or C
  misc_feature (6)..(6): n is a, c, g, or u
  misc_feature (7)..(7): W is A or T/U
  misc_feature (8)..(8): S is G or C
  misc_feature (9)..(9): n is a, c, g, or u
  misc_feature (10)..(10): W is A or T/U
  misc_feature (11)..(11): S is G or C
  misc_feature (12)..(12): n is a, c, g, or u
  misc_feature (15)..(15): n is a, c, g, or u
  misc_feature (21)..(21): n is a, c, g, or u
  misc_feature (24)..(24): n is a, c, g, or u
  misc_feature (27)..(27): n is a, c, g, or u
  misc_feature (30)..(30): n is a, c, g, or u
  misc_feature (33)..(33): n is a, c, g, or u
  misc_feature (36)..(36): n is a, c, g, or u
  misc_feature (39)..(39): Y is C or T/U
  misc_feature (42)..(42): Y is C or T/U
  misc_feature (45)..(45): n is a, c, g, or u
  misc_feature (48)..(48): n is a, c, g, or u
  misc_feature (51)..(52): Y is C or T/U
  misc_feature (54)..(54): n is a, c, g, or u
  misc_feature (57)..(57): R is A or G
  misc_feature (60)..(60): n is a, c, g, or u
  misc_feature (61)..(61): M is A or C
  misc_feature (63)..(63): n is a, c, g, or u
  misc_feature (66)..(66): n is a, c, g, or u
  misc_feature (69)..(69): Y is C or T/U
  misc_feature (70)..(70): Y is C or T/U
  misc_feature (72)..(72): n is a, c, g, or u
  misc_feature (73)..(73): Y is C or T/U
  misc_feature (75)..(75): n is a, c, g, or u
  misc_feature (78)..(78): R is A or G
  misc_feature (81)..(81): Y is C or T/U
  misc_feature (84)..(84): Y is C or T/U
  misc_feature (87)..(87): R is A or G
  misc_feature (90)..(90): Y is C or T/U
gaywsnwsnwsnggnuggacngcnggngcngcngcnuayuaygunggnuayyuncarccnmgnacnuuyyunyunaaruayaaygaraay

SEQ ID NO: 239 - RNA, 90 nt, artificial sequence
  RNA encoding Covid-19 surface peptide position 391-420
  misc_feature (3)..(3): Y is C or T/U
  misc_feature (6)..(6): Y is C or T/U
  misc_feature (9)..(9): n is a, c, g, or u
  misc_feature (12)..(12): Y is C or T/U
  misc_feature (15)..(15): n is a, c, g, or u
  misc_feature (18)..(18): Y is C or T/U
  misc_feature (21)..(21): n is a, c, g, or u
  misc_feature (24)..(24): Y is C or T/U
  misc_feature (25)..(25): W is A or T/U
  misc_feature (26)..(26): S is G or C
  misc_feature (27)..(27): n is a, c, g, or u
  misc_feature (30)..(30): Y is C or T/U
  misc_feature (33)..(33): n is a, c, g, or u
  misc_feature (36)..(36): H is A or C or T/U
  misc_feature (37)..(37): M is A or C
  misc_feature (39)..(39): n is a, c, g, or u
  misc_feature (42)..(42): n is a, c, g, or u
  misc_feature (45)..(45): Y is C or T/U
  misc_feature (48)..(48): R is A or G
  misc_feature (51)..(51): M is A or C
  misc_feature (54)..(54): n is a, c, g, or u
  misc_feature (57)..(57): R is A or G
  misc_feature (60)..(60): H is A or C or T/U
  misc_feature (63)..(63): n is a, c, g, or u
  misc_feature (66)..(66): n is a, c, g, or u
  misc_feature (69)..(69): n is a, c, g, or u
  misc_feature (72)..(72): R is A or G
  misc_feature (75)..(75): n is a, c, g, or u
  misc_feature (78)..(78): n is a, c, g, or u
  misc_feature (81)..(81): R is A or G
  misc_feature (84)..(84): H is A or C or T/U
  misc_feature (87)..(87): n is a, c, g, or u
  misc_feature (90)..(90): Y is C or T/U
ugyuuyacnaaygunuaygcngaywsnuuygunauhmgnggngaygargunmgncarauhgcnccnggncaracnggnaarauhgcngay

SEQ ID NO: 240 - RNA, 90 nt, artificial sequence
  RNA encoding Covid-19 surface peptide position 683-712
  misc_feature (1)..(1): M is A or C
  misc_feature (3)..(3): n is a, c, g, or u
  misc_feature (6)..(6): n is a, c, g, or u
  misc_feature (7)..(7): M is A or C
  misc_feature (9)..(9): n is a, c, g, or u
  misc_feature (10)..(10): W is A or T/U
  misc_feature (11)..(11): S is G or C
  misc_feature (12)..(12): n is a, c, g, or u
  misc_feature (15)..(15): n is a, c, g, or u
  misc_feature (18)..(18): n is a, c, g, or u
  misc_feature (19)..(19): W is A or T/U
  misc_feature (20)..(20): S is G or C
  misc_feature (21)..(21): n is a, c, g, or u
  misc_feature (24)..(60): R is A or G
  misc_feature (25)..(25): S is G or C
  misc_feature (30)..(30): H is A or C or T/U
  misc_feature (33)..(33): H is A or C or T/U
  misc_feature (39)..(39): Y is C or T/U
  misc_feature (46)..(46): W is A or T/U
  misc_feature (47)..(47): S is G or C
  misc_feature (49)..(49): Y is C or T/U
  misc_feature (63)..(63): Y is C or T/U
  misc_feature (64)..(64): W is A or T/U
  misc_feature (65)..(65): S is G or C
  misc_feature (66)..(66): n is a, c, g, or u
  misc_feature (69)..(69): n is a, c, g, or u
  misc_feature (72)..(72): n is a, c, g, or u
  misc_feature (75)..(75): Y is C or T/U
  misc_feature (76)..(76): W is A or T/U
  misc_feature (78)..(78): S is G or C
  misc_feature (81)..(81): Y is C or T/U
  misc_feature (84)..(84): Y is C or T/U
  misc_feature (85)..(85): W is A or T/U
  misc_feature (86)..(86): S is G or C
  misc_feature (87)..(87): n is a, c, g, or u
  misc_feature (90)..(90): H is A or C or T/U
mgngcnmgnwsngungcnwsncarwsnauhauhgcnuayacnaugwsnyunggngcngaraaywsngungcnuaywsnaayaaywsnauh

SEQ ID NO: 241 - RNA, 90 nt, artificial sequence
  RNA encoding Covid-19 surface peptide position 701-730
  misc_feature (3)..(3): n is a, c, g, or u
  misc_feature (6)..(6): R is A or G
  misc_feature (9)..(9): Y is C or T/U
  misc_feature (10)..(10): W is A or T/U
  misc_feature (11)..(11): S is G or C
  misc_feature (12)..(12): n is a, c, g, or u
  misc_feature (15)..(15): n is a, c, g, or u
  misc_feature (18)..(18): n is a, c, g, or u
  misc_feature (21)..(21): Y is C or T/U
  misc_feature (22)..(22): W is A or T/U
  misc_feature (23)..(23): S is G or C
  misc_feature (24)..(24): n is a, c, g, or u
  misc_feature (27)..(27): Y is C or T/U
  misc_feature (30)..(30): Y is C or T/U
  misc_feature (31)..(31): W is A or T/U
  misc_feature (32)..(32): S is G or C
  misc_feature (33)..(33): n is a, c, g, or u
  misc_feature (36)..(36): H is A or C or T/U
  misc_feature (39)..(39): n is a, c, g, or u
  misc_feature (42)..(42): H is A or C or T/U
  misc_feature (45)..(45): n is a, c, g, or u
  misc_feature (48)..(48): n is a, c, g, or u
  misc_feature (51)..(51): Y is C or T/U
  misc_feature (54)..(54): Y is C or T/U
  misc_feature (57)..(57): n is a, c, g, or u
  misc_feature (60)..(60): H is A or C or T/U
  misc_feature (61)..(61): W is A or T/U
  misc_feature (62)..(62): S is G or C
  misc_feature (63)..(63): n is a, c, g, or u
  misc_feature (66)..(66): n is a, c, g, or u
  misc_feature (69)..(69): n is a, c, g, or u
  misc_feature (72)..(72): n is a, c, g, or u
  misc_feature (75)..(75): R is A or G
  misc_feature (78)..(78): H is A or C or T/U
  misc_feature (79)..(79): Y is C or T/U
  misc_feature (81)..(81): n is a, c, g, or u
  misc_feature (84)..(84): n is a, c, g, or u
  misc_feature (87)..(87): W is A or T/U
  misc_feature (89)..(89): S is G or C
  misc_feature (90)..(90): n is a, c, g, or u
gcngaraaywsngungcnuaywsnaayaaywsnauhgcnauhccnacnaayuuyacnauhwsngunacnacngarauhyunccngunwsn

SEQ ID NO: 242 - RNA, 90 nt, artificial sequence
  RNA encoding Covid-19 surface peptide position 893-922
  misc_feature (3)..(3): n is a, c, g, or u
  misc_feature (4)..(4): Y is C or T/U
  misc_feature (6)..(6): n is a, c, g, or u
  misc_feature (15)..(15): n is a, c, g, or u
  misc_feature (18)..(18): Y is C or T/U
  misc_feature (21)..(21): n is a, c, g, or u
  misc_feature (27)..(27): R is A or G
  misc_feature (33)..(33): n is a, c, g, or u
  misc_feature (36)..(36): Y is C or T/U
  misc_feature (37)..(37): M is A or C
  misc_feature (39)..(39): n is a, c, g, or u
  misc_feature (42)..(42): Y is C or T/U
  misc_feature (45)..(45): Y is C or T/U
  misc_feature (48)..(48): n is a, c, g, or u
  misc_feature (51)..(51): H is A or C or T/U
  misc_feature (54)..(54): n is a, c, g, or u
  misc_feature (57)..(57): n is a, c, g, or u
  misc_feature (60)..(60): n is a, c, g, or u
  misc_feature (63)..(63): R is A or G
  misc_feature (66)..(66): Y is C or T/U
  misc_feature (69)..(69): n is a, c, g, or u
  misc_feature (70)..(70): Y is C or T/U
  misc_feature (72)..(72): n is a, c, g, or u
  misc_feature (75)..(75): Y is C or T/U
  misc_feature (78)..(78): R is A or G
  misc_feature (81)..(81): Y is C or T/U
  misc_feature (84)..(84): R is A or G
  misc_feature (87)..(87): R is A or G
  misc_feature (88)..(88): Y is C or T/U
  misc_feature (90)..(90): n is a, c, g, or u
gcnyuncarauhccnuuygcnaugcarauggcnuaymgnuuyaayggnauhggngunacncaraaygunyunuaygaraaycaraaryun

SEQ ID NO: 243 - RNA, 90 nt, artificial sequence
  RNA encoding Covid-19 surface peptide position 898-927
  misc_feature (3)..(3): Y is C or T/U
  misc_feature (6)..(6): n is a, c, g, or u
  misc_feature (12)..(12): R is A or G
  misc_feature (18)..(18): n is a, c, g, or u
  misc_feature (21)..(21): Y is C or T/U
  misc_feature (22)..(22): M is A or C
  misc_feature (24)..(24): n is a, c, g, or u
  misc_feature (27)..(27): Y is C or T/U
  misc_feature (30)..(30): Y is C or T/U
  misc_feature (33)..(33): n is a, c, g, or u
  misc_feature (36)..(36): H is A or C or T/U
  misc_feature (39)..(39): n is a, c, g, or u
  misc_feature (42)..(42): n is a, c, g, or u
  misc_feature (45)..(45): n is a, c, g, or u
  misc_feature (48)..(48): R is A or G
  misc_feature (51)..(51): Y is C or T/U
  misc_feature (54)..(54): n is a, c, g, or u
  misc_feature (55)..(55): Y is C or T/U
  misc_feature (57)..(57): n is a, c, g, or u
  misc_feature (60)..(60): Y is C or T/U
  misc_feature (63)..(63): R is A or G
  misc_feature (66)..(66): Y is C or T/U
  misc_feature (69)..(69): R is A or G
  misc_feature (72)..(72): R is A or G
  misc_feature (73)..(73): Y is C or T/U
  misc_feature (75)..(75): n is a, c, g, or u
  misc_feature (78)..(78): H is A or C or T/U
  misc_feature (81)..(81): n is a, c, g, or u
  misc_feature (84)..(84): Y is C or T/U
  misc_feature (86)..(86): R is A or G
  misc_feature (90)..(90): Y is C or T/U
uuygcnaugcarauggcnuaymgnuuyaayggnauhggngunacncaraaygunyunuaygaraaycaraaryunauhgcnaaycaruuy

SEQ ID NO: 244 - RNA, 90 nt, artificial sequence
  RNA encoding Covid-19 surface peptide position 1091-1120
  misc_feature (1)..(1): M is A or C
  misc_feature (3)..(3): n is a, c, g, or u
  misc_feature (6)..(6): R is A or G
  misc_feature (9)..(9): n is a, c, g, or u
  misc_feature (12)..(12): n is a, c, g, or u
  misc_feature (15)..(15): Y is C or T/U
  misc_feature (18)..(18): n is a, c, g, or u
  misc_feature (21)..(21): n is a, c, g, or u
  misc_feature (24)..(24): Y is C or T/U
  misc_feature (27)..(27): n is a, c, g, or u
  misc_feature (30)..(30): n is a, c, g, or u
  misc_feature (33)..(33): Y is C or T/U
  misc_feature (39)..(39): Y is C or T/U
  misc_feature (42)..(42): n is a, c, g, or u
  misc_feature (45)..(45): n is a, c, g, or u
  misc_feature (48)..(48): R is A or G
  misc_feature (49)..(49): M is A or C
  misc_feature (51)..(51): n is a, c, g, or u
  misc_feature (54)..(54): Y is C or T/U
  misc_feature (57)..(57): Y is C or T/U
  misc_feature (60)..(60): Y is C or T/U
  misc_feature (63)..(63): R is A or G
  misc_feature (66)..(66): n is a, c, g, or u
  misc_feature (69)..(69): R is A or G
  misc_feature (72)..(72): H is A or C or T/U
  misc_feature (75)..(75): H is A or C or T/U
  misc_feature (78)..(78): n is a, c, g, or u
  misc_feature (81)..(81): n is a, c, g, or u
  misc_feature (84)..(84): Y is C or T/U
  misc_feature (87)..(87): Y is C or T/U
  misc_feature (90)..(90): n is a, c, g, or u
mgngarggngunuuygunwsnaayggnacncayugguuygunacncarmgnaayuuyuaygarccncarauhauhacnacngayaayacn

SEQ ID NO: 245 - RNA, 90 nt, artificial sequence
  RNA encoding Covid-19 nucleocapsid peptide position 36-65
  misc_feature (1)..(1): M is A or C
  misc_feature (3)..(3): n is a, c, g, or u
  misc_feature (4)..(4): W is A or T/U
  misc_feature (5)..(5): S is G or C
  misc_feature (6)..(6): n is a, c, g, or u
  misc_feature (9)..(9): R is A or G
  misc_feature (12)..(12): R is A or G
  misc_feature (13)..(13): M is A or C
  misc_feature (15)..(15): n is a, c, g, or u
  misc_feature (16)..(16): M is A or C
  misc_feature (18)..(18): n is a, c, g, or u
  misc_feature (21)..(21): n is a, c, g, or u
  misc_feature (24)..(24): R is A or G
  misc_feature (27)..(27): n is a, c, g, or u
  misc_feature (28)..(28): Y is C or T/U
  misc_feature (30)..(30): n is a, c, g, or u
  misc_feature (33)..(33): n is a, c, g, or u
  misc_feature (36)..(36): Y is C or T/U
  misc_feature (39)..(39): Y is C or T/U
  misc_feature (42)..(42): n is a, c, g, or u
  misc_feature (45)..(45): n is a, c, g, or u
  misc_feature (46)..(46): W is A or T/U
  misc_feature (47)..(47): S is G or C
  misc_feature (48)..(48): n is a, c, g, or u
  misc_feature (54)..(54): Y is C or T/U
  misc_feature (57)..(57): n is a, c, g, or u
  misc_feature (60)..(60): n is a, c, g, or u
  misc_feature (61)..(61): Y is C or T/U
  misc_feature (63)..(63): n is a, c, g, or u
  misc_feature (66)..(66): n is a, c, g, or u
  misc_feature (69)..(69): R is A or G
  misc_feature (72)..(72): Y is C or T/U
  misc_feature (75)..(75): n is a, c, g, or u
  misc_feature (78)..(78): R is A or G
  misc_feature (81)..(81): R is A or G
  misc_feature (84)..(85): Y is C or T/U
  misc_feature (87)..(87): n is a, c, g, or u
  misc_feature (90)..(90): R is A or G
mgnwsnaarcarmgnmgnccncarggnyunccnaayaayacngcnwsnugguuyacngcnyunacncarcayggnaargargayyunaar

SEQ ID NO: 246 - RNA, 90 nt, artificial sequence
  RNA ecoding Covid-19 nucleocapsid peptide position 255-284
  misc_feature (1)..(1): W is A or T/U
  misc_feature (2)..(2): S is G or C
  misc_feature (3)..(3): n is a, c, g, or u
  misc_feature (6)..(6): R is A or G
  misc_feature (9)..(9): R is A or G
  misc_feature (12)..(12): n is a, c, g, or u
  misc_feature (13)..(13): M is A or C
  misc_feature (15)..(15): n is a, c, g, or u
  misc_feature (18)..(18): R is A or G
  misc_feature (21)..(21): R is A or G
  misc_feature (22)..(22): M is A or C
  misc_feature (24)..(24): n is a, c, g, or u
  misc_feature (27)..(27): n is a, c, g, or u
  misc_feature (30)..(30): n is a, c, g, or u
  misc_feature (33)..(33): n is a, c, g, or u
  misc_feature (39)..(39): n is a, c, g, or u
  misc_feature (42)..(42): Y is C or T/U
  misc_feature (45)..(45): Y is C or T/U
  misc_feature (48)..(48): n is a, c, g, or u
  misc_feature (51)..(51): n is a, c, g, or u
  misc_feature (54)..(54): R is A or G
  misc_feature (57)..(57): n is a, c, g, or u
  misc_feature (60)..(60): Y is C or T/U
  misc_feature (63)..(63): n is a, c, g, or u
  misc_feature (64)..(64): M is A or C
  misc_feature (66)..(66): n is a, c, g, or u
  misc_feature (67)..(67): M is A or C
  misc_feature (69)..(69): n is a, c, g, or u
  misc_feature (72)..(72): n is a, c, g, or u
  misc_feature (75)..(75): n is a, c, g, or u
  misc_feature (78)..(78): R is A or G
  misc_feature (81)..(81): R is A or G
  misc_feature (84)..(84): n is a, c, g, or u
  misc_feature (87)..(87): R is A or G
  misc_feature (90)..(90): n is a, c, g, or u
wsnaaraarccnmgncaraarmgnacngcnacnaargcnuayaaygunacncargcnuuyggnmgnmgnggnccngarcaracncarggn

SEQ ID NO: 247 - RNA, 90 nt, artificial sequence
  RNA encoding Covid-19 nucleocapsid peptide position 290-319
  misc_feature (3)..(3): R is A or G
  misc_feature (4)..(4): Y is C or T/U
  misc_feature (6)..(6): n is a, c, g, or u
  misc_feature (9)..(9): H is A or C or T/U
  misc_feature (10)..(10): M is A or C
  misc_feature (12)..(12): n is a, c, g, or u
  misc_feature (15)..(15): R is A or G
  misc_feature (18)..(18): n is a, c, g, or u
  misc_feature (21)..(21): n is a, c, g, or u
  misc_feature (24)..(24): Y is C or T/U
  misc_feature (27)..(27): Y is C or T/U
  misc_feature (30)..(30): R is A or G
  misc_feature (33)..(33): Y is C or T/U
  misc_feature (39)..(39): n is a, c, g, or u
  misc_feature (42)..(42): R is A or G
  misc_feature (45)..(45): H is A or C or T/U
  misc_feature (48)..(48): n is a, c, g, or u
  misc_feature (54)..(54): Y is C or T/U
  misc_feature (57)..(57): n is a, c, g, or u
  misc_feature (60)..(60): n is a, c, g, or u
  misc_feature (61)..(61): W is A or T/U
  misc_feature (62)..(62): S is G or C
  misc_feature (63)..(63): n is a, c, g, or u
  misc_feature (66)..(66): n is a, c, g, or u
  misc_feature (67)..(67): W is A or T/U
  misc_feature (68)..(68): S is G or C
  misc_feature (69)..(69): n is a, c, g, or u
  misc_feature (72)..(72): n is a, c, g, or u
  misc_feature (75)..(75): Y is C or T/U
  misc_feature (78)..(78): Y is C or T/U
  misc_feature (81)..(81): n is a, c, g, or u
  misc_feature (85)..(85): W is A or T/U
  misc_feature (86)..(86): S is G or C
  misc_feature (87)..(87): n is a, c, g, or u
  misc_feature (88)..(88): M is A or C
  misc_feature (90)..(90): n is a, c, g, or u
garyunauhmgncarggnacngayuayaarcayuggccncarauhgcncaruuygcnccnwsngcnwsngcnuuyuuyggnaugwsnmgn

SEQ ID NO: 248 - RNA, 90 nt, artificial sequence
  RNA encoding Covid-19 nucleocapsid peptide position 384-413
  misc_feature (3)..(3): R is A or G
  misc_feature (3)..(3): M is A or C
  misc_feature (6)..(6): n is a, c, g, or u
  misc_feature (9)..(9): R is A or G
  misc_feature (11)..(11): R is A or G
  misc_feature (15)..(15): R is A or G
  misc_feature (21)..(21): R is A or G
  misc_feature (24)..(24): n is a, c, g, or u
  misc_feature (27)..(27): n is a, c, g, or u
  misc_feature (30)..(30): n is a, c, g, or u
  misc_feature (31)..(31): Y is C or T/U
  misc_feature (33)..(33): n is a, c, g, or u
  misc_feature (34)..(34): Y is C or T/U
  misc_feature (36)..(36): n is a, c, g, or u
  misc_feature (39)..(39): n is a, c, g, or u
  misc_feature (42)..(42): n is a, c, g, or u
  misc_feature (45)..(45): n is a, c, g, or u
  misc_feature (48)..(49): Y is C or T/U
  misc_feature (51)..(51): n is a, c, g, or u
  misc_feature (54)..(54): Y is C or T/U
  misc_feature (57)..(57): Y is C or T/U
  misc_feature (60)..(60): Y is C or T/U
  misc_feature (61)..(61): W is A or T/U
  misc_feature (62)..(62): S is G or C
  misc_feature (63)..(63): n is a, c, g, or u
  misc_feature (66)..(66): R is A or G
  misc_feature (69)..(69): R is A or G
  misc_feature (70)..(70): Y is C or T/U
  misc_feature (72)..(72): n is a, c, g, or u
  misc_feature (75)..(75): R is A or G
  misc_feature (78)..(78): R is A or G
  misc_feature (79)..(79): W is A or T/U
  misc_feature (80)..(80): S is G or C
  misc_feature (81)..(81): n is a, c, g, or u
  misc_feature (85)..(85): W is A or T/U
  misc_feature (86)..(86): S is G or C
  misc_feature (87)..(87): n is a, c, g, or u
  misc_feature (88)..(88): W is A or T/U
  misc_feature (89)..(89): S is G or C
  misc_feature (90)..(90): n is a, c, g, or u
carmgncaraaraarcarcaracngunacnyunyunccngcngcngayyungaygayuuywsnaarcaryuncarcarwsnaugwsnwsn

SEQ ID NO: 249 - RNA, 90 nt, artificial sequence
  RNA encoding covid-19 membrane peptide position 93-122
  misc_feature (1)..(1): Y is C or T/U
  misc_feature (3)..(3): n is a, c, g, or u
  misc_feature (4)..(4): W is A or T/U
  misc_feature (5)..(5): S is G or C
  misc_feature (6)..(6): n is a, c, g, or u
  misc_feature (9)..(9): Y is C or T/U
  misc_feature (11)..(11): Y is C or T/U
  misc_feature (15)..(15): H is A or C or T/U
  misc_feature (18)..(18): n is a, c, g, or u
  misc_feature (19)..(19): W is A or T/U
  misc_feature (20)..(20): S is G or C
  misc_feature (21)..(21): n is a, c, g, or u
  misc_feature (24)..(24): Y is C or T/U
  misc_feature (25)..(25): M is A or C
  misc_feature (27)..(27): n is a, c, g, or u
  misc_feature (28)..(28): Y is C or T/U
  misc_feature (30)..(30): n is a, c, g, or u
  misc_feature (33)..(33): Y is C or T/U
  misc_feature (36)..(36): M is A or C
  misc_feature (39)..(39): n is a, c, g, or u
  misc_feature (42)..(42): n is a, c, g, or u
  misc_feature (43)..(43): M is A or C
  misc_feature (45)..(45): n is a, c, g, or u
  misc_feature (46)..(46): W is A or T/U
  misc_feature (47)..(47): S is G or C
  misc_feature (48)..(48): n is a, c, g, or u
  misc_feature (55)..(55): W is A or T/U
  misc_feature (56)..(56): S is G or C
  misc_feature (57)..(57): n is a, c, g, or u
  misc_feature (60)..(60): Y is C or T/U
  misc_feature (63)..(63): Y is C or T/U
  misc_feature (66)..(66): n is a, c, g, or u
  misc_feature (69)..(69): R is A or G
  misc_feature (72)..(72): n is a, c, g, or u
  misc_feature (75)..(75): Y is C or T/U
  misc_feature (78)..(78): H is A or C or T/U
  misc_feature (79)..(79): Y is C or T/U
  misc_feature (81)..(81): n is a, c, g, or u
  misc_feature (82)..(82): Y is C or T/U
  misc_feature (84)..(84): n is a, c, g, or u
  misc_feature (87)..(87): Y is C or T/U
  misc_feature (90)..(90): n is a, c, g, or u
yunwsnuayuuyauhgcnwsnuuymgnyunuuygcnmgnacnmgnwsnauguggwsnuuyaayccngaracnaayauhyunyunaaygun

SEQ ID NO: 250 - RNA, 90 nt, artificial sequence
  RNA encoding Covid-19 envelope peptide position 45-74
  misc_feature (3)..(3): Y is C or T/U
  misc_feature (6)..(6): H is A or C or T/U
  misc_feature (9)..(9): n is a, c, g, or u
  misc_feature (12)..(12): Y is C or T/U
  misc_feature (15)..(15): n is a, c, g, or u
  misc_feature (16)..(16): W is A or T/U
  misc_feature (17)..(17): S is G or C
  misc_feature (18)..(18): n is a, c, g, or u
  misc_feature (19)..(19): Y is C or T/U
  misc_feature (21)..(21): n is a, c, g, or u
  misc_feature (24)..(24): n is a, c, g, or u
  misc_feature (27)..(27): R is A or G
  misc_feature (30)..(30): n is a, c, g, or u
  misc_feature (31)..(31): W is A or T/U
  misc_feature (32)..(32): S is G or C
  misc_feature (33)..(33): n is a, c, g, or u
  misc_feature (36)..(36): Y is C or T/U
  misc_feature (39)..(39): Y is C or T/U
  misc_feature (42)..(42): n is a, c, g, or u
  misc_feature (45)..(45): Y is C or T/U
  misc_feature (46)..(46): W is A or T/U
  misc_feature (47)..(47): S is G or C
  misc_feature (48)..(48): n is a, c, g, or u
  misc_feature (49)..(49): M is A or C
  misc_feature (51)..(51): n is a, c, g, or u
  misc_feature (54)..(54): n is a, c, g, or u
  misc_feature (57)..(57): R is A or G
  misc_feature (60)..(61): Y is C or T/U
  misc_feature (63)..(63): n is a, c, g, or u
  misc_feature (66)..(66): Y is C or T/U
  misc_feature (67)..(67): W is A or T/U
  misc_feature (68)..(68): S is G or C
  misc_feature (69)..(69): n is a, c, g, or u
  misc_feature (70)..(70): W is A or T/U
  misc_feature (71)..(71): S is G or C
  misc_feature (72)..(72): n is a, c, g, or u
  misc_feature (73)..(73): M is A or C
  misc_feature (75)..(75): n is a, c, g, or u
  misc_feature (78)..(78): n is a, c, g, or u
  misc_feature (81)..(81): n is a, c, g, or u
  misc_feature (84)..(85): Y is C or T/U
  misc_feature (87)..(87): Y is C or T/U
  misc_feature (90)..(90): n is a, c, g, or u
aayauhgunaaygunwsnyungunaarccnwsnuuyuaygunuaywsnmgngunaaraayyunaaywsnwsnmgngunccngayyunyun

SEQ ID NO: 251 - DNA, 90 bp, artificial sequence
  DNA encoding Covid-19 surface peptide position 22-51
  misc_feature (3)..(3): n is a, c, g, or u
  misc_feature (6)..(6): R is A or G
  misc_feature (9)..(9): n is a, c, g, or u
  misc_feature (12)..(12): n is a, c, g, or u
  misc_feature (15)..(15): n is a, c, g, or u
  misc_feature (18)..(18): n is a, c, g, or u
  misc_feature (21)..(21): Y is C or T/U
  misc_feature (24)..(24): n is a, c, g, or u
  misc_feature (27)..(27): Y is C or T/U
  misc_feature (28)..(28): W is A or T/U,
  misc_feature (29)..(29): S is G or C
  misc_feature (30)..(30): n is a, c, g, or u
  misc_feature (33)..(33): Y is C or T/U
  misc_feature (36)..(36): n is a, c, g, or u
  misc_feature (37)..(37): M is A or C
  misc_feature (39)..(39): n is a, c, g, or u
  misc_feature (42)..(42): n is a, c, g, or u
  misc_feature (45)..(45): n is a, c, g, or u
  misc_feature (48)..(48): Y is C or T/U
  misc_feature (51)..(51): Y is C or T/U
  misc_feature (54)..(54): n is a, c, g, or u
  misc_feature (57)..(57): Y is C or T/U
  misc_feature (60)..(60): Y is C or T/U
  misc_feature (63)..(63): n is a, c, g, or u
  misc_feature (66)..(66): Y is C or T/U
  misc_feature (67)..(67): M is A or C
  misc_feature (69)..(69): n is a, c, g, or u
  misc_feature (70)..(70): W is A or T/U,
  misc_feature (71)..(71): S is G or C
  misc_feature (72)..(72): n is a, c, g, or u
  misc_feature (73)..(73): W is A or T/U,
  misc_feature (74)..(74): S is G or C
  misc_feature (75)..(75): n is a, c, g, or u
  misc_feature (78)..(78): n is a, c, g, or u
  misc_feature (79)..(79): Y is C or T/U
  misc_feature (81)..(81): n is a, c, g, or u
  misc_feature (84)..(84): Y is C or T/U
  misc_feature (85)..(85): W is A or T/U,
  misc_feature (86)..(86): S is G or C
  misc_feature (87)..(87): n is a, c, g, or u
  misc_feature (90)..(90): n is a, c, g, or u
acncarytnccnccngcntayacnaaywsnttyacnmgnggngtntaytayccngayaargtnttymgnwsnwsngtnytncaywsnacn

SEQ ID NO: 252 - DNA, 90 bp, artificial sequence
  DNA encoding Covid-19 surface peptide position 35-64
  misc_feature (3)..(3): n is a, c, g, or u
  misc_feature (6)..(6): n is a, c, g, or u
  misc_feature (9)..(9): Y is C or T/U
  misc_feature (12)..(12): Y is C or T/U
  misc_feature (15)..(15): n is a, c, g, or u
  misc_feature (18)..(18): Y is C or T/U
  misc_feature (21)..(21): R is A or G
  misc_feature (24)..(24): n is a, c, g, or u
  misc_feature (27)..(27): Y is C or T/U
  misc_feature (28)..(28): M is A or C
  misc_feature (30)..(30): n is a, c, g, or u
  misc_feature (31)..(31): W is A or T/U
  misc_feature (32)..(32): S is G or C
  misc_feature (33)..(33): n is a, c, g, or u
  misc_feature (34)..(34): W is A or T/U
  misc_feature (35)..(35): S is G or C
  misc_feature (36)..(36): n is a, c, g, or u
  misc_feature (39)..(39): n is a, c, g, or u
  misc_feature (40)..(40): Y is C or T/U
  misc_feature (42)..(42): n is a, c, g, or u
  misc_feature (45)..(45): Y is C or T/U
  misc_feature (46)..(46): W is A or T/U
  misc_feature (47)..(47): S is G or C
  misc_feature (48)..(48): n is a, c, g, or u
  misc_feature (51)..(51): n is a, c, g, or u
  misc_feature (54)..(54): R is A or G
  misc_feature (57)..(58): Y is C or T/U
  misc_feature (60)..(60): n is a, c, g, or u
  misc_feature (63)..(64): Y is C or T/U
  misc_feature (66)..(66): n is a, c, g, or u
  misc_feature (69)..(69): n is a, c, g, or u
  misc_feature (72)..(72): Y is C or T/U
  misc_feature (75)..(75): Y is C or T/U
  misc_feature (76)..(76): W is A or T/U
  misc_feature (77)..(77): S is G or C
  misc_feature (78)..(78): n is a, c, g, or u
  misc_feature (81)..(81): Y is C or T/U
  misc_feature (84)..(84): n is a, c, g, or u
  misc_feature (87)..(87): n is a, c, g, or u
ggngtntaytayccngayaargtnttymgnwsnwsngtnytncaywsnacncargayytnttyytnccnttyttywsnaaygtnacntgg

SEQ ID NO: 253 - DNA, 90 bp, artificial sequence
  DNA encoding Covid-19 surface peptide position 76-105
  misc_feature (3)..(3): n is a, c, g, or u
  misc_feature (6)..(6): R is A or G
  misc_feature (7)..(7): M is A or C
  misc_feature (9)..(9): n is a, c, g, or u
  misc_feature (12)..(12): Y is C or T/U
  misc_feature (15)..(15): Y is C or T/U
  misc_feature (18)..(18): Y is C or T/U
  misc_feature (21)..(21): n is a, c, g, or u
  misc_feature (24)..(24): n is a, c, g, or u
  misc_feature (25)..(25): Y is C or T/U
  misc_feature (27)..(27): n is a, c, g, or u
  misc_feature (30)..(30): n is a, c, g, or u
  misc_feature (33)..(33): Y is C or T/U
  misc_feature (36)..(36): Y is C or T/U
  misc_feature (39)..(39): Y is C or T/U
  misc_feature (42)..(42): n is a, c, g, or u
  misc_feature (45)..(45): n is a, c, g, or u
  misc_feature (48)..(48): Y is C or T/U
  misc_feature (51)..(51): Y is C or T/U
  misc_feature (54)..(54): n is a, c, g, or u
  misc_feature (55)..(55): W is A or T/U
  misc_feature (56)..(56): S is G or C
  misc_feature (57)..(57): n is a, c, g, or u
  misc_feature (60)..(60): n is a, c, g, or u
  misc_feature (63)..(63): R is A or G
  misc_feature (66)..(66): R is A or G
  misc_feature (67)..(67): W is A or T/U
  misc_feature (68)..(68): S is G or C
  misc_feature (69)..(69): n is a, c, g, or u
  misc_feature (72)..(72): Y is C or T/U
  misc_feature (75)..(75): H is A or C or T/U
  misc_feature (78)..(78): H is A or C or T/U
  misc_feature (79)..(79): M is A or C
  misc_feature (81)..(81): n is a, c, g, or u
  misc_feature (84)..(84): n is a, c, g, or u
  misc_feature (90)..(90): H is A or C or T/U
acnaarmgnttygayaayccngtnytnccnttyaaygayggngtntayttygcnwsnacngaraarwsnaayathathmgnggntggath

SEQ ID NO: 254 - DNA, 90 bp, artificial sequence
  DNA encoding Covid-19 surface peptide position 98-127
  misc_feature (1)..(1): W is A or T/U
  misc_feature (2)..(2): S is G or C
  misc_feature (3)..(3): n is a, c, g, or u
  misc_feature (6)..(6): Y is C or T/U
  misc_feature (9)..(9): H is A or C or T/U
  misc_feature (12)..(12): H is A or C or T/U
  misc_feature (13)..(13): M is A or C
  misc_feature (15)..(15): n is a, c, g, or u
  misc_feature (18)..(18): n is a, c, g, or u
  misc_feature (24)..(24): H is A or C or T/U
  misc_feature (27)..(27): Y is C or T/U
  misc_feature (30)..(30): n is a, c, g, or u
  misc_feature (33)..(33): n is a, c, g, or u
  misc_feature (36)..(36): n is a, c, g, or u
  misc_feature (37)..(37): Y is C or T/U
  misc_feature (39)..(39): n is a, c, g, or u
  misc_feature (42)..(42): Y is C or T/U
  misc_feature (43)..(43): W is A or T/U
  misc_feature (44)..(44): S is G or C
  misc_feature (45)..(45): n is a, c, g, or u
  misc_feature (48)..(48): R is A or G
  misc_feature (51)..(51): n is a, c, g, or u
  misc_feature (54)..(54): R is A or G
  misc_feature (55)..(55): W is A or T/U
  misc_feature (56)..(56): S is G or C
  misc_feature (57)..(57): n is a, c, g, or u
  misc_feature (58)..(58): Y is C or T/U
  misc_feature (60)..(60): n is a, c, g, or u
  misc_feature (61)..(61): Y is C or T/U
  misc_feature (63)..(63): n is a, c, g, or u
  misc_feature (65)..(65): H is A or C or T/U
  misc_feature (69)..(69): n is a, c, g, or u
  misc_feature (72)..(72): Y is C or T/U
  misc_feature (75)..(75): Y is C or T/U
  misc_feature (78)..(78): n is a, c, g, or u
  misc_feature (81)..(81): Y is C or T/U
  misc_feature (87)..(87): n is a, c, g, or u
  misc_feature (90)..(90): n is a, c, g, or u
wsnaayathathmgnggntggathttyggnacnacnytngaywsnaaracncarwsnytnytnathgtnaayaaygcnacnaaygtngtn

SEQ ID NO: 255 - DNA, 90 bp, artificial sequence
  DNA encoding Covid-19 surface peptide position 253-282
  misc_feature (3)..(3): Y is C or T/U
  misc_feature (4)..(4): W is A or T/U
  misc_feature (5)..(5): S is G or C
  misc_feature (6)..(6): n is a, c, g, or u
  misc_feature (7)..(7): W is A or T/U
  misc_feature (8)..(8): S is G or C
  misc_feature (9)..(9): n is a, c, g, or u
  misc_feature (10)..(10): W is A or T/U
  misc_feature (11)..(11): S is G or C
  misc_feature (12)..(12): n is a, c, g, or u
  misc_feature (15)..(15): n is a, c, g, or u
  misc_feature (21)..(21): n is a, c, g, or u
  misc_feature (24)..(24): n is a, c, g, or u
  misc_feature (27)..(27): n is a, c, g, or u
  misc_feature (30)..(30): n is a, c, g, or u
  misc_feature (33)..(33): n is a, c, g, or u
  misc_feature (36)..(36): n is a, c, g, or u
  misc_feature (39)..(39): Y is C or T/U
  misc_feature (42)..(42): Y is C or T/U
  misc_feature (45)..(45): n is a, c, g, or u
  misc_feature (48)..(48): n is a, c, g, or u
  misc_feature (51)..(52): Y is C or T/U
  misc_feature (54)..(54): n is a, c, g, or u
  misc_feature (57)..(57): R is A or G
  misc_feature (60)..(60): n is a, c, g, or u
  misc_feature (61)..(61): M is A or C
  misc_feature (63)..(63): n is a, c, g, or u
  misc_feature (66)..(66): n is a, c, g, or u
  misc_feature (69)..(69): Y is C or T/U
  misc_feature (70)..(70): Y is C or T/U
  misc_feature (72)..(72): n is a, c, g, or u
  misc_feature (73)..(73): Y is C or T/U
  misc_feature (75)..(75): n is a, c, g, or u
  misc_feature (78)..(78): R is A or G
  misc_feature (81)..(81): Y is C or T/U
  misc_feature (84)..(84): Y is C or T/U
  misc_feature (87)..(87): R is A or G
  misc_feature (90)..(90): Y is C or T/U
gaywsnwsnwsnggntggacngcnggngcngcngcntaytaygtnggntayytncarccnmgnacnttyytnytnaartayaaygaraay

SEQ ID NO: 256 - DNA, 90 bp, artificial sequence
  DNA encoding Covid-19 surface peptide position 391-420
  misc_feature (3)..(3): Y is C or T/U
  misc_feature (6)..(6): Y is C or T/U
  misc_feature (9)..(9): n is a, c, g, or u
  misc_feature (12)..(12): Y is C or T/U
  misc_feature (15)..(15): n is a, c, g, or u
  misc_feature (18)..(18): Y is C or T/U
  misc_feature (21)..(21): n is a, c, g, or u
  misc_feature (24)..(24): Y is C or T/U
  misc_feature (25)..(25): W is A or T/U
  misc_feature (26)..(26): S is G or C
  misc_feature (27)..(27): n is a, c, g, or u
  misc_feature (30)..(30): Y is C or T/U
  misc_feature (33)..(33): n is a, c, g, or u
  misc_feature (36)..(36): H is A or C or T/U
  misc_feature (37)..(37): M is A or C
  misc_feature (39)..(39): n is a, c, g, or u
  misc_feature (42)..(42): n is a, c, g, or u
  misc_feature (45)..(45): Y is C or T/U
  misc_feature (48)..(48): R is A or G
  misc_feature (51)..(51): M is A or C
  misc_feature (54)..(54): n is a, c, g, or u
  misc_feature (57)..(57): R is A or G
  misc_feature (60)..(60): H is A or C or T/U
  misc_feature (63)..(63): n is a, c, g, or u
  misc_feature (66)..(66): n is a, c, g, or u
  misc_feature (69)..(69): n is a, c, g, or u
  misc_feature (72)..(72): R is A or G
  misc_feature (75)..(75): n is a, c, g, or u
  misc_feature (78)..(78): n is a, c, g, or u
  misc_feature (81)..(81): R is A or G
  misc_feature (84)..(84): H is A or C or T/U
  misc_feature (87)..(87): n is a, c, g, or u
  misc_feature (90)..(90): Y is C or T/U
tgyttyacnaaygtntaygcngaywsnttygtnathmgnggngaygargtnmgncarathgcnccnggncaracnggnaarathgcngay

SEQ ID NO: 257 - DNA, 90 bp, artificial sequence
  DNA encoding Covid-19 surface peptide position 638-712
  misc_feature (1)..(1): M is A or C
  misc_feature (3)..(3): n is a, c, g, or u
  misc_feature (6)..(6): n is a, c, g, or u
  misc_feature (7)..(7): M is A or C
  misc_feature (9)..(9): n is a, c, g, or u
  misc_feature (10)..(10): W is A or T/U
  misc_feature (11)..(11): S is G or C
  misc_feature (12)..(12): n is a, c, g, or u
  misc_feature (15)..(15): n is a, c, g, or u
  misc_feature (18)..(18): n is a, c, g, or u
  misc_feature (19)..(19): W is A or T/U
  misc_feature (20)..(20): S is G or C
  misc_feature (21)..(21): n is a, c, g, or u
  misc_feature (24)..(60): R is A or G
  misc_feature (25)..(25): S is G or C
  misc_feature (30)..(30): H is A or C or T/U
  misc_feature (33)..(33): H is A or C or T/U
  misc_feature (39)..(39): Y is C or T/U
  misc_feature (46)..(46): W is A or T/U
  misc_feature (47)..(47): S is G or C
  misc_feature (49)..(49): Y is C or T/U
  misc_feature (63)..(63): Y is C or T/U
  misc_feature (64)..(64): W is A or T/U
  misc_feature (65)..(65): S is G or C
  misc_feature (66)..(66): n is a, c, g, or u
  misc_feature (69)..(69): n is a, c, g, or u
  misc_feature (72)..(72): n is a, c, g, or u
  misc_feature (75)..(75): Y is C or T/U
  misc_feature (76)..(76): W is A or T/U
  misc_feature (78)..(78): S is G or C
  misc_feature (81)..(81): Y is C or T/U
  misc_feature (84)..(84): Y is C or T/U
  misc_feature (85)..(85): W is A or T/U
  misc_feature (86)..(86): S is G or C
  misc_feature (87)..(87): n is a, c, g, or u
  misc_feature (90)..(90): H is A or C or T/U
mgngcnmgnwsngtngcnwsncarwsnathathgcntayacnatgwsnytnggngcngaraaywsngtngcntaywsnaayaaywsnath

SEQ ID NO: 258 - DNA, 90 bp, artificial sequence
  DNA encoding Covid-19 surface peptide position 701-730
  misc_feature (3)..(3): n is a, c, g, or u
  misc_feature (6)..(6): R is A or G
  misc_feature (9)..(9): Y is C or T/U
  misc_feature (10)..(10): W is A or T/U
  misc_feature (11)..(11): S is G or C
  misc_feature (12)..(12): n is a, c, g, or u
  misc_feature (15)..(15): n is a, c, g, or u
  misc_feature (18)..(18): n is a, c, g, or u
  misc_feature (21)..(21): Y is C or T/U
  misc_feature (22)..(22): W is A or T/U
  misc_feature (23)..(23): S is G or C
  misc_feature (24)..(24): n is a, c, g, or u
  misc_feature (27)..(27): Y is C or T/U
  misc_feature (30)..(30): Y is C or T/U
  misc_feature (31)..(31): W is A or T/U
  misc_feature (32)..(32): S is G or C
  misc_feature (33)..(33): n is a, c, g, or u
  misc_feature (36)..(36): H is A or C or T/U
  misc_feature (39)..(39): n is a, c, g, or u
  misc_feature (42)..(42): H is A or C or T/U
  misc_feature (45)..(45): n is a, c, g, or u
  misc_feature (48)..(48): n is a, c, g, or u
  misc_feature (51)..(51): Y is C or T/U
  misc_feature (54)..(54): Y is C or T/U
  misc_feature (57)..(57): n is a, c, g, or u
  misc_feature (60)..(60): H is A or C or T/U
  misc_feature (61)..(61): W is A or T/U
  misc_feature (62)..(62): S is G or C
  misc_feature (63)..(63): n is a, c, g, or u
  misc_feature (66)..(66): n is a, c, g, or u
  misc_feature (69)..(69): n is a, c, g, or u
  misc_feature (72)..(72): n is a, c, g, or u
  misc_feature (75)..(75): R is A or G
  misc_feature (78)..(78): H is A or C or T/U
  misc_feature (79)..(79): Y is C or T/U
  misc_feature (81)..(81): n is a, c, g, or u
  misc_feature (84)..(84): n is a, c, g, or u
  misc_feature (87)..(87): W is A or T/U
  misc_feature (89)..(89): S is G or C
  misc_feature (90)..(90): n is a, c, g, or u
gcngaraaywsngtngcntaywsnaayaaywsnathgcnathccnacnaayttyacnathwsngtnacnacngarathytnccngtnwsn

SEQ ID NO: 259 - DNA, 90 bp, artificial sequence
  DNA encoding Covid-19 surface peptide position 893-922
  misc_feature (3)..(3): n is a, c, g, or u
  misc_feature (4)..(4): Y is C or T/U
  misc_feature (6)..(6): n is a, c, g, or u
  misc_feature (15)..(15): n is a, c, g, or u
  misc_feature (18)..(18): Y is C or T/U
  misc_feature (21)..(21): n is a, c, g, or u
  misc_feature (27)..(27): R is A or G
  misc_feature (33)..(33): n is a, c, g, or u
  misc_feature (36)..(36): Y is C or T/U
  misc_feature (37)..(37): M is A or C
  misc_feature (39)..(39): n is a, c, g, or u
  misc_feature (42)..(42): Y is C or T/U
  misc_feature (45)..(45): Y is C or T/U
  misc_feature (48)..(48): n is a, c, g, or u
  misc_feature (51)..(51): H is A or C or T/U
  misc_feature (54)..(54): n is a, c, g, or u
  misc_feature (57)..(57): n is a, c, g, or u
  misc_feature (60)..(60): n is a, c, g, or u
  misc_feature (63)..(63): R is A or G
  misc_feature (66)..(66): Y is C or T/U
  misc_feature (69)..(69): n is a, c, g, or u
  misc_feature (70)..(70): Y is C or T/U
  misc_feature (72)..(72): n is a, c, g, or u
  misc_feature (75)..(75): Y is C or T/U
  misc_feature (78)..(78): R is A or G
  misc_feature (81)..(81): Y is C or T/U
  misc_feature (84)..(84): R is A or G
  misc_feature (87)..(87): R is A or G
  misc_feature (88)..(88): Y is C or T/U
  misc_feature (90)..(90): n is a, c, g, or u
gcnytncarathccnttygcnatgcaratggcntaymgnttyaayggnathggngtnacncaraaygtnytntaygaraaycaraarytn

SEQ ID NO: 260 - DNA, 90 bp, artificial sequence
  DNA encoding Covid-19 surface peptide position 898-927
  misc_feature (3)..(3): Y is C or T/U
  misc_feature (6)..(6): n is a, c, g, or u
  misc_feature (12)..(12): R is A or G
  misc_feature (18)..(18): n is a, c, g, or u
  misc_feature (21)..(21): Y is C or T/U
  misc_feature (22)..(22): M is A or C
  misc_feature (24)..(24): n is a, c, g, or u
  misc_feature (27)..(27): Y is C or T/U
  misc_feature (30)..(30): Y is C or T/U
  misc_feature (33)..(33): n is a, c, g, or u
  misc_feature (36)..(36): H is A or C or T/U
  misc_feature (39)..(39): n is a, c, g, or u
  misc_feature (42)..(42): n is a, c, g, or u
  misc_feature (45)..(45): n is a, c, g, or u
  misc_feature (48)..(48): R is A or G
  misc_feature (51)..(51): Y is C or T/U
  misc_feature (54)..(54): n is a, c, g, or u
  misc_feature (55)..(55): Y is C or T/U
  misc_feature (57)..(57): n is a, c, g, or u
  misc_feature (60)..(60): Y is C or T/U
  misc_feature (63)..(63): R is A or G
  misc_feature (66)..(66): Y is C or T/U
  misc_feature (69)..(69): R is A or G
  misc_feature (72)..(72): R is A or G
  misc_feature (73)..(73): Y is C or T/U
  misc_feature (75)..(75): n is a, c, g, or u
  misc_feature (78)..(78): H is A or C or T/U
  misc_feature (81)..(81): n is a, c, g, or u
  misc_feature (84)..(84): Y is C or T/U
  misc_feature (86)..(86): R is A or G
  misc_feature (90)..(90): Y is C or T/U
ttygcnatgcaratggcntaymgnttyaayggnathggngtnacncaraaygtnytntaygaraaycaraarytnathgcnaaycartty

SEQ ID NO: 261 - DNA, 90 bp, artificial sequence
  DNA encoding Covid-19 nucleocapsid peptide position 1091-1120
  misc_feature (1)..(1): M is A or C
  misc_feature (3)..(3): n is a, c, g, or u
  misc_feature (6)..(6): R is A or G
  misc_feature (9)..(9): n is a, c, g, or u
  misc_feature (12)..(12): n is a, c, g, or u
  misc_feature (15)..(15): Y is C or T/U
  misc_feature (18)..(18): n is a, c, g, or u
  misc_feature (21)..(21): n is a, c, g, or u
  misc_feature (24)..(24): Y is C or T/U
  misc_feature (27)..(27): n is a, c, g, or u
  misc_feature (30)..(30): n is a, c, g, or u
  misc_feature (33)..(33): Y is C or T/U
  misc_feature (39)..(39): Y is C or T/U
  misc_feature (42)..(42): n is a, c, g, or u
  misc_feature (45)..(45): n is a, c, g, or u
  misc_feature (48)..(48): R is A or G
  misc_feature (49)..(49): M is A or C
  misc_feature (51)..(51): n is a, c, g, or u
  misc_feature (54)..(54): Y is C or T/U
  misc_feature (57)..(57): Y is C or T/U
  misc_feature (60)..(60): Y is C or T/U
  misc_feature (63)..(63): R is A or G
  misc_feature (66)..(66): n is a, c, g, or u
  misc_feature (69)..(69): R is A or G
  misc_feature (72)..(72): H is A or C or T/U
  misc_feature (75)..(75): H is A or C or T/U
  misc_feature (78)..(78): n is a, c, g, or u
  misc_feature (81)..(81): n is a, c, g, or u
  misc_feature (84)..(84): Y is C or T/U
  misc_feature (87)..(87): Y is C or T/U
  misc_feature (90)..(90): n is a, c, g, or u
mgngarggngtnttygtnwsnaayggnacncaytggttygtnacncarmgnaayttytaygarccncarathathacnacngayaayacn

SEQ ID NO: 262 - DNA, 90 bp, artificial sequence
  DNA encoding Covid-19 nucleocapsid peptide position 36-65
  misc_feature (1)..(1): M is A or C
  misc_feature (3)..(3): n is a, c, g, or u
  misc_feature (4)..(4): W is A or T/U
  misc_feature (5)..(5): S is G or C
  misc_feature (6)..(6): n is a, c, g, or u
  misc_feature (9)..(9): R is A or G
  misc_feature (12)..(12): R is A or G
  misc_feature (13)..(13): M is A or C
  misc_feature (15)..(15): n is a, c, g, or u
  misc_feature (16)..(16): M is A or C
  misc_feature (18)..(18): n is a, c, g, or u
  misc_feature (21)..(21): n is a, c, g, or u
  misc_feature (24)..(24): R is A or G
  misc_feature (27)..(27): n is a, c, g, or u
  misc_feature (28)..(28): Y is C or T/U
  misc_feature (30)..(30): n is a, c, g, or u
  misc_feature (33)..(33): n is a, c, g, or u
  misc_feature (36)..(36): Y is C or T/U
  misc_feature (39)..(39): Y is C or T/U
  misc_feature (42)..(42): n is a, c, g, or u
  misc_feature (45)..(45): n is a, c, g, or u
  misc_feature (46)..(46): W is A or T/U
  misc_feature (47)..(47): S is G or C
  misc_feature (48)..(48): n is a, c, g, or u
  misc_feature (54)..(54): Y is C or T/U
  misc_feature (57)..(57): n is a, c, g, or u
  misc_feature (60)..(60): n is a, c, g, or u
  misc_feature (61)..(61): Y is C or T/U
  misc_feature (63)..(63): n is a, c, g, or u
  misc_feature (66)..(66): n is a, c, g, or u
  misc_feature (69)..(69): R is A or G
  misc_feature (72)..(72): Y is C or T/U
  misc_feature (75)..(75): n is a, c, g, or u
  misc_feature (78)..(78): R is A or G
  misc_feature (81)..(81): R is A or G
  misc_feature (84)..(85): Y is C or T/U
  misc_feature (87)..(87): n is a, c, g, or u
  misc_feature (90)..(90): R is A or G
mgnwsnaarcarmgnmgnccncarggnytnccnaayaayacngcnwsntggttyacngcnytnacncarcayggnaargargayytnaar

SEQ ID NO: 263 - DNA, 90 bp, artificial sequence
  DNA encoding Covid-19 nucleocapsid peptide position 255-284
  misc_feature (1)..(1): W is A or T/U
  misc_feature (2)..(2): S is G or C
  misc_feature (3)..(3): n is a, c, g, or u
  misc_feature (6)..(6): R is A or G
  misc_feature (9)..(9): R is A or G
  misc_feature (12)..(12): n is a, c, g, or u
  misc_feature (13)..(13): M is A or C
  misc_feature (15)..(15): n is a, c, g, or u
  misc_feature (18)..(18): R is A or G
  misc_feature (21)..(21): R is A or G
  misc_feature (22)..(22): M is A or C
  misc_feature (24)..(24): n is a, c, g, or u
  misc_feature (27)..(27): n is a, c, g, or u
  misc_feature (30)..(30): n is a, c, g, or u
  misc_feature (33)..(33): n is a, c, g, or u
  misc_feature (39)..(39): n is a, c, g, or u
  misc_feature (42)..(42): Y is C or T/U
  misc_feature (45)..(45): Y is C or T/U
  misc_feature (48)..(48): n is a, c, g, or u
  misc_feature (51)..(51): n is a, c, g, or u
  misc_feature (54)..(54): R is A or G
  misc_feature (57)..(57): n is a, c, g, or u
  misc_feature (60)..(60): Y is C or T/U
  misc_feature (63)..(63): n is a, c, g, or u
  misc_feature (64)..(64): M is A or C
  misc_feature (66)..(66): n is a, c, g, or u
  misc_feature (67)..(67): M is A or C
  misc_feature (69)..(69): n is a, c, g, or u
  misc_feature (72)..(72): n is a, c, g, or u
  misc_feature (75)..(75): n is a, c, g, or u
  misc_feature (78)..(78): R is A or G
  misc_feature (81)..(81): R is A or G
  misc_feature (84)..(84): n is a, c, g, or u
  misc_feature (87)..(87): R is A or G
  misc_feature (90)..(90): n is a, c, g, or u
wsnaaraarccnmgncaraarmgnacngcnacnaargcntayaaygtnacncargcnttyggnmgnmgnggnccngarcaracncarggn

SEQ ID NO: 264 - DNA, 90 bp, artificial sequence
  DNA encoding Covid-19 nucleocapsid peptide position 290-319
  misc_feature (3)..(3): R is A or G
  misc_feature (4)..(4): Y is C or T/U
  misc_feature (6)..(6): n is a, c, g, or u
  misc_feature (9)..(9): H is A or C or T/U
  misc_feature (10)..(10): M is A or C
  misc_feature (12)..(12): n is a, c, g, or u
  misc_feature (15)..(15): R is A or G
  misc_feature (18)..(18): n is a, c, g, or u
  misc_feature (21)..(21): n is a, c, g, or u
  misc_feature (24)..(24): Y is C or T/U
  misc_feature (27)..(27): Y is C or T/U
  misc_feature (30)..(30): R is A or G
  misc_feature (33)..(33): Y is C or T/U
  misc_feature (39)..(39): n is a, c, g, or u
  misc_feature (42)..(42): R is A or G
  misc_feature (45)..(45): H is A or C or T/U
  misc_feature (48)..(48): n is a, c, g, or u
  misc_feature (54)..(54): Y is C or T/U
  misc_feature (57)..(57): n is a, c, g, or u
  misc_feature (60)..(60): n is a, c, g, or u
  misc_feature (61)..(61): W is A or T/U
  misc_feature (62)..(62): S is G or C
  misc_feature (63)..(63): n is a, c, g, or u
  misc_feature (66)..(66): n is a, c, g, or u
  misc_feature (67)..(67): W is A or T/U
  misc_feature (68)..(68): S is G or C
  misc_feature (69)..(69): n is a, c, g, or u
  misc_feature (72)..(72): n is a, c, g, or u
  misc_feature (75)..(75): Y is C or T/U
  misc_feature (78)..(78): Y is C or T/U
  misc_feature (81)..(81): n is a, c, g, or u
  misc_feature (85)..(85): W is A or T/U
  misc_feature (86)..(86): S is G or C
  misc_feature (87)..(87): n is a, c, g, or u
  misc_feature (88)..(88): M is A or C
  misc_feature (90)..(90): n is a, c, g, or u
garytnathmgncarggnacngaytayaarcaytggccncarathgcncarttygcnccnwsngcnwsngcnttyttyggnatgwsnmgn

SEQ ID NO: 265 - DNA, 90 bp, artificial sequence
  DNA encoding Covid-19 nucleocapsid peptide position 384-413
  misc_feature (3)..(3): R is A or G
  misc_feature (3)..(3): M is A or C
  misc_feature (6)..(6): n is a, c, g, or u
  misc_feature (9)..(9): R is A or G
  misc_feature (11)..(11): R is A or G
  misc_feature (15)..(15): R is A or G
  misc_feature (21)..(21): R is A or G
  misc_feature (24)..(24): n is a, c, g, or u
  misc_feature (27)..(27): n is a, c, g, or u
  misc_feature (30)..(30): n is a, c, g, or u
  misc_feature (31)..(31): Y is C or T/U
  misc_feature (33)..(33): n is a, c, g, or u
  misc_feature (34)..(34): Y is C or T/U
  misc_feature (36)..(36): n is a, c, g, or u
  misc_feature (39)..(39): n is a, c, g, or u
  misc_feature (42)..(42): n is a, c, g, or u
  misc_feature (45)..(45): n is a, c, g, or u
  misc_feature (48)..(49): Y is C or T/U
  misc_feature (51)..(51): n is a, c, g, or u
  misc_feature (54)..(54): Y is C or T/U
  misc_feature (57)..(57): Y is C or T/U
  misc_feature (60)..(60): Y is C or T/U
  misc_feature (61)..(61): W is A or T/U
  misc_feature (62)..(62): S is G or C
  misc_feature (63)..(63): n is a, c, g, or u
  misc_feature (66)..(66): R is A or G
  misc_feature (69)..(69): R is A or G
  misc_feature (70)..(70): Y is C or T/U
  misc_feature (72)..(72): n is a, c, g, or u
  misc_feature (75)..(75): R is A or G
  misc_feature (78)..(78): R is A or G
  misc_feature (79)..(79): W is A or T/U
  misc_feature (80)..(80): S is G or C
  misc_feature (81)..(81): n is a, c, g, or u
  misc_feature (85)..(85): W is A or T/U
  misc_feature (86)..(86): S is G or C
  misc_feature (87)..(87): n is a, c, g, or u
  misc_feature (88)..(88): W is A or T/U
  misc_feature (89)..(89): S is G or C
  misc_feature (90)..(90): n is a, c, g, or u
carmgncaraaraarcarcaracngtnacnytnytnccngcngcngayytngaygayttywsnaarcarytncarcarwsnatgwsnwsn

SEQ ID NO: 266 - DNA, 90 bp, artificial sequence
  DNA encoding Covid-19 membrane peptide position 93-122
  misc_feature (1)..(1): Y is C or T/U
  misc_feature (3)..(3): n is a, c, g, or u
  misc_feature (4)..(4): W is A or T/U
  misc_feature (5)..(5): S is G or C
  misc_feature (6)..(6): n is a, c, g, or u
  misc_feature (9)..(9): Y is C or T/U
  misc_feature (11)..(11): Y is C or T/U
  misc_feature (15)..(15): H is A or C or T/U
  misc_feature (18)..(18): n is a, c, g, or u
  misc_feature (19)..(19): W is A or T/U
  misc_feature (20)..(20): S is G or C
  misc_feature (21)..(21): n is a, c, g, or u
  misc_feature (24)..(24): Y is C or T/U
  misc_feature (25)..(25): M is A or C
  misc_feature (27)..(27): n is a, c, g, or u
  misc_feature (28)..(28): Y is C or T/U
  misc_feature (30)..(30): n is a, c, g, or u
  misc_feature (33)..(33): Y is C or T/U
  misc_feature (36)..(36): M is A or C
  misc_feature (39)..(39): n is a, c, g, or u
  misc_feature (42)..(42): n is a, c, g, or u
  misc_feature (43)..(43): M is A or C
  misc_feature (45)..(45): n is a, c, g, or u
  misc_feature (46)..(46): W is A or T/U
  misc_feature (47)..(47): S is G or C
  misc_feature (48)..(48): n is a, c, g, or u
  misc_feature (55)..(55): W is A or T/U
  misc_feature (56)..(56): S is G or C
  misc_feature (57)..(57): n is a, c, g, or u
  misc_feature (60)..(60): Y is C or T/U
  misc_feature (63)..(63): Y is C or T/U
  misc_feature (66)..(66): n is a, c, g, or u
  misc_feature (69)..(69): R is A or G
  misc_feature (72)..(72): n is a, c, g, or u
  misc_feature (75)..(75): Y is C or T/U
  misc_feature (78)..(78): H is A or C or T/U
  misc_feature (79)..(79): Y is C or T/U
  misc_feature (81)..(81): n is a, c, g, or u
  misc_feature (82)..(82): Y is C or T/U
  misc_feature (84)..(84): n is a, c, g, or u
  misc_feature (87)..(87): Y is C or T/U
  misc_feature (90)..(90): n is a, c, g, or u
ytnwsntayttyathgcnwsnttymgnytnttygcnmgnacnmgnwsnatgtggwsnttyaayccngaracnaayathytnytnaaygtn

SEQ ID NO: 267 - DNA, 90 bp, artificial sequence
  DNA encoding Covid-19 envelope peptide position 45-74
  misc_feature (3)..(3): Y is C or T/U
  misc_feature (6)..(6): H is A or C or T/U
  misc_feature (9)..(9): n is a, c, g, or u
  misc_feature (12)..(12): Y is C or T/U
  misc_feature (15)..(15): n is a, c, g, or u
  misc_feature (16)..(16): W is A or T/U
  misc_feature (17)..(17): S is G or C
  misc_feature (18)..(18): n is a, c, g, or u
  misc_feature (19)..(19): Y is C or T/U
  misc_feature (21)..(21): n is a, c, g, or u
  misc_feature (24)..(24): n is a, c, g, or u
  misc_feature (27)..(27): R is A or G
  misc_feature (30)..(30): n is a, c, g, or u
  misc_feature (31)..(31): W is A or T/U
  misc_feature (32)..(32): S is G or C
  misc_feature (33)..(33): n is a, c, g, or u
  misc_feature (36)..(36): Y is C or T/U
  misc_feature (39)..(39): Y is C or T/U
  misc_feature (42)..(42): n is a, c, g, or u
  misc_feature (45)..(45): Y is C or T/U
  misc_feature (46)..(46): W is A or T/U
  misc_feature (47)..(47): S is G or C
  misc_feature (48)..(48): n is a, c, g, or u
  misc_feature (49)..(49): M is A or C
  misc_feature (51)..(51): n is a, c, g, or u
  misc_feature (54)..(54): n is a, c, g, or u
  misc_feature (57)..(57): R is A or G
  misc_feature (60)..(61): Y is C or T/U
  misc_feature (63)..(63): n is a, c, g, or u
  misc_feature (66)..(66): Y is C or T/U
  misc_feature (67)..(67): W is A or T/U
  misc_feature (68)..(68): S is G or C
  misc_feature (69)..(69): n is a, c, g, or u
  misc_feature (70)..(70): W is A or T/U
  misc_feature (71)..(71): S is G or C
  misc_feature (72)..(72): n is a, c, g, or u
  misc_feature (73)..(73): M is A or C
  misc_feature (75)..(75): n is a, c, g, or u
  misc_feature (78)..(78): n is a, c, g, or u
  misc_feature (81)..(81): n is a, c, g, or u
  misc_feature (84)..(85): Y is C or T/U
  misc_feature (87)..(87): Y is C or T/U
  misc_feature (90)..(90): n is a, c, g, or u
aayathgtnaaygtnwsnytngtnaarccnwsnttytaygtntaywsnmgngtnaaraayytnaaywsnwsnmgngtnccngayytnytn

(19) United States
(12) Patent Application Publication
(10) Pub. No.: US 2023/0158137 A1
(43) Pub. Date: May 25, 2023

(54) CORONAVIRUS VACCINE

(71) Applicant: PepTC Vaccines Limited, London (GB)

(72) Inventors: Zsolt Csiszovszki, Budapest (HU); Orsolya Lörincz, Budapest (HU); Levente Molnár, Felsopakony (HU); Péter Páles, Budapest (HU); Katalin Pántya, Szigetszentmiklós (HU); Eszter Somogyi, Balatonalmádi (HU); József Tóth, Budapest (HU); Eniko Rita Toke, Felsopakony (HU)

(21) Appl. No.: 17/995,299

(22) PCT Filed: Apr. 1, 2021
(86) PCT No.: PCT/GB2021/050829

§ 371 Date: Sep. 30, 2022

Related U.S. Application Data
(63) Continuation of application No. 16/842,669, filed on Apr. 7, 2020, now Pat. No. 10,973,909.

(30) Foreign Application Priority Data
Apr. 3, 2020 (GB) 2004974.8
Oct. 12, 2020 (GB) 2016172.5

(51) Int. Cl. A61K 39/215; A61K 39/39; C07K 14/73; C07K 14/74; C07K 14/705

(52) U.S. Cl. CPC A61K 39/215; A61K 39/39; C07K 14/70514; C07K 14/70539; C07K 14/70517

(57) ABSTRACT

The disclosure relates to polypeptides, vaccines and pharmaceutical compositions that find use in the prevention or treatment of Coronaviridae or SARS-CoV-2 infection. The disclosure also relates to methods of treating or preventing Coronaviridae or SARS-CoV-2 infection in a subject. The polypeptides and vaccines comprise B cell epitopes and cytotoxic and helper T cell epitopes that are immunogenic in a high percentage of subjects in the human population.

### FIELD

The invention relates to polypeptides that find use in the prevention or treatment of Coronaviridae viral infection.

### BACKGROUND

Preliminary studies suggest that SARS-CoV-2 is similar to SARS-CoV, for which previous research data exist on protective immune responses. Various reports related to SARS-CoV suggest a protective role of both humoral and cell-mediated immune responses. Antibody responses generated against the spike (S) and nucleocapsid (N) protein of SARS-CoV were particularly prevalent in SARS-CoV-infected patients. While being effective, the antibody response was found to be short-lived in convalescent SARS-CoV patients. In contrast, T cell responses have been shown to provide long-term memory post-infection in recovered patients.

One of the challenges of producing an effective vaccine is that there is tremendous variability in the way the immune systems of different human subjects interact with the different antigens expressed by an infecting virus. The present inventors have previously shown that the immune responses of an individual subject are predicted by the ability of single antigen T cell epitopes to be recognised by multiple HLA alleles of the subject. T cell epitopes that are restricted to multiple HLA alleles of a subject act as genetic biomarkers that predict peptide-specific T cell responses of individual patients. These genetic biomarkers are referred to as “personal epitopes” or “PEPIs”. Multi-HLA allele-binding PEPIs induce T cell responses at a significantly higher rate than T cell epitopes that are restricted to a single HLA allele of a vaccinated subject. The identification of T cell epitopes in the polypeptides of a vaccine composition that are multi-HLA allele-binding PEPIs for subjects in a model human population has been shown to predict the immune response rates reported in clinical trials (WO 2018/158456, WO 2018/158457 and WO 2018/158455).

A second challenge in the development of an effective vaccine is the continuing evolution of the virus through mutation and the potential for infecting virus heterogeneity.

A third challenge is the need to quickly develop, safety test, and verify efficacy of a vaccine for the new emergent SARS-CoV-2 coronavirus, and subsequently manufacture the vaccine on a very large scale, to meet immediate population demands. Conventional vaccine development is a complex and challenging process. Peptide vaccines provide several advantages in comparison to conventional vaccines made of dead or attenuated pathogens, inactivated toxins, and recombinant subunits. Short polypeptides can be synthesized rapidly and peptide vaccine production is relatively inexpensive. Additionally, peptide vaccines avoid the inclusion of unnecessary components possessing high reactogenicity to the host, such as lipopolysaccharides, lipids, and toxins. The safety and immunogenicity of peptide vaccines with Montanide adjuvant has been proven in multiple clinical trials involving over 6,000 patients and over 200 healthy volunteers.

Peptide vaccine development strategy typically targets the selection of a combination of HLA allele-restricted epitopes that seek to maximize population coverage globally. According to this approach, multiple peptides are selected having different HLA binding specificities to afford increased coverage of the patient population targeted by peptide (epitope)-based vaccines, taking also in consideration that different HLA types are expressed at dramatically different frequencies in different ethnicities. SF Ahmed et al., for example, propose a screened set of T cell epitopes estimated to provide broad coverage of global population as well as in China against SARS-CoV-2 (Ahmed et al. Viruses, 12(3). 2020). They used HLA-restricted SARS-CoV-derived epitopes and the publicly available IEDB Population Coverage Tool (http://tools.iedb.org/population) to guide experimental efforts towards the development of vaccines against SARS-CoV-2.

This approach attempts to take in consideration HLA polymorphism and frequency in different ethnic populations. In practice, however, most often HLA-restricted epitopes do not induce an immune response in HLA-matched individuals, and clinical trials result in lower immune response rates than expected (Slingluff C L. Cancer J 2011; 17(5): 343-50). In addition peptides recognized by CD8 T cells have been shown to be both selective and extremely sensitive; one amino acid change can alter the specific epitope into a non-immunogenic peptide.

Other approaches include the whole sequence of S protein in mRNA or pDNA vectors. (Smith T R F et al. under review 10.21203/rs.3.rs-16261/v1; Safety and Immunogenicity Study of 2019-nCoV Vaccine (mRNA-1273) for Prophylaxis SARS CoV-2 Infection, NCT04283461).

Accordingly, there is an immediate need for a vaccine that is effective in a high proportion of the global human population, robust to viral antigen mutation, and could proceed rapidly through the necessary steps for clinical validation and manufacture.

### SUMMARY OF THE INVENTION

The inventors have focused efforts on the development of a global peptide vaccine against SARS-CoV-2 that addresses the dual challenges of heterogeneity in the immune responses of different individuals and potential heterogeneity in the infecting virus. The peptide design concept detailed here merges shared personal epitope design with the further selection of B cell epitope sequences resulting in overlapping, multi-HLA binding epitopes within an individual aiming to induce CD4+, CD8+ and antibody-producing B-cell responses. Accordingly, the inventors have identified 30mer polypeptide fragments of the conserved regions of the presently known SARS-CoV-2 viral antigens that comprise (i) maximum CD8+ personal epitopes (PEPIs) in a model population of human subjects having HLA genotypes that are representative of the global population; (ii) maximum CD4+ personal epitopes (PEPIs) in the global population; and (iii) linear B cell epitopes. Peptide vaccines comprising the polypeptides identified by the inventors are predicted to induce cytotoxic T cell, helper T cell and B cell responses in a surprisingly high proportion of subjects in the human population. Even higher response rates in the human population, and continued efficacy against an evolving heterologous infecting virus, can be achieved by combining more than one of the antigen fragments identified by the inventors, preferably by combining antigen fragments identified by the inventors from different SARS-CoV-2 structural proteins.

Accordingly, in a first aspect the disclosure provides a polypeptide or a panel of polypeptides of up to 50 amino acids in length, or up to 60 amino acids in length, wherein the polypeptide comprises or consists of an amino acid sequence selected from SEQ ID NOs: 1 to 17.

In a further aspect, the disclosure provides a panel of polypeptides of up to 50 amino acids in length, or up to 60 amino acids in length, wherein each polypeptide comprises or consists of a different amino acid sequence selected from SEQ ID NOs: 1 to 17.

One or more of the polypeptides may consist of a fragment of a Coronaviridae, Beta-coronaviridae or SARS-CoV-2 protein. Each of the polypeptides may comprise an amino acid sequence selected from SEQ ID NOs: 1 to 17 that is a fragment of a different Coronaviridae, Beta-coronaviridae or SARS-CoV-2 protein. The panel of polypeptides may include at least one sequence from at least two, three or all four of the following groups: (a) SEQ ID NOs: 1 to 11 (fragments of SARS-CoV-2 surface protein); (b) SEQ ID NOs: 12 to 15 (fragments of SARS-CoV-2 nucleocapsid protein); (c) SEQ ID NO: 16 (fragment of SARS-CoV-2 membrane protein); and (d) SEQ ID NO: 17 (fragment of SARS-CoV-2 envelope protein). In some cases, the panel of peptides may comprise at least 3, 4, 5, 6, 7, 8, 9, 10, 11, 12, 13, 14, 15, 16 or 17 different polypeptides, each comprising a different amino acid sequence selected from SEQ ID NOs: 1 to 17. In one embodiment, the panel comprises the amino acid sequences of SEQ ID NOs: 2, 5, 7, 9, 12, 13, 14, 15, 16, and 17. In one embodiment, the panel comprises the amino acid sequences of SEQ ID NOs: 2, 5, 9, 12, 13, 14, 15, 16, and 17. In one embodiment, the panel comprises the amino acid sequences of SEQ ID NOs: 6, and 9 to 17. In one embodiment, the panel of polypeptides comprises ten polypeptides, wherein each of the ten polypeptide comprises or consists of a different amino acid sequence selected from SEQ ID NOs: 2, 5, 7, 9, 12, 13, 14, 15, 16, and 17. In another embodiment, the panel of polypeptides comprises nine polypeptides, wherein each of the nine polypeptide comprises or consists of a different amino acid sequence selected from SEQ ID NOs: 2, 5, 9, 12, 13, 14, 15, 16, and 17. In another embodiment, the panel of polypeptides comprises ten polypeptides wherein each of the ten polypeptide comprises or consists of a different amino acid sequence selected from SEQ ID NOs: 6, and 9 to 17.

In a further aspect, the present disclosure provides a pharmaceutical composition or kit having the polypeptide or panel of polypeptides described above as active ingredient(s). In some cases, the pharmaceutical composition or kit may comprise ribonucleic acid that encodes each of the polypeptide(s).

In a further aspect, the present disclosure provides a method of vaccination, providing immunotherapy or inducing immune responses in a subject, the method comprising administering to the subject the polypeptide, panel of polypeptides or pharmaceutical composition described above. In some cases the method is a method of treating a Coronaviridae infection, Beta-coronaviridae infection, SARS-CoV-2 infection, or a SARS-CoV infection, a disease or condition associated with a Coronaviridae or Beta-coronaviridae infection, COVID-19 or Severe Acute Respiratory Syndrome (SARS) in the subject. In some cases the method is a method of preventing a Coronaviridae infection, Beta-coronaviridae infection, a SARS-CoV-2 or a SARS-CoV infection, or of preventing the development of a disease or condition associated with a Coronaviridae or Beta-coronaviridae infection, or of COVID-19 or SARS in the subject. In some cases, at least one, or at least two, three, four, five, six or each of the polypeptides comprises a fragment of a Coronaviridae protein, a Beta-coronaviridae protein, a SARS-CoV-2, or a SARS-CoV protein that is a CD8+ T cell epitope predicted to be restricted to at least two, or in some cases three or at least three, HLA class I alleles of the subject. In some cases, at least one, or at least two, three, four, five, six or each of the polypeptides comprises a fragment of a Coronaviridae protein, a Beta-coronaviridae protein, a SARS-CoV-2, or a SARS-CoV protein that is a CD4+ T cell epitope predicted to be restricted to at least two, or in some cases at least three, or in some cases four or at least four, HLA class II alleles of the subject. In some cases, at least one, or at least two, three, four, five, six or each of the polypeptides comprises a linear B cell epitope.

In further aspects, the disclosure provides
a peptide, panel of polypeptides or pharmaceutical composition as described above for use in a method of vaccination, providing immunotherapy or inducing a cytotoxic T cell response in a subject, or in a method of treating or preventing or a Coronaviridae infection, a Beta-coronaviridae infection, a SARS-CoV-2 infection or a SARS-CoV infection, or treating or preventing the development of a disease or condition associated with a Coronaviridae or Beta-coronaviridae infection, or COVID-19 or SARS in a subject, optionally wherein the subject is as described above; and
use of a polypeptide, a panel of polypeptides or one or more polynucleotides or cells encoding a polypeptide or panel of polypeptides as described above in the manufacture of a medicament for vaccination, providing immunotherapy or inducing a cytotoxic T cell response in a subject, or in a method of treating or preventing Coronaviridae infection, a Beta-coronaviridae infection, SARS-CoV-2 infection or a SARS-CoV infection, treating or preventing the development of a disease or condition associated with a Coronaviridae or Beta-coronaviridae infection, or or COVID-19 or SARS in a subject, optionally wherein the subject is as described above.

In some cases, the methods described herein may comprise the step of determining the HLA class I and/or class II genotype of the subject.

The disclosure will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “a peptide” includes two or more such peptides.

Section headings are used herein for convenience only and are not to be construed as limiting in any way.

### DESCRIPTION OF THE FIGURES

FIG. 1. Immune responses measured by enriched ELISPOT assay upon vaccination with PolyPEPI1018 vaccine. A) Number of vaccine antigens with immune response plotted by patients. Dark grey solid bars: decreased or no change compared to pre-vaccination, dark grey striped bars: response boosted compared to pre-vaccination (at least twofold increase), light grey striped bars: de novo induced vaccine-specific immune responses. B) PolyPEPI1018 vaccine-specific CD4 T cell responses detected pre-vaccination (black), after one vaccination (dark grey), after two doses (light grey), and after 3 doses (white), C) Time course of the immune response through baseline (pre-vaccination) and week 12 measured after a single vaccination for the eight patients who had data for all three points. Each line represents a single patient (n=8).

FIG. 2. Average distribution of vaccine-specific polyfunctional CD8 (A) and CD4 (B) T cell responses (n=9, measured after a single dose of PolyPEPI1018 vaccine).

FIG. 3. TIC detected TILs in the pre-vaccination (PRE) and 38 week (POST) sample of patient 010007.

FIG. 4. Clinical response of patients. A) Swimmer plot of the 11 enrolled patients with response to first line therapy and RECIST assessment results during the trial, B) Spider plot showing the changes of the target lesion volumes (summed) during the OBERTO-101 trial. Each data point is compared to the lesion size measured at baseline (pre-vaccination), C) Kaplan-Meier analysis of the progression-free survival of the single- and multiple dose groups.

FIG. 5. Target lesion size changes of the responder patients. A) Patient 020004 receiving a single dose, having two target lesions at baseline, B) Patient 010004 receiving multiple doses, having three target lesions at baseline, C) Patient 010007 receiving multiple doses, having one target lesion at baseline, and D) Patient 010002 receiving multiple doses, having one target lesion at baseline.

FIG. 6. Predicted (A-B) and measured (C-D) multiantigenic immune response (AGP) tends to correlate with tumor volume reduction (A and C) and PFS (B and D) in the multiple dose group (n=5).

FIG. 7. Comparison of predicted vaccine induced immune response rates (CD8) for randomly selected Epitope Vaccine proposed by SF Ahmed et al. and 10 peptides of PolyPEPI-SCoV-2 vaccine in ˜16,000 subjects of 16 ethnicities.

FIG. 8. Comparison of predicted vaccine induced immune response rates (CD8) for all 59 peptides selected by SF Ahmed et al. or 10 peptides of PolyPEPI-SCoV-2 vaccine in ˜16,000 subjects of 16 ethnicities.

FIG. 9. Proportion of subjects having both CD4 and CD8 T cells against at least 2 peptides of PolyPEPI-SCoV-2 vaccine. Prediction was performed in the ˜16,000 subject cohort of 16 ethnicities.

FIG. 10. Proportion of subjects (in the ˜16,000 cohort) having immune response against≥1-10 vaccine epitopes induced by randomly selected Epitope Vaccine proposed by SF Ahmed et al. and 10 peptides of PolyPEPI-SCoV-2 vaccine.

FIG. 11. Proportion of subjects (in the ˜16,000 cohort) having immune response against≥1-10 vaccine epitopes induced by all 59 peptides of the Epitope Vaccine proposed by SF Ahmed et al. or the 10 peptides of PolyPEPI-SCoV-2 vaccine.

FIG. 12. Hotspot analysis of SARS-CoV-2 Spike-1 protein in the ethnically diverse in silico human cohort. Analysis was performed by predicting≥3 HLA allele binding personal epitopes (PEPIs) for each subject. Left panel: Each row along the vertical axis represents one subject in the model population, while the horizontal axis represents the SARS-CoV-2 S-1 protein subunit sequence. Vertical bands represent the most frequent epitopes, i.e., the dominant immunogenic protein regions (hotspots) or PEPIs for most subjects. CEU, Central European; CHB, Chinese; JPT, Japanese; YRI, African; Mix, mixed ethnicity subjects. Colors represent the number of epitopes restricted to a person: light grey, 3; radium grey, 4; black, >5. A PEPI was defined as an epitope restricted to ≥3 alleles of a person. Right panel, average number of epitopes/PEPIs found for subjects of different ethnicities.

FIG. 13. IFN-γ+T cell responses elicited by PolyPEPI-SCoV-2 vaccination in two animal models. Fold change in PolyPEPI-SCoV-2 vaccine-induced T cell responses in BALB/c (A) and humanized (Hu-mice) (B) mice compared with the respective control cohorts receiving Vehicle only. Vaccine-induced T cell responses specific for SARS-CoV-2 protein-derived vaccine peptides after two doses detected at day 28 in BALB/c (C) and humanized (Hu-mice) (D). Test conditions: S-pool contains the three peptides derived from S protein; N-pool contains the four peptides derived from N protein; M and E are the peptides derived from M and E proteins, respectively, in both the 9-mer and 30-mer pools. Results were compared to Vehicle (DMSO/Water emulsified with Montanide) control group of the same time point. Ex vivo ELISpot assays were performed by stimulation with 9-mer and 30-mer peptides. Mice received two doses of vaccine or Vehicle at days 0 and 14. Each cohort comprised six animals at each timepoint. Spot forming unit (SFU) represents unstimulated background corrected values given for 2*10⁵ splenocytes. t-test was used to calculate significance.

FIG. 14. The PolyPEPI-SCoV-2 treatment increases IFN-γ-producing T cells in mice. PolyPEPI-SCoV-2 vaccinated mice are shown with dark grey dots, and compared to Vehicle (DMSO/Water emulsified with Montanide) control animals shown in light grey dots. IFN-γ production was analyzed by ex vivo ELISpot in the spleen after re-stimulation with peptides at day 14 (A, BALB/c; and D, Hu-mice), day 21 (B, BALB/c; and E, Hu-mice), and day 28 (C, BALB/c; and F, Hu-mice). Condition 1, S-pool; Spike-specific 30-mer pool of S2, S5, and S9 peptides. Condition 2, N-pool; Nucleoprotein-specific 30-mer pool of N1, N2, N3, and N4 peptides. Condition 3, M1 Membrane-specific 30-mer peptide. Condition 4, E1 Envelope-specific 30-mer peptide. Condition 5, S-pool; Spike-specific 9-mer pool of s2, s5, and s9 HLA class I PEPI hotspot fragment of the corresponding 30-mers. Condition 6, N-pool; Nucleoprotein-specific 9-mer pool of n1, n2, n3, and n4 HLA class I PEPI hotspot fragment of the corresponding 30-mers. Condition 7, ml Membrane-specific 9-mer HLA class I PEPI hotspot fragment of the corresponding 30-mer. Condition 8, el Envelope-specific 9-mer HLA class I PEPI hotspot fragment of the corresponding 30-mer. Condition 9, unstimulated control. Individual spot forming cell (SFC) values and means are shown and represent spots per 2*10⁵ splenocytes. n=6 mice per group were analyzed. Statistical analysis was performed by Mann-Whitney test. *, p<0.05; **, p<0.01.

FIG. 15. Th1 dominant immune response and no induction of significant Th2 cytokines with PolyPEPI-SCoV-2 in mice models. Average CD4⁺ and CD8⁺ T cells producing IL2, TNF-α, IFN-γ, IL-5 or IL10 in immunized and Vehicle control groups for both BALB/c (A) and Hu-mice models (B), using ICS assay. Mean+/−SEM are shown. 2*10⁵ cells were analyzed, gated for CD45⁺ cells, CD3⁺ T cells, CD4⁺ or CD8⁺ T cells. The average percent was obtained by pooling the background subtracted values of the 4 stimulation conditions (30-mer S-pool, N-pool, E1 and M1 peptides) for each cytokine for CD4⁺ and CD8⁺ T cells.

FIG. 16. Th1/Th2 balance for BALB/c mice at day 28. Average CD4⁺ and CD8⁺ T cells producing IL2, TNF-α, IFN-γ (Th1 cytokines) and IL10 (Th2 cytokine) for each immunized mice (n=6) using ICS assay. 2*10⁵ cells were analyzed, gated for CD45⁺ cells, CD3⁺ T cells, CD4⁺ or CD8⁺ T cells. The average percent was obtained by pooling the background subtracted values of the 4 stimulation conditions (30-mer S-pool, N-pool, E1 and M1 peptides) for each cytokine for CD4⁺ and CD8⁺ T cells.

FIG. 17. Vaccine-induced IgG production measured from the plasma of BALB/c mice (A) and Hu-mice (B) models. Mice received two doses of PolyPEPI-SCoV-2 vaccine or Vehicle at days 0 and 14. Each cohort comprised six animals. t-tests were used to calculate significance. *, p<0.05

FIG. 18. Cytokine production by COVID-19 convalescents' T cells reactive to PolyPEPI-SCoV-2 peptides determined ex vivo from their PBMC by intracellular staining assay. (A) Cytokine profile of CD4⁺ and CD8⁺ T cells+ obtained by stimulations with 9-mer and 30-mer peptides (n=17); (B) Th1 dominance in vaccine-specific T cells stimulated with 30-mer peptides.

FIG. 19. PolyPEPI-SCoV-2-specific T cell responses from COVID-19 convalescent donors. A. Highly specific vaccine-derived 9-mer peptide-reactive CD8⁺ T cell responses and 30-mer peptide-reactive CD4⁺ T cell responses detected by ex vivo FluoroSpot assay. B. Enriched ELISpot results with 30-mer peptide pools, 9-mer peptide pools, and commercial SNMO peptide pool-activated T cells. C. Enriched ELISpot results with CD8⁺ T cells activated by individual 9-mer peptides corresponding to each of the 30-mer peptides with the same name (Table 9 bold). dSFU, delta spot forming units, calculated as non-stimulated background corrected spot counts per 10⁶ PBMC.

FIG. 20. IFN-γ⁺ T cell responses detected for COVID-19 convalescent donors against the 9-mer peptides (PEPI hotspots) of PolyPEPI-SCoV-2 vaccine measured by enriched ELISpot assay. s2, s5, and s9 are the three S-specific 9-mer peptide sequences derived from the Spike-specific vaccine 30-mers. n1-n4 are the four Nucleoprotein-specific 9-mer peptide sequences derived from the N-specific vaccine 30-mers. el and ml are Envelope and Membrane-specific 9-mer peptide sequences derived from the E or M-specific vaccine 30-mers, respectively (Table 9 Bold). dSFU, delta spot forming units calculated as non-stimulated background corrected spot counts per 10⁶ PBMC. Average and individual data for each subject are presented. PBMC, peripheral blood mononuclear cells.

FIG. 21. Magnitude and breadth of COVID-19 convalescent donors' T cell responses relative to time from symptom onset. A) Magnitude of PolyPEPI-SCoV-2-reactive T cell responses B) Breadth of vaccine peptide-reactive CD8⁺ T cell responses from convalescent donors, detected with enriched ELISpot assay. dSFU stands for delta spot forming units, calculated as non-stimulated background corrected spot counts per 10⁶ PBMC. Statistical analysis: Pearson correlation analysis. R-Pearson correlation coefficient.

FIG. 22. Correlation between SARS-CoV-2-specific antibody levels and PolyPEPI-SCoV-2-specific IFN-γ-producing CD4⁺ T cells in COVID-19 convalescent individuals. A) T cell responses reactive to 30-mer pool of PolyPEPI-SCoV-2 peptides were plotted against the IgG-S1 (EUROIMMUN). B) Average T cell responses reactive to S-1 protein-derived 30-mer peptides (S2 and S5) was plotted against IgG-S1 (EUROIMMUN). C) T cell responses reactive to 30-mer N peptide pool comprising N1, N2, N3 and N4 was plotted against total IgG-N measured with Roche Elecsys® assay. D) T cell responses reactive to 30-mer pool of PolyPEPI-SCoV-2 peptides were plotted against the IgA antibody amounts measured by DiaPro IgA ELISA assay. Correlation analysis was performed by Pearson's test. R-correlation coefficient.

FIG. 23. Predicted global coverage in a large population with different ethnicities. A) Proportion of subjects having HLA class I PEPIs against at least one of the nine PolyPEPI-SCoV-2 vaccine peptides B) Proportion of subjects having both HLA class I and class II PEPIs against at least two peptides in the PolyPEPI-SCoV-2 vaccine. C) Theoretical global coverage estimated based on the frequency of six selected HLA alleles (A*02:01, A*01:01, A*03:01, A*11:01, A*24:02, and B*07:02), as proposed by Ferretti et al.(27)

FIG. 24. Allele frequency distributions in the model population representative for global allele frequencies. HLA allele frequencies in the Model Population represent similar distribution as the allele frequencies of >8 million HLA-genotyped subjects in the CIWD database. CIWD 3.0: Common (>=1 in 10,000), Intermediate (>=1 in 100,000) and Well Documented (>=5 occurrence) HLA database 3.0 (released in 2020). R-Pearson correlation coefficient. Related to FIG. 20.

FIG. 25. Correlation between multiple autologous allele-binding epitopes and CD8+ T cell responses in COVID-19 convalescents. Matching predicted multiple autologous HLA binding epitopes (n=9) with the same peptide-reactive CD8+ T cell responses in n=15 donors (135 data points). Numbers denote dSFU determined by enriched FluoroSpot assay. Colour codes refer to the predicted number of autologous HLA alleles binding the specific peptides. dSFU, delta spot forming units calculated as non-stimulated background corrected spot counts per 10⁶ PBMC.

FIG. 26. SARS-CoV-2 S1-protein specific epitope generation capacity of individuals with different ethnicities based on their complete HLA genotype. Related to FIG. 12. For each of S2, S5, S9, N1, N2, N3, N4, M1 and E1, going from left to right, the bars correspond to “All” “CEU”, “CHB”, “JPT”, “YRI” and “MIX” respectively.

FIG. 27. Multi-peptide response rate in the Model Population (N=433) (A) and in the large population, N=16,000 (B) predicted for shared SARS-CoV epitopes from the 17 30-mer peptides originally designed for SARS-CoV-2.

FIG. 28. Multi-antigen (multi-protein) response rate in the Model Population (N=433) (A) and in the large population, N=16,000 (B) predicted for shared SARS-CoV epitopes from the 17 30-mer peptides originally designed for SARS-CoV-2.

FIG. 29. Correlation between multiple autologous HLA allele-binding epitopes and PolyPEPI-SCoV-2-specific IFN-γ-producing CD8+ T cell responses in COVID-19 convalescent individuals. (A) Correlation between multiple autologous HLA allele-binding epitopes and magnitude of T cell responses. Rs—Spearman coefficient (confirmed by Pearson correlation analysis, too) (B) Magnitude of CD8+ T cell responses detected for PEPIs (binding≥3 autologous HLA class I alleles) and for non-PEPIs (binding≤3 autologous HLA class I alleles) by enriched FluoroSpot assay, (p=0.008, t-test). Median and individual data for each subject are presented, n=15 (C) Variable dependency analysis using 2×2 contingency table and Fisher Exact test. (D) Confirmation of Personal Epitopes (PEPIs) by IFN-γ producing CD8+ T cells for each subject [Positive Predictive Value (PPV)=True positive/Total predicted=37/44 (84%)]. dSFU, delta spot forming units calculated as non-stimulated background corrected spot counts per 10⁶ PBMC. PBMC, peripheral blood mononuclear cells.

### DESCRIPTION OF THE SEQUENCES

SEQ ID NOs: 1 to 17 set forth 30 mer T cell epitopes described in Table 6A.

SEQ ID NOs: 18-233 set forth various sequences as disclosed herein.

SEQ ID NOs: 234 to 267 set forth the corresponding RNA or DNA sequences encoding the peptides of SEQ ID Nos: 1 to 17, as shown in Table 15.

### DETAILED DESCRIPTION

### HLA Genotypes

HLAs are encoded by the most polymorphic genes of the human genome. Each person has a maternal and a paternal allele for the three HLA class I molecules (HLA-A*, HLA-B*, HLA-C*) and four HLA class II molecules (HLA-DP*, HLA-DQ*, HLA-DRB1*, HLA-DRB3*/4*/5*). Practically, each person expresses a different combination of 6 HLA class I and 8 HLA class II molecules that present different epitopes from the same protein antigen.

The nomenclature used to designate the amino acid sequence of the HLA molecule is as follows: gene name*allele:protein number, which, for instance, can look like: HLA-A*02:25. In this example, “02” refers to the allele. In most instances, alleles are defined by serotypes—meaning that the proteins of a given allele will not react with each other in serological assays. Protein numbers (“25” in the example above) are assigned consecutively as the protein is discovered. A new protein number is assigned for any protein with a different amino acid sequence (e.g. even a one amino acid change in sequence is considered a different protein number). Further information on the nucleic acid sequence of a given locus may be appended to the HLA nomenclature.

The HLA class I genotype or HLA class II genotype of an individual may refer to the actual amino acid sequence of each class I or class II HLA of an individual, or may refer to the nomenclature, as described above, that designates, minimally, the allele and protein number of each HLA gene. An HLA genotype may be determined using any suitable method. For example, the sequence may be determined via sequencing the HLA gene loci using methods and protocols known in the art. Alternatively, the HLA set of an individual may be stored in a database and accessed using methods known in the art.

Some subjects may have two HLA alleles that encode the same HLA molecule (for example, two copies for HLA-A*02:25 in case of homozygosity). The HLA molecules encoded by these alleles bind all of the same T cell epitopes. For the purposes of this disclosure “binding to at least two HLA molecules of the subject” as used herein includes binding to the HLA molecules encoded by two identical HLA alleles in a single subject. In other words, “binding to at least two HLA molecules of the subject” and the like could otherwise be expressed as “binding to the HLA molecules encoded by at least two HLA alleles of the subject”.

### Polypeptides

The disclosure relates to polypeptides that are derived from SARS-CoV-2 antigens and that are immunogenic for a high proportion of the human population.

As used herein, the term “polypeptide” refers to a full-length protein, a portion of a protein, or a peptide characterized as a string of amino acids. As used herein, the term “peptide” refers to a short polypeptide comprising between 2, or 3, or 4, or 5, or 6, or 7, or 8, or 9, or 10, or 11, or 12, or 13, or 14, or 15 and 10, or 11, or 12, or 13, or 14, or 15, or 20, or 25, or 30, or 35, or 40, or 45, or 50 or 55 or 60 amino acids.

The terms “fragment” or “fragment of a polypeptide” as used herein refer to a string of amino acids or an amino acid sequence typically of reduced length relative to the or a reference polypeptide and comprising, over the common portion, an amino acid sequence identical to the reference polypeptide. Such a fragment according to the disclosure may be, where appropriate, included in a larger polypeptide of which it is a constituent. In some cases the fragment may comprise the full length of the polypeptide, for example where the whole polypeptide, such as a 9 amino acid peptide, is a single T cell epitope. In some cases the fragments referred to herein may be between 8 or 9 and 20, or 25, or 30, or 35, or 40, or 45, or 50 amino acids.

As used herein, the term “epitope” or “T cell epitope” refers to a sequence of contiguous amino acids contained within a protein antigen that possess a binding affinity for (is capable of binding to) one or more HLAs. An epitope is HLA- and antigen-specific (HLA-epitope pairs, predicted with known methods), but not subject specific. An epitope, a T cell epitope, a polypeptide, a fragment of a polypeptide or a composition comprising a polypeptide or a fragment thereof is “immunogenic” for a specific human subject if it is capable of inducing a T cell response (a cytotoxic T cell response or a helper T cell response) in that subject. In some cases the helper T cell response is a Th1-type helper T cell response. The terms “T cell response” and “immune response” are used herein interchangeably, and refer to the activation of T cells and/or the induction of one or more effector functions following recognition of one or more HLA-epitope binding pairs. In some cases an “immune response” includes an antibody response, because HLA class II molecules stimulate helper responses that are involved in inducing both long lasting CTL responses and antibody responses. Effector functions include cytotoxicity, cytokine production and proliferation. According to the present disclosure, an epitope, a T cell epitope, or a fragment of a polypeptide is immunogenic for a specific subject if it is capable of binding to at least two, or in some cases at least three, class I or at least two, or in some cases at least three or at least four class II HLAs of the subject.

A “personal epitope” (or “PEPI”) is a fragment of a polypeptide consisting of a sequence of contiguous amino acids of the polypeptide that is a T cell epitope capable of binding to one or more HLA class I molecules of a specific human subject. In other cases a “PEPI” is a fragment of a polypeptide consisting of a sequence of contiguous amino acids of the polypeptide that is a T cell epitope capable of binding to one or more HLA class II molecules of a specific human subject. In other words, a “PEPI” is a T cell epitope that is recognised by the HLA set of a specific individual, and is consequently specific to the subject in addition to the HLA and the antigen. In contrast to an “epitope”, which is specific only to HLA and the antigen, PEPIs are specific to an individual because different individuals have different HLA molecules which each bind to different T cell epitopes.

“PEPI1” as used herein refers to a peptide, or a fragment of a polypeptide, that can bind to one HLA class I molecule (or, in specific contexts, HLA class II molecule) of an individual. “PEPI1+” refers to a peptide, or a fragment of a polypeptide, that can bind to one or more HLA class I molecule of an individual. “PEPI2” refers to a peptide, or a fragment of a polypeptide, that can bind to two HLA class I (or II) molecules of an individual. “PEPI2+” refers to a peptide, or a fragment of a polypeptide, that can bind to two or more HLA class I (or II) molecules of an individual. “PEPI3” refers to a peptide, or a fragment of a polypeptide, that can bind to three HLA class I (or II) molecules of an individual. “PEPI3+” refers to a peptide, or a fragment of a polypeptide, that can bind to three or more HLA class I (or II) molecules of an individual. “PEPI4” refers to a peptide, or a fragment of a polypeptide, that can bind to three HLA class I (or II) molecules of an individual. “PEPI4+” refers to a peptide, or a fragment of a polypeptide, that can bind to three or more HLA class I (or II) molecules of an individual.

Generally speaking, epitopes presented by HLA class I molecules are about nine amino acids long and epitopes presented by HLA class II molecules are about fifteen amino acids long. For the purposes of this disclosure, however, an epitope may be more or less than nine (for HLA Class I) or fifteen (for HLA Class II) amino acids long, as long as the epitope is capable of binding HLA. For example, an epitope that is capable of binding to class I HLA may be between 7, or 8 or 9 and 9 or 10 or 11 amino acids long. An epitope that is capable of binding to a class II HLA may be between 13, or 14 or 15 and 15 or 16 or 17 amino acids long.

A given HLA of a subject will only present to T cells a limited number of different peptides produced by the processing of protein antigens in an antigen presenting cell (APC). As used herein, “display” or “present”, when used in relation to HLA, references the binding between a peptide (epitope) and an HLA. In this regard, to “display” or “present” a peptide is synonymous with “binding” a peptide.

Using techniques known in the art, it is possible to determine the epitopes that will bind to a known HLA. Any suitable method may be used, provided that the same method is used to determine multiple HLA-epitope binding pairs that are directly compared. For example, biochemical analysis may be used. It is also possible to use lists of epitopes known to be bound by a given HLA. It is also possible to use predictive or modelling software to determine which epitopes may be bound by a given HLA. Examples are provided in Table 1. In some cases a T cell epitope is capable of binding to a given HLA if it has an IC50 or predicted IC50 of less than 5000 nM, less than 2000 nM, less than 1000 nM, or less than 500 nM.

TABLE 1
Example software for determining epitope-HLA binding

| EPITOPE PREDICTION TOOLS | WEB ADDRESS |
|---|---|
| BIMAS, NIH | www-bimas.cit.nih.gov/molbio/hla_bind/ |
| PPAPROC, Tubingen Univ. | |
| MHCPred, Edward Jenner Inst, of | |
| Vaccine Res. | |
| EpiJen, Edward Jenner Inst, of | http://www.ddg-pharmfac.net/ |
| Vaccine Res. | epijen/EpiJen/EpiJen.htm |
| NetMHC, Center for Biological | http://www.cbs.dtu.dk/services/NetMHC/ |
| Sequence Analysis | |
| SVMHC, Tubingen Univ. | http://abi.inf.uni-tuebingen.de/Services/SVMHC/ |
| SYFPEITHI, Biomedical Informatics, | http://www.syfpeithi.de/bin/MHCServer.dll/ |
| Heidelberg | EpitopePrediction.htm |
| ETK EPITOOLKIT, Tubingen Univ. | http://etk.informatik.uni-tuebingen.de/epipred/ |
| PREDEP, Hebrew Univ. Jerusalem | http://margalit.huji.ac.il/Teppred/mhc-bind/index.html |
| RANKPEP, MIF Bioinformatics | http://bio.dfci.harvard.edu/RANKPEP/ |
| IEDB, Immune Epitope Database | http://tools.immuneepitope.org/main/html/ |
| | tcell_tools.html |
| EPITOPE DATABASES | WEB ADDRESS |
| MHCBN, Institute of Microbial | http://www.imtech.res.in/raghava/mhcbn/ |
| Technology, Chandigarh, INDIA | http://www.syfpeithi.de/ |
| SYFPEITHI, Biomedical Informatics, | |
| Heidelberg | |
| AntiJen, Edward Jenner Inst, of | http://www.ddg- |
| Vaccine Res. | pharmfac.net/antijen/AntiJen/antijenhomepage.htm |
| EPIMHC database of MHC ligands, | http://immunax.dfci.harvard.edu/epimhc/ |
| MIF Bioinformatics | |
| IEDB, Immune Epitope Database | http://www.iedb.org/ |

The peptides of the disclosure may comprise or consist of one or more fragments of one or more Coronaviridae, a Beta-coronaviridae or SARS-CoV-2 antigens selected from surface glycoprotein, alias Spike, nucleocapsid phosphoprotein, envelope protein and membrane glycoprotein. Reference sequences are provided herein.

In some cases the amino acid sequence is flanked at the N and/or C terminus by additional amino acids that are not part of the sequence of the Coronaviridae, Beta-coronaviridae or SARS-CoV-2 antigen, in other words that are not the same sequence of consecutive amino acids found adjacent to the selected fragments in the target polypeptide antigen. In some cases the sequence is flanked by up to 20, 19, 18, 17, 16, 15, 14, 13, 12, 11, 10, 9, 8, 7, 6, 5, 4, 3, 2 or 1 additional amino acids at the N and/or C terminus.

The inventors have previously found that the presence in a cancer vaccine of at least two polypeptide fragments (epitopes) that can bind to at least three HLA class I of an individual (≥2 PEPI3+) is predictive for a clinical response. In other words, if ≥2 PEPI3+ can be identified within the active ingredient polypeptide(s) of a vaccine, then an individual is a likely clinical responder.

Without wishing to be bound by theory, the inventors believe that one reason for the increased likelihood of deriving clinical benefit from a vaccine/immunotherapy comprising at least two multiple-HLA binding PEPIs, is that diseased cell populations, such as cancer or tumor cells or cells infected by viruses or pathogens such as HIV, are often heterogeneous both within and between effected subjects. In addition, the likelihood of developing resistance is decreased when more multiple HLA-binding PEPIs are included or targeted by a vaccine because a patient is less likely to develop resistance to the composition through mutation of the target PEPI(s).

Likewise, in the context of a vaccine for a viral infection, where the viral infection may be heterologous, it is advantageous to administer to a subject vaccine peptide(s) that are predicted to comprise multiple subject-specific multi-HLA allele-binding PEPIs (for treatment of a subject having a known HLA genotype) or multiple population bestEPIs, i.e. amino acid sequences that are or comprise multi-HLA allele-binding PEPIs in a high proportion of the target population. Including more bestEPI sequences also increases the total proportion of human subjects that will respond to treatment. Accordingly, in some cases, the panel of polypeptides comprises at least 2, 3, 4, 5, 6, 7, 8, 9, 10, 11, 12, 13, 14, 15, 16 or 17 polypeptides, each comprising a different amino acid sequence selected from SEQ ID NOs: 1 to 17. In some cases the combination of polypeptide excludes one or more of the following combinations: SEQ ID NOs: 1 and 2; SEQ ID NOs: 3 and 4; SEQ ID NOs: 7 and 8; and/or SEQ ID NOs: 9 and 10; and/or excludes one or more of the following combinations: SEQ ID NOs: 2 and 3; and/or SEQ ID NOs: 13 and 14.

The polypeptides may be or comprise fragments of the same or different viral antigens. Different viral structural proteins may tend to mutate at different rates. Hence, in some cases each polypeptide comprises an amino acid sequence selected from SEQ ID NOs: 1 to 17 that is a fragment of a different Coronaviridae, a Beta-coronaviridae or SARS-CoV-2 protein. In some cases the panel of polypeptides includes at least one sequence from at least two, three or all four of the following groups: (a) SEQ ID NOs: 1 to 11 (fragments of SARS-CoV-2 surface protein), optionally excluding the combination of SEQ ID NOs: 1 and 2, SEQ ID NOs: 2 and 3, SEQ ID NOs: 3 and 4, SEQ ID NOs: 7 and 8, and/or SEQ ID NOs: 9 and 10; (b) SEQ ID NOs: 12 to 15 (fragments of SARS-CoV-2 nucleocapsid protein), optionally excluding the combination of SEQ ID NOs: 13 and 14; (c) SEQ ID NO: 16 (fragment of SARS-CoV-2 membrane protein); and (d) SEQ ID NO: 17 (fragment of SARS-CoV-2 envelope protein). In some cases the combination of polypeptides comprises or consists of ten polypeptides comprising or consisting of the amino acid sequences of SEQ ID NOs: 2, 5, 7, 9, 12, 13, 14, 15, 16, and 17. In another embodiment, the panel of polypeptides comprises nine polypeptides comprising or consisting of the amino acid sequences of SEQ ID NOs: 2, 5, 9, 12, 13, 14, 15, 16, and 17. In another embodiment, the panel of polypeptides comprises ten polypeptides comprising or consisting of the amino acid sequences of SEQ ID NOs: 6, and 9 to 17.

### Selection of Polypeptides and Patients

The peptides described herein may be used to induce T cell responses or provide vaccination or immunotherapy in a subject in need therefore. The peptides may be used to treat or prevent a Coronaviridae infection, a Beta-coronaviridae infection SARS-CoV-2 infection, SARS-CoV infection, disease or condition associated with a Coronaviridae or Beta-coronaviridae infection, COVID-19 or SARS in a subject. More than one peptide will typically be selected for treatment of a subject. In some cases, the peptide(s) used for treatment may be selected based on (i) the disease or condition to be treated in the subject; (ii) the HLA genotype of the subject; and/or (iii) the genetic background of the subject (e.g. nationality or ethnic group).

The Coronaviridae infections (and associated disease) that may be treated according to the present invention include any wherein the virus expresses at least one antigen that comprises the amino acid sequence of any one of SEQ ID NOs: 1 to 17 (or the bestEPI sequences within SEQ ID NOs: 1 to 17 shown in Table 6A). Typically the virus expresses one or more antigens that together comprise at least two, or more typically at least 3, 4, 5, 6, 7, 8, 9 or 10 different sequences selected from SEQ ID NOs: 1 to 17 (or the bestEPI sequences). More typically, the virus expresses two or more different antigens, each of which comprises sequences selected from SEQ ID NOs: 1 to 17 (or the bestEPI sequences). Suitable polypeptides of the invention or pharmaceutical compositions or kits of the invention as described herein for treatment of a particular Coronaviridae are those that match the sequence of fragments of the antigens expressed by the particular virus. The skilled person can readily identify and select such polypeptides based on the disclosure and Examples provided herein.

Polypeptide antigens, and particularly short peptides derived from polypeptide antigens, that are commonly used in vaccination and immunotherapy, induce immune responses in only a fraction of human subjects. The peptides of the present disclosure are specifically selected to induce immune responses in a high proportion of the global population. However, but they may not be effective in all individuals due to HLA genotype heterogeneity.

The present inventors have discovered that multiple HLA expressed by an individual generally need to present the same peptide in order to trigger a T cell response. Therefore the fragments of a polypeptide antigen (epitopes) that are predicted to be immunogenic for a specific individual (PEPIs) are those that can bind to multiple class I (activate cytotoxic T cells) or class II (activate helper T cells) HLAs expressed by that individual. In general, a cytotoxic T-cell response in a subject to a specific vaccine peptide is best predicted by the presence in the vaccine peptide of ≥1 PEPI3+ (epitope that binds to three or more class I HLA alleles of the subject). A helper T cell response is generally best predicted by ≥1 PEPI3+ or ≥1 PEPI4+ (epitope that binds to three or more or four or more class II HLA alleles of the subject).

Accordingly, the present disclosure provides a method of predicting that a human subject will have a T cell response (cytotoxic and/or helper) to administration of a panel of polypeptides or a pharmaceutical composition as described herein. The method may comprise (A) (i) determining that the panel of polypeptides or the active ingredient polypeptide(s) of the pharmaceutical composition comprise a T cell epitope that is restricted to at least three HLA class I molecules of the subject; and (ii) predicting that the subject will have a cytotoxic (CD8+) T cell response to administration of the panel of polypeptides or the pharmaceutical composition; and/or (B) (i) determining that the panel of polypeptides or the active ingredient polypeptide(s) of the pharmaceutical composition comprise a T cell epitope that is restricted to at least three, or in some cases at least four HLA class II molecules of the subject; and (ii) predicting that the subject will have a helper (CD4+) T cell response to administration of the panel of polypeptides or the pharmaceutical composition.

The present disclosure also provides a method of determining a probability that a specific human subject will have a T cell response (cytotoxic/CD8+ or helper/CD4+) to administration of a panel of polypeptides or pharmaceutical composition described herein, wherein the method comprises identifying T cell epitopes in the polypeptides or active ingredient polypeptides that are restricted to at least three HLA class I or at least three or at least four HLA class II of the subject, and wherein (A) (a) a higher number T cell epitopes that are restricted to at least three HLA class I of the subject; and/or (b) a higher number of T cell epitopes that are both (I) restricted to at least three HLA class I of the subject; and (II) fragments of different SARS-CoV-2 structural proteins, corresponds to a higher probability of a cytotoxic/CD8+ T cell response in the subject; and/or (B) (a) a higher number T cell epitopes that are restricted to at least three or at least four HLA class II of the subject; and/or (b) a higher number of T cell epitopes that are both (I) restricted to at least three or at least four HLA class II of the subject; and (II) fragments of different SARS-CoV-2 structural proteins, corresponds to a higher probability of a helper/CD4+ T cell response in the subject.

In some cases the subject may be predicted to have a cytotoxic T cell response, or higher than a predetermined threshold probability of having a cytotoxic T cell response to administration of the panel of peptides or the pharmaceutical composition, and the method further comprises selecting or recommending administration of the pharmaceutical composition as a method of treating the subject, and optionally further comprises treating the subject by administering the panel of polypeptides or the pharmaceutical composition to the subject.

The present disclosure also provides a method of treatment as described herein, wherein the subject receiving treatment has been predicted to have a cytotoxic or helper T cell response to administration of the panel of polypeptides or the pharmaceutical composition using a method described herein, or higher than a predetermined threshold probability of having a cytotoxic T or helper cell response to administration of the panel of polypeptides or the pharmaceutical composition using a method described herein. The method may comprise selecting peptides that are predicted to be immunogenic for a specific subject using a method described herein. A pharmaceutical composition of kit comprising the peptides so selected for the subject as active ingredients may be regarded as personalised for the subject (i.e. a personalised medicine). The method may further comprise administration to the subject.

The inventors have further discovered that the presence in a vaccine or immunotherapy composition of at least two T cell epitopes that (i) correspond to fragments of one or more target polypeptide antigens, and (ii) can bind to at least three HLA class I alleles of an individual is predictive for a clinical response. A “clinical response” or “clinical benefit” as used herein may be the prevention or a delay in the onset of a disease or condition, the amelioration of one or more symptoms, the induction or prolonging of remission, or the delay of a relapse or recurrence or deterioration, or any other improvement or stabilisation in the disease status of a subject. A clinical response may be also the prevention of infections caused by different mutated variants of Coronaviridae viruses.

Accordingly, some aspects of the disclosure relate to a method of predicting that a specific human subject will have a clinical response to a method of treatment as described herein or to administration of a panel of peptides or pharmaceutical composition as described herein, or of determining a probability of a clinical response. The method is similar to that described herein for predicting a T cell response, but a clinical response is predicted by determining that the panel of polypeptides or the active ingredient polypeptide(s) of the pharmaceutical composition comprise two different T cell epitopes that are each restricted to at least three HLA class I molecules of the subject.

### Pharmaceutical Compositions, Methods of Treatment and Modes of Administration

In some aspects the disclosure relates to a pharmaceutical composition or kit comprising one or more of the peptides, polynucleic acids, vectors or cells as described herein. Such pharmaceutical compositions or kits may be for use in a method of inducing an immune response, treating, vaccinating or providing immunotherapy to a subject. The pharmaceutical composition or kit may be a vaccine or immunotherapy composition or kit. The methods of treatment described herein may comprise administering the pharmaceutical composition to the subject.

The term “active ingredient” as used herein refers to a polypeptide that is intended to induce an immune response and may include a polypeptide product of a vaccine or immunotherapy composition that is produced in vivo after administration to a subject. For a DNA or RNA immunotherapy composition, the polypeptide may be produced in vivo by the cells of a subject to whom the composition is administered. For a cell-based composition, the polypeptide may be processed and/or presented by cells of the composition, for example autologous dendritic cells or antigen presenting cells pulsed with the polypeptide or comprising an expression construct encoding the polypeptide. The pharmaceutical composition or kit may comprise a polynucleotide or cell encoding one or more active ingredient polypeptides.

The pharmaceutical compositions or kits described herein may comprise, in addition to one or more peptides, nucleic acids, vectors or cells, a pharmaceutically acceptable excipient, carrier, diluent, buffer, stabiliser, preservative, adjuvant or other materials well known to those skilled in the art. Such materials are preferably non-toxic and preferably do not interfere with the pharmaceutical activity of the active ingredient(s). The pharmaceutical carrier or diluent may be, for example, water containing solutions and water/oil emulsions. The precise nature of the carrier or other material may depend on the route of administration, e.g. oral, intravenous, cutaneous or subcutaneous, nasal, intramuscular, intradermal, and intraperitoneal routes.

The pharmaceutical compositions of the disclosure may comprise one or more “pharmaceutically acceptable carriers”. These are typically large, slowly metabolized macromolecules such as proteins, saccharides, polylactic acids, polyglycolic acids, polymeric amino acids, amino acid copolymers, sucrose (Paoletti, 2001, Vaccine, 19:2118-2126), trehalose (WO 00/56365), lactose and lipid aggregates (such as oil droplets or liposomes). Such carriers are well known to those of ordinary skill in the art. The pharmaceutical compositions may also contain diluents, such as water, saline, glycerol, etc. Additionally, auxiliary substances, such as wetting or emulsifying agents, pH buffering substances, and the like, may be present. Sterile pyrogen-free, phosphate buffered physiologic saline is a typical carrier (Gennaro, 2000, Remington: The Science and Practice of Pharmacy, 20th edition, ISBN:0683306472).

The pharmaceutical compositions of the disclosure may be lyophilized or in aqueous form, i.e. solutions or suspensions. Liquid formulations of this type allow the compositions to be administered direct from their packaged form, without the need for reconstitution in an aqueous medium, and are thus ideal for injection. The pharmaceutical compositions may be presented in vials, or they may be presented in ready filled syringes. The syringes may be supplied with or without needles. A syringe will include a single dose, whereas a vial may include a single dose or multiple doses.

Liquid formulations of the disclosure are also suitable for reconstituting other medicaments from a lyophilized form. Where a pharmaceutical composition is to be used for such extemporaneous reconstitution, the disclosure provides a kit, which may comprise two vials, or may comprise one ready-filled syringe and one vial, with the contents of the syringe being used to reconstitute the contents of the vial prior to injection.

The pharmaceutical compositions of the disclosure may include an antimicrobial, particularly when packaged in a multiple dose format. Antimicrobials may be used, such as 2-phenoxyethanol or parabens (methyl, ethyl, propyl parabens). Any preservative is preferably present at low levels. Preservative may be added exogenously and/or may be a component of the bulk antigens which are mixed to form the composition (e.g. present as a preservative in pertussis antigens).

The pharmaceutical compositions of the disclosure may comprise detergent e.g. Tween (polysorbate), DMSO (dimethyl sulfoxide), DMF (dimethylformamide). Detergents are generally present at low levels, e.g. <0.01%, but may also be used at higher levels, e.g. 0.01-50%.

The pharmaceutical compositions of the disclosure may include sodium salts (e.g. sodium chloride) and free phosphate ions in solution (e.g. by the use of a phosphate buffer).

In certain embodiments, the pharmaceutical composition may be encapsulated in a suitable vehicle either to deliver the peptides into antigen presenting cells or to increase the stability. As will be appreciated by a skilled artisan, a variety of vehicles are suitable for delivering a pharmaceutical composition of the disclosure. Non-limiting examples of suitable structured fluid delivery systems may include nanoparticles, liposomes, microemulsions, micelles, dendrimers and other phospholipid-containing systems. Methods of incorporating pharmaceutical compositions into delivery vehicles are known in the art.

In order to increase the immunogenicity of the composition, the pharmacological compositions may comprise one or more adjuvants and/or cytokines.

Suitable adjuvants include an aluminum salt such as aluminum hydroxide or aluminum phosphate, but may also be a salt of calcium, iron or zinc, or may be an insoluble suspension of acylated tyrosine, or acylated sugars, or may be cationically or anionically derivatised saccharides, polyphosphazenes, biodegradable microspheres, monophosphoryl lipid A (MPL), lipid A derivatives (e.g. of reduced toxicity), 3-O-deacylated MPL [3D-MPL], quil A, Saponin, QS21, Freund's Incomplete Adjuvant (Difco Laboratories, Detroit, Mich.), Merck Adjuvant 65 (Merck and Company, Inc., Rahway, N.J.), AS-2 (Smith-Kline Beecham, Philadelphia, Pa.), CpG oligonucleotides, bioadhesives and mucoadhesives, microparticles, liposomes, polyoxyethylene ether formulations, polyoxyethylene ester formulations, muramyl peptides or imidazoquinolone compounds (e.g. imiquamod and its homologues). Human immunomodulators suitable for use as adjuvants in the disclosure include cytokines such as interleukins (e.g. IL-1, IL-2, IL-4, IL-5, IL-6, IL-7, IL-12, etc), macrophage colony stimulating factor (M-CSF), tumour necrosis factor (TNF), granulocyte, macrophage colony stimulating factor (GM-CSF) may also be used as adjuvants.

In some embodiments, the compositions comprise an adjuvant selected from the group consisting of Montanide ISA-51 (Seppic, Inc., Fairfield, N.J., United States of America), QS-21 (Aquila Biopharmaceuticals, Inc., Lexington, Mass., United States of America), GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), Freunds adjuvant (complete and incomplete), mineral gels, aluminum hydroxide (Alum), lysolecithin, pluronic polyols, polyanions, oil emulsions, dinitrophenol, diphtheria toxin (DT). In a particular embodiment the adjuvant is Montanide adjuvant.

By way of example, the cytokine may be selected from the group consisting of a transforming growth factor (TGF) such as but not limited to TGF-α and TGF-β; insulin-like growth factor-I and/or insulin-like growth factor-II; erythropoietin (EPO); an osteoinductive factor; an interferon such as but not limited to interferon-α, -β, and -γ; a colony stimulating factor (CSF) such as but not limited to macrophage-CSF (M-CSF); granulocyte-macrophage-CSF (GM-CSF); and granulocyte-CSF (G-CSF). In some embodiments, the cytokine is selected from the group consisting of nerve growth factors such as NGF-β; platelet-growth factor; a transforming growth factor (TGF) such as but not limited to TGF-α. and TGF-β; insulin-like growth factor-I and insulin-like growth factor-II; erythropoietin (EPO); an osteoinductive factor; an interferon (IFN) such as but not limited to IFN-α, IFN-0, and IFN-γ; a colony stimulating factor (CSF) such as macrophage-CSF (M-CSF); granulocyte-macrophage-CSF (GM-CSF); and granulocyte-CSF (G-CSF); an interleukin (Il) such as but not limited to IL-1, IL-1.alpha., IL-2, IL-3, IL-4, IL-5, IL-6, IL-7, IL-8, IL-9, IL-10, IL-11, IL-12; IL-13, IL-14, IL-15, IL-16, IL-17, IL-18; LIF; kit-ligand or FLT-3; angiostatin; thrombospondin; endostatin; a tumor necrosis factor (TNF); and LT.

It is expected that an adjuvant or cytokine can be added in an amount of about 0.01 mg to about 10 mg per dose, preferably in an amount of about 0.2 mg to about 5 mg per dose. Alternatively, the adjuvant or cytokine may be at a concentration of about 0.01 to 50%, preferably at a concentration of about 2% to 30%.

In certain aspects, the pharmaceutical compositions of the disclosure are prepared by physically mixing the adjuvant and/or cytokine with peptides described herein under appropriate sterile conditions in accordance with known techniques to produce the final product.

Examples of suitable compositions of polypeptide fragments and methods of administration are provided in Esseku and Adeyeye (2011) and Van den Mooter G. (2006). Vaccine and immunotherapy composition preparation is generally described in Vaccine Design (“The subunit and adjuvant approach” (eds Powell M. F. & Newman M. J. (1995) Plenum Press New York). Encapsulation within liposomes, which is also envisaged, is described by Fullerton, U.S. Pat. No. 4,235,877.

In some embodiments, the compositions disclosed herein are prepared as a (ribo)nucleic acid vaccine. In some embodiments, the nucleic acid vaccine is a DNA vaccine. In some embodiments, DNA vaccines, or gene vaccines, comprise a plasmid with a promoter and appropriate transcription and translation control elements and a nucleic acid sequence encoding one or more polypeptides of the disclosure. In some embodiments, the plasmids also include sequences to enhance, for example, expression levels, intracellular targeting, or proteasomal processing. In some embodiments, DNA vaccines comprise a viral vector containing a nucleic acid sequence encoding one or more polypeptides of the disclosure. In additional aspects, the compositions disclosed herein comprise one or more nucleic acids encoding peptides determined to have immunoreactivity with a biological sample. For example, in some embodiments, the compositions comprise one or more nucleotide sequences encoding 1, 2, 3, 4, 5, 6, 7, 8, 9, 10, 11, 12, 13, 14, 15, 16, 17, 18, 19, 20, or more peptides comprising a fragment that is a T cell epitope capable of binding to at least three HLA class I molecules and/or at least three or four HLA class II molecules of a patient. In some embodiments the DNA or gene vaccine also encodes immunomodulatory molecules to manipulate the resulting immune responses, such as enhancing the potency of the vaccine, stimulating the immune system or reducing immunosuppression. Strategies for enhancing the immunogenicity of DNA or gene vaccines include encoding of xenogeneic versions of antigens, fusion of antigens to molecules that activate T cells or trigger associative recognition, priming with DNA vectors followed by boosting with viral vector, and utilization of immunomodulatory molecules. In some embodiments, the DNA vaccine is introduced by a needle, a gene gun, an aerosol injector, with patches, via microneedles, by abrasion, among other forms. In some forms the DNA vaccine is incorporated into liposomes or other forms of nanobodies. In some embodiments, the DNA vaccine includes a delivery system selected from the group consisting of a transfection agent; protamine; a protamine liposome; a polysaccharide particle; a cationic nanoemulsion; a cationic polymer; a cationic polymer liposome; a cationic nanoparticle; a cationic lipid and cholesterol nanoparticle; a cationic lipid, cholesterol, and PEG nanoparticle; a dendrimer nanoparticle. In some embodiments, the DNA vaccines is administered by inhalation or ingestion. In some embodiments, the DNA vaccine is introduced into the blood, the thymus, the pancreas, the skin, the muscle, a tumor, or other sites.

In some embodiments, the compositions disclosed herein are prepared as an RNA vaccine. In some embodiments, the RNA is non-replicating mRNA or virally derived, self-amplifying RNA. In some embodiments, the non-replicating mRNA encodes the peptides disclosed herein and contains 5′ and 3′ untranslated regions (UTRs). In some embodiments, the virally derived, self-amplifying RNA encodes not only the peptides disclosed herein but also the viral replication machinery that enables intracellular RNA amplification and abundant protein expression. In some embodiments, the RNA is directly introduced into the individual. In some embodiments, the RNA is chemically synthesized or transcribed in vitro. In some embodiments, the mRNA is produced from a linear DNA template using a T7, a T3, or an Sp6 phage RNA polymerase, and the resulting product contains an open reading frame that encodes the peptides disclosed herein, flanking UTRs, a 5′ cap, and a poly(A) tail. In some embodiments, various versions of 5′ caps are added during or after the transcription reaction using a vaccinia virus capping enzyme or by incorporating synthetic cap or anti-reverse cap analogues. In some embodiments, an optimal length of the poly(A) tail is added to mRNA either directly from the encoding DNA template or by using poly(A) polymerase. The RNA may encode one or more peptides comprising a fragment that is a T cell epitope capable of binding to at least three HLA class I and/or at least three or four HLA class II molecules of a patient. he fragments are derived from an antigen that is expressed in a coronaviridae. In some embodiments, the RNA includes signals to enhance stability and translation. In some embodiments, the RNA also includes unnatural nucleotides to increase the half-life or modified nucleosides to change the immunostimulatory profile. In some embodiments, the RNAs is introduced by a needle, a gene gun, an aerosol injector, with patches, via microneedles, by abrasion, among other forms. In some forms the RNA vaccine is incorporated into liposomes or other forms of nanobodies that facilitate cellular uptake of RNA and protect it from degradation. In some embodiments, the RNA vaccine includes a delivery system selected from the group consisting of a transfection agent; protamine; a protamine liposome; a polysaccharide particle; a cationic nanoemulsion; a cationic polymer; a cationic polymer liposome; a cationic nanoparticle; a cationic lipid and cholesterol nanoparticle; a cationic lipid, cholesterol, and PEG nanoparticle; a dendrimer nanoparticle; and/or naked mRNA; naked mRNA with in vivo electroporation; protamine-complexed mRNA; mRNA associated with a positively charged oil-in-water cationic nanoemulsion; mRNA associated with a chemically modified dendrimer and complexed with polyethylene glycol (PEG)-lipid; protamine-complexed mRNA in a PEG-lipid nanoparticle; mRNA associated with a cationic polymer such as polyethylenimine (PEI); mRNA associated with a cationic polymer such as PEI and a lipid component; mRNA associated with a polysaccharide (for example, chitosan) particle or gel; mRNA in a cationic lipid nanoparticle (for example, 1,2-dioleoyloxy-3-trimethylammoniumpropane (DOTAP) or dioleoylphosphatidylethanolamine (DOPE) lipids); mRNA complexed with cationic lipids and cholesterol; or mRNA complexed with cationic lipids, cholesterol and PEG-lipid. In some embodiments, the RNA vaccine is administered by inhalation or ingestion. In some embodiments, the RNA is introduced into the blood, the thymus, the pancreas, the skin, the muscle, a tumor, or other sites, and/or by an intradermal, intramuscular, subcutaneous, intranasal, intranodal, intravenous, intrasplenic, intratumoral or other delivery route.

Polynucleotide or oligonucleotide components may be naked nucleotide sequences or be in combination with cationic lipids, polymers or targeting systems. They may be delivered by any available technique. For example, the polynucleotide or oligonucleotide may be introduced by needle injection, preferably intradermally, subcutaneously or intramuscularly. Alternatively, the polynucleotide or oligonucleotide may be delivered directly across the skin using a delivery device such as particle-mediated gene delivery. The polynucleotide or oligonucleotide may be administered topically to the skin, or to mucosal surfaces for example by intranasal, oral, or intrarectal administration.

Uptake of polynucleotide or oligonucleotide constructs may be enhanced by several known transfection techniques, for example those including the use of transfection agents. Examples of these agents include cationic agents, for example, calcium phosphate and DEAE-Dextran and lipofectants, for example, lipofectam and transfectam. The dosage of the polynucleotide or oligonucleotide to be administered can be altered.

Accordingly, the invention provides a vaccine or pharmaceutical composition or kit comprising one or more polynucleotides (polynucleic acids) or polyribonucleotides (ribopolynucleic acids) that encode one or more, or at least one (or at least 2, 3, 4, 5, 6, 7, 8, 9 or 10) polypeptide sequences selected from SEQ ID NO:s 1 to 17. The polynucleotide(s) or ribopolynucleotide(s) may encode one or more fragments of one or more Coronaviridae proteins, or Beta-conornaviridae proteins, SARS-CoV-2 proteins, or SARS-CoV proteins, or proteins of any Coronaviridae that express one or more proteins that comprise one or more (or 2, or 3, 4, 5, 6, 7, 8, 9, or 10 or more) amino acid sequences selected from SEQ ID NOs: 1 to 17. The fragments comprise the sequence selected from SEQ ID NOs: 1 to 17 and are typically up to 50 amino acids in length. The polynucleotide(s) or polyribonucleotide(s) may comprise at least one (or at least 2, 3, 4, 5, 6, 7, 8, 9 or 10) of the sequence selected from SEQ ID NOs: 234 to 267. Typically, the polynucleotide(s) or polyribonucleotide(s) together comprise different sequences selected from SEQ ID NOs: 234 to 250 or 251 to 267 encoding different amino acid sequences selected from SEQ ID NOs: 1 to 17. More typically, the polynucleotide(s) or ribopolynucleotide(s) encode different amino acid sequences selected from SEQ ID NOs: 1 to 17 that are fragments of different proteins expressed by a Coronaviridae. For example, the polynucleotide(s) or ribopolynucleotide(s) may together comprise at least one sequence selected from each of at least two, at least three, or all four of the following groups: (a) SEQ ID NOs: 234 to 244 or SEQ ID NOs: 251 to 261; (b) SEQ ID NOs: 245 to 248 or SEQ ID NOs: to 262 to 265; (c) SEQ ID NO: 249 or SEQ ID NO: 266; and (d) SEQ ID NO: 250 or SEQ ID NO: 267. The polynucleotide(s) or ribopolynucleotide(s) may together comprise at least one (or at least 2, 3, 4, 5, 6, 7, 8, 9 or all) of the sequences in one of the following lists: SEQ ID NOs: 236, 238, 242, 245, 246, 247, 248, 249 and 250; SEQ ID NOs: 236, 238, 240, 242, 245, 246, 247, 248, 249 and 250; SEQ ID NOs: 239, 242, 243, 244, 245, 246, 247, 248, 249 and 250; SEQ ID NOs: 252, 255, 259, 262, 263, 264, 265, 266 and 267; SEQ ID NOs: 252, 255, 257, 259, 262, 263, 264, 265, 266 and 267; and SEQ ID NOs: 256; 259, 260, 261, 262, 263, 264, 265, 266 and 267. The polynucleotide(s) or ribopolynucleotide(s) may encode any panel of polypeptides of the invention as described herein. The polynucleotide may be DNA. The polyribonucleotide may be RNA. For example, the polyribonucleotide may be mRNA.

The invention also encompasses cell-based compositions. The one or more polypeptides or panels of polypeptides are presented on the cell surface, particularly in the body of the patient after administration. The cells may in some cases be (autologous) dendritic cells or antigen presenting cells. The cells may be pulsed with the polypeptide or comprise one or more expression constructs/cassettes encoding the polypeptide(s). The expression construct(s)/cassette(s) may comprise/express any of the polynucleotide(s) or ribopolynucleotide(s) described herein above.

The term “treatment” as used herein includes therapeutic and prophylactic treatment. Administration is typically in a “prophylactically effective amount” or a “therapeutically effective amount” (as the case may be, although prophylaxis may be considered therapy), this being sufficient to result in a clinical response or to show clinical benefit to the individual, e.g. an effective amount to prevent or delay onset of the disease or condition, to ameliorate one or more symptoms, to induce or prolong remission, or to delay relapse or recurrence.

The dose may be determined according to various parameters, especially according to the substance used; the age, weight and condition of the individual to be treated; the route of administration; and the required regimen. The amount of antigen in each dose is selected as an amount which induces an immune response. A physician will be able to determine the required route of administration and dosage for any particular individual. The dose may be provided as a single dose or may be provided as multiple doses, for example taken at regular intervals, for example 2, 3 or 4 doses administered hourly. Typically, peptides, polynucleotides or oligonucleotides are typically administered in the range of 1 pg to 1 mg, more typically 1 pg to 10 μg for particle mediated delivery and 1 μg to 1 mg, more typically 1-100 g, more typically 5-50 μg for other routes. Generally, it is expected that each dose will comprise 0.01-3 mg of antigen. An optimal amount for a particular vaccine can be ascertained by studies involving observation of immune responses in subjects.

Examples of the techniques and protocols mentioned above can be found in Remington's Pharmaceutical Sciences, 20th Edition, 2000, pub. Lippincott, Williams & Wilkins.

In some cases the method of treatment may comprise administration to a subject of more than one peptide, polynucleic acid or vector. These may be administered together/simultaneously and/or at different times or sequentially. The use of combinations of different peptides, optionally targeting different antigens, may be important to overcome the challenges of viral heterogeneity and HLA heterogeneity of individuals. The use of peptides of the disclosure in combination expands the group of individuals who can experience clinical benefit from vaccination. Multiple pharmaceutical compositions, manufactured for use in one regimen, may define a drug product. In some cases different peptides, polynucleic acids or vectors of a single treatment may be administered to the subject within a period of, for example, 1 year, or 6 months, or 3 months, or 60 or 50 or 40 or 30 days.

Routes of administration include but are not limited to intranasal, oral, subcutaneous, intradermal, and intramuscular. The subcutaneous administration is particularly preferred. Subcutaneous administration may for example be by injection into the abdomen, lateral and anterior aspects of upper arm or thigh, scapular area of back, or upper ventrodorsal gluteal area.

The compositions of the disclosure may also be administered in one, or more doses, as well as, by other routes of administration. For example, such other routes include, intracutaneously, intravenously, intravascularly, intraarterially, intraperitnoeally, intrathecally, intratracheally, intracardially, intralobally, intramedullarly, intrapulmonarily, and intravaginally. Depending on the desired duration of the treatment, the compositions according to the disclosure may be administered once or several times, also intermittently, for instance on a monthly basis for several months or years and in different dosages.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

One or more compositions of the disclosure may be administered, or the methods and uses for treatment according to the disclosure may be performed, alone or in combination with other pharmacological compositions or treatments, for example other immunotherapy, vaccine or anti-virals. The other therapeutic compositions or treatments may be administered either simultaneously or sequentially with (before or after) the composition(s) or treatment of the disclosure.

In some cases the method of treatment is a method of vaccination or a method of providing immunotherapy. As used herein, “immunotherapy” is the treatment of a disease or condition by inducing or enhancing an immune response in an individual. In certain embodiments, immunotherapy refers to a therapy that comprises the administration of one or more drugs to an individual to elicit T cell responses. In a specific embodiment, immunotherapy refers to a therapy that comprises the administration or expression of polypeptides that contain one or more PEPIs to an individual to elicit a T cell response to recognize and kill cells that display the one or more PEPIs on their cell surface in conjunction with a class I HLA. In another embodiment, immunotherapy refers to a therapy that comprises the administration or expression of polypeptides that contain one or more PEPIs presented by class II HLAs to an individual to elicit a T helper response to provide co-stimulation to cytotoxic T cells that recognize and kill diseased cells that display the one or more PEPIs on their cell surface in conjunction with a class I HLAs. In still another specific embodiment, immunotherapy refers to a therapy that comprises administration of one or more drugs to an individual that re-activate existing T cells to kill target cells and/or virus.

The invention encompasses methods of treating or preventing a Coronaviridae infection or a disease or condition associated with a Coronaviridae infection in a subject. A disease or condition associated with Coronaviridae infection includes any disease or condition, symptom or other disease attribute that is caused by, e.g. directly caused by the infection.

In some cases the Coronaviridae is a Beta-Coronaviridae, such as SARS-CoV-2 or a variant or mutant strain thereof. The Coronaviridae may be SARS-CoV. Specifically, the Coronaviridae is one that expresses one or more antigens/polypeptides that comprise one or more amino acid sequences selected from SEQ ID NOs: 1 to 17 as described herein, or one or more of the bestEPI sequences show in bold and/or underlined in Table 6A. More specifically, a specific Coronaviridae may be treated using a composition or kit, wherein the active ingredients polypeptides comprise one or more (typically 2 or more, or 3, 4, 5, 6, 7, 8, or 9 or more) sequences selected from SEQ ID NOs: 1 to 17 (or the bestEPI sequences) that are found in the antigens expressed by the specific virus. Specific compositions that are particularly suitable for or optimised for treating or preventing disease caused by a SARS-CoV-2 or SARS-CoV infection are described herein. However, the skilled person is able to use the present disclosure to identify other compositions or kits having polypeptides comprising different combinations of the amino acid sequences of SEQ ID NOs: 1 to 17 as active ingredients to use in the prevention or treatment of other Coronaviridae. A suitable treatment for a particular Coronaviridae and/or patient may be selected as described herein above and in the Examples below.

### Further Embodiments of the Disclosure—(1)

1. A polypeptide vaccine, comprising a polypeptide comprising an amino acid sequence selected from the group consisting of SEQ ID NOs: 1 to 17, and a pharmaceutically-acceptable adjuvant, diluent, carrier, preservative, excipient, buffer, stabilizer, or combination thereof.

2. The polypeptide vaccine of item 1, comprising two or more polypeptides, each polypeptide comprising a different amino acid sequence selected from the group consisting of SEQ ID NOs: 1 to 17.

3. The polypeptide vaccine of item 1, comprising at least one polypeptide from at least two of the following groups:

(a) SEQ ID NOs: 1 to 11;

(b) SEQ ID NOs: 12 to 15;

(c) SEQ ID NO: 16; and

(d) SEQ ID NO: 17.

4. The polypeptide vaccine of item 1, comprising at least two polypeptides, wherein each polypeptide comprises a different one of the amino acid sequences of SEQ ID NOs: 2, 5, 7, 9, 12, 13, 14, 15, 16, and 17, or wherein each polypeptide comprises a different one of the amino acid sequences of SEQ ID NOs: 2, 5, 9, 12, 13, 14, 15, 16, and 17, or wherein each polypeptide comprises a different one of the amino acid sequences of SEQ ID NOs: 6 and 9 to 17.

5. The polypeptide vaccine of item 1, comprising at least four polypeptides, wherein each polypeptide comprises a different one of the amino acid sequences of SEQ ID NOs: 2, 5, 7, 9, 12, 13, 14, 15, 16, and 17, or wherein each polypeptide comprises a different one of the amino acid sequences of SEQ ID NOs: 2, 5, 9, 12, 13, 14, 15, 16, and 17, or wherein each polypeptide comprises a different one of the amino acid sequences of SEQ ID NOs: 6 and 9 to 17.

6. The polypeptide vaccine of item 1, comprising at least six polypeptides, wherein each polypeptide comprises a different one of the amino acid sequences of SEQ ID NOs: 2, 5, 7, 9, 12, 13, 14, 15, 16, and 17, or wherein each polypeptide comprises a different one of the amino acid sequences of SEQ ID NOs: 2, 5, 9, 12, 13, 14, 15, 16, and 17, or wherein each polypeptide comprises a different one of the amino acid sequences of SEQ ID NOs: 6 and 9 to 17.

7. The polypeptide vaccine of item 1, comprising at least eight polypeptides, wherein each polypeptide comprises a different one of the amino acid sequences of SEQ ID NOs: 2, 5, 7, 9, 12, 13, 14, 15, 16, and 17, or wherein each polypeptide comprises a different one of the amino acid sequences of SEQ ID NOs: 2, 5, 9, 12, 13, 14, 15, 16, and 17, or wherein each polypeptide comprises a different one of the amino acid sequences of SEQ ID NOs: 6 and 9 to 17.

8. The polypeptide vaccine of item 1, comprising at least ten polypeptides, wherein each polypeptide comprises a different one of the amino acid sequences of SEQ ID NOs: 2, 5, 7, 9, 12, 13, 14, 15, 16, and 17, or wherein each polypeptide comprises a different one of the amino acid sequences of SEQ ID NOs: 2, 5, 9, 12, 13, 14, 15, 16, and 17, or wherein each polypeptide comprises a different one of the amino acid sequences of SEQ ID NOs: 6 and 9 to 17.

9. The polypeptide vaccine of item 1, wherein one or more of the polypeptides comprises a fragment of a Coronaviridae protein that is a CD8+ T cell epitope that is restricted to at least two HLA class I alleles of the individual.

10. The polypeptide of item 1, wherein one or more of the polypeptides comprises a fragment of a Coronaviridae protein that is a CD4+ T cell epitope restricted to at least two HLA class II alleles of the individual.

11. The polypeptide of item 1, wherein one or more of the polypeptides comprises a linear B cell epitope.

12. A method treating or preventing a Coronaviridae infection in an individual in need thereof, comprising administering to the individual a polypeptide vaccine of item 1.

13. The method of item 12, wherein the Coronaviridae infection is a SARS-CoV-2 infection.

### Further Embodiments of the Disclosure—(2)

1. An immunogenic composition comprising (a) at least two distinct polypeptides, each polypeptide consisting of at least 30 amino acids and no more than 60 amino acids and comprising an amino acid sequence selected from the group consisting of SEQ ID NOs: 1 to 17, and (b) a pharmaceutically-acceptable compound that increases immunogenicity of the polypeptides.
2. The immunogenic composition of item 1, wherein the composition comprises:

(a) at least one distinct polypeptide consisting of at least 30 amino acids and no more than 60 amino acid residues and comprising an amino sequence selected from SEQ ID NOs: 1 to 11;

(b) at least one distinct polypeptide consisting of at least 30 amino acids and no more than 60 amino acids and comprising an amino sequence selected from SEQ ID NOs: 12 to 15;

(c) at least one distinct polypeptide consisting of at least 30 amino acids and no more than 60 amino acids and comprising an amino sequence selected from SEQ ID NO: 16; and

(d) at least one distinct polypeptide consisting of at least 30 amino acids and no more than 60 amino acids and comprising an amino sequence selected from SEQ ID NO: 17.

3. The immunogenic composition of item 1, wherein the distinct amino acid sequence is selected from the group consisting of SEQ ID NOs: 2, 5, 7, 9, 12, 13, 14, 15, 16, and 17.
4. The immunogenic composition of item 3, wherein the amino acid sequences are selected from the group consisting of SEQ ID NOs: 2, 5, 7, 9, 12, 13, 14, 15, 16, and 17.
5. The immunogenic composition of item 1, wherein said composition comprises six distinct polypeptides, each polypeptide consisting of at least 30 amino acids and no more than 60 amino acids and comprising a distinct amino acid sequence selected from the group consisting of SEQ ID NOs: 2, 5, 7, 9, 12, 13, 14, 15, 16, and 17.
6. The immunogenic composition of item 1, wherein said composition comprises eight distinct polypeptides, each polypeptide consisting of at least 30 amino acids and no more than 60 amino acids and comprising a distinct amino acid sequence selected from the group consisting of SEQ ID NOs: 2, 5, 7, 9, 12, 13, 14, 15, 16, and 17.
7. The immunogenic composition of item 1, wherein said composition comprises ten distinct polypeptides, each polypeptide consisting of at least 30 amino acids and no more than 60 amino acids and comprising a distinct amino acid sequence selected from the group consisting of SEQ ID NOs: 2, 5, 7, 9, 12, 13, 14, 15, 16, and 17.
8. The immunogenic composition of item 1, wherein the at least one polypeptide comprises a fragment of a Coronaviridae protein that is a CD8+ T cell epitope that is restricted to at least two HLA class I alleles of an individual.
9. The immunogenic composition of item 1, wherein the at least one polypeptide comprises a fragment of a Coronaviridae protein that is a CD4+ T cell epitope restricted to at least two HLA class II alleles of an individual.
10. The immunogenic composition of item 1, wherein the at least one polypeptide comprises a linear B cell epitope.
11. A method of stimulating an immune response against a SARS-CoV-2 infection in an individual in need thereof, comprising administering to the individual the immunogenic composition of item 1.
12. The immunogenic composition of item 1, wherein said composition comprises a polypeptide consisting of a sequence according to SEQ ID NO: 2, a polypeptide consisting of a sequence according to SEQ ID NO: 5, a polypeptide consisting of a sequence according to SEQ ID NO: 7, a polypeptide consisting of a sequence according to SEQ ID NO: 9, a polypeptide consisting of a sequence according to SEQ ID NO: 12, a polypeptide consisting of a sequence according to SEQ ID NO: 13, a polypeptide consisting of a sequence according to SEQ ID NO: 14, a polypeptide consisting of a sequence according to SEQ ID NO: 15, a polypeptide consisting of a sequence according to SEQ ID NO: 16, and a polypeptide consisting of a sequence according to SEQ ID NO: 17.

### EXAMPLES

### Example 1—Safety of the PolyPEPI1018 Peptide Vaccine in Colorectal Cancer Patients

Colorectal cancer vaccine PolyPEPI1018 has been designed to optimise population PEPI3+, as described in U.S. Pat. No. 10,213,497. Vaccination with PolyPEPI1018 was safe and well tolerated. Most of the adverse events related to the vaccination were injection site reactions and mild flu like symptoms, as expected. Only one grade three serious adverse event occurred that was recorded possibly related to the treatment (non-infectious encephalitis) however, both the safety review team and the medical monitor classified the event as non-related. Table 2 collects the adverse events documented as related to the vaccination in the trial.

TABLE 2
Adverse events recorded in 11 patients as related
or possibly related to the vaccination.

| | Grade 1-2 | Grade 3 | Grade 4 |
|---|---|---|---|
| Adverse event | Number of patients (%) | | |
| Anemia | 1 (9%) | — | — |
| Arthralgia | 1 (9%) | — | — |
| Constipation | 1 (9%) | — | — |
| Fatigue | 1 (9%) | — | — |
| Myalgia | 1 (9%) | — | — |
| Non-infectious acute encephalitis | — | 1 (9%) | — |
| Site 1 burning feeling | 1 (9%) | — | — |
| Superficial thrombophlebitis | 1 (9%) | — | — |
| Vomiting | 1 (9%) | — | — |
| Erythema | 1 (9%) | — | — |
| Injection site reactions* | 4 (36%) | — | — |

*Raised erythematous patches, subcutaneous nodularity, subcutaneous nodules posterior arms and upper legs

### Example 2—Immunogenicity of the PolyPEPI1018 Vaccine in Colorectal Cancer Patients

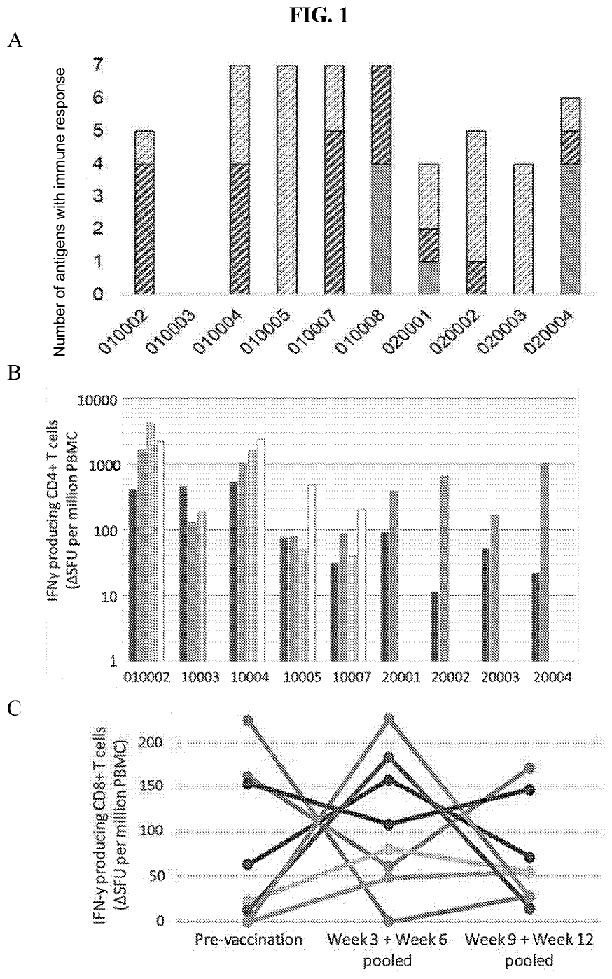

Ten of the 11 patients had sufficient PMBC samples to be analyzed by immunoassays. Seven of ten patients had pre-existing immune response against at least one PolyPEPI1018 vaccine antigen and in seven of ten patients' immune response were boosted against at least one vaccine antigen. In eight of ten patients de novo immune response was induced against at least one vaccine antigen (FIG. 1A). Pre-existing immune response was found against all seven target antigens which confirms the vaccine design strategy and target antigen selection. All ten patients had vaccine-specific CD4 T cell response, in nine of them response was boosted after vaccination (FIG. 1B).

Eight patients had samples available from pre-vaccination through week 12 to analyze the kinetics of immune responses. Patients show varying kinetic patterns through the time course with most of them having a peak at the week 3+week 6 combined time point (FIG. 1C) that aligns the observations made with immune response kinetics after viral rechallange. Nascimbeni et al found in chimpanzees that the first peak of CD8 T cell response against most of the HCV-specific test peptides was measured 5-6 weeks after intravenous re-challenge and around 3-4 weeks after intrahepatic re-challenge with HCV (Nascimbeni et al., J Virol, 77, 4781-93. 2003).

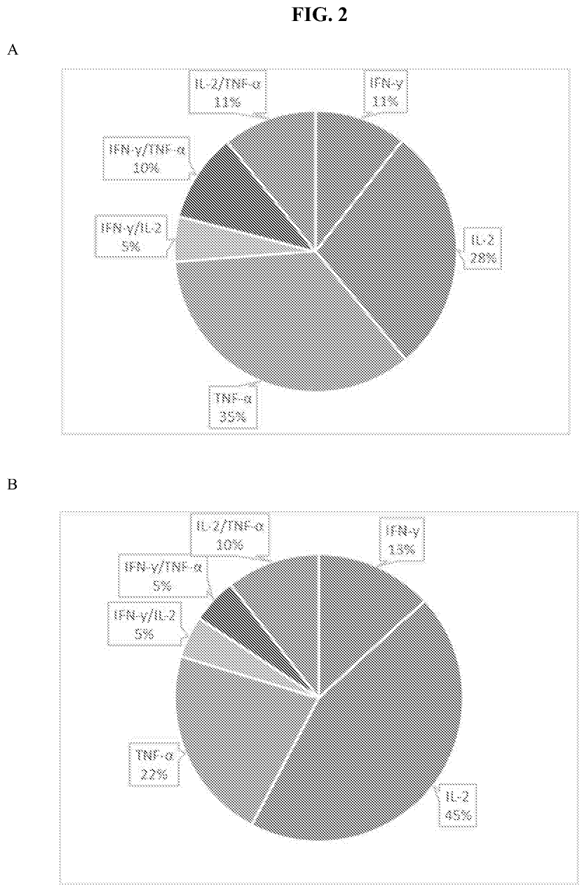

In addition to the enriched ELISPOT assay, we also performed direct ex vivo ELISPOT and quantitative intracellular cytokine staining (ICS) assay with flow cytometry as detection method to investigate effector type T cell responses. In pre-vaccination samples ex vivo ELISPOT found positive vaccine-specific CD8 response for three, and CD4 response for one of nine analyzed patients. As a consequence of the vaccination CD8 responses in five and CD4 responses in seven patients were boosted or newly induced (Table 3). In all five patients whose CD8 response was boosted or de novo induced, vaccination also induced CD4 response. As measured by ex vivo ICS, vaccine-specific CD8 and CD4 T cell responses were polyfunctional, and the frequency of functional CD8 and CD4 cells increased upon vaccination (Table 3). In the CD8 T cell fraction TNF-α positive cells dominated while the most frequent cytokine detected in the CD4 pool was IL-2 (FIG. 2).

TABLE 3
Ex vivo detected vaccine-specific effector T cell responses and
tumor infiltrating lymphocytes.

| | | | Ex vivo ICS | | | Tumor |
|---|---|---|---|---|---|---|
| | Ex vivo | | measured max. | | Tumor | infiltrating |
| | ELISPOT | | increase in | | infiltrating | lymphocytes |
| | measured | | functional | | lymphocytes | max. |
| | T cell | | T cell frequency | | Increase | increase in |
| | responses | | compared to | | in core | invasive |
| Patient | Pre/Post | | pre-vaccination | | tumor | margin |
| ID | CD8+ | CD4+ | CD8+ | CD4+ | CD3/CD8 | CD3/CD8 |
| 020001 | +/− | −/+ | 0.031% | 0.004% | NT | NT |
| 020002 | −/− | −/+ | 0.013% | 0.005% | no increase | no increase |
| 020003 | +/++ | −/+ | 0.567% | 0.663% | NT | NT |
| 020004 | +/+ | +/++ | 0.524% | 0.163% | no increase | 442%/— |
| 010002 | −/+ | −/+ | 0.360% | 0.132% | —/32% | 129%/39% |
| 010003 | −/+ | −/+ | NT | NT | NT | NT |
| 010004 | −/+ | −/+ | 0.018% | 0.266% | NT | NT |
| 010005 | −/− | −/− | 1.648% | 1.018% | NT | NT |
| 010007 | −/+ | −/+ | 0.377% | 0.183% | 132%/202% | no IM |
| 010008 | NT | NT | 0.623% | 0.800% | NT | NT |

“++” stands for boost: ≥2× pre-existing response.
ICS percentages shown are summed vaccine-specific IFN-γ, IL-2, TNF-α single or double positive T cell frequencies.
NT—not tested

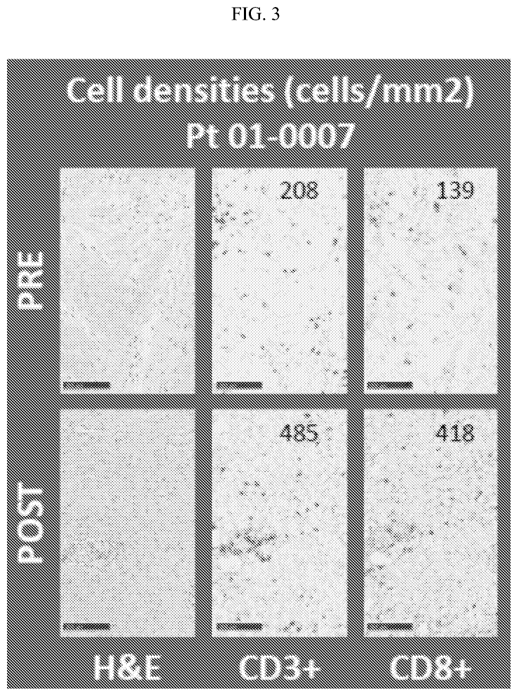

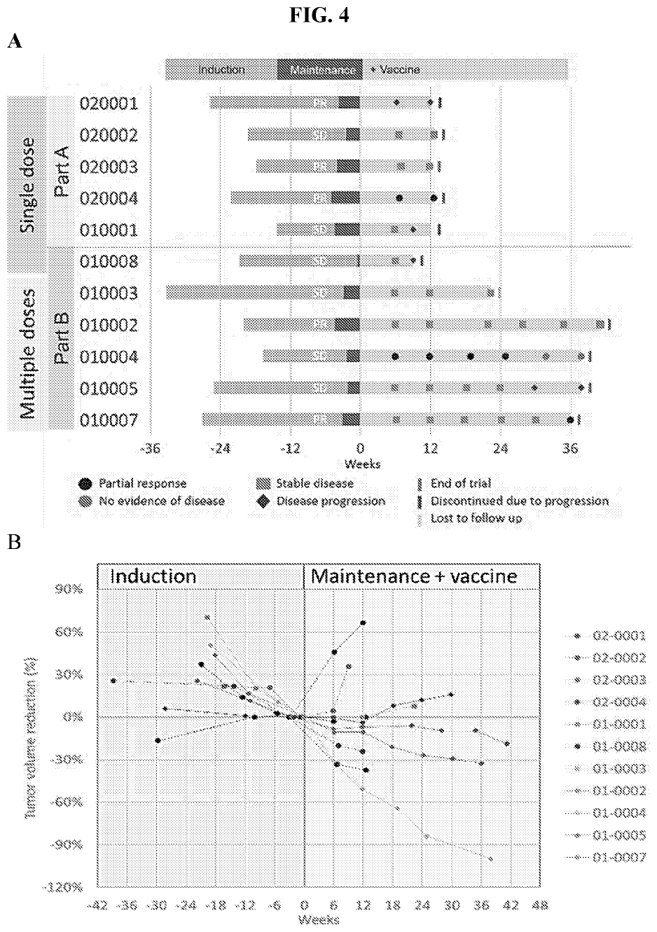

Four of the 11 patients had sufficient tumor samples to analyze tumor infiltrating lymphocytes (TILs). Vaccination induced recruitment of TILs to the invasive margin and core tumor area for three of the four tested patients'. For two patients who experienced clinical benefit post vaccination CD8 T cells accumulated in the core tumor (Table 3) suggesting that the vaccine is able to turn the cold tumor into hot. In FIG. 3 the pre- and post-vaccination (38 weeks) IHC pictures of patient 010007's biopsies are shown, where the CD8+ TTLs increased by more than 200%.

### Example 3—Efficacy of the PolyPEPI1018 Vaccine in Colorectal Cancer Patients

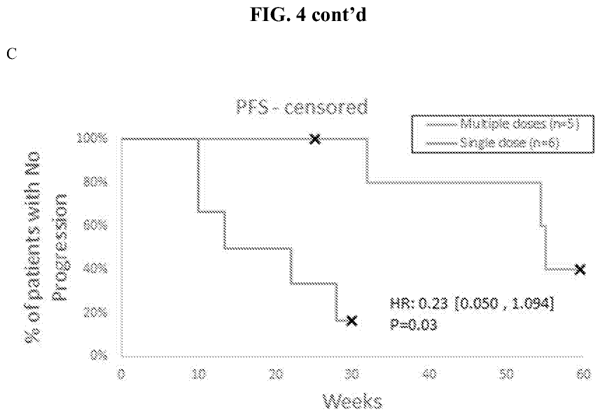

Of the 11 patients, three patients had objective tumor response according to RECIST. Among the patients receiving only a single dose (n=6), one achieved partial response and two stable disease by week 12, resulting in 17% objective response rate (ORR) and 50% disease control rate (DCR) (FIGS. 4A and 4B). In the sub-group receiving multiple doses two patients achieved partial response and three stable disease, that is 40% ORR and 80% DCR. Median PFS (including post-trial follow-up data) of the single and multiple dose groups were 4.5 months and 12.2 months, respectively (p=0.03) (FIG. 4C). In comparison the mPFS of the 5-FU plus Bevacizumab maintenance therapy in the MODUL trial with a comparable patient population (n=148) was 7.39 months. (Grothey et al., Annals of Oncology 29, 2018)

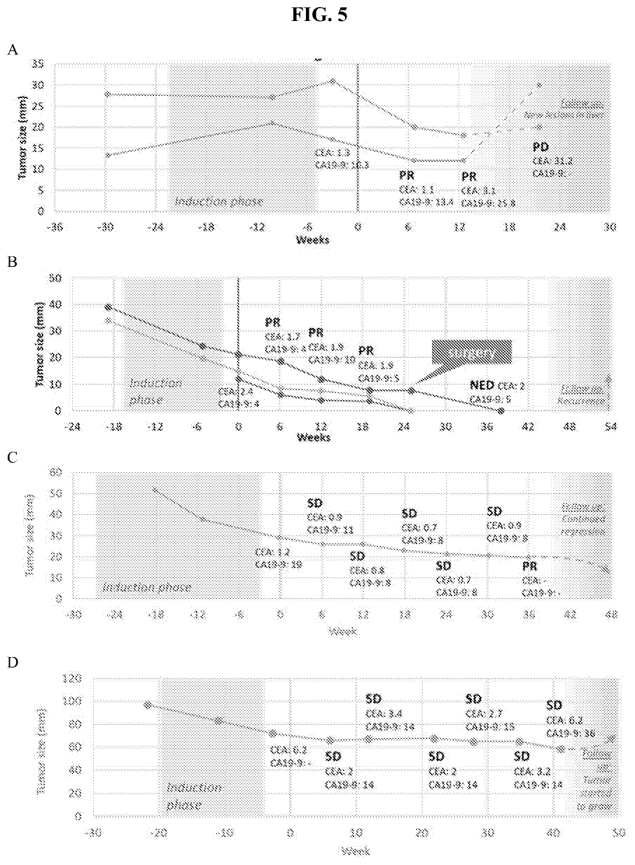

Patient 020004 was partial responder during the first line chemotherapy. After receiving a single dose of vaccine he continued remission and 6 weeks after vaccination his target lesion size decreased by more than 30% (FIG. 5A). This result suggests that the partial response achieved may be contributed in part to the induction phase. Patient 010004 was stable disease during the induction phase and achieved partial response already at week 6 (6 weeks after first vaccination). Tumor shrinkage continued throughout the further vaccinations, two of the three target lesions completely disappeared by week 24 and on week 26 curative surgery was performed to remove the third, remaining lesion (FIG. 5B). Patient 010007 after having stable disease at first line therapy, had a very slow remission during the vaccination with a slight slope and achieved partial response only on his last visit at week 38 (FIG. 5C). Similarly to Patient 010004, the shrinkage of the target lesion allowed the curative surgery for this patient too, and the pathologic analysis did not find cancer cells in the primary tumor and no residual metastasis in the liver. In addition to the latter three patients experiencing objective response, Patient 010002 also has to be highlighted. He achieved stable disease (19% target lesion shrinkage) as best response and the duration of response was 53 weeks (˜12 months). The elongated tumor responses for this patient and the patients with objective response suggests the antitumor activity of the PolyPEPI1018 vaccine.

### Example 4 Retrospective and Prospective Validation of the PEPI Test

The PEPI Test was developed to predict a subject's T-cell responses. (Toke et al., Journal of Clinical Oncology 37, 2019) The PEPI Test identifies a subject's antigen-specific personal epitopes (PEPIs) that bind to at least three HLA class I alleles of the subject. The input of the PEPI Test is the subject's 6 HLA Class I alleles and the amino acid sequence of the antigen in question. The antigens are scanned with overlapping 9-mer peptides to identify peptides that bind to the subject's HLA class I alleles. The PEPI Test obtains HLA-peptide pairs from the Epitope Database (EPDB), which was assembled by the inclusion of peptides with a binding cut-off≤5 (IEDB Percentile Rank). According to the retrospective validation of the PEPI Test with the immunogenicity data of 6 clinical trials, an identified PEPI induces immune response with 84% probability (Table 4). Performance evaluation study (validation) was carried out by retrospective analysis of six clinical trials, conducted on 71 cancer- and 9 HIV-infected patients. (Bagarazzi et al., Sci Transl Med 4, 2012, Bioley et al., Clin Cancer Res 15, 2009, Gudmundsdotter et al., Vaccine 29, 2011, Kakimi et al., Int J Cancer 129, 2011, Valmori et al., Proc Natl Acad Sci USA 104, 2007, Wada et al., J Immunother 37, 2014, Yuan et al., Proc Natl Acad Sci USA 108, 2011) We created study cohorts by randomization of the available patient data and did not exclude any patient for reason other than data availability. We did not consider exclusions from the original clinical trials since our study does not aim to retrospectively analyze these clinical trials. We did not obtain any personal data of patients. Instead, we used patient identifiers, as published in peer reviewed publications, with their HLA genotypes. Antigen sequences were obtained from publicly available protein sequence databases or peer reviewed publications. The available 157 datasets originating from 6 clinical trials involving 80 patients, were randomized with a standard random number generator to create two independent cohorts for training and validation studies. The training and validation cohorts involved different datasets of the same patient population. 76 datasets of 48 patients were included in the training cohort and 81 datasets of 51 patients in the validation cohort. Using the training dataset we determined the PEPI Count≥1 as cut-off value for the prediction of immune responses.

TABLE 4
Retrospective validation of PEPI Test (n = 81). The diagnostic performance
characteristics were obtained by comparing the PEPI Test results
(positive if PEPI Count ≥ 1) with the antigen-specific CTL responses measured by
bioassays in the clinical trials: True positive (A): 46, True
negative (D): 11, False positive (B): 9, False negative (C): 15.

| Performance characteristic | | Description | Result |
|---|---|---|---|
| Positive predictive value | 100% [A/(A + | The likelihood that an individual with a | 84% |
| | (PPV)B)] | positive PEPI Test result has antigen- | |
| | | specific CTL responses after treatment with | |
| | | immunotherapy. | |
| Sensitivity | 100% [A/ | The proportion of subjects with antigen- | 75% |
| | (A + C)] | specific CTL responses after treatment with | |
| | | immunotherapy whose PEPI Test results are | |
| | | positive (at least one antigen-specific PEPI). | |
| Specificity | 100% [D/ | The proportion of subjects without antigen- | 55% |
| | (B + D)] | specific CTL responses after treatment with | |
| | | immunotherapy whose PEPI Test results are | |
| | | negative (no antigen-specific PEPI). | |
| Negative predictive value | 100% [D/ | The likelihood that an individual with a | 42% |
| (NPV) | (C + D)] | negative PEPI Test result does not have | |
| | | antigen-specific CTL responses after | |
| | | treatment with immunotherapy. | |
| Overall percent agreement | 100% [(A + D)/ | The percentage of results that are true | 70% |
| (OPA) | N] | results, whether positive or negative. | |
| Fisher’s exact (p) | | | 0.01 |

The PEPI Test in silico tool used for the design of the PolyPEPI1018 vaccine was prospectively validated using the immunogenicity data of the 10 patients who were eligible for the immune analysis. 70 datasets (7 target antigens×10 patients) were used to assess the PEPI Test capability to predict an antigen-specific CTL response. For each dataset it was determined if PEPI Test is able to predict immune response. The overall percentage agreement was 64%, with Positive predictive value of 79%, representing the probability that the patients with predicted PEPI will produce CD8 T cell specific immune response against the analyzed antigen(s) (Table 5). Clinical trial data were significantly correlated with the retrospective trial results (p=0.01).

### Example 5 Demonstration of the Feasibility of a Possible Companion Diagnostic (CDx)

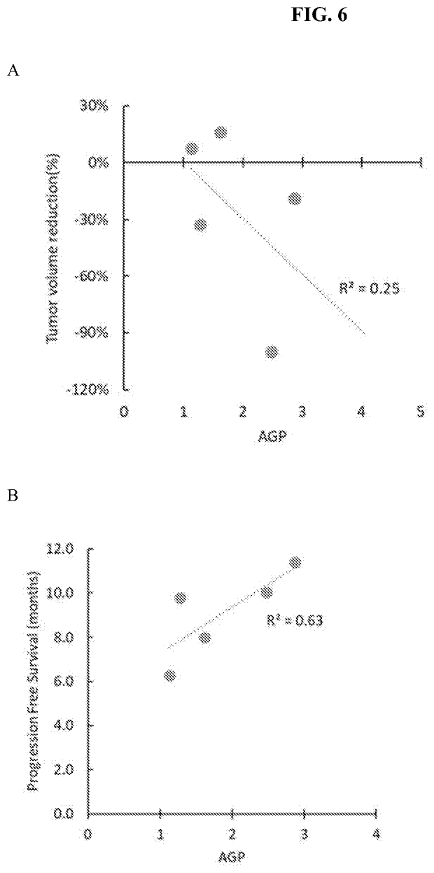

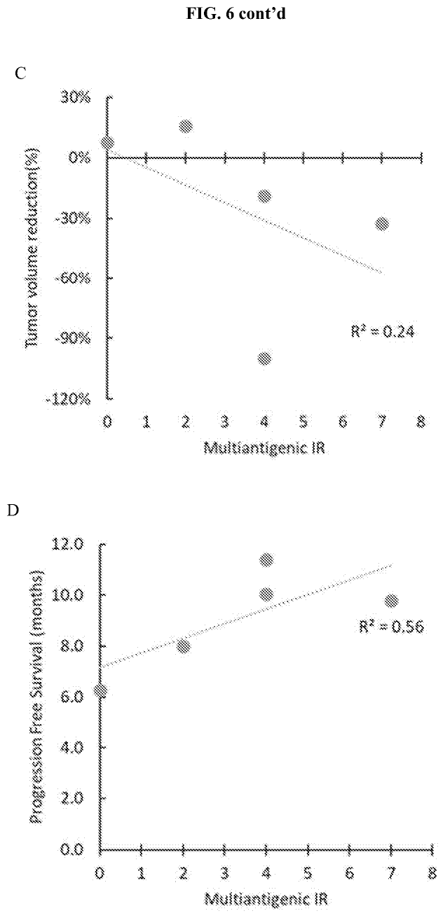

One objective of the OBERTO-101 trial was to define a biomarker that is intended to predict clinical efficacy in addition to the PEPI Test predicting immunogenicity. It is already clear from the literature that the immune response measured cannot be directly correlated to the clinical responses measured by RECIST, however correlation was already found between the multi-antigenic immune response rate and the objective response rate of cancer vaccine clinical trials (Klebanoff et al., Immunological reviews 239, 2011, Lorincz et al., Annals of Oncology 30, 2019). A candidate biomarker can be the AGP (Antigens with PEPI), which not only takes into consideration the cardinality of target antigens included in the vaccine but also the expression probability of each vaccine antigen. The AGP count for a subject indicates the expected number of antigens that the vaccine is able to “hit” with a PEPI. In the multiple dose group of the OBERTO-101 study we investigated the AGP as potential biomarker and found tendencies of association with both tumor volume reduction and PFS (FIGS. 6A and B). Significance could not be determined due to the low sample number. We found similar association patterns with the measured multiantigenic immune responses as well (FIGS. 6C and D).

### Example 6—PolyPEPI-SCoV-2 Vaccine Design

The SARS-CoV genome has a size of ˜30 kilobases which, like other coronaviruses, encodes for multiple structural and non-structural proteins. The structural proteins include the spike (S) protein, the envelope (E) protein, the membrane (M) protein, and the nucleocapsid (N) protein.

The PolyPEPI-SCoV-2 vaccine disclosed herein is composed of one or more 30 amino acid long peptides capable of inducing positive, desirable T cell (both CD8 cytotoxic and CD4 helper) responses and B cell mediated antibody responses against one or more, and preferably all 4 of the structural viral antigens in a high proportion of individuals in the global population.

A total of 19 whole genome sequences of COVID-19 were downloaded on 28 Mar. 2020 from the NCBI database. (https://www.ncbi.nm.nih.gov/genome/genomes/86693)

The accession IDs are the following: NC_045512.2, MN938384.1, MN975262.1, MN985325.1, MN988713.1, MN994467.1, MN994468.1, MN997409.1, MN988668.1, MN988669.1, MN996527.1, MN996528.1, MN996529.1, MN996530.1, MN996531.1, MT135041.1, MT135043.1, MT027063.1, MT027062.1. The first ID represents the GenBank reference sequence. Four structural protein sequences (Surface glycoprotein, Envelope protein, Membrane glycoprotein, Nucleocapsid phosphoprotein) of translated coding sequences were aligned and compared with a multiple sequence alignment. Of the 19 sequences 15 were completely the same. However, we obtained single amino acid changes in 4 nucleocapsid proteins. These replacements are the following: MN988713.1: Nucleocapsid 194 S->X, MT135043.1: Nucleocapsid 343 D->V, MT027063.1: Nucleocapsid 194 S->L, MT027062.1: Nucleocapsid 194 S->L. None of these changes affected the epitopes that have been selected for targets in the present vaccine polypeptides.

Seventeen peptide fragments were selected from the conserved regions of the presently known viral antigen sequences for SARS-CoV-2 structural proteins. The fragments were selected to maximise multi-HLA class I-binding PEPI3+ and multi-HLA class II-binding PEPI4+, i.e. shared personal epitopes, in a model population. The peptides were also designed to incorporate linear B cell epitopes. Specifically, 9mer sequences in the conserved regions of the four target antigens that are PEPI2+ in the highest proportion of subjects in the model population were selected. These 9mers were extended to incorporate nearby linear B-cell epitopes in the conserved sequence of the target antigens. 30mer fragments of the target antigens that incorporate both the 9mer “bestEPIs” and linear B cell epitopes were then selected to maximise the proportion of subjects in the model population having a HLA class II-binding PEPI4+ in the 30mer fragment. The model population comprises ˜16,000 HLA-genotyped subjects obtained from a bone-marrow transplant biobank, with about 1,000 subjects from each of 16 different ethnic groups. The sequences of the selected 30 mer peptide fragments and HLA class I-binding epitopes that are PEPI3+ and HLA class II-binding epitopes that are PEPI4+ in the highest proportion of subjects in the model population are shown in Table 6A.

TABLE 6A
List of PolyPEPI-SCoV-2 peptide sequences.
Bold/italic: 9mer HLAI bestEPI sequences,
underlined: 15mer bestEPI sequences.

| SEQ | | | | | | HLAH |
|---|---|---|---|---|---|---|
| ID | | COVID-19 | | HLAI | HLAH | (CD4, |
| no. | TREOS ID | pos. | Peptide (30mer) | (CD8) | (CD4) | P3) |
| 1 | CORONA-01 | Surface(22-51) | TQLPPA [symbol] YYPDKVFRSSVLHST | 68% | 41% | 78% |
| 2 | CORONA-02 | Surface(35-64) | GVYYPDKVFRSSVLH [symbol] FSNVTW | 71% | 94% | 99% |
| 3 | CORONA-03 | Surface(76-105) | TK [symbol] NDGVYFASTEKSNIIRGWI | 46% | 12% | 24% |
| 4 | CORONA-04 | Surface(98-127) | SNIIRGWIFGTTLDSKTQSLL [symbol] | 52% | 28% | 57% |
| 5 | CORONA-05 | Surface(253-282) | DSSSGWTAGAAAYYVG [symbol] KYNEN | 84% | 97% | 100% |
| 6 | CORONA-06 | Surface(391-420) | CFT [symbol] RGDEVRQIAPGQTGKIAD | 57% | 40% | 73% |
| 7 | CORONA-07 | Surface(683-712) | RARSVASQ [symbol] GAENSVAYSNNSI | 70% | 61% | 87% |
| 8 | CORONA-08 | Surface(701-730) | AENSVAYSNNSIAIPTN [symbol] LPVS | 62% | 33% | 57% |
| 9 | CORONA-09 | Surface(893-922)* | ALQIP [symbol] NGIGVTQNVLYENQKL | 93% | 99% | 100% |
| 10 | CORONA-10 | Surface(898-927)* | [symbol] NGIGVTQNVLYENQKLIANQE | 89% | 45% | 81% |
| 11 | CORONA-11 | Surface(1091-1120) | REGV [symbol] VTQRNFYEPQIITTDNT | 67% | 51% | 87% |
| 12 | CORONA-12 | Nucleocapsid(36-65)* | RSKQRRPQGLPN [symbol] TQHGKEDLK | 36% | 36% | 66% |
| 13 | CORONA-13 | Nucleocapsid(255-284)* | SKKPRQKRTAT [symbol] GRRGPEQTQG | 48% | 22% | 48% |
| 14 | CORONA-14 | Nucleocapsid(290-319)* | ELIRQGTDYKHWPQIAQ [symbol] GMSR | 54% | 50% | 76% |
| 15 | CORONA-15 | Nucleocapsid(384-413)* | QRQKKQQTVTLLPAADLDD [symbol] SS | 23% | 36% | 70% |
| 16 | CORONA-16 | Membrane(93-122) | LSYFIASF [symbol] WSFNPETNILLNV | 90% | 100% | 100% |
| 17 | CORONA-17 | Envelope(45-74) | NIVNVSLVKPSF [symbol] NSSRVPDLL | 46% | 100% | 100% |

*B cell epitope containing peptides, B cell epitopes are listed in Table 6B

TABLE 6B
Linear B cell epitopes

| SEQ | | Corona | | |
|---|---|---|---|---|
| ID | | virus | B cell epitopes | |
| No | TREOS ID | part | IEDB ID | SEQ |
| 18 | CORONA-09 | Surface | 3176 | AMQMAYRF |
| | | (893-922) | | |
| 19 | CORONA-10 | Surface | 3176 | AMQMAYRF |
| | | (898-927) | | |
| 20 | CORONA-12 | Nucleo- | 55683 | RRPQGLPNN |
| | | capsid | | TASWFT |
| | | (36-65) | | |
| 21 | | | 21065 | GLPNNTASWF |
| | | | | TALTQHGK |
| 22 | CORONA-12 | Nucleo- | 55683 | RRPQGLPNNT |
| | | capsid | | ASWFT |
| | | (36-65) | | |
| 23 | | | 21065 | GLPNNTASWF |
| | | | | TALTQHGK |
| 24 | CORONA-14 | Nucleo- | 28371 | IRQGTDYKHW |
| | | capsid | | PQIAQFA |
| | | (290-319) | | |
| 25 | | | 31166 | KHWPQIAQF |
| | | | | APSASAFF |
| 26 | | | 50965 | QGTDYKHW |
| 27 | CORONA-15 | Nucleo- | 37640 | LLPAAD |
| | | capsid | | |
| | | (384-413) | | |

Reference: Preliminary Identification of Potential Vaccine Targets or the COVID-19 Coronavirus (SARS-CoV-2) Based on SARS-CoV Immunological Studies. Table 4. SARS-CoV-derived linear B cell epitopes from S (23; 20 of which are located in subunit S2) and N (22) proteins that are identical in SARS-CoV-2 (45 epitopes in total).

TABLE 6C
Helper table for Best HLAI and HLAII PEPIs:

| TREOS | SEQ | Best | SEQ | Best |
|---|---|---|---|---|
| ID | ID No | HLAI | ID No | HLAII |
| CORONA-01 | 28 | YTNSFTRGV | 44 | YYPDKVFRSSVLHST |
| CORONA-02 | 29 | STQDLFLPF | 45 | STQDLFLPFFSNVTW |
| CORONA-03 | 30 | RFDNPVLPF | 46 | DGVYFASTEKSNIIR |
| CORONA-04 | 31 | IVNNATNVV | 47 | KTQSLLIVNNATNVV |
| CORONA-05 | 32 | YLQPRTFLL | 48 | AAAYYVGYLQPRTFL |
| CORONA-06 | 33 | NVYADSFVI | 49 | CFTNVYADSFVIRGD |
| CORONA-07 | 34 | SIIAYTMSL | 50 | SQSIIAYTMSLGAEN |
| CORONA-08 | 35 | FTISVTTEI | 51 | TNFTISVTTEILPVS |
| CORONA-09 | 36 | FAMQMAYRF | 52 | ALQIPFAMQMAYRFN |
| CORONA-10 | | | 53 | FAMQMAYRFNGIGVT |
| CORONA-11 | 37 | FVSNGTHWF | 54 | HWFVTQRNFYEPQII |
| CORONA-12 | 38 | NTASWFTAL | 55 | NNTASWFTALTQHGK |
| CORONA-13 | 39 | KAYNVTQAF | 56 | TATKAYNVTQAFGRR |
| CORONA-14 | 40 | FAPSASAFF | 57 | QIAQFAPSASAFFGM |
| CORONA-15 | 41 | FSKQLQQSM | 58 | KKQQTVTLLPAADLD |
| CORONA-16 | 42 | RLFARTRSM | 59 | LSYFIASFRLFARTR |
| CORONA-17 | 43 | YVYSRVKNL | 60 | KPSFYVYSRVKNLNS |

### Example 7—Comparison of PolyPEPI-SCoV-2 and State of Art Vaccine

As suggested in the article “Preliminary Identification of Potential Vaccine Targets for the COVID-19 Coronavirus (SARS-CoV-2) Based on SARS-CoV Immunological Studies” (Ahmed et al), we modelled the possible efficacy (immunogenicity) of a vaccine based on the targets identified therein. The result was compared to a selection of PolyPEPI-SCov-2 vaccine peptides as described herein.

SF Ahmed et al identified 61 T-cell epitopes associated with 19 HLAI alleles to provide estimated accumulated population coverage of 96.29% based on global allele frequencies. (Ahmed et al. Viruses, 12(3). 2020) The following T-cell epitopes shown in Table 7 were suggested as potential targets for a vaccine (Table 3 of the article; 2 of 61 were only 8 mer epitopes, we excluded from the simulation).

TABLE 7
Adopted from SF Ahmed et al: Set of the
SARS-CoV-derived spike (S) and
nucleocapsid (N) protein T cell
epitopes (obtained from positive
MHC binding assays) that are identical
in SARS-CoV-2 and that maximize estimated
population coverage globally.

| | Global | Accumulated | | |
|---|---|---|---|---|
| | Accumulated | Population | | SEQ |
| | Population | Coverage in | | ID |
| HLA Allele | Coverage2 (%) | China (%) | Epitopes | No. |
| HLA-A*02:01 | 39.08 | 14.62 | FIAGLIAIV | 65 |
| | | | GLIAIVMVTI | 66 |
| | | | IITTDNTFV | 67 |
| | | | ALNTLVKQL | 68 |
| | | | LITGRLQSL | 69 |
| | | | LLLQYGSFC | 70 |
| | | | LQYGSFCT | 71 |
| | | | NLNESLIDL | 72 |
| | | | RLDKVEAEV | 73 |
| | | | RLNEVAKNL | 74 |
| | | | RLQSLQTYV | 75 |
| | | | VLNDILSRL | 76 |
| | | | VVFLHVTYV | 77 |
| | | | ILLNKHID | 78 |
| | | | FPRGQGVPI | 79 |
| | | | LLLLDRLNQ | 80 |
| | | | GMSRIGMEV | 81 |
| | | | ILLNKHIDA | 82 |
| | | | ALNTPKDHI | 83 |
| | | | LALLLLDRL | 84 |
| | | | LLLDRLNQL | 85 |
| | | | LLLLDRLNQL | 86 |
| | | | LQLPQGTTL | 87 |
| | | | AQFAPSASA | 88 |
| | | | TTLPKGFYA | 89 |
| | | | VLQLPQGTTL | 90 |
| HLA-A*24:02 | 55.48 | 36.11 | GYQPYRVVVL | 91 |
| | | | PYRWVLSF | 92 |
| | | | LSPRWYFYY | 93 |
| HLA-A*01:01 | 66.78 | 39.09 | DSFKEELDKY | 94 |
| | | | LIDLQELGKY | 95 |
| | | | PYRWVLSF | 96 |
| | | | GTTLPKGFY | 97 |
| | | | VTPSGTWLTY | 98 |
| HLA-A*03:01 | 76.14 | 41.68 | GSFCTQLNR | 99 |
| | | | GVVFLHVTY | 100 |
| | | | AQALNTLVK | 101 |
| | | | MTSCCSCLK | 102 |
| | | | ASANLAATK | 103 |
| | | | SLIDLQELGK | 104 |
| | | | SVLNDILSR | 105 |
| | | | TQNVLYENQK | 106 |
| | | | CMTSCCSCLK | 107 |
| | | | VQIDRLITGR | 108 |
| | | | KTFPPTEPK | 109 |
| | | | KTFPPTEPKK | 110 |
| | | | LSPRWYFYY | ill |
| | | | ASAFFGMSR | 112 |
| | | | ATEGALNTPK | 113 |
| | | | QLPQGTTLPK | 114 |
| | | | QQQGQTVTK | 115 |
| | | | QQQQGQTVTK | 116 |
| | | | SASAFFGMSR | 117 |
| | | | SQASSRSSSR | 118 |
| | | | TPSGTWLTY | 119 |
| HLA-A*11:01 | 83.39 | 73.43 | GSFCTQLNR | 120 |
| | | | GVVFLHVTY | 121 |
| | | | AQALNTLVK | 122 |
| | | | MTSCCSCLK | 123 |
| | | | ASANLAATK | 124 |
| | | | SLIDLQELGK | 125 |
| | | | SVLNDILSR | 126 |
| | | | TQNVLYENQK | 127 |
| | | | CMTSCCSCLK | 128 |
| | | | VQIDRLITGR | 129 |
| | | | KTFPPTEPK | 130 |
| | | | KTFPPTEPKK | 131 |
| | | | LSPRWYFYY | 132 |
| | | | ASAFFGMSR | 133 |
| | | | ATEGALNTPK | 134 |
| | | | QLPQGTTLPK | 135 |
| | | | QQQGQTVTK | 136 |
| | | | QQQQGQTVTK | 137 |
| | | | SASAFFGMSR | 138 |
| | | | SQASSRSSSR | 139 |
| | | | TPSGTWLTY | 140 |
| HLA-A*68:01 | 85.71 | 74.25 | GSFCTQLNR | 141 |
| | | | GVVFLHVTY | 142 |
| | | | AQALNTLVK | 143 |
| | | | MTSCCSCLK | 144 |
| | | | ASANLAATK | 145 |
| | | | SLIDLQELGK | 146 |
| | | | SVLNDILSR | 147 |
| | | | TQNVLYENQK | 148 |
| | | | CMTSCCSCLK | 149 |
| | | | VQIDRLITGR | 150 |
| | | | KTFPPTEPK | 151 |
| | | | KTFPPTEPKK | 152 |
| | | | LSPRWYFYY | 153 |
| | | | ASAFFGMSR | 154 |
| | | | ATEGALNTPK | 155 |
| | | | QLPQGTTLPK | 156 |
| | | | QQQGQTVTK | 157 |
| | | | QQQQGQTVTK | 158 |
| | | | SASAFFGMSR | 159 |
| | | | SQASSRSSSR | 160 |
| | | | TPSGTWLTY | 161 |
| HLA-A*23:01 | 87.72 | 74.87 | GYQPYRVVVL | 162 |
| | | | PYRWVLSF | 163 |
| | | | LSPRWYFYY | 164 |
| HLA-A*31:01 | 89.55 | 76.93 | GSFCTQLNR | 165 |
| | | | GVVFLHVTY | 166 |
| | | | AQALNTLVK | 167 |
| | | | MTSCCSCLK | 168 |
| | | | ASANLAATK | 169 |
| | | | SLIDLQELGK | 170 |
| | | | SVLNDILSR | 171 |
| | | | TQNVLYENQK | 172 |
| | | | CMTSCCSCLK | 173 |
| | | | VQIDRLITGR | 174 |
| | | | KTFPPTEPK | 175 |
| | | | KTFPPTEPKK | 176 |
| | | | LSPRWYFYY | 177 |
| | | | ASAFFGMSR | 178 |
| | | | ATEGALNTPK | 179 |
| | | | QLPQGTTLPK | 180 |
| | | | QQQGQTVTK | 181 |
| | | | QQQQGQTVTK | 182 |
| | | | SASAFFGMSR | 183 |
| | | | SQASSRSSSR | 184 |
| | | | TPSGTWLTY | 185 |
| HLA-B*07:02 | 90.89 | 77.61 | FPNITNLCPF | 186 |
| | | | APHGVVFLHV | 187 |
| | | | FPRGQGVPI | 188 |
| | | | APSASAFFGM | 189 |
| HLA-B*08:01 | 92.85 | 78.41 | FPRGQGVPI | 190 |
| HLA-B*35:01 | 93.53 | 79.23 | FPNITNLCPF | 191 |
| | | | APHGVVFLHV | 192 |
| | | | FPRGQGVPI | 193 |
| | | | APSASAFFGM | 194 |
| HLA-B*15:01 | 94.18 | 82.26 | LQIPFAMQM | 195 |
| | | | RVDFCGKGY | 196 |
| HLA-B*51:01 | 94.72 | 83.73 | FPNITNLCPF | 197 |
| | | | APHGVVFLHV | 198 |
| | | | FPRGQGVPI | 199 |
| | | | APSASAFFGM | 200 |
| HLA-B*18:01 | 95.23 | 83.88 | YEQYIKWPWY | 201 |
| HLA-B*27:05 | 95.55 | 84 | GRLQSLQTY | 202 |
| | | | RVDFCGKGY | 203 |
| | | | VRFPNITNL | 204 |
| HLA-A*33:01 | 95.79 | 85.28 | MTSCCSCLK | 205 |
| | | | SLIDLQELGK | 206 |
| | | | CMTSCCSCLK | 207 |
| | | | VQIDRLITGR | 208 |
| | | | SASAFFGMSR | 209 |
| | | | SQASSRSSSR | 210 |
| HLA-B*58:01 | 95.99 | 86.45 | LQIPFAMQM | 211 |
| | | | RVDFCGKGY | 212 |
| HLA-C*15:02 | 96.17 | 87.22 | LQIPFAMQM | 213 |
| | | | RVDFCGKGY | 214 |
| HLA-C*14:02 | 96.29 | 88.11 | VRFPNITNL | 215 |

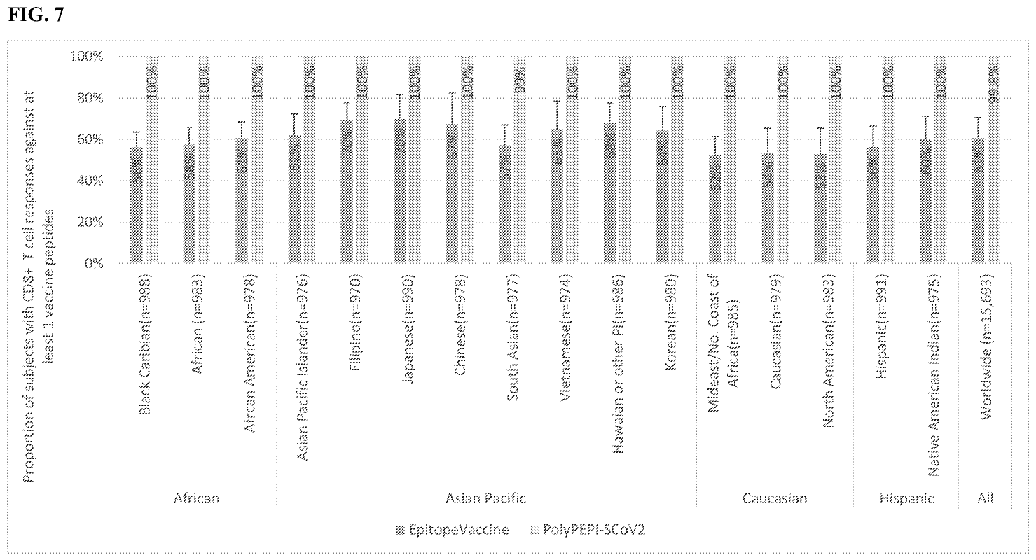

Ahmed et al suggest that the estimated maximum population coverage might be achieved by selecting at least one epitope for each listed HLA allele (ie 19 sequences). Accordingly, we made a random selection from this T-cell epitope set, selecting one epitope for each HLA allele (exactly as suggested by the authors). Because these are promiscuous HLA-binding epitopes, therefore sometimes we selected the same epitope for more than one HLA allele. This was repeated 30 times and the selected epitopes were compared to 10 peptides selected for PolyPEPI-SCoV-2 (SEQ ID NOs: 2, 5, 7, 9, 12, 13, 14, 15, 16, 17). The in-silico comparison was performed on our ˜16,000 HLA-genotyped subjects database obtained from a bone-marrow transplant biobank. Our database contains data from 16 ethnic groups (about 1,000 subject per group). We computed the proportion of subjects with CD8+ immune response against at least one epitope. The worldwide (global) coverage of the PolyPEPI-SCoV-2 is 99.8%, compared to the simulated vaccine (random epitope selection), where the average coverage was 61% (±9.9%), for some of the ethnic groups (eg Caucasians) achieving lower protection than for others (eg Japanese) (FIG. 7).

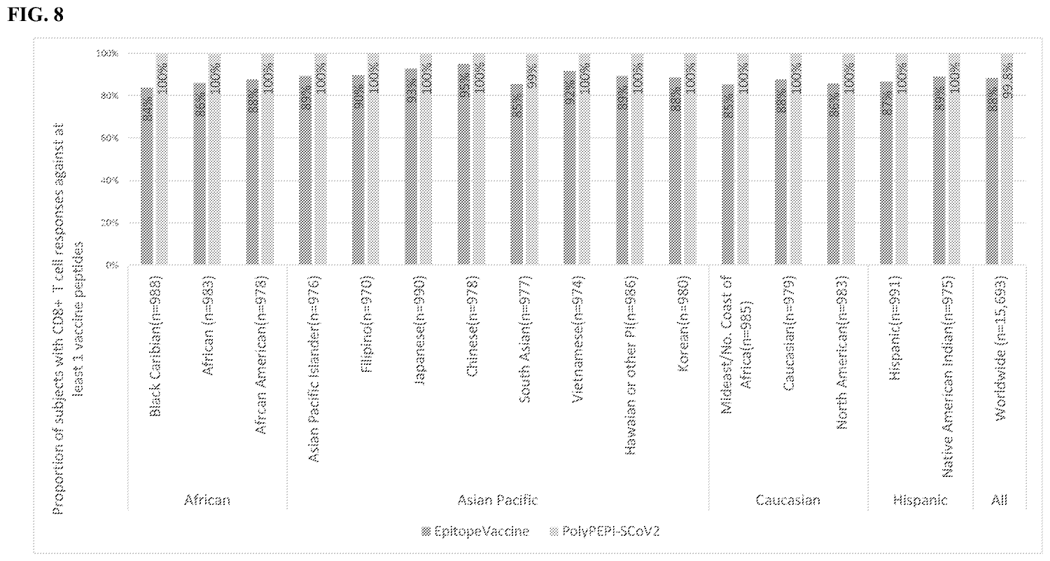

A further special (not practical) situation was modelled, where all T-cell epitopes listed in Ahmed et al (n=59) were selected into the vaccine. In this case the worldwide coverage increased to up to 88% but still not reaching the level of PolyPEPI-SCoV-2 (FIG. 8). This showed uniform coverage between the ethnic groups also for the Epitope vaccine.

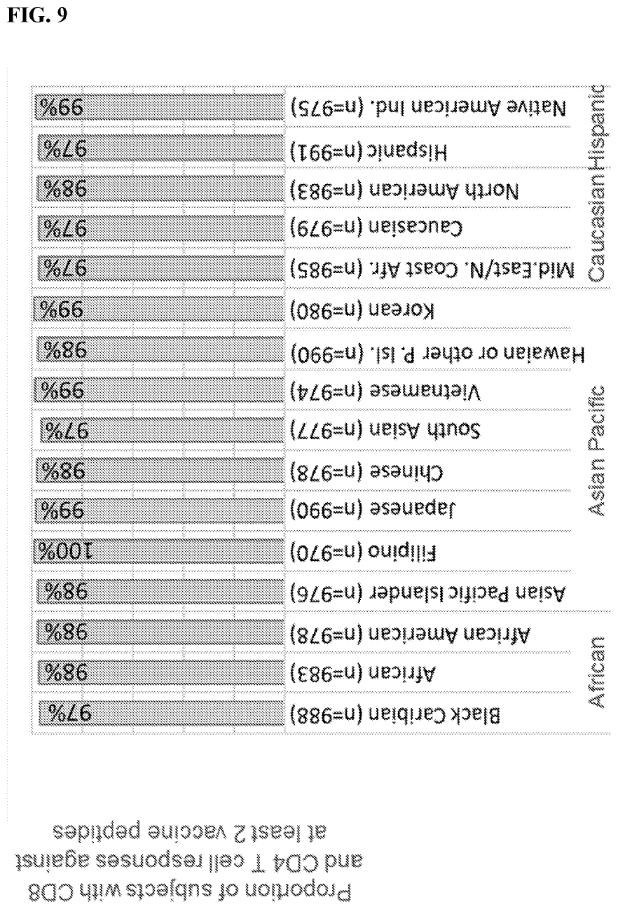

We also modelled the ability of the PolyPEPI-SCoV-2 vaccine (same 10 peptides selected) to induce HLA class II restricted CD4 responses (HLA class II PEPIs) in addition to CD8 response (FIG. 9). In each ethnic cohort at least 97% of the subjects elicited both CD8 and CD4 T cell responses against at least 2 peptides of the PolyPEPI-SCoV-2 vaccine.

### Example 8—Comparison of Number of Immunogenic Epitopes of PolyPEPI-SCoV-2 and State of Art Peptide Vaccine

Based on the previous dataset derived from Ahmed et al, we computed the number of immunogenic epitopes in each subject in the model population. The distribution of this number shows the strengths of the vaccine against potential mutations.

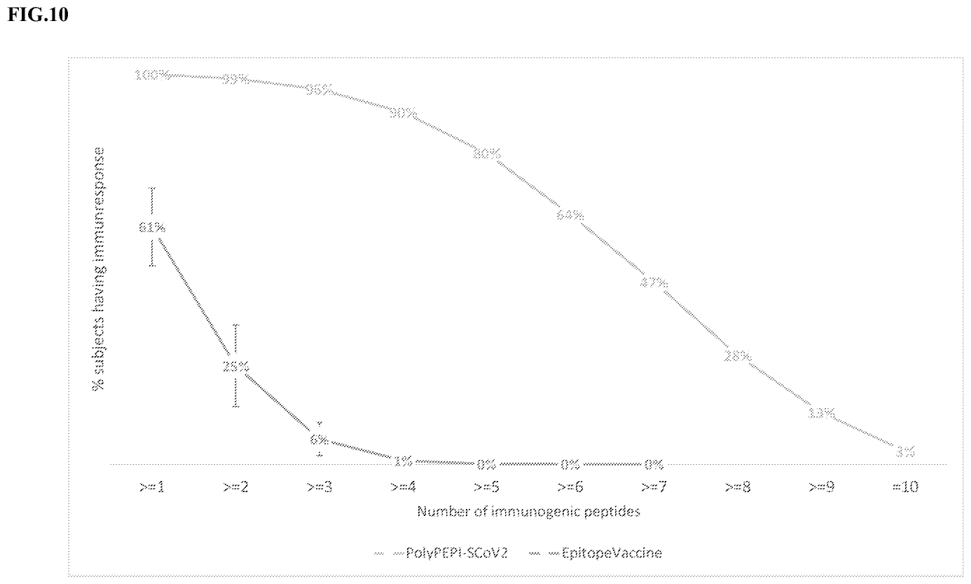

FIG. 10 shows that 61% (±9.9%) of the subjects have immune response against at least one of the vaccine's epitopes, but only 25% (±10.4%) of the subjects have response against at least 2 epitopes from 19. This means, if the virus is mutated on one particular epitope, the other epitope still can generate immune response (for a fraction of subjects). In contrast, 99% of the model population treated with PolyPEPI-SCoV-2 have response against at least 2 epitopes. The gap is even bigger for at least 3 target epitopes (96% for PolyPEPI-SCoV-2 vs. 6% for EpitopeVaccine).

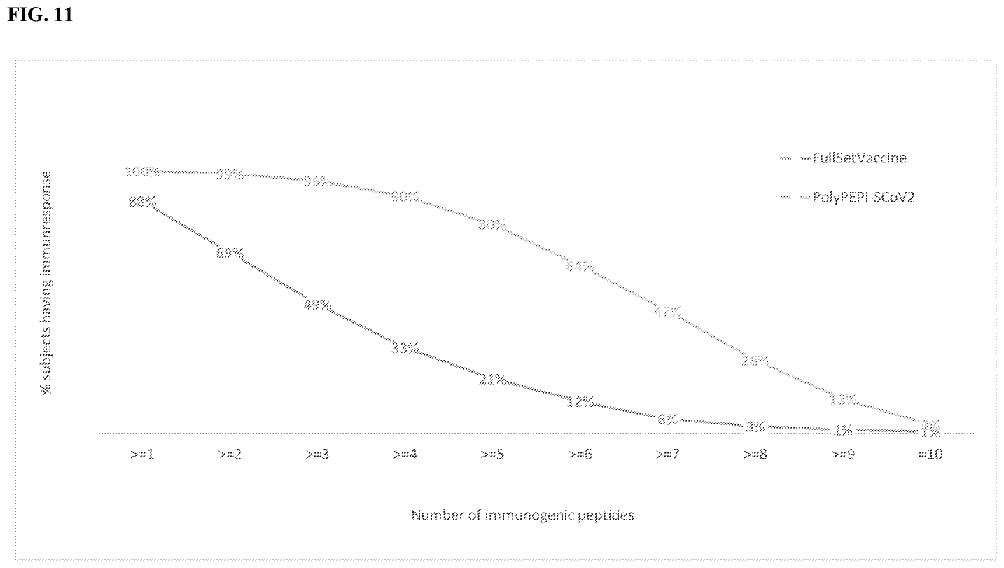

For the vaccine containing all 59 epitopes the situation would be somewhat better: 69% of subjects can have immune response against 2 or more epitopes (FIG. 11), but this is still a smaller proportion of the population compared with PolyPEPI-SCoV-2 vaccine (10 peptides).

### Example 9

Modelling of COVID-19 infection and projections warn of rapid evolution which could undermine attempts to vaccinate against and treat infection. There is an urgent need to project how transmission of the novel betacoronavirus SARS-CoV-2 will unfold in coming years. These dynamics will depend on seasonality, the duration of immunity, and the strength of cross-immunity to/from the other human coronaviruses. Using data from the United States, the inventors measured how these factors affect transmission of human coronaviruses HCoV-OC43 and HCoV-HKU1. (Kissler et al. 2020 https://doi.org/10.1101/2020.03.04.20031112). The design of the vaccine peptides and compositions described herein are robust to rapid virus evolution and cover global population by the selection of multiple immunogenic but conserved sequences, preferably derived from multiple structural proteins.

It is anticipated that as the virus continues to evolve and as more data is collected, additional mutations will be observed. Such mutations will not affect the global coverage of the polypeptides and multi-peptide vaccine described herein, provided that mutations occur outside of the identified epitope regions. Even if mutations do occur within any of the epitope regions selected, then the remaining immunogenic epitopes still provide robust protection against the virus, since the majority of subjects will retain a broad repertoire of virus-specific memory T cell clones, For example, for the ten peptide vaccine comprising polypeptides of SEQ ID NOs: 2, 5, 7, 9, 12, 13, 14, 15, 16, and 17, 94% of patients are predicted to have immune responses against at least 3 vaccine peptides and 85% and 71% against 4 and 5 peptides, respectively.

### Example 10

### Summary

This example describes the development of a global peptide vaccine against SARS-CoV-2 that addresses the dual challenges of heterogeneity in the immune responses of different individuals and potential heterogeneity of the infecting virus. In this example, “PolyPEPI-SCoV-2” is a multi-peptide vaccine containing nine 30-mer peptides derived from all four major structural proteins of the SARS-CoV-2 virus as described below. Vaccine peptides were selected based on their frequency as HLA class I and class II personal epitopes (PEPIs) restricted to multiple autologous HLA alleles of individuals in an in silico cohort of 433 subjects of different ethnicities. PolyPEPI-SCoV-2 vaccine administered with Montanide ISA 51VG adjuvant generated robust CD8⁺ and CD4⁺ T cell responses against all four structural proteins of the virus, as well as binding antibodies upon subcutaneous injection into BALB/c and CD34⁺ transgenic mice. In addition, PolyPEPI-SCoV-2-specific, polyfunctional CD8⁺ and CD4⁺ T cells were detected ex vivo in each of the 17 asymptomatic/mild COVID-19 convalescents' blood investigated, 1-5 months after symptom onset. The PolyPEPI-SCoV-2-specific T cell repertoire used for recovery from COVID-19 was extremely diverse: donors had an average of seven different peptide-specific T cells, against the SARS-CoV-2 proteins; 87% of donors had multiple targets against at least three SARS-CoV-2 proteins and 53% against all four. In addition, PEPIs determined based on the complete class I HLA-genotype of the convalescent donors were validated, with 84% accuracy, to predict PEPI-specific CD8⁺ T cell responses measured for the individuals. Extrapolation of the above findings to a US bone marrow donor cohort of 16,000 HLA-genotyped individuals with 16 different ethnicities (n=1,000 each ethnic group) suggest that PolyPEPI-SCoV-2 vaccination in a general population will likely elicit broad, multiantigenic CD8⁺ and CD4⁺ T cell responses in 98% of individuals, independent of ethnicity, including Black, Asian, and Minority Ethnic (BAME) cohorts. PolyPEPI-SCoV-2 administered with Montanide ISA 51 VG generated robust, Th1-biased CD8⁺ and CD4⁺ T cell responses against all represented proteins, as well as binding antibodies upon subcutaneous injection into BALB/c and hCD34⁺ transgenic mice modeling human immune system.

### Introduction

The pandemic caused by the novel coronavirus SARS-CoV-2 is still evolving after its outbreak in December 2019. According to World Health Organization (WHO), at least two-thirds of the vaccine candidates under clinical development are designed to generate primarily neutralizing antibodies against the viral Spike (S) protein (1), but lessons learned from the SARS-CoV and MERS epidemic as well as COVID-19 convalescents indicate potential challenges for this vaccine design strategy.(2, 3) Potential issues are dual: waning antibody levels and inefficient T cell response generation against only the Spike protein.

Patients infected with the previous SARS-CoV virus endemic in 2003 and MERS endemic in 2012 often had transient (detected only for up to 3-6 years) humoral immunity. Even more, antibodies generated by a low-risk experimental infection with a common cold coronavirus declined within 1 year and did not protect against re-challenge.(4, 5) Similarly, with SARS-CoV-2, immune responses associated with the natural course of SARS-CoV-2 viral infection suggest that anti-spike IgG antibody responses are usually weak (except for the fortunately less frequent severe cases) and their durability lasts for up to 3 months in most cases, or decline by up to 70% within this time period. (6). In addition, 2-9% of individuals do not seroconvert even 2 months after infection with SARS-CoV-2, (7) suggesting that individuals reached immunity using another arm of the adaptive immune system, T cells. Indeed, it could be concluded that virtually all subjects with a history of SARS-CoV-2 infection mount T cell responses against the virus, including seronegatives and subjects with severe disease.(2, 8-10) T cell responses are diverse, i.e., directed against the whole antigenic repertoire of the virus, and less dominated by the Spike protein. Specifically, several studies reported that despite being the largest structural protein, Spike-specific T cell responses accounted for only 25-27% of the totality of CD4⁺ and CD8⁺ T cells elicited by the natural infection. Furthermore, this diversity is associated with asymptomatic/mild disease as recovered patients had more CD8⁺ T cells against Membrane (M) and Nucleoprotein (N) proteins rather than S, while T cell intensity and diversity does not increase with disease severity, as demonstrated for MERS, SARS-CoV-1 and SARS-CoV-2.(6, 9, 11, 12) Indeed, in COVID-19 patients, low CD8⁺ T-cell counts are a predictor of higher risk for death, and patients with severe disease or who died had exhausted T cells.(2, 13) It was proposed that detectable virus-specific CD8⁺ T cell responses at earlier times after infection contribute to lower viral load and therefore lower antibody levels, explaining why these patients have more favorable outcomes.(12) In support of this, it was recently reported that mapping of SARS-CoV-2-specific T cell receptors was possible soon after viral exposure and prior to any antibody detection.(14)

These observations also suggest that achieving elevated numbers of diverse virus-specific memory T cells prior to infection (by vaccination), may contribute to virus- and viral reservoir elimination in the early-stage of SARS-CoV-2 infection. These expectations are supported by animal challenge studies demonstrating that reactivated T cells provided protection from lethal dose infection with SARS-CoV-1.(9, 15) Remarkably, memory T cells against the N protein of the SARS-CoV-1 virus were reported for 23/23 patients tested 17 years after their recovery from SARS.(9) Other reports also supported the durability of memory T cells elicited by coronavirus infections.(16, 17)

Therefore, vaccine candidates under clinical development aiming to generate T cell responses against the viral S protein will likely only generate a fraction of the convalescent's immune responses, and therefore less likely induce robust memory T cell responses. Vaccine technologies using whole viruses, multiple large proteins could theoretically solve the issue related to lack of diversity. However, these have the limitation of inclusion of unnecessary antigenic load that not only contributes little to the protective immune response, but complicates the situation by inducing allergenic and/or reactogenic responses.(18-23) Similarly, the replication-deficient viral constructs encoding target antigens could trigger unspecific immune responses against the viral vector, especially with repeated doses.(24)

Peptide vaccines are an alternative subunit vaccine strategy that relies on use of short peptide fragments, epitopes, capable of inducing positive, desirable T cell and B cell mediated immune responses.

The core problem that afflicts peptide vaccine design, however, is that each human has a uniquely endowed immune response profile. Indeed, for SARS-CoV-2, the disease course varies according to genetic diversity represented by different ethnicities, especially Black, Asian and Minority Ethnic (BAME) groups; however, the reason is not yet well understood.(25, 26) Genetic diversity could be captured by genetic variance in human leukocyte antigen (HLA) alleles, which are critical components of the viral antigen (epitope) presentation pathway, that triggers the cytotoxic T cells (CTLs) capable of recognizing and killing cancer or infected cells in the body. To capture this heterogeneity in the design of a global vaccine against SARS-CoV-2, viral antigen epitope prediction based on frequent human HLA alleles has been used widely.(27) However, in reality, these epitope mapping studies have a low yield in terms of validated T cell responses. For example, in one study, 100 SARS-CoV-2-derived epitopes predicted for the 10 most prevalent HLA class I alleles were tested and only 12 were confirmed as dominant epitopes, i.e., recognized by >50% of COVID-19 donor CD8⁺ T cells. This is consistent with immune response rates observed in the field for several infectious and cancer vaccine clinical trials, as well as for the relatively low confirmation level of personalized mutational neoantigen-based epitopes.(28-30)

To overcome these limitations of peptide vaccine design, we developed PolyPEPI-SCoV-2 digitally using an ethnically diverse in silico human cohort of individuals with complete HLA genotypes, instead using single HLA alleles. Multiple so called Personal Epitopes (PEPIs) were selected, restricted to not only one but multiple autologous HLA alleles of each individual but that are also shared among a high proportion of subjects in the ethnically diverse population. Notably, this in silico human cohort together with the PEPI concept previously retrospectively predicted the immune response rates of 79 vaccine clinical trials, as well as the remarkable immunogenicity (80% CD8⁺ T cell responses against at least three out of six antigens) of our PolyPEPI1018 cancer vaccine in a clinical trial conducted in metastatic colorectal cancer patients.(31-33) CD8⁺ T cell responses generated by PEPIs in a personalized poly-peptide mixture prepared for a patient with breast cancer proved to be long-lasting as they were detected 14 months (436 days) after last vaccination against four tumor antigens.(34)

Consistent with the apparent long term memory T cell formation capacity of SARS-CoV-2 during the natural course of infections, the present polypeptide vaccine is designed to (1) induce robust and broad immune responses in each subject by targeting all four structural proteins of SARS-CoV-2; (2) address and overcome the potential virus evolution effect by ensuring multiple immunogenic target in each patient; and (3) address different sensitivities of human ethnicities by personal epitope coverage of the peptides. The design and preclinical characterization of the vaccine candidate against COVID-19 is described herein. Immunogenicity and tolerability were confirmed in two mice models, resulting in the induction of robust CD4⁺ and CD8⁺ T cell responses boosted by the second dose, as well as humoral responses. In convalescent COVID-19 blood samples, vaccine-specific immune cells were detected against all peptides and in all subjects, representing important components of the SARS-CoV-2-induced immune repertoire leading to recovery from infection. Peptide vaccines are a safe and economical technology compared to traditional vaccines made of dead or attenuated viruses and recombinant proteins. Synthetic peptide manufacturing at multi-kilogram scale is relatively inexpensive and definitely more mature than mRNA production. The technology enables not only identification of the antigen targets for a specific disease/pathogen but, more importantly, computational determination of the antigens that immune systems of individuals in large cohorts can respond to.

### Materials and Methods

### Patients/Donors

Donors were recruited based on their clinical history of SARS-CoV-1 or SARS-CoV-2 infection. Blood samples were collected from convalescent individuals (n=15) at an independent medical research center in The Netherlands under an approved protocol (NL57912.075.16.) or collected by PepTC Vaccines Ltd (n=2). Sera and PBMC samples from non-exposed individuals (n=5) were collected before 2018 and were provided by Nexelis-IMXP (Belgium). All donors provided written informed consent. The study was conducted in accordance with the Declaration of Helsinki. Blood samples from COVID-19 convalescent patients (n=17; 16 with asymptomatic/mild disease and one with severe disease) were obtained 17-148 days after symptom onset. Surprisingly, one positive IgM antibody response was found among the healthy donors, which was excluded from further analysis. Demographic and baseline information of the subjects are provided in Table 8.

HLA genotyping of the convalescent donor patients from The Netherlands was done by IMGM laboratories GmbH (Martinsried, Germany) using next-generation sequencing. HLA genotyping of the two PepTC donors was performed from buccal swabs by Laboratory Corporation (LabCorp; Burlington, Va., USA) using next-generation sequencing (Illumina) and HLA allele interpretation was based on IMGT/HLA database version 3.38.0. HLA genotyping of the convalescent donor patients from The Netherlands was done by IMGM laboratories GmbH (Martinsried, Germany) using next-generation sequencing. This cohort uses a total of 46 different HLA class I alleles (15 HLA-A*, 18 HLA-B* and 13 HLA-C*) and 35 different HLA class II alleles (14 DRB1, 12 DQB1 and 9 DPB1). HLA-genotype data of the subjects is provided in Table 8B.

### Animals

CD34⁺ transgenic humanized mice (Hu-mice). Female NOD/Shi-scid/IL-2Rγ null immunodeficient mice (Charles River Laboratories, France) were humanized using hematopoietic stem cells (CD34⁺) isolated from human cord blood. Only mice with a humanization rate (hCD45/total CD45)>50% were used during the study. Experiments were carried out with 20-23-week-old female mice.

BALB/c mice. Experiments were carried out with 6-8 week old female BALB/c mice (Janvier, France).

TABLE 8
Donor baseline and demographic information.

| | | | | | Time from | | | | | |
|---|---|---|---|---|---|---|---|---|---|---|
| | | | | | first | | | | | |
| | | | | Blood | symptom | IgA | IgM | IgG | IgG-S1 | IgG-N |
| | | Complaints from/to: | | collection | to blood | DiaPro ELISA | | | EUROIMMUN | ROCHE |
| Donor ID | Gender | (as reported by the donors) | Complaints** | date | collection | S/Co | S/Co | S/Co | S/Co | COI |
| IMXP00394 | Female | 30 Mar. 2020-20 Apr. 2020 | a, b, c, d, h, i, j | 4 Aug. 2020 | 126 days | 0.36 | 5.065 | 5.752 | 4.48 | 54.38 |
| IMXP00714 | Male | 1 May 2020-15 May 2020 | a, b, c, h, i, j, k | 27 Jul. 2020 | 87 days | 1.324 | 8.524 | 11.524 | 5.35 | 73.06 |
| IMXP00739 | Female | 30 Apr. 2020 | j | 2 Jun. 2020 | 63 days | 0.929 | 8.841 | 11.967 | 4.54 | 77.61 |
| IMXP00756 | Female | 2 Apr. 2020-12 Apr. 2020 | b, c, d, f, i, j | 9 Jun. 2020 | 68 days | 0.989 | 4.606 | 12.193 | 3.56 | 78.47 |
| IMXP00757 | Female | 29 Feb. 2020-14 Apr. 2020 | a, b, c, d, e, h, i, j | 9 Jun. 2020 | 101 days | 1.154 | 5.847 | 8.701 | 7.62 | 29.47 |
| IMXP00758 | Female | 2 Apr. 2020-30 Apr. 2020 | c, d, h, i, i | 15 Jun. 2020 | 74 days | 1.356 | 7.757 | 11.774 | 5.79 | 121.9 |
| IMXP00759* | Male | 13 Mar. 2020-28 Mar. 2020 | a, c, d, f, h, i, j k | 15 Jun. 2020 | 94 days | 6.307 | 10.666 | 13.838 | 9.27 | 87.09 |
| IMXP00762 | Female | 15 Mar. 2020-19 Mar. 2020 | b, c, j | 29 Jun. 2020 | 106 days | 1.251 | 7.314 | 4.46 | 7.25 | 131.5 |
| IMXP00764 | Female | 16 Mar. 2020-2 Apr. 2020 | a, b, e, h, i, j, k | 6 Jul. 2020 | 115 days | 5.161 | 9.739 | 11.677 | 1.32 | 46.59 |
| IMXP00765 | Female | 29 Mar. 2020-15 May 2020 | a, d, e, h, i, j, k | 7 Jul. 2020 | 100 days | 0.565 | 2.948 | 1.54 | 1.32 | 13.4 |
| IMXP00766 | Female | 20 Jun. 2020-23 Jun. 2020 | b, c, h, j | 7 Jul. 2020 | 17 days | 0.771 | 4.648 | 3.973 | 4.14 | 6.25 |
| IMXP00767 | Female | 10 Apr. 2020-10 May 2020 | d, e, f, i, k | 7 Jul. 2020 | 88 days | 0.88 | 5.402 | 3.459 | 2.37 | 52.29 |
| IMXP00771 | Female | 18 Mar. 2020-1 Apr. 2020 | a, d, i, j | 28 Jul. 2020 | 131 days | 0.791 | 7.775 | 8.322 | 4.04 | 119.4 |
| IMXP00772 | Female | 30 Mar. 2020-30 Apr. 2020 | g, k | 28 Jul. 2020 | 120 days | 1.105 | 4.256 | 2.54 | 1.26 | 10.87 |
| IMXP00776 | Female | 9 Mar. 2020-14 Mar. 2020 | c, e, i, j, k | 4 Aug. 2020 | 148 days | 1.012 | 9.196 | 10.887 | 2.26 | 88.64 |
| PTC1 | Male | 15 Apr. 2020 | e | 13 Jul. 2020 | 89 days | 0.53 | 0.41 | 2.63 | NA | 18.96 |
| PTC2 | Female | 15 Apr. 2020 | e | 13 Jul. 2020 | 89 days | 0.45 | 0.35 | 1.49 | NA | 26.09 |

All donors were caucasoid, with mild/asymptomatic disease and no hospitalization (except one, marked with *)
S/Co, sample/control ratio; values were determined according to the manufacturer’s instructions, and test results are interpreted as negative in S/Co <0.9, not conclusive if S/CO = 0.9-1.1, and positive if S/Co >1.1. COI, cut-off index; values were determined according to the manufacturer’s instructions, and test results are interpreted as negative in COI <0.9, inconclusive with COI 0.9-1.1, and positive if COI >1.1. NA, data not available.
Italic, negative or inconclusive values.
**Complaints: a, cough; b, sore throat; c, fever; d, short of breath; e, stomach/intestinal complaints; f, chest pain; g, sore eyes; h, odor or taste loss; i, headache; j, fatigue; k, other complaints (pulmonary embolism and cardiac arrest for IMXP00759; leg pain, arm pain, muscle pain, pain in the eyes).

TABLE 8B
Complete HLA genotype of convalescent donors

| Nr. | Donor ID | HLA-A | | HLA-B | | HLA-C | | DRB1 | | DQB1 | | DPB1 | |
|---|---|---|---|---|---|---|---|---|---|---|---|---|---|
| 1 | IMXP00394 | A*11: | A*24: | B*35: | B*55: | C*03: | C*12: | DRB1*01: | DRB1*13: | DQB1*05: | DQB1*06: | DPB1*04: | DPB1*04: |
| | | 01 | 02 | 03 | 01 | 03 | 03 | 01 | 01 | 01 | 03 | 01 | 02 |
| 2 | IMXP00714 | A*01: | A*02: | B*07: | B*44: | C*04: | C*07: | DRB1*07: | DRB1*15: | DQB1*02: | DQB1*06: | DPB1*01: | DPB1*04: |
| | | 01 | 01 | 02 | 03 | 01 | 02 | 01 | 01 | 02 | 02 | 01 | 01 |
| 3 | IMXP00739 | A*03: | A*03: | B*07: | B*35: | C*04: | C*07: | DRB1*14: | DRB1*15: | DQB1*05: | DQB1*06: | DPB1*02: | DPB1*10: |
| | | 01 | 01 | 02 | 03 | 01 | 02 | 54 | 01 | 03 | 02 | 01 | 01 |
| 4 | IMXP00756 | A*02: | A*11: | B*15: | B*55: | C*03: | C*03: | DRB1*14: | DRB1*15: | DQB1*05: | DQB1*06: | DPB1*04: | DPB1*04: |
| | | 01 | 01 | 01 | 01 | 03 | 04 | 54 | 02 | 03 | 01 | 01 | 01 |
| 5 | IMXP00757 | A*02: | A*31: | B*40: | B*44: | C*03: | C*05: | DRB1*04: | DRB1*15: | DQB1*03: | DQB1*06: | DPB1*04: | DPB1*04: |
| | | 01 | 01 | 01 | 02 | 04 | 01 | 01 | 01 | 01 | 02 | 01 | 01 |
| 6 | IMXP00758 | A*01: | A*11: | B*08: | B*44: | C*05: | C*07: | DRB1*03: | DRB1*12: | DQB1*02: | DQB1*03: | DPB1*01: | DPB1*02: |
| | | 01 | 01 | 01 | 02 | 01 | 01 | 01 | 01 | 01 | 01 | 01 | 01 |
| 7 | IMXP00759 | A*24: | A*30: | B*13: | B*57: | C*06: | C*06: | DRB1*07: | DRB1*07: | DQB1*02: | DQB1*03: | DPB1*04: | DPB1*17: |
| | | 02 | 01 | 02 | 01 | 02 | 02 | 01 | 01 | 02 | 03 | 01 | 01 |
| 8 | IMXP00762 | A*02: | A*30: | B*15: | B*51: | C*12: | C*14: | DRB1*07: | DRB1*07: | DQB1*02: | DQB1*02: | DPB1*04: | DPB1*04: |
| | | 05 | 02 | 03 | 01 | 03 | 02 | 01 | 01 | 02 | 02 | 01 | 01 |
| 9 | IMXP00764 | A*01: | A*23: | B*44: | B*49: | C*04: | C*07: | DRB1*07: | DRB1*08: | DQB1*02: | DQB1*04: | DPB1*03: | DPB1*04: |
| | | 01 | 01 | 03 | 01 | 01 | 01 | 01 | 01 | 02 | 02 | 01 | 01 |
| 10 | IMXP00765 | A*02: | A*29: | B*40: | B*44: | C*03: | C*16: | DRB1*07: | DRB1*08: | DQB1*02: | DQB1*04: | DPB1*03: | DPB1*11: |
| | | 01 | 02 | 01 | 03 | 04 | 01 | 01 | 01 | 02 | 02 | 01 | 01 |
| 11 | IMXP00766 | A*03: | A*30: | B*13: | B*27: | C*02: | C*06: | DRB1*07: | DRB1*14: | DQB1*02: | DQB1*05: | DPB1*04: | DPB1*04: |
| | | 01 | 01 | 02 | 05 | 02 | 02 | 01 | 01 | 02 | 03 | 01 | 01 |
| 12 | IMXP00767 | A*01: | A*03: | B*38: | B*51: | C*12: | C*15: | DRB1*04: | DRB1*13: | DQB1*03: | DQB1*06: | DPB1*02: | DPB1*09: |
| | | 01 | 02 | 01 | 01 | 03 | 02 | 02 | 01 | 02 | 03 | 01 | 01 |
| 13 | IMXP00771 | A*02: | A*03: | B*07: | B*35: | C*04: | C*07: | DRB1*08: | DRB1*15: | DQB1*04: | DQB1*06: | DPB1*04: | DPB1*04: |
| | | 01 | 01 | 02 | 03 | 01 | 02 | 01 | 01 | 02 | 02 | 01 | 02 |
| 14 | IMXP00772 | A*02: | A*26: | B*15: | B*55: | C*03: | C*03: | DRB1*13: | DRB1*13: | DQB1*06: | DQB1*06: | DPB1*03: | DPB1*03: |
| | | 01 | 01 | 01 | 01 | 03 | 03 | 01 | 01 | 03 | 03 | 01 | 01 |
| 15 | IMXP00776 | A*24: | A*68: | B*27: | B*35: | C*04: | C*07: | DRB1*04: | DRB1*15: | DQB1*03: | DQB1*06: | DPB1*02: | DPB1*04: |
| | | 02 | 01 | 05 | 01 | 01 | 02 | 01 | 01 | 01 | 02 | 01 | 02 |
| 16 | PTC1 | A*02: | A*24: | B*35: | B*51: | C*01: | C*04: | DRB1*01: | DRB1*08: | DQB1*03: | DQB1*05: | DPB1*04: | DPB1*04: |
| | | 01 | 02 | 03 | 01 | 02 | 01 | 01 | 01 | 02 | 01 | 01 | 02 |
| 17 | PTC2 | A*26: | A*32: | B*37: | B*40: | C*02: | C*06: | DRB1*11: | DRB1*16: | DQB1*03: | DQB1*05: | DPB1*04: | DPB1*10: |
| | | 01 | 01 | 01 | 02 | 02 | 02 | 04 | 02 | 01 | 02 | 01 | 01 |

### Vaccine Design

SARS-CoV-2 structural proteins (S, N, M, E) were screened and nine different 30-mer peptides were selected during a multi-step process. First, sequence diversity analysis was performed (as of 28 Mar. 2020 in the NCBI database).(35) The accession IDs were as follows: NC_045512.2, MN938384.1, MN975262.1, MN985325.1, MN988713.1, MN994467.1, MN994468.1, MN997409.1, MN988668.1, MN988669.1, MN996527.1, MN996528.1, MN996529.1, MN996530.1, MN996531.1, MT135041.1, MT135043.1, MT027063.1, and MT027062.1. The first (bolded) ID represents the Genflank reference sequence. Then, the translated coding sequences of the four structural protein sequences were aligned and compared using a multiple sequence alignment (Clustal Omega, EMBL-EBI, United Kingdom).(36) Of the 19 sequences, 15 were identical; however, single AA changes occurred in four N protein sequences: MN988713.1, N 194 S->X; MT135043.1, N 343 D->V; MT027063.1, N 194 S->L; MT027062.1, N 194 S->L. The resulting AA substitutions affected only two positions of N protein sequence (AA 194 and 343), neither of which occurred in epitopes that have been selected as targets for vaccine development (SEQ ID NOs: 2, 5, 9, and 12-17). Only one (H49Y) of the thirteen reported single-letter changes in the viral S protein (D614G, S943P, L5F, L8V, V367F, G476S, V483A, H49Y, Y145H/del, Q239K, A831V, D839Y/N/E, P1263L), has been involved in the PolyPEPI-SCoV-2 vaccine, but the prevalence of this variant is decreasing among later virus isolates.(37) Further details on peptide selection are provided in the Results section and the resulting composition of the nine selected 30-mer peptides is shown in Table 9.

Cross-Reactivity with Human Coronavirus Strains

The sequence of PolyPEPI-SCoV-2 vaccine was compared with that of SARS-CoV, MERS-CoV and common (seasonal) human coronavirus strains to reveal possible cross-reactive regions. According to Centers for Disease Control and Prevention (CDC), common coronaviral infections in the human population are caused by four coronavirus groups: alpha coronavirus 229E and NL63, and beta coronavirus OC43 and HKU1.(38) Pairwise alignment of the structural proteins was also performed using Uniprot (39) with the following reference sequence IDs: 229E: P15423 (S), P15130 (N), P19741 (E), P15422 (M); NL63: Q6Q1S2 (S), Q6Q1R8 (N), Q6Q1S0 (E), Q6Q1R9 (M); OC43: P36334 (S), P33469 (N), Q04854 (E), Q01455 (M); HKU1 (Isolate N1): Q5MQD0 (S), Q5MQC6 (N), Q5MQC8 (E), Q5MQC7 (M). In addition, the coronavirus strains were aligned with the nine 30-mer peptides comprising the PolyPEPI-SCoV-2 vaccine. For the minimum requirement of an epitope, eight AA-long regions were screened (sliding window) as regions responsible for potential cross-reactivity. In addition, shorter (and longer) length matching consecutive peptide fragments were recorded and reported during the analysis.

### In Silico Human Cohorts

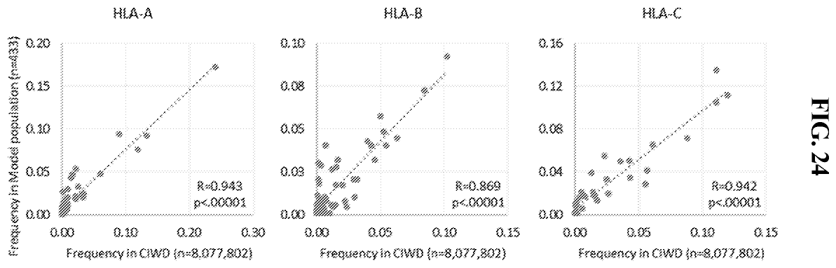

The Model Population is a cohort of 433 individuals, representing several ethnic groups worldwide, for whom complete HLA class I genotypes were available (2×HLA-A, 2×HLA-B, 2×HLA-C). The Model Population was assembled from 90 Yoruban African (YRI), 90 European (CEU), 45 Chinese (CHB), 45 Japanese (JPT), 67 subjects with mixed ethnicity (US, Canada, Australia, New Zealand), and 96 subjects from an HIV database (MIX).(40-43) HLA genotypes were determined using PCR techniques, Affymetrix 6.0 and Illumina 1.0 Million SNP mass arrays, and high-resolution HLA typing of the six HLA genes by Reference Strand-mediated Conformational Analysis (RSCA) or sequencing-based typing (SBT).(44-46) Characterization of the model population was reported previously.(31). This cohort uses a total of 152 different HLA class I alleles (49 HLA-A*, 71 HLA-B* and 32 HLA-C*) representative for 97.4% of the current global Common, Intermediate and Well-Documented (CIWD) alleles, well-representing also major ethnicities (database 3.0 released 2020) (Table 8A) (Hurley et al. 2020 The frequency of the A*, B* and C* alleles of the Model population correlates with the frequency documented for >8 million HLA-genotyped subjects of the CIWD database (R=0.943, 0.869, 0.942, respectively, p<0.00001) (FIG. 24).

TABLE 8A
HLA coverage of alleles represented in Model Population.

| | | AFA | API | EURO | MENA | HIS | NAM | UNK | Total |
|---|---|---|---|---|---|---|---|---|---|
| HLA | | N = | N = | N = | N = | N = | N = | N = | N = |
| Class | Description | 195,223 | 650,553 | 5,983,418 | 202,042 | 351,200 | 33,607 | 661,759 | 8,077,802 |
| HLA- | Allele Count by | 388,476 | 1,291,125 | 11,929,417 | 402,447 | 700,632 | 66,971 | 1,320,493 | 16,099,561 |
| A | Population Group | | | | | | | | |
| | in CIWD | | | | | | | | |
| | Covered by Model | 356,264 | 1,210,978 | 11,192,793 | 380,568 | 621,447 | 60,270 | 1,204,657 | 15,026,977 |
| | population's HLA | | | | | | | | |
| | set (n = 49) | | | | | | | | |
| | Coverage | 98.2% | 95.9% | 99.3% | 97.6% | 97.0% | 96.7% | 98.0% | 98.7% |
| HLA- | Allele Count by | 388,579 | 1,298,351 | 11,941,489 | 402,160 | 700,912 | 66,967 | 1,320,714 | 16,119,172 |
| B | Population Group | | | | | | | | |
| | in CIWD | | | | | | | | |
| | Covered by Model | 356,687 | 1,169,460 | 10,821,481 | 358,189 | 580,452 | 57,823 | 1,176,597 | 14,520,689 |
| | population's HLA | | | | | | | | |
| | set (n = 71) | | | | | | | | |
| | Coverage | 96.4% | 91.8% | 96.1% | 91.5% | 88.6% | 91.3% | 94.2% | 95.1% |
| HLA- | Allele Count by | 389,619 | 1,255,403 | 11,827,887 | 403,229 | 690,043 | 67,072 | 1,302,662 | 15,935,915 |
| C | Population Group | | | | | | | | |
| | in CIWD | | | | | | | | |
| | Covered by Model | 343,565 | 1,132,914 | 10,400,481 | 364,466 | 583,484 | 55,031 | 1,132,848 | 14,012,789 |
| | population's HLA | | | | | | | | |
| | set (n = 32) | | | | | | | | |
| | Coverage | 98.9% | 94.8% | 99.2% | 96.0% | 96.2% | 96.2% | 98.5% | 98.5% |

| | |
|---|---|
| HLA-A-B-C coverage by Model population’s HLA set (n = 152): | 97.4% |

African/African American (AFA),
Asian/Pacific Islands (API),
European/European descent (EURO),
Middle East/North coast of Africa (MENA),
South or Central America/Hispanic/Latino (HIS),
Native American populations (NAM),
Unknown/Not asked/Multiple ancestries/Other (UNK).
CIWD 3.0: Common (>=1 in 10,000), Intermediate (>=1 in 100,000) and Well Documented (>=5 occurrence) HLA database 3.0 (released in 2020). Related to FIG. 24.

A second model cohort of 356 individuals with characterized HLA class II genotypes (2×HLA-DRB, 2×HLA-DP, and 2×HLA-DQ) at four-digit allele resolution was obtained from the dbMHC database(47), an online available repository operated by the National Center for Biotechnology Information (NCBI) developed for evaluating the allelic composition of cDNA or genomic sequences. Sampling was performed for a wide range of ethnicities in many countries around the world. In total, 356 subjects in this database had HLA class II genotype data with sufficient resolution (2×HLA-DRB, 2×HLA-DP, and 2×HLA-DQ with at least four-digit resolution). HLA genotyping was performed by SBT.

Large, US Cohort (n=16,000)

The database comprising data from 16,000 individuals was created by obtaining 1,000 donors from each of 16 ethnic groups (500 male and 500 female) from the National Marrow Donor Program (NMDP).(48) The 16 ethnic groups were: African, African American, Asian Pacific Islander, Filipino, Black Caribbean, Caucasian, Chinese, Hispanic, Japanese, Korean, Native American Indian, South Asian, Vietnamese, US, Mideast/North coast of Africa, Hawaiian, and other Pacific Islander. HLA genotyping was performed by NMDP recruitment labs using sequence-specific oligonucleotide (SSO) and sequence specific primer (SSP) methods with an average “typing resolution score” >0.7.(49)

### Peptides and PolyPEPI-SCoV-2 Vaccine Preparation

The 9-mer (s2, s5, s9, n1, n2, n3, n4, el, ml) and 30-mer (S2, S5, S7, N1, N2, N3, N4, E1, M1) peptides were manufactured by Intavis Peptide Services GmbH&Co. KG (Tübingen, Germany) and PEPScan (Lelystad, The Netherlands) using solid-phase peptide synthesis. Amino acid sequences are provided in Table 9 for both 9-mer test peptides (Table 9, bold) and the 30-mer vaccine peptides. The peptide vaccine for the animal study was prepared by dissolving equal masses of the nine 30-mer peptides in DMSO to achieve at a concentration of 1 mg/mL and then diluted with purified water to a final concentration of 0.2 mg/mL and stored frozen until use. Ready-to-inject vaccine preparations were prepared by emulsifying equal volumes of thawed peptide mix solution and Montanide ISA 51 VG adjuvant (Seppic, Paris, France) following the standard two-syringe protocol provided by the manufacturer.

### Epitope Prediction and Analysis

Prediction of ≥3HLA class I allele binding epitopes (PEPIs) within each individual was performed using an Immune Epitope Database (IEDB)-based epitope prediction method. The antigens were scanned with overlapping 9-mer and 15-mer peptides to identify peptides that bind to the subject's HLA class I alleles. Selection parameters were validated with an in-house set of 427 HLA-peptide pairs that had been characterized experimentally by using direct binding assays (327 binding and 100 non-binding HLA-epitope pairs). Both specificity and sensitivity resulted in 93% for the prediction of true HLA allele-epitope pairs. HLA class II epitope predictions were performed by NetMHCpan (2.4) prediction algorithm, ≥4 HLA class II binding epitopes per individual are defined as HLA class II PEPI.

### Preclinical Mice Study Design

Thirty-six Hu-mice and 36 BALB/c mice received PolyPEPI-SCoV-2 vaccine (0.66 mg/kg/peptide in 200 μL solution; n=18) or 20% DMSO/water emulsified in Montanide ISA 51 VG adjuvant (200 μL vehicle; n=18) administered subcutaneously on days 0 and 14; the follow up period ended on day 28. Samples from days 14, 21, and 28 were analyzed (n=6 per cohort). The studies were performed at the Transcure Bioservices facility (Archamps, France). The mice were monitored daily for unexpected signs of distress. Complete clinical scoring was performed weekly by monitoring coat (score 0-2), movement (0-3), activity (0-3), paleness (0-2), and bodyweight (0-3); a normal condition was scored 0.

All procedures described in this study have been reviewed and approved by the local ethic committee (CELEAG) and validated by the French Ministry of Research. Vaccination-induced T cell responses were assessed by ex vivo ELISpot and intracellular cytokine staining assays (ICS) of mice splenocytes (detailed below). Antibody responses were investigated by the measurement of total IgG in plasma samples (detailed below).

### ELISpot/FluoroSpot Assays

Ex vivo ELISPot assays for animal studies were performed as follows. IFN-γ-producing T cells were identified using 2×10⁵ splenocytes stimulated for 20 h/peptide (10 g/ml, final concentration). Splenocytes were treated with 9-mer peptides (a pool of four N-specific peptides, N-pool (n1, n2, n3, n4), a pool of three S-specific peptides, S-pool (s2, s5, s9), an E protein-derived peptide, el or a M protein-derived peptide, ml)) or with 30-mer peptides pooled the same way as 9-mers (N-pool comprising peptides N1, N2, N3, and N4), S-pool comprising peptides S2, S5 and S9, and individual peptides E1 and M1. ELISpot assays were performed using Human IFN-γ ELISpot PRO kit (ALP; ref 3321-4APT-2) from mabTech for Hu-mice cohorts and Mouse IFN-γ ELISpot PRO kit (ALP; ref 3321-4APT-10) from mabTech for Balb/c mice cohorts, according to the manufacturer's instructions. Unstimulated (DMSO) assay control background spot forming unit (SFU) was subtracted from each data point and then the delta SFU (dSFU) was calculated. PMA/Ionomycin (Invitrogen) was used as a positive control.

Ex vivo FluoroSpot assays for convalescent donor testing were performed by Nexelis-IMXP (Belgium) as follows: IFN-γ/IL-2 FluoroSpot plates were blocked with RPMI-10% FBS, then peptides (5 μg/mL final concentration) or peptide pools (5 μg/mL per peptide final concentration) were added to the relevant wells. PBMCs were retrieved from cryogenic storage and thawed in culture medium. Then, 200,000 PBMC cells/well were plated in triplicate (stimulation conditions) or 6-plicates (reference conditions) and incubated overnight at 37° C., 5% CO₂ before development. Development of the FluoroSpot plates was performed according to the manufacturer's recommendations. After removing cells, detection antibodies diluted in PBS containing 0.1% BSA were added to the wells and the FluoroSpot plates were incubated for 2 hours at room temperature. Before read-out using the Mabtech IRIS™ automated FluoroSpot reader, the FluoroSpot plates were emptied and dried at room temperature for 24 h protected from light. All data were acquired with a Mabtech IRIS™ reader and analyzed using Mabtech Apex™ software. Unstimulated (DMSO) negative control, CEF positive control (T-cell epitopes derived from CMV, EBV and influenza, Mabtech, Sweden), and a commercial SARS-CoV-2 peptide pool (SARS-CoV-2 S N M O defined peptide pool (3622-1)—Mabtech, Sweden) were included as assay controls. Ex vivo FluoroSpot results were considered positive when the test result was higher than DMSO negative control after subtracting non-stimulated control (dSFU).

Enriched ELISpot assays for convalescent donor testing were performed by Nexelis-IMXP (Belgium) as follows: PBMCs were retrieved from cryogenic storage and thawed in culture medium. The PBMCs were seeded at 4,000,000 cells/24-well in presence of the peptide pools (5 μg/ml per peptide) and incubated for 7 days at 37° C., 5% CO₂. On days 1 and 4 of culture, the media were refreshed and supplemented with 5 ng/mL IL-7 or 5 ng/mL IL-7 and 4 ng/ml IL-2 (R&D Systems), respectively. After 7 days of culture, the PBMCs were harvested and rested for 16 h. The rested PBMCs were then counted using Trypan Blue Solution, 0.4% (VWR) and the Cellometer K2 Fluorescent Viability Cell Counter (Nexcelom), and seeded on the IFN-γ7Granzyme-B/TNF-α FluoroSpot plates (Mabtech) at 200,000 cells/well in RPMI 1640 with 10% Human Serum HI, 2 mM L-glutamin, 50 μg/ml gentamycin and 100 μM β-ME into the relevant FluoroSpot wells containing peptide (5 μg/mL), or peptide pool (5 μg/mL per peptide). The FluoroSpot plates were incubated overnight at 37° C., 5% CO₂ before development. All data were acquired with a Mabtech IRIS™ reader and analyzed using Mabtech Apex™ software. DMSO, medium only, a commercial COVID peptide pool (SARS-CoV-2 S N M O defined peptide pool [3622-1]—Mabtech), and CEF were included as assay controls at a concentration of 1 μg/ml. The positivity criterion was >1.5-fold the unstimulated control after subtracting the background (dSFU).

### Intracellular Cytokine Staining (ICS) Assay

Ex vivo ICS assays for preclinical mice studies were performed as follows: splenocytes were removed from the ELISpot plates after 20 h of stimulation, transferred to a conventional 96-well flat bottom plate, and incubated for 4 h with BD GolgiStop™ according to the manufacturer's recommendations. Flow-cytometry was performed using a BD Cytofix/Cytoperm Plus Kit with BD GolgiStop™ protein transport inhibitor (containing monensin; Cat. No. 554715), following the manufacturer's instructions. Flow cytometry analysis and cytokine profile determination were performed on an Attune NxT Flow cytometer (Life Technologies). A total of 2×10⁵ cells were analyzed, gated for CD45⁺, CD3⁺, CD4⁺, or CD8⁺ T cells. Counts below 25 were evaluated as 0. Spot counts≥25 were background corrected by subtracting unstimulated (DMSO) control. PMA/Ionomycin (Invitrogen) was used as a positive control. As an assay control, Mann-Whitney test was used to compare negative control (unstimulated) and positive control (PMA/ionomycin) for each cytokine. When a statistical difference between controls was determined, the values corresponding to the other stimulation conditions were analyzed. The following stains were used for Hu-mice cohorts: MAb 11 502932 (Biolegend), MP4-25D2 500836 (Biolegend), 4S.B3 502536 (Biolegend), HI30 304044 (Biolegend), SK7 344842 (Biolegend), JES6-5H4 503806 (Biolegend), VIT4 130-113-218 (Miltenyi), JES1-39D10 500904 (Biolegend), SK1 344744 (Biolegend), JES10-5A2 501914 (Biolegend), JES3-19F1 554707 (BD), and NA 564997 (BD). The following stains were used for BALB/c mice cohorts: 11B11 562915 (BD), MP6-XT22 506339 (Biolegend), XMG1.2 505840 (Biolegend), 30-F11 103151 (Biolegend), 145-2C11 100355 (Biolegend), JES6-5H4 503806 (Biolegend), GK1.5 100762 (Biolegend), JES1-39D10 500904 (Biolegend), 53-6.7 100762 (Biolegend), eBiol3A 25-7133-82 (Thermo Scientific), JESS-16E3 505010 (Biolegend), and NA 564997 (BD).

Ex vivo ICS assays for convalescent donor testing were performed by Nexelis-IMXP (Belgium). Briefly, after thawing 200,000 PBMC cells/well, PBMCs were seeded in sterile round-bottom 96-well plates in RPMI total with 10% human serum HI, 2 mM L-glutamine, 50 μg/mL gentamycin, and 100 μM 2-ME in the presence of peptides (5 g/mL) or peptide pool (5 μg/mL per peptide). After a 2-hour incubation, BD GolgiPlug™ (BD Biosciences) was added to the 96-well plates at a concentration of 1 l/ml in culture medium. After a 10-h incubation, plates were centrifuged (800 g, 3 min, 8° C.) and incubated for 10 min at 37° C. and Zombie NIR Viability dye (Biolegend) was added to each well. Plates were incubated at room temperature for 15 min, shielded from the light. After incubation, PBS/0.1% BSA was added per well and the plates were centrifuged (800 g, 3 min, 8° C.). Thereafter, cells were incubated in FcR blocking reagent at 4° C. for 5 min, and then staining mixture (containing anti-CD3, Biolegend, anti-CD4, and anti-CD8 antibodies; BD Biosciences) was added to each well. After 30 min of incubation at 4° C., washing, and centrifugation (800 g, 3 min, 8° C.), cells were permeabilized and fixed according to the manufacturer's recommendations (BD Biosciences). After fixation, cytokine staining mixture (containing anti-IFN-γ, anti-IL-2, anti-IL-4, anti-IL-10 and anti-TNF-α antibodies, Biolegend) was added to each well. Plates were incubated at 4° C. for 30 min and then washed twice before acquisition. All flow cytometry data were acquired with LSRFortessa™ X-20 and analyzed using FlowJo V10 software. DMSO negative control was subtracted from each data point obtained using test peptides or pools.

### Antibody ELISA

ELISAs for mouse studies were performed for the quantitative measurement of total mouse IgG production in plasma samples using IgG (Total) Mouse Uncoated ELISA Kit (Invitrogen, #88-50400-22) for BALC/c cohorts and IgG (Total) Human Uncoated ELISA Kit (Invitrogen, #88-50550-22) for Hu-mice cohorts according to the manufacturer's instructions. Analyses were performed using samples harvested at days 14, 21, and 28 (n=6 per group per time point). Absorbance were read on an Epoch Microplate Reader (Biotech) and analyzed using Gen5 software.

ELISAs for convalescent donor testing were performed by Mikromikomed Kft (Budapest, Hungary) using a DiaPro COVID-19 IgM Enzyme Immunoassay for the determination of IgM antibodies to COVID-19 in human serum and plasma, DiaPro COVID-19 IgG Enzyme Immunoassay for the determination of IgG antibodies to COVID-19 in human serum and plasma, and DiaPro COVID-19 IgA Enzyme Immunoassay for the determination of IgA antibodies to COVID-19 in human serum and plasma, according to the manufacturer's instructions (Dia.Pro Diagnostic Bioprobes S.r.l., Italy). For the determination of total N-specific Ig antibodies, Roche Elecsys® Anti-SARS-CoV-2 Immunoassay for the qualitative detection of antibodies (including IgG) against SARS-CoV-2 was used with a COBAS e411 analyzer (disk system; ROCHE, Switzerland) according to the manufacturer's instructions. EUROIMMUN ELISA assays were performed to determine S1-specific IgG levels in convalescent donors via the independent medical research center. The Anti-SARS-CoV-2 ELISA plates are coated with recombinant S1 structural protein from SARS-CoV-2 to which antibodies against SARS-CoV-2 bind. This antigen was selected for its relatively low homology to other coronaviruses, notably SARS-CoV-1. The immunoassay was performed according to the manufacturer's instructions.

### Pseudoparticle Neutralization Assay

Neutralizing antibodies in mice sera were assessed using a cell-based Pseudoparticle Neutralization Assay (PNA) according to dose range finding: SARS-CoV-2 Pseudoparticle Neutralization Assay Testing. Vero E6 cells expressing the ACE-2 receptor (Vero C1008 (ATCC No. CRL-1586, US), were seeded at 20 000 cells/well to reach a cell confluence of 80%. Serum samples and controls (pool of human convalescent serum, internally produced) were diluted in duplicates in cell growth media at a starting dilution of 1/25 or 1/250 (for samples) or 1/100 (for controls), followed by a serial dilution (2-fold dilutions, 5 times). In parallel, SARS-CoV-2 pseudovirus (prepared by Nexelis, using Kerafast system, with Spike from Wuhan Strain (minus 19 C-terminal amino acids) was diluted as to reach the desired concentration (according to pre-determined TU/mL). Pseudovirus was then added to diluted sera samples and pre-incubated for 1 hour at 37° C. with CO2. The mixture was then added to the pre-seeded Vero E6 cell layers and plates were incubated for 18-24 hours at 37° C. with 5% C02. Following incubation and removal of media, ONE-Glo EX Luciferase Assay Substrate, Promega, Cat. E8110) was added to cells and incubated for 3 minutes at room temperature with shaking. Luminescence was measured using a SpectraMax i3x microplate reader and SoftMax Pro v6.5.1 (Molecular Devices). Luminescence results for each dilution were used to generate a titration curve using a 4-parameter logistic regression (4PL) using Microsoft Excel (for Microsoft Office 365). The titer was defined as the reciprocal dilution of the sample for which the luminescence is equal to a pre-determined cut-point of 50, corresponding to 50% neutralization. This cut-point was established using linear regression using 50% flanking points.

### Statistical Analysis

Significance was compared between and among groups using two tailed t-tests or Mann-Whitney tests, as appropriate, p<0.05 was considered significant. Pearson's test was performed to assess correlations. The correlation was considered strong if R>0.7, moderate, if 0.5≤R≤0.7 and weak, if 0.3<R≤0.5.(50)

### Results

### Tailoring PolyPEPI-SCoV-2 to Individuals

During the design of PolyPEPI-SCoV-2, we used the HLA-genotype data of subjects in the in silico human cohort to determine the most immunogenic peptides (i.e., HLA class I PEPI hotspots, 9-mers) of the four selected SARS-CoV-2 structural proteins aimed to induce CD8⁺ T cell responses. The sequences of the identified 9-mer PEPI hotspots were then extended to 30-mers based on the viral protein sequences to maximize the number of HLA class II binding PEPIs (15-mers) aimed to induce CD4⁺ T cell responses.

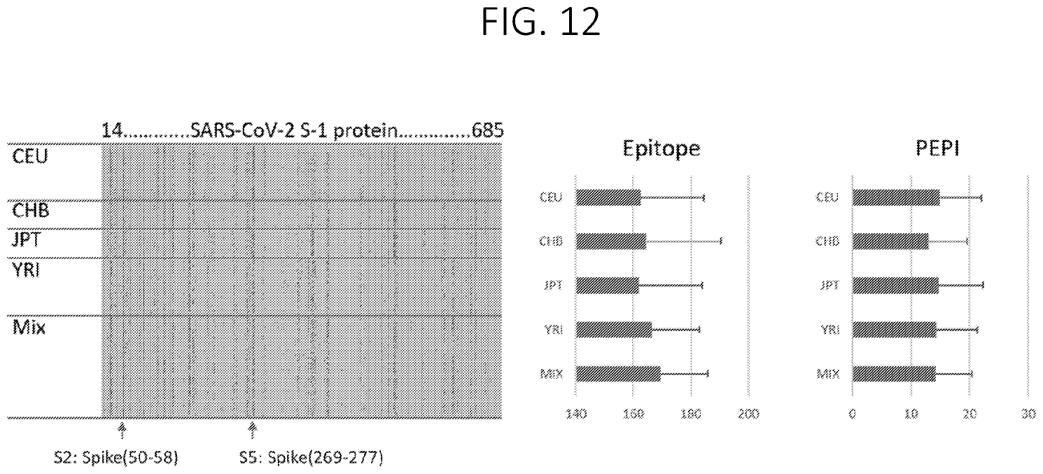

First, we performed epitope predictions for each subject in the in silico human cohorts for each of their HLA class I and class II genotypes (six HLA class I and eight HLA class II alleles) for the AA sequence of the conserved regions of 19 known SARS-CoV-2 viral proteins using 9-mer (HLA class I) and 15-mer (HLA class II) frames, respectively (FIG. 1). Then, we selected the epitopes (PEPIs) that could bind to multiple (≥3) autologous HLA alleles. We identified several HLA-restricted epitopes (average, 166 epitopes only for S-1 protein) for each person. In contrast, PEPIs are represented at much lower level in all ethnicities (average, 14 multi-HLA-binding epitopes, FIG. 12).

From the resulting PEPI list, we identified nine 30-mer polypeptide fragments that comprise overlapping, class I and class II PEPIs shared (frequent) among a high percentage of individuals in the model population, independent of ethnicity (Table 9). To maximize multi-antigenic immune responses at both the individual and population levels, we selected more than one peptide from the large spike (S) and nucleoprotein (N) proteins and only one peptide from the shorter membrane (M) and envelope (E) proteins. From the four structural viral antigens of the SARS-CoV-2 virus, a total of nine 30-mer peptides were selected for the vaccine, also considering the chemical and manufacturability properties of the peptides: three peptides derived from S, four peptides from N, and one peptide derived from each M and E. No peptides were included from the receptor-binding domain of S-1 protein. Overall, each member of the model population had PEPIs for at least two of the nine peptides, and 97% had at least three (Table 9).

TABLE 9
PolyPEPI-SCoV-2 peptides and comprising PEPI frequencies
within the in silica human cohort. Bold: 9-mer HLA class
I PEPI sequences within PolyPEPI-SCoV-2 comprising 30-mer
peptides; underlined: 15-mer HLA class II PEPI sequences.
Percentages show the proportion of individuals from the
model population with at least one HLA class I (CD8⁺ T
cell specific) PEPI or at least one HLA class II
(CD4⁺ T cell specific) PEPI. Peptides labeled †
contain experimentally confirmed B cell epitopes
with antibody (Ig) responses found in convalescent
patients with SARS. N/A: data not available.

| | | | | | | B cell |
|---|---|---|---|---|---|---|
| SARS- | | SEQ | | Class | Class | epitope |
| CoV-2 | Peptide | ID | | I | II | in SARS |
| fragment | ID | No. | Peptide (30-mer) | PEPI | PEPI | (ref) |
| S (35-64) | S2 | 216 | GVYYPDKVFRSSVLHSTQ | 71% | 94% | N/A |
| | | | DLFLPFFSNVTW | | | |
| S (253- | S5 | 217 | DSSSGWTAGAAAYYVGYL | 84% | 97% | N/A |
| 282) | | | QPRTFLLKYNEN | | | |
| S (893- | S9 | 218 | ALQIPFAMQMAYRFNGIG | 93% | 99% | IgM 50% |
| 922)† | | | VTQNVLYENQKL | | | |
| N (36- | N1 | 219 | RSKQRRPQGLPNNTASWF | 36% | 36% | (n = 4)(51) |
| 65)† | | | TALTQHGKEDLK | | | IgG |
| N (255- | N2 | 220 | SKKPRQKRTATKAYNVTQ | 48% | 22% | 62% (n = 42) |
| 284) | | | AFGRRGPEQTQG | | | (52, 53) |
| | | | | | | N/A |
| N (290- | N3 | 221 | EL [symbol] [symbol] IAQF | 54% | 50% | IgG |
| 319)† | | | APSASAFFGMSR | | | 34% |
| | | | | | | (n = 42)(53) |
| | | | | | | IgG,IgM |
| | | | | | | 50% (n = 4)(51) |
| N (384- | N4 | 222 | QRQKKQQTVT [symbol] DLD | 23% | 36% | IgG, IgM |
| 413)† | | | DFSKQLQQSMSS | | | 95% |
| | | | | | | (n = 42)(53) |
| | | | | | | IgG,IgM |
| | | | | | | 75% (n = 4)(51) |
| M (93- | M1 | 223 | LSYFIASFRLFARTRSMW | 90% | 100% | N/A |
| 122) | | | SFNPETNILLNV | | | |
| E (45-74) | E1 | 224 | NIVNVSLVKPSFYVYSRV | 46% | 100% | N/A |
| | | | KNLNSSRVPDLL | | | |

Combined frequency of PolyPEPI-SCoV-2 PEPIs

| | | | |
|---|---|---|---|
| At least one peptide (PEPI Score) | 100% | 100% | |
| At least two peptides | 100% | 100% | N/A |
| At least three peptides | 97% | 100% | |

We identified experimentally confirmed linear B cell epitopes derived from SARS-CoV-1, with 100% sequence identity to the relevant SARS-CoV-2 antigen, to account for the potential B cell production capacity of the long peptides.(54) Three overlapping epitopes located in N protein- and one epitope in S-protein-derived peptides of PolyPEPI-SCoV-2 vaccine were reactive with the sera of convalescent patients with severe acute respiratory syndrome (SARS). This suggests that the above antigenic sites on the S and N protein are important in eliciting a humoral immune response against SARS-CoV-1 and likely against SARS-CoV-2 in humans.

As expected, PolyPEPI-SCoV-2 contains several (eight out of nine) peptides that are cross-reactive with SARS-CoV due to high sequence homology between SARS-CoV-2 and SARS-CoV. Sequence similarity is low between the PolyPEPI-SCoV-2 peptides and common (seasonal) coronavirus strains belonging to alpha coronavirus (229E and NL63), beta coronavirus (OC43, HKU1) and MERS. Therefore, cross-reactivity between the vaccine and prior coronavirus-infected individuals remains low and might involve only the M1 peptide of the vaccine (See Methods and Table 10).

### PolyPEPI-SCoV-2 Vaccine-Induced Broad T Cell Responses in Mice

Preclinical immunogenicity testing of PolyPEPI-SCOV-2 vaccine was performed to measure the induced immune responses after one and two vaccine doses that were administered two weeks apart (days 0 and 14) in a non-humanized BALB/c model and in the humanized immune setting of CD34+ Hu-NCG (Hu-mice) mice. After immunizations, no mice presented any clinical score (score 0, representing no deviation from normal), suggesting the absence of any side effects or immune aversion. In addition, the necropsies performed by macroscopic observation at each timepoint did not reveal any visible organ alteration in spleen, liver, kidneys, stomach and intestine. Repeated vaccine administration was also well tolerated, and no signs of immune toxicity or other systemic adverse events were detected. Together, these data strongly suggest that the formulation used in this study was safe in mice.

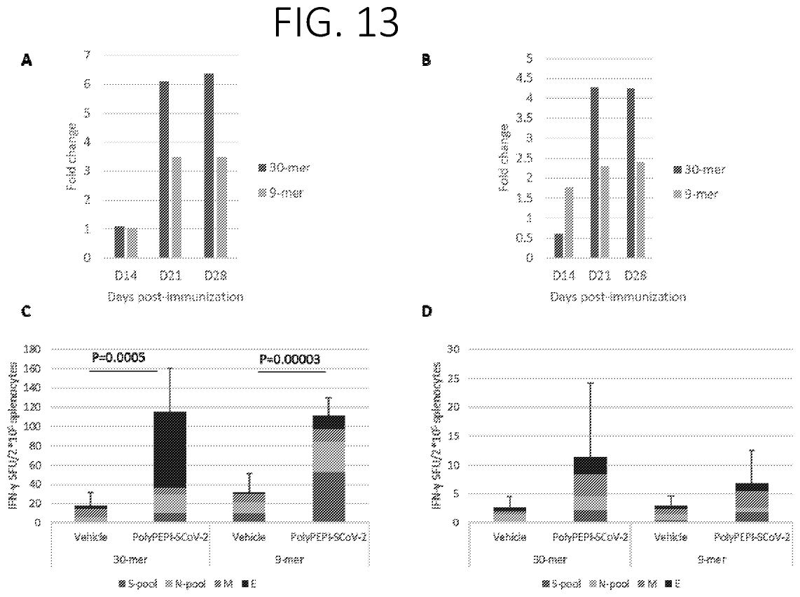

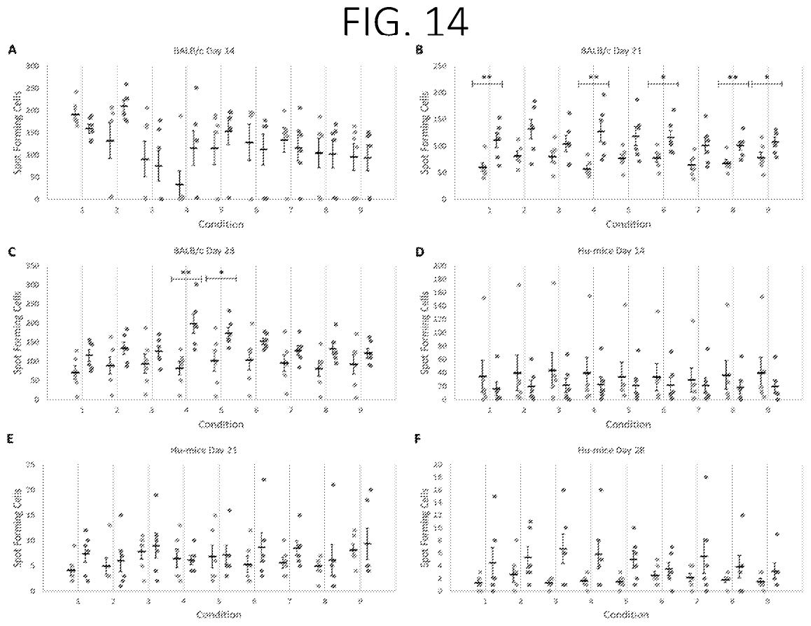

IFN-γ producing vaccine-induced T cells were measured after the first dose at day 14 (1D14) and after the second dose at days 21 (D21) and 28 (1D28). At day 14, PolyPEPI-SCoV-2 treatment did not induce more IFN-γ production by CD8+ T cells than Vehicle (DMSO/Water emulsified with Montanide) treatment, this latter resulting in unusually high response probably due to Montanide mediated unspecific responses. Nevertheless, at days 21 and 28, the second dose of PolyPEPI-SCoV-2 boosted IFN-γ production compared to Vehicle control group by six-fold and 3.5-fold for splenocytes stimulated with the 30-mer and 9-mer pool of peptides, respectively (FIG. 13A). The increased IFN-γ production by T cells from PolyPEPI-SCoV-2-treated mice at days 21 and 28 was confirmed by the increased number of spot-forming cells, which reached statistical significance for the following conditions: 30-mer S-pool, peptide E1, 9-mer N-pool and peptide el at day 21 and peptide E1 and 9-mer S-pool at day 28, respectively (FIG. 14A-C).

In immunodeficient Hu-mice at day 14, PolyPEPI-SCoV-2 treatment increased IFN-γ production by two-fold with splenocytes stimulated with the 9-mer pool of peptides, but no increase was observed with 30-mer-stimulated splenocytes. At days 21 and 28, the second dose of PolyPEPI-SCoV-2 boosted IFN-γ production by two- and four-fold with splenocytes stimulated with the 9-mer and 30-mer pools of peptides, respectively (FIG. 13B). Importantly, both 9-mer detected CD8⁺ T cells and 30-mer-detected CD4⁺ and CD8⁺ T cell responses were equally directed against all four viral proteins targeted by the vaccine in both animal models (FIG. 13C, D and detailed results in FIG. 14D-F).

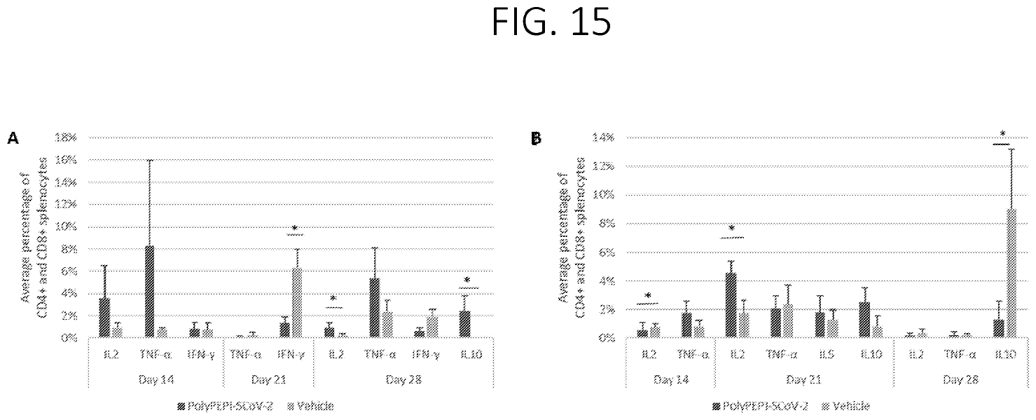

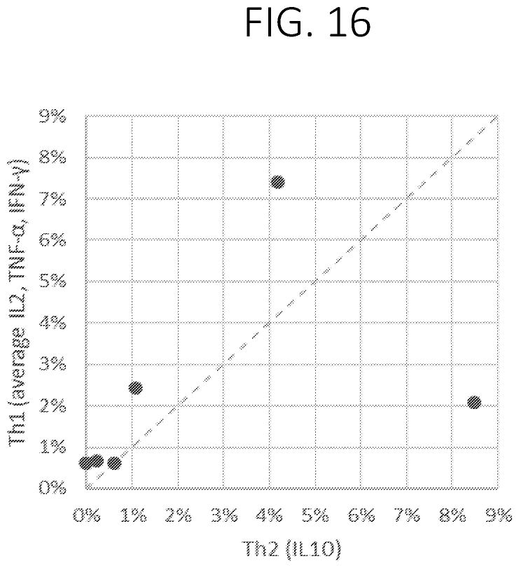

Intracellular staining (ICS) assay was performed to investigate the polarization of the T cell responses elicited by the vaccination. Due to the low frequency of T cells, individual peptide-specific T cells were more difficult to visualize by ICS than by ELISpot, but a clear population of CD4⁺ and CD8⁺ T cells producing Th1-type cytokines of TNF-α and IL-2 were detectable compared to animals receiving only vehicle (DMSO/water emulsified with Montanide) in both BALB/c and Hu-mice models (FIG. 15). For Th2-type cytokines IL-4 and IL-13, analysis did not reveal any specific response at any timepoint. Low levels of IL-5 and/or IL-10 cytokine-producing CD4⁺ T cells were detected for both models but it was significantly different from Vehicle control only for BALB/c mice at day 28. Even for this cohort the Th1/Th2 balance remained shifted towards Th1 for 5 out of 6 mice (one outlier) confirming an overall Th1-skewed T cell response elicited by the vaccine (FIG. 16).

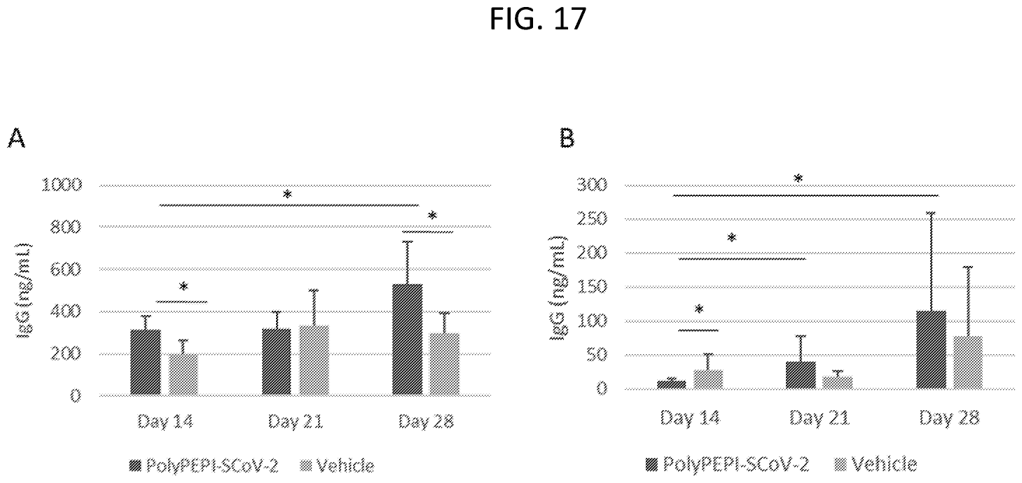

PolyPEPI-SCoV-2 vaccination also induced humoral responses, as measured by total mouse IgG for BALB/c and human IgG for Hu-mice. In BALB/c mice, vaccination resulted in vaccine-induced IgG production after the first dose (day 14) compared with control animals receiving only vehicle. IgG elevation were observed for both BALB/c and Hu-mice models at later time points after the second dose (FIG. 17). As expected, given that PolyPEPI-SCoV-2 peptides do not contain conformational epitopes, vaccination did not result in neutralizing antibodies as assessed from the sera of Hu-mice using neutralization assay with pseudo-particles (data not shown).

### PolyPEPI-SCoV-2 Vaccine Induced Broad T Cell Responses in Two Animal Models

Preclinical immunogenicity testing of PolyPEPI-SCoV-2 vaccine was performed to measure the induced immune responses after one and two vaccine doses that were administered two weeks apart (days 0 and 14) in BALB/c and Hu-mouse models. After immunizations, no mice presented any clinical score at day 14, 21 or 28 (score 0, representing no deviation from normal), suggesting the absence of any side effects or immune aversion (Tables 10A and B). In addition, the necropsies performed by macroscopic observation at each time point did not reveal any visible organ alteration in spleen, liver, kidneys, stomach and intestine (Table 10C). Repeated vaccine administration was also well tolerated, and no signs of immune toxicity or other systemic adverse events were detected. Together, these data strongly suggest that PolyPEPI-SCoV-2 was safe in mice.

Vaccine-induced IFN-γ producing T cells were measured after the first dose at day 14 and after the second dose at days 21 and 28. Vaccine-induced T cells were detected using the nine 30-mer vaccine peptides grouped in four pools according to their source protein: S, N, M, and E, to assess for the CD4+ and CD8+ T cell responses. CD8+ T cell responses were also specifically measured using the short 9-mer test peptides corresponding to the shared HLA class I PEPIs defined above for each of the nine vaccine peptides, in four pools (s, n, m, and e peptides; Table 9 bold).

In BALB/c mice at day 14, PolyPEPI-SCoV-2 vaccination did not induce more IFN-γ production than the Vehicle (DMSO/Water emulsified with Montanide), this latter resulting in unusually high response probably due to Montanide mediated unspecific responses. Nevertheless, at days 21 and 28, the second dose of PolyPEPI-SCoV-2 increased IFN-γ production compared to Vehicle control group by six-fold and 3.5-fold for splenocytes detected with the 30-mer and 9-mer peptides, respectively (FIG. 13A).

In immunodeficient Hu-mice at day 14, PolyPEPI-SCoV-2 vaccination increased IFN-γ production by two-fold with splenocytes specific for the 9-mer pool of peptides, but no increase was observed with 30-mer-stimulated splenocytes. At days 21 and 28, the second dose of PolyPEPI-SCoV-2 boosted IFN-γ production by four- and two-fold with splenocytes detected with the 30-mer and 9-mer pools of peptides, respectively (FIG. 13B). Importantly, both 9-mer-detected CD8+ T cells and 30-mer-detected CD4+ and CD8+ T cell responses were directed against all four viral proteins targeted by the vaccine in both animal models (FIG. 13C-D and FIG. 14D-F). Since the Hu-mouse model was developed by transplanting human CD34+ hematopoietic stem cells to generate human antigen-presenting cells and T- and B-lymphocytes into NOD/Shi-scid/IL-2Rγ null immunodeficient mice, this model provides a real human immune system model (Brehm et al. 2013). Therefore, the robust multi-antigenic CD4+ and CD8+ T cell responses obtained in this model indicate that the vaccination resulted in properly processed and HLA-presented epitopes and subsequent antigen-specific T cell responses by the human immune cells of the Hu-mice.

ICS assay was performed to investigate the polarization of the T cell responses elicited by the vaccination. Due to the low frequency of T cells, individual peptide-specific T cells were more difficult to visualize by ICS than by ELISpot, but a clear population of CD4+ and CD8+ T cells producing Th1-type cytokines of TNF-α and IL-2 were detectable compared to animals receiving Vehicle in both BALB/c and Hu-mouse models (FIG. 15). For IL-4 and IL-13 Th2-type cytokines, analysis did not reveal any specific response at any time point. Low levels of IL-5 and/or IL-10 cytokine-producing CD4+ T cells were detected for both models but it was significantly different from Vehicle control only for BALB/c mice at day 28. Even for this cohort the Th1/Th2 balance remained shifted towards Th1 for 5 out of 6 mice (one outlier) confirming an overall Th1-skewed T cell response elicited by the vaccine (FIG. 16).

PolyPEPI-SCoV-2 vaccination also induced humoral responses, as measured by total mouse IgG for BALB/c and human IgG for Hu-mouse models. In BALB/c mice, vaccination resulted in vaccine-induced IgG production after the first dose (day 14) compared with Vehicle control group. IgG elevation were observed for both BALB/c and Hu-mouse models at later time points after the second dose (FIG. 17). IgG levels measured from the plasma of Hu-mice (average 115 ng/mL, FIG. 17B) were lower than for BALB/c (average 529 ng/mL, FIG. 17A) at D28. This is consistent with the known limitation of the NOD/Shi-scid/IL-2Rγ null immunodeficient mouse regarding its difficulty generating the human humoral responses that lead to class-switching and IgG production (Brehm et al. 2013). Humanization rate of ˜50% in the Hu-mouse model further reduces the theoretically expected IgG levels. Despite these limitations, the dose-dependent human IgG production indicates vaccine-generated human imoral responses. As expected, given that PolyPEPI-SCoV-2 peptides do not contain conformational B cell epitopes, vaccination did not result in measurable neutralizing antibodies as assessed from the sera of Hu-mice using PNA assay. A 50% Neutralizing Antibody Titer (NT50) was undetectable at the assay detection limit of 1:25 dilution, for each tested samples (data not shown).

TABLE 10A
Safety analysis, clinical score data table of BALB/c mice. Clinical safety scores were
established by characterization of five different clnical signs (coat, movement, activity, paleness, body
weight), according to the following specification: Coat: score 0-normal; score 1-lack of grooming,
partial alopecia; score 2-massive alopecia, wounds, bleedings, inflammation. Movement: score 0-
normal; score 2-slow movement, paralysis of one animal; score 3-difficulties to eat and drink,
paralysis to more than one animal. Activity: score 0-normal; score 1-agitated, over-reactive, hypo-
reactive; score 3-prostrated. Paleness: score 0-normal; score 1-slight (no ear vessels visible); score
2-severe (ears plus feet affected). Body weight: score 0-normal; score 2-segmentation of the
vertebral column evident, pelvic bones palpable; score 3-skeletal structure prominent. Maximum
cumulative clinical score allowed: 6. n.a.: not applicable.

| Days after 1^{st} vaccination: | | | −2 | 6 | 12 | 19 | 26 |
|---|---|---|---|---|---|---|---|
| Mouse strain | Mouse ID | treatment | Cumulative clinical score | | | | |
| BALB/c | 1 | PolyPEPI- | 0 | 0 | 0 | n.a. | n.a. |
| | 2 | SCoV-2 | 0 | 0 | 0 | n.a. | n.a. |
| | 3 | | 0 | 0 | 0 | n.a. | n.a. |
| | 4 | | 0 | 0 | 0 | n.a. | n.a. |
| | 5 | | 0 | 0 | 0 | n.a. | n.a. |
| | 6 | | 0 | 0 | 0 | n.a. | n.a. |
| | 7 | | 0 | 0 | 0 | 0 | n.a. |
| | 8 | | 0 | 0 | 0 | 0 | n.a. |
| | 9 | | 0 | 0 | 0 | 0 | n.a. |
| | 10 | | 0 | 0 | 0 | 0 | n.a. |
| | 11 | | 0 | 0 | 0 | 0 | n.a. |
| | 12 | | 0 | 0 | 0 | 0 | n.a. |
| | 13 | | 0 | 0 | 0 | 0 | 0 |
| | 14 | | 0 | 0 | 0 | 0 | 0 |
| | 15 | | 0 | 0 | 0 | 0 | 0 |
| | 16 | | 0 | 0 | 0 | 0 | 0 |
| | 17 | | 0 | 0 | 0 | 0 | 0 |
| | 18 | | 0 | 0 | 0 | 0 | 0 |
| | 19 | Vehicle | 0 | 0 | 0 | n.a. | n.a. |
| | 20 | | 0 | 0 | 0 | n.a. | n.a. |
| | 21 | | 0 | 0 | 0 | n.a. | n.a. |
| | 22 | | 0 | 0 | 0 | n.a. | n.a. |
| | 23 | | 0 | 0 | 0 | n.a. | n.a. |
| | 24 | | 0 | 0 | 0 | n.a | n.a |
| | 25 | | 0 | 0 | 0 | 0 | n.a |
| | 26 | | 0 | 0 | 0 | 0 | n.a |
| | 27 | | 0 | 0 | 0 | 0 | n.a |
| | 28 | | 0 | 0 | 0 | 0 | n.a |
| | 29 | | 0 | 0 | 0 | 0 | n.a |
| | 30 | | 0 | 0 | 0 | 0 | n.a |
| | 31 | | 0 | 0 | 0 | 0 | 0 |
| | 32 | | 0 | 0 | 0 | 0 | 0 |
| | 33 | | 0 | 0 | 0 | 0 | 0 |
| | 34 | | 0 | 0 | 0 | 0 | 0 |
| | 35 | | 0 | 0 | 0 | 0 | 0 |
| | 36 | | 0 | 0 | 0 | 0 | 0 |

TABLE 10B
Safety analysis, clinical score data table of Hu-mice. Clinical safety scores were established by
characterization of five different clinical signs (coat, movement, activity, paleness, body weight), according to the
following specification: Coat: score 0-normal; score 1-lack of grooming, partial alopecia; score 2-massive alopecia,
wounds, bleedings, inflammation. Movement: score 0-normal; score 2-slow movement, paralysis of one animal; score
3-difficulties to eat and drink, paralysis to more than one animal. Activity: score 0-normal; score 1-agitated, over-
reactive, hypo-reactive; score 3-prostrated. Paleness: score 0-normal; score 1-slight (no ear vessels visible); score 2-
severe (ears plus feet affected). Body weight: score 0-normal; score 2-segmentation of the vertebral column evident,
pelvic bones palpable; score 3-skeletal structure prominent. Maximum cumulative clinical score allowed: 6. n.a.: not
applicable.

| Days after 1^{st} vaccination: | | | −7 | −1 | 7 | 13 | 20 | 27 |
|---|---|---|---|---|---|---|---|---|
| Mouse strain | Mouse ID | treatment | Cumulative clinical score | | | | | |
| Hu-mouse | 37 | SARS- | 0 | 0 | 0 | 0 | n.a. | n.a. |
| (Hu-NCG) | 38 | CoV-2 | 0 | 0 | 0 | 0 | n.a. | n.a. |
| | 39 | | 0 | 0 | 0 | 0 | n.a. | n.a. |
| | 40 | | 0 | 0 | 0 | 0 | n.a. | n.a. |
| | 41 | | 0 | 0 | 0 | 0 | n.a. | n.a. |
| | 42 | | 0 | 0 | 0 | 0 | n.a. | n.a. |
| | 43 | | 0 | 0 | 0 | 0 | 0 | n.a. |
| | 44 | | 0 | 0 | 0 | 0 | 0 | n.a. |
| | 45 | | 0 | 0 | 0 | 0 | 0 | n.a. |
| | 46 | | 0 | 0 | 0 | 0 | 0 | n.a. |
| | 47 | | 0 | 0 | 0 | 0 | 0 | n.a. |
| | 48 | | 0 | 0 | 0 | 0 | 0 | n.a. |
| | 49 | | 0 | 0 | 0 | 0 | 0 | 0 |
| | 50 | | 0 | 0 | 0 | 0 | 0 | 0 |
| | 51 | | 0 | 0 | 0 | 0 | 0 | 0 |
| | 52 | | 0 | 0 | 0 | 0 | 0 | 0 |
| | 53 | | 0 | 0 | 0 | 0 | 0 | 0 |
| | 54 | | 0 | 0 | 0 | 0 | 0 | 0 |
| | 55 | Vehicle | 0 | 0 | 0 | 0 | n.a. | n.a. |
| | 56 | | 0 | 0 | 0 | 0 | n.a. | n.a. |
| | 57 | | 0 | 0 | 0 | 0 | n.a. | n.a. |
| | 58 | | 0 | 0 | 0 | 0 | n.a. | n.a. |
| | 59 | | 0 | 0 | 0 | 0 | n.a. | n.a. |
| | 60 | | 0 | 0 | 0 | 0 | n.a | n.a |
| | 61 | | 0 | 0 | 0 | 0 | 0 | n.a |
| | 62 | | 0 | 0 | 0 | 0 | 0 | n.a |
| | 63 | | 0 | 0 | 0 | 0 | 0 | n.a |
| | 64 | | 0 | 0 | 0 | 0 | 0 | n.a |
| | 65 | | 0 | 0 | 0 | 0 | 0 | n.a |
| | 66 | | 0 | 0 | 0 | 0 | 0 | n.a |
| | 67 | | 0 | 0 | 0 | 0 | 0 | 0 |
| | 68 | | 0 | 0 | 0 | 0 | 0 | 0 |
| | 69 | | 0 | 0 | 0 | 0 | 0 | 0 |
| | 70 | | 0 | 0 | 0 | 0 | 0 | 0 |
| | 71 | | 0 | 0 | 0 | 0 | 0 | 0 |
| | 72 | | 0 | 0 | 0 | 0 | 0 | 0 |

TABLE 10C
Safety analysis, necropsy data table. Necropsy
has been performed by macroscopic
observation of spleen, liver, kidneys, stomach and intestine.

| Mouse | Experimental | | |
|---|---|---|---|
| strain | day | Treatment | Necropsy result |
| BALB/c | D14 | PolyPEPI- | No abnormal observation in 6 of 6 |
| | | SCoV-2 | |
| | | Vehicle | No abnormal observation in 6 of 6 |
| | D21 | PolyPEPI- | No abnormal observation in 6 of 6 |
| | | SCoV-2 | |
| | | Vehicle | No abnormal observation in 6 of 6 |
| | D28 | PolyPEPI- | No abnormal observation in 6 of 6 |
| | | SCoV-2 | |
| | | Vehicle | No abnormal observation in 6 of 6 |
| Hu-mouse | D14 | PolyPEPI- | No abnormal observation in 6 of 6 |
| (Hu-NCG) | | SCoV-2 | |
| | | Vehicle | No abnormal observation in 6 of 6 |
| | D21 | PolyPEPI- | No abnormal observation in 6 of 6 |
| | | SCoV-2 | |
| | | Vehicle | No abnormal observation in 6 of 6 |
| | D28 | PolyPEPI- | No abnormal observation in 6 of 6 |
| | | SCoV-2 | |
| | | Vehicle | No abnormal observation in 6 of 6 |

### PolyPEPI-SCoV-2 Peptide-Specific T Cell Responses of COVID-19 Convalescent Donors

Next, we aimed to demonstrate that the robust and broad T cell responses detected in vaccinated animals are relevant in humans by investigating vaccine-specific T cells circulating in the blood of COVID-19 convalescent donors (baseline data are reported in Table 8).

The reactivity of vaccine peptides with convalescent immune components was investigated in 17 convalescent and four healthy donors. Vaccine-reactive CD4⁺ T cells were detected using the nine 30-mer vaccine peptides grouped in four pools according to their source protein: S, N, M, and E peptides. CD8⁺ T cell responses were measured using the 9-mer test peptides corresponding to the dominant and shared HLA class I PEPIs defined for each of the nine vaccine peptides that were also grouped into four pools according their source protein (s, n, m, and e peptides; Table 8 bold), as used in the animal experiments.

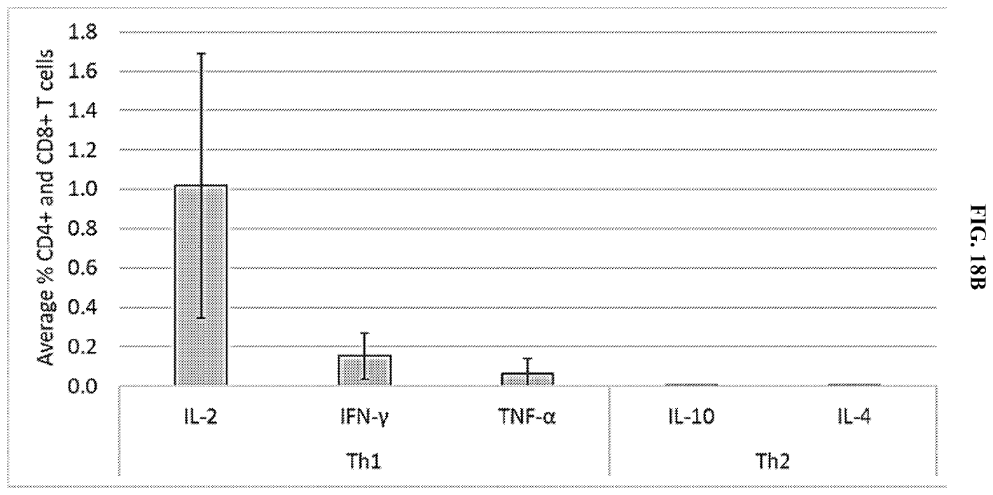

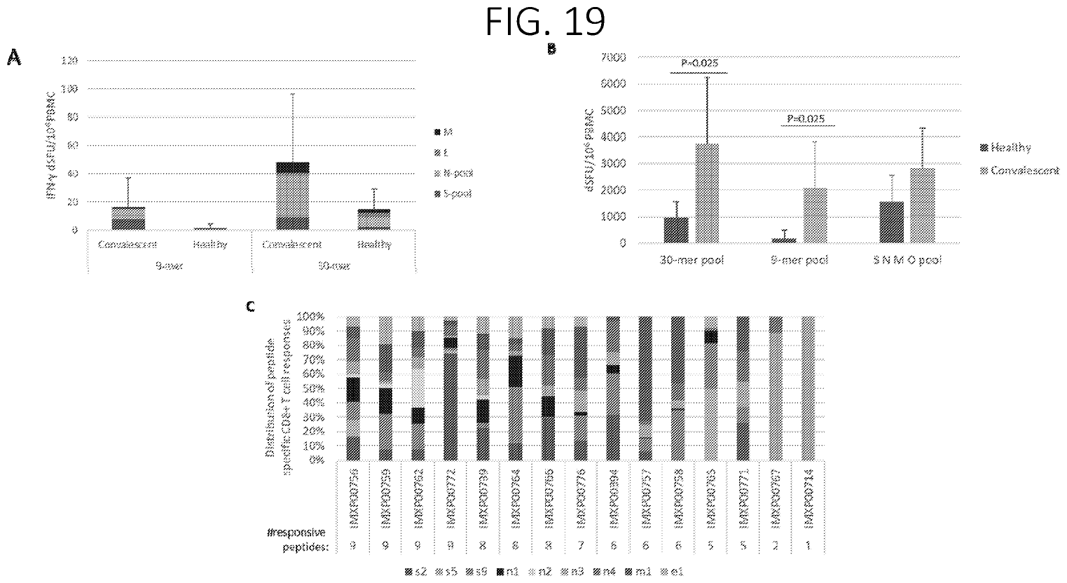

Using ex vivo FluoroSpot assays, which can detect rapidly activating effector phase T cell responses, significant numbers of vaccine-reactive, IFN-γ-expressing T cells were detected with both 30-mer (average dSFU: 48.1, p=0.014) and 9-mer peptides (average dSFU: 16.5, p=0.011) compared with healthy subjects (FIGS. 19A and B). Detailed analysis of the four protein-specific peptide pools revealed that three out of the 17 donors reacted to all four structural antigens with the 30-mer vaccine peptides; 82% of donors reacted to two antigens and 59% to three antigens. Notably, short 9-mer-specific CD8⁺ T cell responses could be also identified against at least one of four antigens in all 17 donors and against at least two antigens in 53% (Table 11).

TABLE 11
Response rate of COVID-19 convalescent donor patients to one,
two, three, or all four viral antigens targeted by the PolyPEPI-
SCoV-2 vaccine, as measured by ex vivo FluoroSpot
assay. Nine-mers are the hotspot HLA class I PEPIs
embedded within each 30-mer vaccine peptide
coresponding to the four structural
proteins: S, Spike; N, Nucleoprotein;
M, membrane; E, envelope proteins.

| Number of | Percentage of subjects | Percentage of subjects |
|---|---|---|
| reactive antigens | responsive to 30-mer | responsive to 9-mer |
| (S, N, M, E) | peptides (N = 17) | peptides (N = 17) |
| 1 | 94% | 100% |
| 2 | 82% | 53% |
| 3 | 59% | 18% |
| 4 | 18% | 6% |

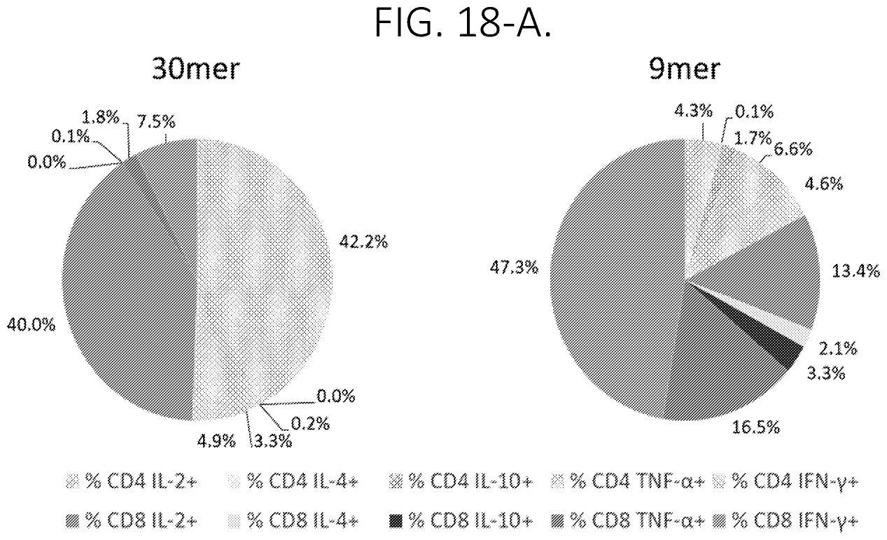

As determined by ICS assays, stimulation with 9-mer peptides resulted in an average T cell make up of 83% CD8⁺ T cells, and 17% CD4⁺ T cells (FIG. 18A). Interestingly, the 30-mer test peptides reacted with both CD4⁺ and CD8⁺ T cells in average ratio of 50:50 (FIG. 18A). Functionality testing of the T cells revealed that CD8⁺ T cells primarily produced IFN-γ, TNF-α, and IL-2 (with small amounts of IL-4 and IL-10), while CD4⁺ T cells were positive for mainly IL-2 and IFN-γ. Recall responses demonstrated clear Th1 cytokine characteristics; Th2 responses were not present in the recall response with 30-mer vaccine peptides (FIG. 18B).

Next, we determined whether the ex vivo detected, available, rapidly activating T cells could also expand in vitro in the presence of vaccine peptides. Using enriched ELISpot, significant numbers of vaccine-reactive, IFN-γ-expressing T cells were detected with both 30-mer (average dSFU=3746, p=0.025) and 9-mer (average dSFU=2088, p=0.028) peptide pools compared with healthy subjects (FIG. 19A). The intensity of the PolyPEPI-SCoV-2-derived T cell responses (30-mer pool) were also evaluated relative to the responses detected with a commercial, large SARS-CoV-2 peptide pool (SMNO) containing 47 long peptides derived from both structural (S, M, N) and non-structural (open reading frame ORF-3a and 7a) proteins. The relative intensities obtained for the two pools were favorable for the vaccine pool among the COVID-19 donors, while more healthy donors reacted to the commercial peptide pool, confirming improved specificity of PolyPEPI-SCoV-2 vaccine (FIG. 19B).

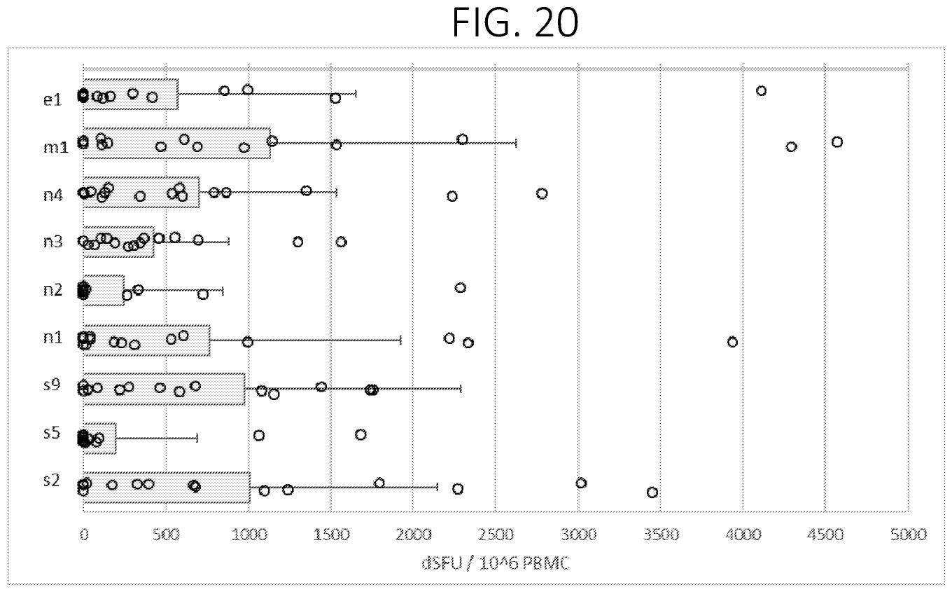

To confirm and further delineate the multispecificity of the PolyPEPI-SCoV-2-specific T cell responses detected ex vivo in COVID-19 recovered individuals, we defined the distinctive peptides targeted by their T cells. We first deconvoluted the peptide pools and tested the CD8⁺ T cell responses specific to each of the 9-mer HLA class I PEPIs corresponding to each vaccine peptide using in vitro expansion (FIG. 20). Analysis revealed that each 9-mer peptide was recognized by several subjects; the highest recognition rate in COVID-19 convalescent donors was observed for n4 (93%), s9 (87%), s2, n1, ml (80%), el (60%), s5, n2 (40%) (FIG. 19C).

Detailed analysis of the nine peptide-specific CD8⁺ T cell responses revealed that 100% of COVID-19-recovered subjects had PolyPEPI-SCoV-2-specific T cells reactivated with at least one peptide, 93% with more than two, 87% with more than five, and 27% had T cell pools specific to all nine vaccine peptides. At the protein level, 87% of subjects had T cells against multiple (three) proteins and eight out of the 15 measured donors (53%) reacted to all four targeted viral proteins (FIG. 19C). These data confirm that PolyPEPI-SCoV-2-peptides are dominant for an individual and shared between COVID-19 subjects, and that the multi-peptide-specific T cell frequencies obtained in the convalescent population were in good alignment with the predicted frequencies based on shared PEPIs for the in silico cohort (Table 9). Moreover, some fragments (epitopes) of our vaccine peptides were independently confirmed by Ferretti et al. as shared immunodominant epitopes in a systematic, laborious T cell epitope screening study involving convalescents.(27)

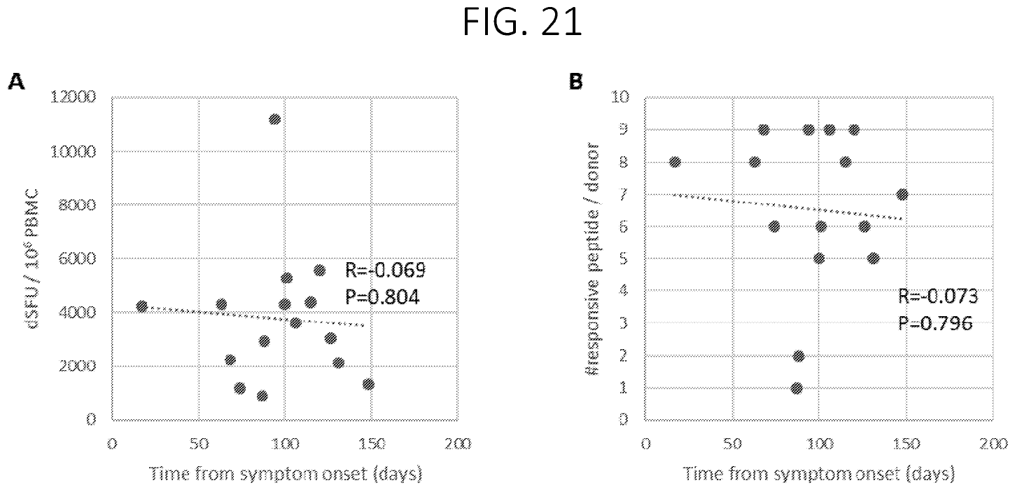

For our cohort of convalescent subjects, the breadth and magnitude of vaccine-specific T cell responses did not correlate with time from symptom onset, suggesting that these T cells are persistent (up to 5 months) (FIG. 21).

To demonstrate that PEPIs (multi-HLA binding epitopes) can generate T cell responses at the individual level, we first determined the complete class I HLA-genotype for each subject and then predicted the peptides that could bind to at least three HLA alleles of a person from the list of nine 9-mer peptides used in the ELISpot assay. For each subject between two and seven peptides out of nine proved to be PEPIs. Among the predicted peptides (PEPIs), 84% were confirmed by ELISpot to generate highly specific T cell responses (Table 12).

TABLE 11
Agreement between PEPI prediction and immune responses measured by enriched ELISpot assay for CO VID-
19 convalescents. Personal epitopes (PEPIs) were predicted as ≥ 3 autologous HLA class I allele binding
9-mer epitopes and compared with the IFN-γ-producing CD8⁺ T cell response measured with identical
9-mer stimulations. True positive values are highlighted in bold letters.

| | | Predicted HLA-class I PEPI peptide ID | |
|---|---|---|---|
| Patient ID | class I HLA genotype | (dSFU by ELISpot) | Matching |
| IMXP00394 | A*11:01,A*24:02,6*35:03 | s9(1156), n1(231) | 2/2 |
| | B*55:01,C*03:03,C*12:03 | | |
| IMXP00714 | A*01:01,A*02:01,6*07:02 | s5(0), s9(0), e1(0) | 0/3 |
| | B*44:03,C*04:01,C*07:02 | | |
| IMXP00739 | A*03:01,A*03:01,B*07:02 | n2(333),e1(1535) | 2/2 |
| | B*35:03,C*04:01,C*07:02 | | |
| IMXP00756 | A*02:01,A*H:01,B*15:01 | s2(2273), s5(1684), s9(1754), n1(2334), | 7/7 |
| | B*55:01,C*03:03,C*03:04 | n2(264), e1(1141), m1(996) | |
| IMXP00757 | A*02:01,A*31:01,B*40:01 | s9(583), e1(4570) | 2/2 |
| | B*44:02,C*03:04,C*05:01 | | |
| IMXP00758 | A*01:01,A*H:01,B*08:01 | s2(20), s9(1738) | 2/2 |
| | B*44:02,C*05:01,C*07:01 | | |
| IMXP00759 | A*24:02,A*30:01,B* 13:02 | s2(1797), s5(93), s9(5143), n4(1353), | 6/6 |
| | B*57:01,C*06:02,C*06:02 | e1(4292), m1(4113) | |
| IMXP00762 | A*02:05,A*30:02,B*15:03 | n3(696) | 1/1 |
| | 6*51:01,C*12:03,C*14:02 | | |
| IMXP00764 | A*01:01,A*23:01,6*44:03 | s2(328), s9(1083) | 2/2 |
| | B*49:01,C*04:01,C*07:01 | | |
| IMXP00765 | A*02:01,A*29:02,B*40:01 | s2(0), s5(1067), s9(677), e1(0) | 2/4 |
| | B*44:03,C*03:04,C*16:01 | | |
| IMXP00766 | A*03:01,A*30:01,B*13:02 | s9(26), e1(688), m1(301) | 3/3 |
| | B*27:05,C*02:02,C*06:02 | | |
| IMXP00767 | A*01:01,A*03:02,B*38:01 | s9(0) | 0/1 |
| | B*51:01,C*12:03,C*15:02 | | |
| IMXP00771 | A*02:01,A*03:01,B*07:02 | s5(0), s9(275), e1(608) | 2/3 |
| | B*35:03,C*04:01,C*07:02 | | |
| IMXP00772 | A*02:01,A*26:01,B*15:01 | s9(83), n1(312), e1(148), m1(118) | 4/4 |
| | B*55:01,C*03:03,C*03:03 | | |
| IMXP00776 | A*24:02,A*68:01,B*27:05 | s9(221), n3(189) | 2/2 |
| | B*35:01,C*04:01,C*07:02 | | |

Correlation Between Multiple Autologous Allele-Binding Epitopes and CD8+ T Cell Responses

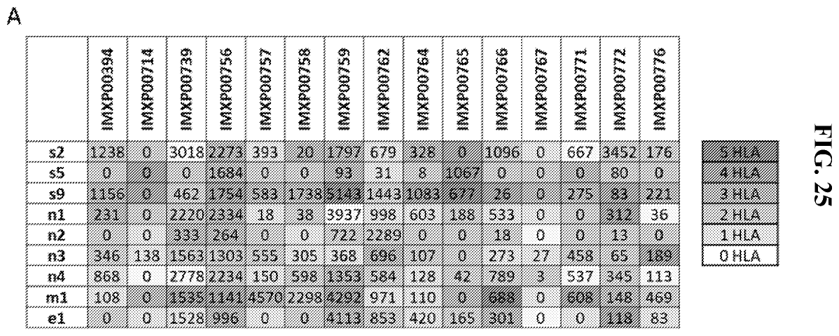

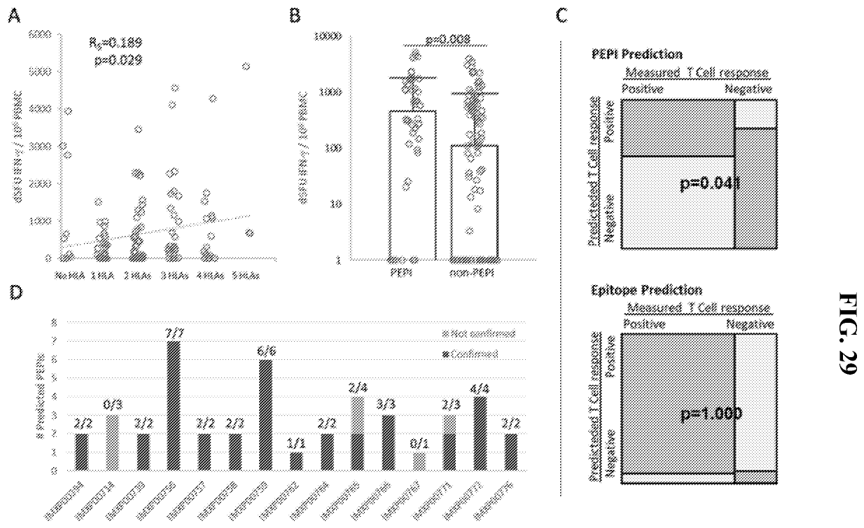

The HLA binding capacity of the immunogenic peptides detected for each subject was investigated. First we determined the complete HLA class I genotype for each subject and then predicted the number of autologous HLA alleles that could bind to each of the nine shared 9-mer peptides used in the FluoroSpot assay. Then we matched the predicted HLA-binding epitopes to the CD8+ T cell responses measured for each peptide in each patient (total 15×9=135 data points, FIG. 25). The magnitude of CD8+ T cell responses tended to correlate with epitopes restricted to multiple autologous HLA alleles (RS=0.188, p=0.028, FIG. 29B). In addition, we observed that the magnitude of CD8+ T cell responses generated by PEPIs (HLA≥3) (median dSFU=458) was significantly higher than those generated by non-PEPIs (HLA<3) (median dSFU=110), (p=0.008) (FIG. 29B).

Across the 135 data points there were 98 positive responses and 37 negative responses recorded. Among the 98 positive responses 37 were generated by PEPIs, while among the 37 negatives only 7 were PEPIs, the others were epitopes restricted to ≤3 autologous HLA alleles (FIG. 25). Overall, the 2×2 contingency table revealed association of T cell responses with PEPIs (p=0.041, Fisher Exact) but not with HLA-restricted epitopes (p=1.000, Fisher Exact) (FIG. 25). For each subject between one and seven peptides out of nine proved to be PEPIs. Among the predicted PEPIs, 37/44 (84%) were confirmed by IFN-γ FluoroSpot assay to generate specific T cell responses in the given subject (FIG. 29D and FIG. 25).

These data demonstrate that subjects' complete HLA-genotype influence their CD8+ T cell responses and multiple autologous allele-binding capacity is a key feature of immunogenic epitopes. PEPIs in general underestimated the subject's overall T cell repertoire, however they precisely predicted subjects' PEPI-specific CD8+ T cell responses.

### Correlation Between PolyPEPI-SCoV-2-Reactive T Cells and SARS-CoV-2-Specific Antibody Responses

T cell-dependent B cell activation is required for antibody production. For each subject, different levels of antibody responses were detected against both S and N antigens of SARS-CoV-2 determined using different commercial kits (Table 8). All subjects tested positive with Euroimmune ELISA (IgG) against viral S-1 and a Roche kit to measure N-related antibodies. All subjects tested positive for DiaPro IgG and IgM (except 2 donors), 7/17 for DiaPro IgA detecting mixed S-1 and N protein-specific antibody responses (Table 8).

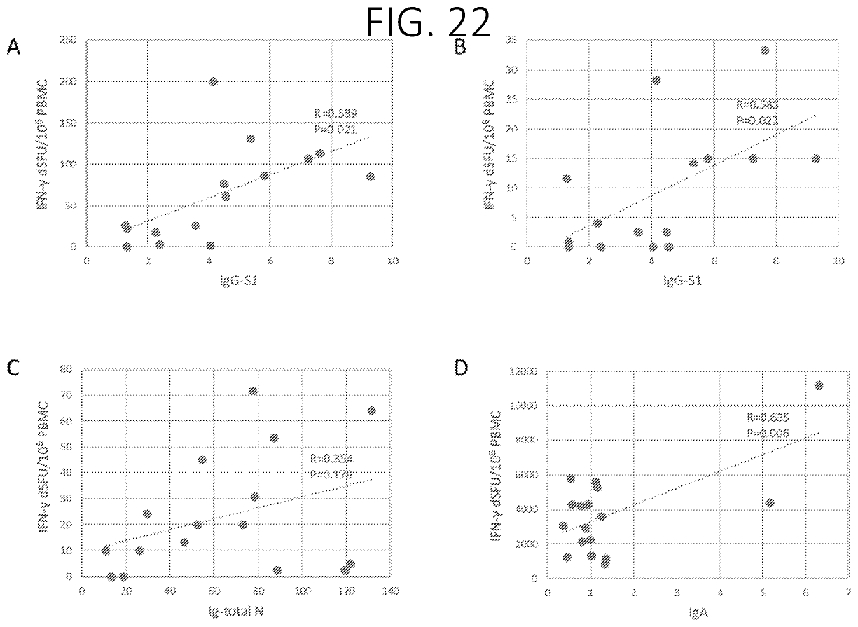

We next evaluated the correlation between PolyPEPI-SCoV-2-specific CD4⁺ T cell reactivities and antibody responses (FIG. 22). The total number of the PolyPEPI-SCoV-2-reactive CD4⁺ T cells correlated with the S-1 protein-specific IgG amount measured by ELISA (R=0.59, p=0.02, FIG. 22A). Next, specific S-1 protein-derived peptides of the PolyPEPI-SCoV-2 vaccine (S2 and S5) were analyzed and the correlation was similar (R=0.585, p=0.02, FIG. 22B). Similarly, T cell responses detected with N protein derived PolyPEPI-SCoV-2 peptides (N1, N2, N3 and N4) presented a weak but not significant correlation with N-specific antibodies detected with Roche kit (FIG. 22C). These data suggest a link between PolyPEPI-SCoV-2-specific CD4⁺ T cell responses and subsequent IgG production for COVID-19 convalescent donors.

Interestingly, IgA production correlated with PolyPEPI-SCoV-2-specific memory CD4⁺ T cell responses (R=0.63, p=0.006, FIG. 6D). T cell responses reactive to the SMNO peptide pool exhibited no correlation with any of the antibody subsets. This suggests that not all CD4⁺ T cells contributed to B-cell responses, consequently to IgG production (data not shown).

### Predicted Immunogenicity in Different Ethnicities

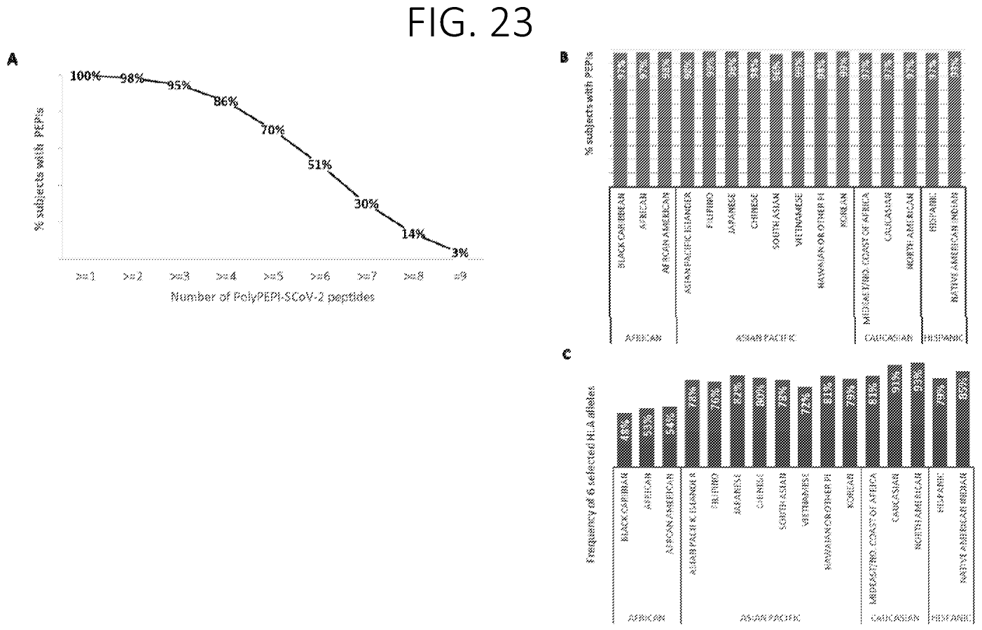

We performed in silico testing of our PolyPEPI-SCoV-2 vaccine in a large cohort of 16,000 HLA-genotyped subjects distributed among 16 different ethnic groups from a US bone marrow donor database.(49) For each subject in this large cohort, we predicted the PolyPEPI-SCoV-2-specific PEPIs based on their complete HLA class I and class II genotype. Most subjects have a broad repertoire of PEPIs that will likely be transformed to virus-specific memory CD8⁺ T cell clones: 98% of subjects were predicted to have PEPIs against at least two vaccine peptides, and 95%, 86%, and 70% against three, four, and five peptides, respectively (FIG. 23A).

In silico testing revealed that ≥98% of subjects in each ethnic group will likely mount robust cellular responses, with both CD8⁺ and CD4⁺ T cell responses against at least two peptides in the vaccine (FIG. 23B). This predicted high response rate is also true for the ethnicities reported to have worse clinical outcomes from COVID-19 (Black, Asian) (25). Based on these data, we expect that the vaccine will provide global coverage, independent of ethnicity and geographic location.

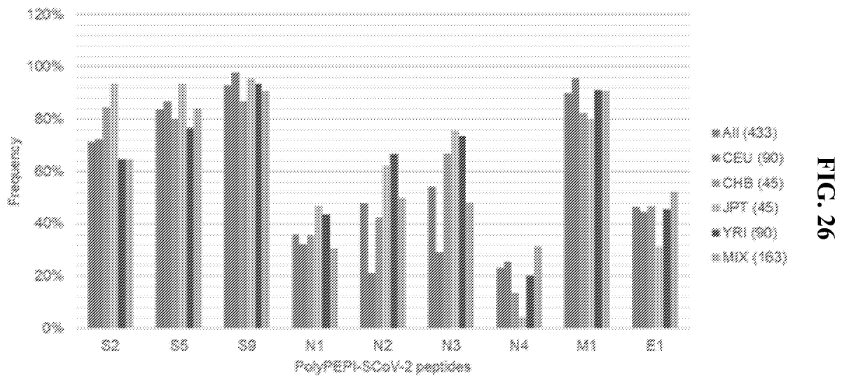

We also used the 16,000 population (and comprising ethnic groups) to assess theoretical global coverage as proposed by others, i.e. filtering the sub-populations having at least one of the six prevalent HLA class I alleles considered to cover 95% of the global population.(27, 55, 56) Using this approach, we observed significant heterogeneity at the ethnicity level. While we confirmed that the selected six HLA alleles are prevalent in the Caucasian and North American cohorts (91-93%), the frequency of these alleles was lower in all other ethnic groups, especially in African populations (48-54%) (FIG. 23C). We concluded that the proposed prevalent HLA allele set may cover the HLA frequency in an ethnically weighted global population, but epitope selection for vaccination purposes based only on these alleles would discriminate some ethnicities. Therefore, we propose using a representative model population that is sensitive to the heterogeneities in the human race and that allows selecting PEPIs shared among individuals across ethnicities. Of note, while we did not observe any difference in SARS-CoV-2 protein-derived epitope generation capacity of the individuals of different ethnicities based on their complete HLA-genotype (FIG. 12) (which does not seem to explain the higher infection and mortality rates observed in BAME), epitopes for subunit vaccines may be carefully selected to address the HLA-genotype profile of BAME groups. Heterogeneity in the frequency of the shared PEPIs in the different ethnic groups were observed, especially for protein N, having high impact on the design of a global vaccine (FIG. 26). Combination of targets with different frequencies inside- and between ethnic groups into a vaccine candidate with high global coverage is feasible by performing “in silico clinical trials” in large populations of real subjects.

Therefore, to maximize multi-antigenic immune responses at both the individual and population/ethnicity levels, and also considering the chemical and manufacturability properties of the peptides, a total of nine 30-mer peptides from four structural proteins of SARS-CoV-2 were selected: three peptides from spike (S), four peptides from nucleoprotein (N), and one peptide from each matrix (M) and envelope (E). No peptides were included from the receptor-binding domain (RBD) of S protein. Overall, each member of the model population had HLA class I PEPIs for at least two of the nine peptides, and 97% had at least three (Table 9). Each subject had multiple class II PEPIs for the vaccine peptides (Table 9). Each subject had multiple class II PEPIs for the vaccine peptides (Table 9).

### Discussion

We demonstrated that PolyPEPI-SCoV-2, a polypeptide vaccine comprising nine synthetic long (30-mer) peptides derived from the four structural proteins of the SARS-CoV-2 virus (S, N, M, E) is safe and highly immunogenic in BALB/c mice and humanized CD34⁺ mice when administered with Montanide ISA 51 VG adjuvant. In addition, the vaccine's immunogenic potential was confirmed in COVID-19 convalescent donors by successfully reactivating PolyPEPI-SCoV-2-specific T cells, which broadly overlap with the T cell immunity generated by SARS-CoV-2 infection.

The present vaccine design concept, targeting multi-antigenic immune responses at both the individual and population level, represents a novel target identification process that has already been used successfully in cancer vaccine development to achieve unprecedented immune response rates that correlate with initial efficacy in the clinical setting.(32) For COVID-19, we focused on selecting fragments of the SARS-CoV-2 proteins that contain overlapping HLA class I and II T cell epitopes shared between ethnically diverse HLA-genotyped individuals and that also generate diverse and broad immune responses against the whole virus structure. Therefore, we selected relatively long 30-mer fragments to favor generation of multiple effector responses (B cells and cytotoxic T cells) and helper T cell responses.

The PolyPEPI-SCoV-2 vaccine elicits the desired multi-antigenic IFN-γ producing T cell responses for both vaccine-specific CD8⁺ and CD4⁺ T cells in vaccinated BALB/c and humanized CD34⁺ mice against all four SARS-CoV-2 proteins, and these responses were more prominent after the booster dose. The recall responses in COVID-19 convalescents comprised both rapidly activating effector-type (ex vivo detected) and expanded (in vitro detected) memory-type CD8⁺ and CD4⁺ T cell responses against all nine peptides, with PolyPEPI-SCoV-2-specific T cells detected in 100% of donors. On the individual level, the PolyPEPI-SCoV-2-specific T cell repertoire used for recovery from COVID-19 is extremely diverse: each donor had an average of seven different peptide-specific T cell pools, with multiple targets against SARS-CoV-2 proteins; 87% of donors had multiple targets against at least three SARS-CoV-2 proteins and 53% against all four, 1-5 months after their disease. In addition, 87% of subjects had CD8⁺ T cells against S protein, which is similar with the immune response rates reported for frontline COVID-19 vaccine candidates in phase I/II clinical trials).(57) However, we found that S-specific (memory) T cells represented only 36% of the convalescents' total T cell repertoire detected with vaccine peptides; the remaining 64% was distributed almost equally among N, M, and E proteins. These data confirm the increasing concern that S-protein based candidate vaccines are not harnessing the full potential of human anti-SARS-CoV-2 immunity, especially since diverse T cell responses were associated with mild/asymptomatic COVID-19.(4)

The interaction between T and B cells is a known mechanism toward both antibody-producing plasma cell production and generation of memory B cells.(59) During the analysis of convalescents' antibody subsets, we found correlations between antigen-specific IgG levels and corresponding peptide-specific CD4⁺ T cell responses. This correlation might represent the link between CD4⁺ T cells and antibody production, a concept also supported by total IgG production in the animal models. Binding IgG antibodies can act in cooperation with the vaccine induced CD8+ killer T cells upon later SARS-CoV-2 exposure of the vaccines. This interplay might result in effective CD8+ T cell mediated direct killing of infected cells and IgG-mediated killing of virus-infected cells and viral particles, inhibiting Th2-dependent immunopathologic processes, too. In this way, it is expected that both intracellular end extracellular virus reservoirs are attacked to help rapid viral clearance even in the absence of neutralizing antibodies.(59, 60)

The present data demonstrates that individuals' anti-SARS-CoV-2 T cell responses reactive to the PolyPEPI-SCoV-2 peptide set are HLA genotype-dependent. Specifically, multiple autologous HLA binding epitopes (PEPIs) determine antigen-specific CD8⁺ T cell responses with 84% accuracy. Although PEPIs generally underestimated the subject's overall T cell repertoire, they are precise target identification “tools” and predictors of PEPI-specific immune responses, overcoming the high false positive rates generally observed in the field using only the epitope-binding affinity as the T cell response predictor.(34, 61) Therefore, by means of validated PEPI prediction of T cell responses based on the complete HLA genotype of (only) Caucasian individuals and careful interpretation of PolyPEPI-SCoV-2 induced immune responses in animals modeling immune responses in humans, our findings could be extrapolated to large cohorts of 16,000 HLA-genotyped individuals and 16 human ethnicities. The ethnic groups represented in this large US cohort covers the composition of the global population but they were not weighted for their global representativeness (n=1,000 subjects for each ethnicity as used intentionally).(62) Based on this, PolyPEPI-SCoV-2 will likely generate meaningful immune responses (both CD8⁺ and CD4⁺ T cell responses against at least two vaccine peptides) in >98% of the global population, independent of ethnicity. In comparison, a T cell epitope-based vaccine design approach based on globally frequent HLA alleles would miss generation of immune responses for ˜50% of Black Caribbean, African, African-American, and Vietnamese ethnicities. Inducing meaningful immune responses by vaccination (broad and polyfunctional memory responses mimicking the heterogeneity of the immunity induced by SARS-CoV-2) uniformly in high percentages of vaccinated subjects is essential to achieve the desired “herd immunity”. It is considered that about 25-50% of the population would have to be immune to the virus to achieve suppression of community transmission.(3) However, first-generation COVID-19 vaccines are being tested to show disease risk reduction of at least 50% but they are not expected to reduce virus transmission to a comparable degree. This fact combined with the likelihood that these vaccines will also not provide long-term immunity suggest that second-generation tools are needed to fight the pandemic. (3)

We believe, focusing on several targets in each subject would better recapitulate the natural T cell immunity induced by the virus, leading to efficient, long-term memory responses. For this purpose, synthetic polypeptide-based platform technology is considered a safe and immunogenic vaccination strategy with several key advantages. The same class of peptide vaccines with Montanide adjuvant were safe and well-tolerated in >6,000 patients and 231 healthy volunteers.(63-69) Studies in SARS suggest that candidate coronavirus vaccines that limit the inclusion of whole viral proteins have more beneficial safety profiles. In addition, efficient Th1-skewed T cell responses and non-conformational (linear) B cell epitopes mitigate the risk of theoretical antibody-mediated disease enhancement and Th2-based immunopathologic processes.

Peptide-based vaccines have had only limited success to date, but this can be attributed to a lack of knowledge regarding which peptides to use. Such uncertainty is reduced by an understanding of how an individual's genetic background is able to respond to specific peptides, as we demonstrated above.

In conclusion, the peptide-based, multi-epitope encoding vaccine design described herein demonstrates safety and exceptionally broad preclinical immunogenicity, and is expected, following careful clinical testing, to provide an effective second-generation vaccine against SARS-CoV-2.

### Example 11-Analysis of Cross-Protection PolyPEPI-SCoV-2 Specific Polypeptides Against SARS

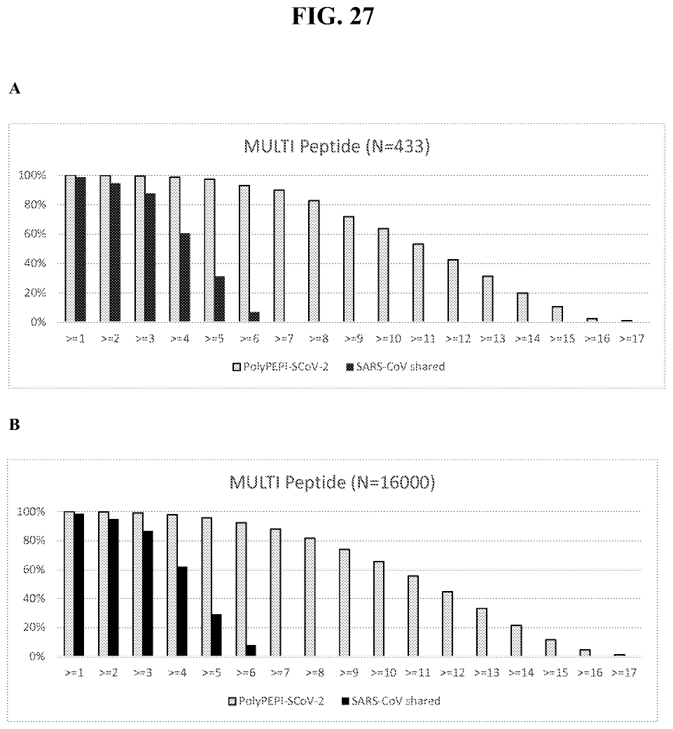

The polypeptide selection for vaccine composition by predicting high frequency of autologous≥3 HLA binding epitopes within the Model Population (MP, N=433) enables the selection of sequences that are shared between different pathogen species. For instance, there are several conservative and 100% identical peptide fragments within the proteome of SARS virus and SARS-CoV-2 (SEQ ID NOs: 6 and 9 to 17). By analyzing these fragments the inventors identified the proper≥3 autologous HLA binding 9-mer PEPIs in high percentage of the individuals from the MP. 99% had at least one predicted shared epitope from the analyzed composition of SEQ ID NO. 1-17 (Table 13 and FIG. 27A) 94% of the MP had at least two HLA class I specific (CD8+ T cell) PEPIs, consequently the composition is predicted to induce multi-peptide immune response in 94% of the MP. In average, 3 or 4 SARS-derived shared peptide was predicted to be reactive with the 17 30-mer peptides. If one peptide represents one T cell clone (as the correlation of the predicted and ELISPOT measured immune response was proven in Example 5, e.g. PPV=0.79), these in silico results could be interpreted as induction of multi-peptide specific immunogenic T cell responses if these 17 peptides would be used for immunization.

TABLE 13
Multi-antigen (multi-protein) response rate in Model Population (N = 433) predicted for shared
SARS-CoV epitopes from the 17 30-mer peptides originally designed for SARS-CoV-2.

| MULTI Peptide (N = 433) | PolyPEPI-SCoV-2 | SARS-CoV shared |
|---|---|---|
| >=1 | 100% | 99% |
| >=2 | 100% | 94% |
| >=3 | 99% | 88% |
| >=4 | 99% | 61% |
| >=5 | 97% | 31% |
| >=6 | 93% | 7% |
| >=7 | 90% | 0% |
| >=8 | 83% | 0% |
| >=9 | 72% | 0% |
| >=10 | 64% | 0% |
| >=11 | 53% | 0% |
| >=12 | 42% | 0% |
| >=13 | 31% | 0% |
| >=14 | 20% | 0% |
| >=15 | 11% | 0% |
| >=16 | 2% | 0% |
| >=17 | 1% | 0% |
| AVG Peptide: | 10.57 | 3.79 |

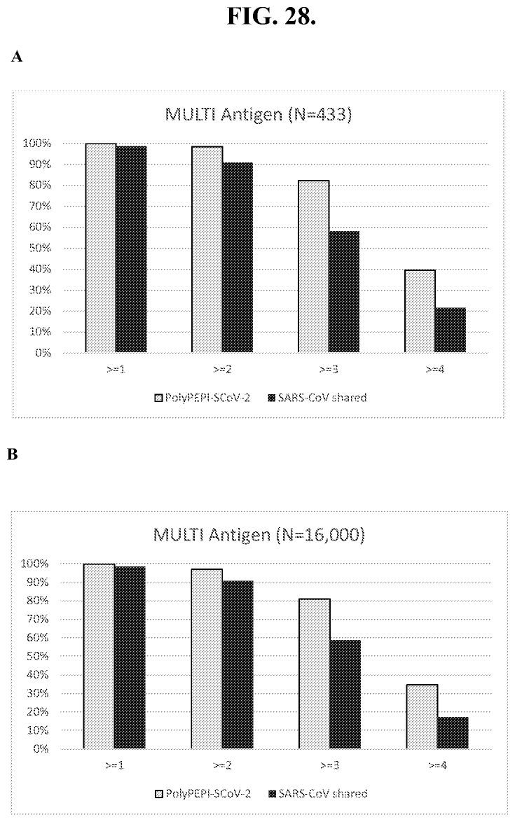

Multi-protein or multi-antigen response against at least two antigens were shared in 91% of individuals, representing good coverage for this composition if used in a SARS cohort. (FIG. 28A). These data suggest that the 17 peptides has the potential to induce multi-peptide and also multi-protein specific T cell responses against SARS.

The results were confirmed in a 16,000 human population, too. 95% of individuals had multi-HLA restricted personal epitopes against at least 2 shared peptides.

TABLE 14
Multi-antigen (multi-protein) response rate in large human population (N = 16,000) predicted
for shared SARS-CoV epitopes from the 17 30-mer peptides originally designed for SARS-CoV-2.

| MULTI Peptide (N=16,000) | PolyPEPI-SCoV-2 | SARS-CoV shared |
|---|---|---|
| l | 100% | 98% |
| >=2 | 100% | 95% |
| >=3 | 99% | 87% |
| >=4 | 98% | 62% |
| >=5 | 96% | 29% |
| >=6 | 92% | 8% |
| >=7 | 88% | 0% |
| >=8 | 82% | 0% |
| >=9 | 74% | 0% |
| >=10 | 65% | 0% |
| >=11 | 56% | 0% |
| >=12 | 45% | 0% |
| >=13 | 33% | 0% |
| >=14 | 21% | 0% |
| >=15 | 11% | 0% |
| >=16 | 5% | 0% |
| >=17 | 1% | 0% |
| AVG Peptide: | 10.65 | 3.79 |

These data represented an opportunity to identify abundant, multi-HLA binding epitopes with shared specificity against different pathogens, e.g. against SARS (FIG. 27B and FIG. 28B).

### Example 12—Nucleic Acid Formulations Encoding and Expressing SEQ ID No. 1-17

The polypeptides SEQ ID NO 1-17 can be manufactured in different formulations, in water soluble or adjuvanted peptide dissolved or emulsified for injections. Also, the peptides could be encoded into mRNA, RNA or DNA formulation and expressed in plasmid DNA or in viral vectors, if the 3 letter amino acid codes is translated into the identical amino acid sequence as the SEQ ID. NO. 1-17. The appropriate vectors for delivering and/or expressing the encoded polypeptides include but are not restricted to adenoviral vectors adeno-associated vectors, lentiviral or retroviral vectors, pox virus-derived vectors, Newcastle disease virus vectors, plant viral vectors like mosaicvirus vectors, and hybrid vectors.

The expressed proteins in antigen presenting cells are processed and presented via similar HLA class I and class II antigen presentation pathways as the polypeptides taken up by APCs upon subcutaneous or intradermal delivery of the vaccine.

TABLE 15
Corresponding RNA and DNA sequnces for the amino acid sequences of SEQ ID NOs: 1-17.
IUPAC nucleotide code abbreviations: A: Adenine; C: Cytosine, G: Guanine,
T: Thymine, U: Uracil, R: A or G, Y: C or T/U, S: G or C, W: A or T/U,
K: G or T/U, M: A or C, B: C or G or T/U, D: A or G or T/U, H: A or C
or T/U, V: A or C or G, N: A or C or T/U or G. T/U represents an
equivalent nucleotide: T in case of DNA sequence and U in case of
RNA sequence.

| | | | | Encodes |
|---|---|---|---|---|
| SEQ ID | | SEQ ID | | sequence |
| No. for | | No. for | | of SEQ ID |
| RNA | RNA | DNA | DNA | No. |
| 234 | ACNCARYUNCCNCCNGCNUAYACNAAYWSNUUYAC | 251 | ACNCARYTNCCNCCNGCNTAYACNAAYWSNT | 1 |
| | NMGNGGNGUNUAYUAYCCNGAYAAR | | TYACNMGNGGNGTNTAYTAYCCNGAYAAR | |
| | GUNUUYMGNWSNWSNGUNYUNCAYWSNACN | | GTNTTYMGNWSNWSNGTNYTNCAYWSNACN | |
| 235 | GGNGUNUAYUAYCCNGAYAARGUNUUYMGNWS | 252 | GGNGTNTAYTAYCCNGAYAARGTNTTYMGN | 2 |
| | NWSNGUNYUNCAYWSNACNCARGAYYUN | | WSNWSNGTNYTNCAYWSNACNCARGAYYTN | |
| | UUYYUNCCNUUYUUYWSNAAYGUNACNUGG | | TTYYTNCCNTTYTTYWSNAAYGTNACNTGG | |
| 236 | ACNAARMGNUUYGAYAAYCCNGUNYUNCCNUUY | 253 | ACNAARMGNTTYGAYAAYCCNGTNYTNCCN | 3 |
| | AAYGAYGGNGUNUAYUUYGCNWSNACN | | TTYAAYGAYGGNGTNTAYTTYGCNWSNACN | |
| | GARAARWSNAAYAUHAUHMGNGGNUGGAUH | | GARAARWSNAAYATHATHMGNGGNTGGATH | |
| 237 | WSNAAYAUHAUHMGNGGNUGGAUHUUYGGNAC | 254 | WSNAAYATHATHMGNGGNTGGATHTTYGG | 4 |
| | NACNYUNGAYWSNAARACNCARWSNYUN | | NACNACNYTNGAYWSNAARACNCARWSNYT | |
| | YUNAUHGUNAAYAAYGCNACNAAYGUNGUN | | N | |
| | | | YTNATHGTNAAYAAYGCNACNAAYGTNGTN | |
| 238 | GAYWSNWSNWSNGGNUGGACNGCNGGNGCNGC | 255 | GAYWSNWSNWSNGGNTGGACNGCNGGNGC | 5 |
| | NGCNUAYUAYGUNGGNUAYYUNCARCCN | | NGCNGCNTAYTAYGTNGGNTAYYTNCARCC | |
| | MGNACNUUYYUNYUNAARUAYAAYGARAAY | | N | |
| | | | MGNACNTTYYTNYTNAARTAYAAYGARAAY | |
| 239 | UGYUUYACNAAYGUNUAYGCNGAYWSNUUYG | 256 | TGYTTYACNAAYGTNTAYGCNGAYWSNTTY | 6 |
| | UNAUHMGNGGNGAYGARGUNMGNCARAUH | | GTNATHMGNGGNGAYGARGTNMGNCARAT | |
| | GCNCCNGGNCARACNGGNAARAUHGCNGAY | | H | |
| | | | GCNCCNGGNCARACNGGNAARATHGCNGAY | |
| 240 | MGNGCNMGNWSNGUNGCNWSNCARWSNAUHAU | 257 | MGNGCNMGNWSNGTNGCNWSNCARWSNAT | 7 |
| | HGCNUAYACNAUGWSNYUNGGNGCNGAR | | HATHGCNTAYACNATGWSNYTNGGNGCNGA | |
| | AAYWSNGUNGCNUAYWSNAAYAAYWSNAUH | | R | |
| | | | AAYWSNGTNGCNTAYWSNAAYAAYWSNATH | |
| 241 | GCNGARAAYWSNGUNGCNUAYWSNAAYAAYWSN | 258 | GCNGARAAYWSNGTNGCNTAYWSNAAYAAY | 8 |
| | AUHGCNAUHCCNACNAAYUUYACNAUH | | WSNATHGCNATHCCNACNAAYTTYACNATH | |
| | WSNGUNACNACNGARAUHYUNCCNGUNWSN | | WSNGTNACNACNGARATHYTNCCNGTNWSN | |
| 242 | GCNYUNCARAUHCCNUUYGCNAUGCARAUGGCNU | 259 | GCNYTNCARATHCCNTTYGCNATGCARATGGCN | 9 |
| | AYMGNUUYAAYGGNAUHGGNGUNACN | | TAYMGNTTYAAYGGNATHGGNGTNACN | |
| | CARAAYGUNYUNUAYGARAAYCARAARYUN | | CARAAYGTNYTNTAYGARAAYCARAARYTN | |
| 243 | UUYGCNAUGCARAUGGCNUAYMGNUUYAAYGGNA | 260 | TTYGCNATGCARATGGCNTAYMGNTTYAAYGG | 10 |
| | UHGGNGUNACNCARAAYGUNYUNUAY | | NATHGGNGTNACNCARAAYGTNYTNTAY | |
| | GARAAYCARAARYUNAUHGCNAAYCARUUY | | GARAAYCARAARYTNATHGCNAAYCARTTY | |
| 244 | MGNGARGGNGUNUUYGUNWSNAAYGGNACNCAY | 261 | MGNGARGGNGTNTTYGTNWSNAAYGGNACNC | 11 |
| | UGGUUYGUNACNCARMGNAAYUUYUAY | | AYTGGTTYGTNACNCARMGNAAYTTYTAY | |
| | GARCCNCARAUHAUHACNACNGAYAAYACN | | GARCCNCARATHATHACNACNGAYAAYACN | |
| 245 | MGNWSNAARCARMGNMGNCCNCARGGNYUNCCN | 262 | MGNWSNAARCARMGNMGNCCNCARGGNYTN | 12 |
| | AAYAAYACNGCNWSNUGGUUYACNGCN | | CCNAAYAAYACNGCNWSNTGGTTYACNGCN | |
| | YUNACNCARCAYGGNAARGARGAYYUNAAR | | YTNACNCARCAYGGNAARGARGAYYTNAAR | |
| 246 | WSNAARAARCCNMGNCARAARMGNACNGCNACNA | 263 | WSNAARAARCCNMGNCARAARMGNACNGCN | 13 |
| | ARGCNUAYAAYGUNACNCARGCNUUY | | ACNAARGCNTAYAAYGTNACNCARGCNTTY | |
| | GGNMGNMGNGGNCCNGARCARACNCARGGN | | GGNMGNMGNGGNCCNGARCARACNCARGGN | |
| 247 | GARYUNAUHMGNCARGGNACNGAYUAYAARCAYU | 264 | GARYTNATHMGNCARGGNACNGAYTAYAARCA | 14 |
| | GGCCNCARAUHGCNCARUUYGCNCCN | | YTGGCCNCARATHGCNCARTTYGCNCCN | |
| | WSNGCNWSNGCNUUYUUYGGNAUGWSNMGN | | WSNGCNWSNGCNTTYTTYGGNATGWSNMGN | |
| 248 | CARMGNCARAARAARCARCARACNGUNACNYUNYU | 265 | CARMGNCARAARAARCARCARACNGTNACNYT | 15 |
| | NCCNGCNGCNGAYYUNGAYGAYUUY | | NYTNCCNGCNGCNGAYYTNGAYGAYTTY | |
| | WSNAARCARYUNCARCARWSNAUGWSNWSN | | WSNAARCARYTNCARCARWSNATGWSNWSN | |
| 249 | YUNWSNUAYUUYAUHGCNWSNUUYMGNYUNUUY | 266 | YTNWSNTAYTTYATHGCNWSNTTYMGNYTNTTY | 16 |
| | GCNMGNACNMGNWSNAUGUGGWSNUUY | | GCNMGNACNMGNWSNATGTGGWSNTTY | |
| | AAYCCNGARACNAAYAUHYUNYUNAAYGUN | | AAYCCNGARACNAAYATHYTNYTNAAYGTN | |
| 250 | AAYAUHGUNAAYGUNWSNYUNGUNAARCCNWSNU | 267 | AAYATHGTNAAYGTNWSNYTNGTNAARCCNWS | 17 |
| | UYUAYGUNUAYWSNMGNGUNAARAAY | | NTTYTAYGTNTAYWSNMGNGTNAARAAY | |
| | YUNAAYWSNWSNMGNGUNCCNGAYYUNYUN | | YTNAAYWSNWSNMGNGTNCCNGAYYTNYTN | |

### Example 13—Correlation Between Multiple Autologous Allele-Binding Epitopes and CD8⁺ T Cell Responses

The inventors investigated the HLA-binding capacity of the immunogenic peptides SEQ ID NO. 2, 5, 9, 12-17 detected for each subject (N=15) from the analyzed COVID-19 convalescent donors.

First the inventors determined the complete HLA class I genotype for each subject and then predicted the number of autologous HLA alleles that could bind to each of the nine shared 9-mer peptides used in the FluoroSpot assay for the detection of peptide-specific IFN-γ producing T cells. The predicted HLA-binding epitopes were then matched to the CD8⁺ T cell responses measured for each peptide in each patient (total 15×9=135 data points, FIG. 25). The magnitude of CD8⁺ T cell responses tended to correlate with epitopes restricted to multiple autologous HLA alleles (Rs=0.189, p=0.029, FIG. 29A). In addition, the magnitude of CD8⁺ T cell responses generated by PEPIs (HLA≥3) (median dSFU=458) was significantly higher than those generated by non-PEPIs (HLA<3) (median dSFU=110), (p=0.008) (FIG. 29B).

Across the 135 data points there were 98 positive responses and 37 negative responses recorded. Among the 98 positive responses 37 were generated by PEPIs, while among the 37 negatives only 7 were PEPIs, the others were epitopes restricted to ≤3 autologous HLA alleles (FIG. 25). Overall, the 2×2 contingency table revealed association of T cell responses with PEPIs (p=0.041, Fisher Exact) but not with HLA-restricted epitopes (p=1.000, Fisher Exact) (FIG. 29C). For each subject between one and seven peptides out of nine proved to be PEPIs. Among the predicted PEPIs, 37/44 (84%) were confirmed by IFN-γ FluoroSpot assay to generate specific T cell responses in the given subject (FIG. 29D and FIG. 25).

These data demonstrate that the subjects' complete HLA-genotype influence their CD8⁺ T cell responses and that multiple autologous allele-binding capacity is a key feature of immunogenic epitopes. PEPIs precisely predicted subjects' PEPI-specific CD8+T cell responses.

### Example 14—Analysis and Identification of Target Peptides for Vaccine Formulations and Design of Universal Vaccine Candidates

Epitope predictions for each subject in the in silico human cohorts (MP, n=433) for each of their HLA class I and class II alleles (six HLA class I and class II alleles) for the AA sequence of the conserved regions of 19 known SARS-CoV-2 viral proteins using 9-mer (HLA class I) and 15-mer (HLA class II) frames (FIG. 1) was described in Example 6.

SARS-CoV-2 structural proteins (S, N, M, E) were screened and nine different 30-mer peptides were selected during a multi-step process. First, sequence diversity analysis was performed (as of 28 Mar. 2020 in the NCBI database) (‘U.S. National Library of Medicine. Severe acute respiratory syndrome coronavirus 2 https://www.ncbi.nlm.nih.gov/genome/browse#!/viruses/86693/’). The accession IDs were as follows: NC_045512.2, MN938384.1, MN975262.1, MN985325.1, MN988713.1, MN994467.1, MN994468.1, MN997409.1, MN988668.1, MN988669.1, MN996527.1, MN996528.1, MN996529.1, MN996530.1, MN996531.1, MT135041.1, MT135043.1, MT027063.1, and MT027062.1. The ID in bold represents the GenBank reference sequence. The translated coding sequences of the four structural protein sequences were aligned and compared using a multiple sequence alignment (Clustal Omega, EMBL-EBI, United Kingdom). Of the 19 sequences, 15 were identical; however, single AA changes occurred in four N protein sequences: MN988713.1, N 194 S->X; MT135043.1, N 343 D->V; MT027063.1, N 194 S->L; MT027062.1, N 194 S->L. The resulting AA substitutions affected only two positions of N protein sequence (AA 194 and 343), neither of which occurred in epitopes that have been selected as targets for vaccine development. Only one (H49Y) of the thirteen reported single-letter changes in the viral S protein (D614G, S943P, L5F, L8V, V367F, G476S, V483A, H49Y, Y145H/del, Q239K, A831V, D839Y/N/E, P1263L), has been involved in the PolyPEPI-SCoV-2 vaccine, but the prevalence of this variant is decreasing among later virus isolates (Korber et al. 2020). Recent report (February 2021) established 4 different lineage by analyzing 45,494 complete SARS-CoV-2 genome sequences in the world. Most frequent circulating mutations from this report identified 11 missense amino acid mutations, one in S protein (D614G), three located in N protein (R203K with two different DNA substitutions and G204R), and further seven mutations in NSP2, NSP12, NSP13, ORF3a and ORF8 (Wang et al. 2021) None of these amino acid positions were included in the nine 30-mers, supporting the proper selection of the conservative regions and intention to identify universal vaccine candidate peptides. Additionally, none of PolyPEPI-SCoV-2 peptides is affected by the emerging mutant SARS-CoV-2 strains: B.1.1.7 (UK, 17 mutations), B.1.351 (South Africa, 9 mutations) or B.1.1.28.1 (Brazil, 16 mutations), either (Thomson et al. 2020; Rambaut A 2020; O'Toole Å 2021a, 2021b).

This analysis of the emerged mutants from the period March 2020-February 2021 does not affect the sequence regions of the analyzed composition of SEQ ID NO. 2, 5, 9, 12-17. (the same composition as used for the preclinical immunogenicity testings of the peptides), suggesting that the present selection method can be used to design universal compositions.

### REFERENCES FOR EXAMPLE 10

1. Draft landscape of COVID-19 candidate vaccines. https://www.mabtech.com/products/sars-cov-2-s-n-m-o-defined-peptide-pool-3622-1-0.
2. M. Hellerstein, What are the roles of antibodies versus a durable, high quality T-cell response in protective immunity against SARS-CoV-2? Vaccine X 6, 100076 (2020).
3. M. Peiris, G. M. Leung, What can we expect from first-generation COVID-19 vaccines?The Lancet, (2020).
4. K. A. Callow, H. F. Parry, M. Sergeant, D. A. Tyrrell, The time course of the immune response to experimental coronavirus infection of man. Epidemiol Infect 105, 435-446 (1990).
5. Q. X. Long, X. J. Tang, Q. L. Shi, Q. Li, H. J. Deng, J. Yuan, J. L. Hu, W. Xu, Y. Zhang, F. J. Lv, K. Su, F. Zhang, J. Gong, B. Wu, X. M. Liu, J. J. Li, J. F. Qiu, J. Chen, A. L. Huang, Clinical and immunological assessment of asymptomatic SARS-CoV-2 infections. Nat Med 26, 1200-1204 (2020).
6. T. Sekine, A. Perez-Potti, O. Rivera-Ballesteros, K. Stråalin, J.-B. Gorin, A. Olsson, S. Llewellyn-Lacey, H. Kamal, G. Bogdanovic, S. Muschiol, D. J. Wullimann, T. Kammann, J. Emgard, T. Parrot, E. Folkesson, O. Rooyackers, L. I. Eriksson, A. Sönnerborg, T. Allander, J. Albert, M. Nielsen, J. Klingstrom, S. Gredmark-Russ, N. K. Björkström, J. K. Sandberg, D. A. Price, H.-G. Ljunggren, S. Aleman, M. Buggert, Robust T cell immunity in convalescent individuals with asymptomatic or mild COVID-19. (2020).
7. H. M. Staines, D. E. Kirwan, D. J. Clark, E. R. Adams, Y. Augustin, R. L. Byrne, M. Cocozza, A. I. Cubas-Atienza, L. E. Cuevas, M. Cusinato, B. M. O. Davies, M. Davis, P. Davis, A. Duvoix, N. M. Eckersley, D. Forton, A. Fraser, G. Garrod, L. Hadcocks, Q. Hu, M. Johnson, G. A. Kay, K. Klekotko, Z. Lewis, J. Mensah-Kane, S. Menzies, I. Monahan, C. Moore, G. Nebe-von-Caron, S. I. Owen, C. Sainter, A. A. Sall, J. Schouten, C. Williams, J. Wilkins, K. Woolston, J. R. A. Fitchett, S. Krishna, T. Planche, Dynamics of IgG seroconversion and pathophysiology of COVID-19 infections. (2020).
8. A. Grifoni, D. Weiskopf, S. I. Ramirez, J. Mateus, J. M. Dan, C. R. Moderbacher, S. A. Rawlings, A. Sutherland, L. Premkumar, R. S. Jadi, D. Marrama, A. M. de Silva, A. Frazier, A. F. Carlin, J. A. Greenbaum, B. Peters, F. Krammer, D. M. Smith, S. Crotty, A. Sette, Targets of T Cell Responses to SARS-CoV-2 Coronavirus in Humans with COVID-19 Disease and Unexposed Individuals. Cell 181, 1-13 (2020).
9. N. L. Bert, A. T. Tan, K. Kunasegaran, C. Y. L. Tham, M. Hafezi, A. Chia, M. Chng, M. Lin, N. Tan, M. Linster, W. N. Chia, M. I. C. Chen, L.-F. Wang, E. E. Ooi, S. Kalimuddin, P. A. Tambyah, J. G.-H. Low, Y.-J. Tan, A. Bertoletti, Different pattern of pre-existing SARS-COV-2 specific T cell immunity in SARS-recovered and uninfected individuals. (2020).
10. Y. Peng, A. J. Mentzer, G. Liu, X. Yao, Z. Yin, D. Dong, W. Dejnirattisai, T. Rostron, P. Supasa, C. Liu, C. Lopez-Camacho, J. Slon-Campos, Y. Zhao, D. I Stuart, G. C. Paesen, J. M. Grimes, A. A. Antson, O. W. Bayfield, D. Hawkins, D. S. Ker, B. Wang, L. Turtle, K. Subramaniam, P. Thomson, P. Zhang, C. Dold, J. Ratcliff, P. Simmonds, T. de Silva, P. Sopp, D. Wellington, U. Rajapaksa, Y. L. Chen, M. Salio, G. Napolitani, W. Paes, P. Borrow, B. M. Kessler, J. W. Fry, N. F. Schwabe, M. G. Semple, J. K. Baillie, S. C. Moore, P. J. M. Openshaw, M. A. Ansari, S. Dunachie, E. Barnes, J. Frater, G. Kerr, P. Goulder, T. Lockett, R. Levin, Y. Zhang, R. Jing, L. P. Ho, T. c. C. Oxford Immunology Network Covid-19 Response, I. C. Investigators, R. J. Cornall, C. P. Conlon, P. Klenerman, G. R. Screaton, J. Mongkolsapaya, A. McMichael, J. C. Knight, G. Ogg, T. Dong, Broad and strong memory CD4(+) and CD8(+) T cells induced by SARS-CoV-2 in UK convalescent individuals following COVID-19. NatImmunol, (2020).
11. A. Nelde, T. Bilich, J. S. Heitmann, Y. Maringer, H. R. Salih, M. Roerden, M. Lubke, J. Bauer, J. Rieth, M. Wacker, A. Peter, S. Horber, B. Traenkle, P. D. Kaiser, U. Rothbauer, M. Becker, D. Junker, G. Krause, M. Strengert, N. Schneiderhan-Marra, M. F. Templin, T. O. Joos, D. J. Kowalewski, V. Stos-Zweifel, M. Fehr, M. Graf, L.-C. Gruber, D. Rachfalski, B. PreuB, I. Hagelstein, M. Marklin, T. Bakchoul, C. Gouttefangeas, O. Kohlbacher, R. Klein, S. Stevanovid, H.-G. Rammensee, J. S. Walz, SARS-CoV-2 T-cell epitopes define heterologous and COVID-19-induced T-cell recognition. (2020).
12. J. Zhao, A. N. Alshukairi, S. A. Baharoon, W. A. Ahmed, A. A. Bokhari, A. M. Nehdi, L. A. Layqah, M. G. Alghamdi, M. M. Al Gethamy, A. M. Dada, I. Khalid, M. Boujelal, S. M. Al Johani, L. Vogel, K. Subbarao, A. Mangalam, C. Wu, P. Ten Eyck, S. Perlman, J. Zhao, Recovery from the Middle East respiratory syndrome is associated with antibody and T-cell responses. Sci Immunol 2, (2017).
13. B. Diao, C. Wang, Y. Tan, X. Chen, Y. Liu, L. Ning, L. Chen, M. Li, Y. Liu, G. Wang, Z. Yuan, Z. Feng, Y. Zhang, Y. Wu, Y. Chen, Reduction and Functional Exhaustion of T Cells in Patients With Coronavirus Disease 2019 (COVID-19). Front Immunol 11, 827 (2020).
14. T. M. Snyder, R. M. Gittelman, M. Klinger, D. H. May, E. J. Osborne, R. Taniguchi, H. J. Zahid, I. M. Kaplan, J. N. Dines, M. N. Noakes, R. Pandya, X. Chen, S. Elasady, E. Svejnoha, P. Ebert, M. W. Pesesky, P. De Almeida, H. O'Donnell, Q. DeGottardi, G. Keitany, J. Lu, A. Vong, R. Elyanow, P. Fields, J. Greissl, L. Baldo, S. Semprini, C. Cerchione, M. Mazza, O. M. Delmonte, K. Dobbs, G. Carreno-Tarragona, S. Barrio, L. Imberti, A. Sottini, E. Quiros-Roldan, C. Rossi, A. Biondi, L. R. Bettini, M. D′Angio, P. Bonfanti, M. F. Tompkins, C. Alba, C. Dalgard, V. Sambri, G. Martinelli, J. D. Goldman, J. R. Heath, H. C. Su, L. D. Notarangelo, J. Martinez-Lopez, J. M. Carlson, H. S. Robins, Magnitude and Dynamics of the T-Cell Response to SARS-CoV-2 Infection at Both Individual and Population Levels. medRxiv, (2020).
15. J. Zhao, J. Zhao, S. Perlman, T cell responses are required for protection from clinical disease and for virus clearance in severe acute respiratory syndrome coronavirus-infected mice. J Virol 84, 9318-9325 (2010).
16. R. Channappanavar, C. Fett, J. Zhao, D. K. Meyerholz, S. Perlman, Virus-specific memory CD8 T cells provide substantial protection from lethal severe acute respiratory syndrome coronavirus infection. J Virol 88, 11034-11044 (2014).
17. R. Channappanavar, J. Zhao, S. Perlman, T cell-mediated immune response to respiratory coronaviruses. Immunol Res 59, 118-128 (2014).
18. W. Li, M. D. Joshi, S. Singhania, K. H. Ramsey, A. K. Murthy, Peptide Vaccine: Progress and Challenges. Vaccines (Basel) 2, 515-536 (2014).
19. M. Black, A. Trent, M. Tirrell, C. Olive, Advances in the design and delivery of peptide subunit vaccines with a focus on toll-like receptor agonists. Expert review of vaccines 9, 157-173 (2010).
20. A. L. Thompson, H. F. Staats, Cytokines: the future of intranasal vaccine adjuvants. Clin Dev Immunol 2011, 289597 (2011).
21. N. Petrovsky, J. C. Aguilar, Vaccine adjuvants: current state and future trends. Immunol Cell Biol 82, 488-496 (2004).
22. D. Sesardic, Synthetic peptide vaccines. J Med Microbiol 39, 241-242 (1993).
23. M. S. Bijker, C. J. Melief, R. Offringa, S. H. van der Burg, Design and development of synthetic peptide vaccines: past, present and future. Expert Rev Vaccines 6, 591-603 (2007).
24. S. Nayak, R. W. Herzog, Progress and prospects: immune responses to viral vectors. Gene Ther 17, 295-304 (2010).
25. D. Pan, S. Sze, J. S. Minhas, M. N. Bangash, N. Pareek, P. Divall, C. M. Williams, M. R. Oggioni, I B. Squire, L. B. Nellums, W. Hanif, K. Khunti, M. Pareek, The impact of ethnicity on clinical outcomes in COVID-19: A systematic review. EClinicalMedicine 23, 100404 (2020).
26. R. W. Aldridge, D. Lewer, S. V. Katikireddi, R. Mathur, N. Pathak, R. Burns, E. B. Fragaszy, A. M. Johnson, D. Devakumar, I. Abubakar, A. Hayward, Black, Asian and Minority Ethnic groups in England are at increased risk of death from COVID-19: indirect standardisation of NHS mortality data. Wellcome Open Res 5, 88 (2020).
27. A. P. Ferretti, T. Kula, Y. Wang, D. M. Nguyen, A. Weinheimer, G. S. Dunlap, Q. Xu, N. Nabilsi, C. R. Perullo, A. W. Cristofaro, H. J. Whitton, A. Virbasius, K. J. Olivier, L. B. Baiamonte, A. T. Alistar, E. D. Whitman, S. A. Bertino, S. Chattopadhyay, G. MacBeath, COVID-19 Patients Form Memory CD8+ T Cells that Recognize a Small Set of Shared Immunodominant Epitopes in SARS-CoV-2. medRxiv, 2020.2007.2024.20161653 (2020).
28. U. Sahin, E. Derhovanessian, M. Miller, B. P. Kloke, P. Simon, M. Lower, V. Bukur, A. D. Tadmor, U. Luxemburger, B. Schrors, T. Omokoko, M. Vormehr, C. Albrecht, A. Paruzynski, A. N. Kuhn, J. Buck, S. Heesch, K. H. Schreeb, F. Muller, I. Ortseifer, I. Vogler, E. Godehardt, S. Attig, R. Rae, A. Breitkreuz, C. Tolliver, M. Suchan, G. Martic, A. Hohberger, P. Sorn, J. Diekmann, J. Ciesla, O. Waksmann, A. K. Bruck, M. Witt, M. Zillgen, A. Rothermel, B. Kasemann, D. Langer, S. Bolte, M. Diken, S. Kreiter, R. Nemecek, C. Gebhardt, S. Grabbe, C. Holler, J. Utikal, C. Huber, C. Loquai, O. Tureci, Personalized RNA mutanome vaccines mobilize poly-specific therapeutic immunity against cancer. Nature 547, 222-226 (2017).
29. P. A. Ott, Z. Hu, D. B. Keskin, S. A. Shukla, J. Sun, D. J. Bozym, W. Zhang, A. Luoma, A. Giobbie-Hurder, L. Peter, C. Chen, O. Olive, T. A. Carter, S. Li, D. J. Lieb, T. Eisenhaure, E. Gjini, J. Stevens, W. J. Lane, I. Javeri, K. Nellaiappan, A. M. Salazar, H. Daley, M. Seaman, E. I. Buchbinder, C. H. Yoon, M. Harden, N. Lennon, S. Gabriel, S. J. Rodig, D. H. Barouch, J. C. Aster, G. Getz, K. Wucherpfennig, D. Neuberg, J. Ritz, E. S. Lander, E. F. Fritsch, N. Hacohen, C. J. Wu, An immunogenic personal neoantigen vaccine for patients with melanoma. Nature 547, 217-221 (2017).
30. A. W. Purcell, J. McCluskey, J. Rossjohn, More than one reason to rethink the use of peptides in vaccine design. Nat Rev Drug Discov 6, 404-414 (2007).
31. O. Lorincz, J. Toth, M. Megyesi, K. Pintya, I. Miklos, E. Somogyi, Z. Csiszovszki, P. Pales, L. Molnar, E. R. Töke, 1935PComputational model to predict response rate of clinical trials. Annals of Oncology 30, (2019).
32. J. Hubbard, C. Cremolini, R. Graham, R. Moretto, J. Mitchell, J. Wessling, E. Toke, Z. Csiszovszki, O. Lörincz, L. Molnár, E. Somogyi, M. Megyesi, K. Pántya, J. Tóth, P. Páles, I. Miklós, A. Falcone, P329 PolyPEPI1018 off-the shelf vaccine as add-on to maintenance therapy achieved durable treatment responses in patients with microsatellite-stable metastatic colorectal cancer patients (MSS mCRC). Journal for ImmunoTherapy of Cancer 7 (34th Annual Meeting & Pre-Conference Programs of the Society for Immunotherapy of Cancer (SITC 2019): part 1), 282 (2019).
33. J. Hubbard, C. Cremolini, R. P. Graham, R. Moretto, J. Mitchell, J. Wessling, E. R. Töke, Z. Csiszovszki, O. Lorincz, L. Molnar, E. Somogyi, M. Megyesi, K. Pintya, J. Toth, P. Pales, I. Miklos, A. Falcone, 606PEvaluation of safety, immunogenicity and preliminary efficacy of PolyPEPI1018 vaccine in subjects with metastatic colorectal cancer (mCRC) with a predictive biomarker. Annals of Oncology 30, (2019).
34. E. Somogyi, Z. Csiszovszki, O. Lorincz, J. Toth, L. Molnar, W. Schönharting, S. Urban, T. Röhnisch, K. Pintya, P. Pales, M. Megyesi, E. R. Töke, 1181PDPersonal antigen selection calculator (PASCal) for the design of personal cancer vaccines. Annals of Oncology 30, (2019).
35. U.S. National Library of Medicine. Severe acute respiratory syndrome coronavirus 2 https://www.nchi.nlm.nih.gov/genome/browse#!/viruses/86693/.
36. F. Madeira, Y. M. Park, J. Lee, N. Buso, T. Gur, N. Madhusoodanan, P. Basutkar, A. R. N. Tivey, S. C. Potter, R. D. Finn, R. Lopez, The EMBL-EBI search and sequence analysis tools APIs in 2019. Nucleic Acids Res 47, W636-W641 (2019).
37. B. Korber, W. M. Fischer, S. Gnanakaran, H. Yoon, J. Theiler, W. Abfalterer, B. Foley, E. E. Giorgi, T. Bhattacharya, M. D. Parker, D. G. Partridge, C. M. Evans, T. M. Freeman, T. I. de Silva, C. C. LaBranche, D. C. Montefiori, Spike mutation pipeline reveals the emergence of a more transmissible form of SARS-CoV-2. (2020).
38. Centers for Disease Control and Prevention. Coronavirus https://www.edc.gov/coronavirus/types.html.
39. T. U. Consortium, UniProt: a worldwide hub of protein knowledge. Nucleic Acids Research 47, D506-D515 (2018).
40. C. International HapMap, D. M. Altshuler, R. A. Gibbs, L. Peltonen, D. M. Altshuler, R. A. Gibbs, L. Peltonen, E. Dermitzakis, S. F. Schaffner, F. Yu, L. Peltonen, E. Dermitzakis, P. E. Bonnen, D. M. Altshuler, R. A. Gibbs, P. I. de Bakker, P. Deloukas, S. B. Gabriel, R. Gwilliam, S. Hunt, M. Inouye, X. Jia, A. Palotie, M. Parkin, P. Whittaker, F. Yu, K. Chang, A. Hawes, L. R. Lewis, Y. Ren, D. Wheeler, R. A. Gibbs, D. M. Muzny, C. Barnes, K. Darvishi, M. Hurles, J. M. Kom, K. Kristiansson, C. Lee, S. A. McCarrol, J. Nemesh, E. Dermitzakis, A. Keinan, S. B. Montgomery, S. Pollack, A. L. Price, N. Soranzo, P. E. Bonnen, R. A. Gibbs, C. Gonzaga-Jauregui, A. Keinan, A. L. Price, F. Yu, V. Anttila, W. Brodeur, M. J. Daly, S. Leslie, G. McVean, L. Moutsianas, H. Nguyen, S. F. Schaffner, Q. Zhang, M. J. Ghori, R. McGinnis, W. McLaren, S. Pollack, A. L. Price, S. F. Schaffner, F. Takeuchi, S. R. Grossman, I. Shlyakhter, E. B. Hostetter, P. C. Sabeti, C. A. Adebamowo, M. W. Foster, D. R. Gordon, J. Licinio, M. C. Manca, P. A. Marshall, I. Matsuda, D. Ngare, V. O. Wang, D. Reddy, C. N. Rotimi, C. D. Royal, R. R. Sharp, C. Zeng, L. D. Brooks, J. E. McEwen, Integrating common and rare genetic variation in diverse human populations. Nature 467, 52-58 (2010).
41. J. Robinson, J. A. Halliwell, J. D. Hayhurst, P. Flicek, P. Parham, S. G. Marsh, The IPD and IMGT/HLA database: allele variant databases. Nucleic Acids Res 43, D423-431 (2015).
42. J. Robinson, J. A. Halliwell, H. McWilliam, R. Lopez, S. G. Marsh, IPD—the Immuno Polymorphism Database. Nucleic Acids Res 41, D1234-1240 (2013).
43. K. Yusim, B. T. M. Korber, C. Brander, D. Barouch, R. de Boer, B. F. Haynes, R. Koup, J. P. Moore, B. D. Walker, D. I. Watkins, in HIV Molecular Immunology 2014. (Los Alamos National Laboratory, Theoretical Biology and Biophysics, https://www.hiv.lanl.gov/content/immunology.compendium.html, 2014), vol. LA-UR 15-20754.
44. G. Pawelec, S. G. Marsh, ESTDAB: a collection of immunologically characterised melanoma cell lines and searchable databank. Cancer Immunol Immunother 55, 623-627 (2006).
45. J. Robinson, C. H. Roberts, I. A. Dodi, J. A. Madrigal, G. Pawelec, L. Wedel, S. G. Marsh, The European searchable tumour line database. Cancer Immunol Immunother 58, 1501-1506 (2009).
46. J. R. Arguello, A. M. Little, E. Bohan, J. M. Goldman, S. G. Marsh, J. A. Madrigal, High resolution HLA class I typing by reference strand mediated conformation analysis (RSCA). Tissue Antigens 52, 57-66 (1998).
47. W. Helmberg, R. Dunivin, M. Feolo, The sequencing-based typing tool of dbMHC: typing highly polymorphic gene sequences. Nucleic Acids Res 32, W173-175 (2004).
48. L. Gragert, A. Madbouly, J. Freeman, M. Maiers, Six-locus high resolution HLA haplotype frequencies derived from mixed-resolution DNA typing for the entire US donor registry. Hum Immunol 74, 1313-1320 (2013).
49. V. Paunic, L. Gragert, J. Schneider, C. Muller, M. Maiers, Charting improvements in US registry HLA typing ambiguity using a typing resolution score. Human Immunology 77, 542-549 (2016).
50. H. Akoglu, User's guide to correlation coefficients. Turk J Emerg Med 18, 91-93 (2018).
51. J. P. Guo, M. Petric, W. Campbell, P. L. McGeer, SARS corona virus peptides recognized by antibodies in the sera of convalescent cases. Virology 324, 251-256 (2004).
52. S. J. Liu, C. H. Leng, S. P. Lien, H. Y. Chi, C. Y. Huang, C. L. Lin, W. C. Lian, C. J. Chen, S. L. Hsieh, P. Chong, Immunological characterizations of the nucleocapsid protein based SARS vaccine candidates. Vaccine 24, 3100-3108 (2006).
53. Y. He, Y. Zhou, H. Wu, Z. Kou, S. Liu, S. Jiang, Mapping of antigenic sites on the nucleocapsid protein of the severe acute respiratory syndrome coronavirus. J Clin Microbiol 42, 5309-5314 (2004).
54. S. F. Ahmed, A. A. Quadeer, M. R. McKay, Preliminary Identification of Potential Vaccine Targets for the COVID-19 Coronavirus (SARS-CoV-2) Based on SARS-CoV Immunological Studies. Viruses 12, (2020).
55. M. Maiers, L. Gragert, W. Klitz, High-resolution HLA alleles and haplotypes in the United States population. Hum Immunol 68, 779-788 (2007).
56. Faviel F. Gonzalez-Galarza, A. McCabe, Eduardo J M. d. Santos, J. Jones, L. Takeshita, Nestor D. Ortega-Rivera, Glenda M. D. Cid-Pavon, K. Ramsbottom, G. Ghattaoraya, A. Alfirevic, D. Middleton, Andrew R. Jones, Allele frequency net database (AFND) 2020 update: gold-standard data classification, open access genotype data and new query tools. Nucleic Acids Research 48, D783-D788 (2019).
57. M. J. Mulligan, K. E. Lyke, N. Kitchin, J. Absalon, A. Gurtman, S. Lockhart, K. Neuzil, V. Raabe, R. Bailey, K. A. Swanson, P. Li, K. Koury, W. Kalina, D. Cooper, C. Fontes-Garfias, P. Y. Shi, O. Tureci, K. R. Tompkins, E. E. Walsh, R. Frenck, A. R. Falsey, P. R. Dormitzer, W. C. Gruber, U. Sahin, K. U. Jansen, Phase 1/2 study of COVID-19 RNA vaccine BNT162b1 in adults. Nature, (2020).
58. SARS-CoV-2 S N M O defined peptide pool. https://www.mabtech.com/products/sars-cov-2-s-a-m-o-defined-peptide-pool-3622-1-0.
59. D. C. Parker, T cell-dependent B cell activation. Annu Rev Immunol 11, 331-360 (1993).
60. T. Kar, U. Narsaria, S. Basak, D. Deb, F. Castiglione, D. M. Mueller, A. P. Srivastava, A candidate multi-epitope vaccine against SARS-CoV-2. Sci Rep 10, 10895 (2020).
61. E. R. Toke, M. Megyesi, L. Molnar, J. Tóth, O. Lőrincz, S. H. van der Burg, M. Welters, C. J. Melief, W. Schönharting, S. Urban, T. Roehnisch, U. Heine, E. Somogyi, Z. Csiszovszki, K. Pántya, P. Páles, I. Miklós, F. Lori, J. Lisziewicz, Prediction the clinical outcomes of cancer patients after peptide vaccination. Journal of Clinical Oncology 37, e14295 (2019).
62. Demographics of the world. https://www.quora.com/What-are-all-the-races-and-their-world-population-demographics-the-entire-world.
63. S. Ascarateil, A. Puget, M.-E. Koziol, Safety data of Montanide ISA 51 VG and Montanide ISA 720 VG, two adjuvants dedicated to human therapeutic vaccines. Journal for ImmunoTherapy of Cancer 3, (2015).
64. J. Atsmon, E. Kate-Ilovitz, D. Shaikevich, Y. Singer, I. Volokhov, K. Y. Haim, T. Ben-Yedidia, Safety and immunogenicity of multimeric-001—a novel universal influenza vaccine. J Clin Immunol 32, 595-603 (2012).
65. B. S. Graham, M. J. McElrath, M. C. Keefer, K. Rybczyk, D. Berger, K. J. Weinhold, J. Ottinger, G. Ferarri, D. C. Montefiori, D. Stablein, C. Smith, R. Ginsberg, J. Eldridge, A. Duerr, P. Fast, B. F. Haynes, Immunization with cocktail of HIV-derived peptides in montanide ISA-51 is immunogenic, but causes sterile abscesses and unacceptable reactogenicity. PLoS One 5, e 11995 (2010).
66. S. Herrera, O. L. Fernández, O. Vera, W. Cárdenas, O. Ramirez, R. Palacios, M. Chen-Mok, G. Corradin, M. Ardvalo-Herrera, Phase I safety and immunogenicity trial of Plasmodium vivax CS derived long synthetic peptides adjuvanted with montanide ISA 720 or montanide ISA 51. The American journal of tropical medicine and hygiene 84, 12-20 (2011).
67. G. Pialoux, J. L. Excler, Y. Rivibre, G. Gonzalez-Canali, V. Feuillie, P. Coulaud, J. C. Gluckman, T. J. Matthews, B. Meignier, M. P. Kieny, et al., A prime-boost approach to HIV preventive vaccine using a recombinant canarypox virus expressing glycoprotein 160 (MN) followed by a recombinant glycoprotein 160 (MN/LAI). The AGIS Group, and l'Agence Nationale de Recherche sur le SIDA. AIDS Res Hum Retroviruses 11, 373-381 (1995).
68. O. Pleguezuelos, S. Robinson, G. A. Stoloff, W. Caparrós-Wanderley, Synthetic Influenza vaccine (FLU-v) stimulates cell mediated immunity in a double-blind, randomised, placebo-controlled Phase I trial. Vaccine 30, 4655-4660 (2012).
69. Y. Wu, R. D. Ellis, D. Shaffer, E. Fontes, E. M. Malkin, S. Mahanty, M. P. Fay, D. Narum, K. Rausch, A. P. Miles, J. Aebig, A. Orcutt, O. Muratova, G. Song, L. Lambert, D. Zhu, K. Miura, C. Long, A. Saul, L. H. Miller, A. P. Durbin, Phase 1 trial of malaria transmission blocking vaccine candidates Pfs25 and Pvs25 formulated with montanide ISA 51. PLoS One 3, e2636 (2008).

### Other References

Ahmed, S. F., Quadeer, A. A., & McKay, M. R. (2020). Preliminary Identification of Potential Vaccine Targets for the COVID-19 Coronavirus (SARS-CoV-2) Based on SARS-CoV Immunological Studies. Viruses, 12(3). doi:10.3390/v12030254
Bagarazzi, M. L., Yan, J., Morrow, M. P., Shen, X., Parker, R. L., Lee, J. C., Giffear, M., Pankhong, P., Khan, A. S., Broderick, K. E., Knott, C., Lin, F., Boyer, J. D., Draghia-Akli, R., White, C. J., Kim, J. J., Weiner, D. B. & Sardesai, N.Y., 2012. Immunotherapy against HPV16/18 generates potent TH1 and cytotoxic cellular immune responses. Sci Transl Med, 4, 155ra138.
Bioley, G., Guillaume, P., Luescher, I., Yeh, A., Dupont, B., Bhardwaj, N., Mears, G., Old, L. J., Valmori, D. & Ayyoub, M., 2009. HLA class I—associated immunodominance affects CTL responsiveness to an ESO recombinant protein tumor antigen vaccine. Clin Cancer Res, 15, 299-306.
Esseku F, Adeyeye M C. Bacteria and pH-sensitive polysaccharide-polymer films for colon targeted delivery. Crit Rev Ther Drug Carrier Syst 2011; 28: 395-445.
Gennaro, 2000, Remington: The Science and Practice of Pharmacy, 20th edition, ISBN:0683306472
Grothey, A., Tabemero, J., Arnold, D., De Gramont, A., Ducreux, M. P., O'dwyer, P. J., Van Cutsem, E., Bosanac, I., Srock, S., Mancao, C., Gilberg, F., Winter, J. & Schmoll, H.-J., 2018. LBA19Fluoropyrimidine (FP)+bevacizumab (BEV)+atezolizumab vs FP/BEV in BRAFwt metastatic colorectal cancer (mCRC): Findings from Cohort 2 of MODUL—a multicentre, randomized trial of biomarker-driven maintenance treatment following first-line induction therapy. Annals of Oncology, 29.
Gudmundsdotter, L., Wahren, B., Haller, B. K., Boberg, A., Edback, U., Bernasconi, D., Butto, S., Gaines, H., Imami, N., Gotch, F., Lori, F., Lisziewicz, J., Sandstrom, E. & Hejdeman, B., 2011. Amplified antigen-specific immune responses in HIV-1 infected individuals in a double blind DNA immunization and therapy interruption trial. Vaccine, 29, 5558-66.
Kakimi, K., Isobe, M., Uenaka, A., Wada, H., Sato, E., Doki, Y., Nakajima, J., Seto, Y., Yamatsuji, T., Naomoto, Y., Shiraishi, K., Takigawa, N., Kiura, K., Tsuji, K., Iwatsuki, K., Oka, M., Pan, L., Hoffman, E. W., Old, L. J. & Nakayama, E., 2011. A phase I study of vaccination with NY-ESO-lf peptide mixed with Picibanil OK-432 and Montanide ISA-51 in patients with cancers expressing the NY-ESO-1 antigen. Int J Cancer, 129, 2836-46.
Kissler S M, Tedijanto C, Goldstein E, Grad Y H, Lipsitch M. The copyright holder for this preprint. https://doi.org/10.1101/2020.03.04.20031112
Klebanoff, C. A., Acquavella, N., Yu, Z. & Restifo, N. P., 2011. Therapeutic cancer vaccines: are we there yet? Immunological reviews, 239, 27-44.
Lorincz, O., Toth, J., Megyesi, M., Pintya, K., Miklos, I., Somogyi, E., Csiszovszki, Z., Pales, P., Molnar, L. & Töke, E. R., 2019. 1935PComputational model to predict response rate of clinical trials. Annals of Oncology, 30.
Nascimbeni, M., Mizukoshi, E., Bosmann, M., Major, M. E., Mihalik, K., Rice, C. M., Feinstone, S. M. & Rehermann, B., 2003. Kinetics of CD4+ and CD8+ memory T-cell responses during hepatitis C virus rechallenge of previously recovered chimpanzees. J Virol, 77, 4781-93.
Paoletti L C. Potency of clinical group B streptococcal conjugate vaccines. Vaccine Volume 19, Issues 15-16, 28 Feb. 2001, Pages 2118-2126
Powell M. F. & Newman M. J. The subunit and adjuvant approach (book, 1995) Plenum Press New York
Slingluff C L, Jr. The present and future of peptide vaccines for cancer: single or multiple, long or short, alone or in combination? Cancer J 2011; 17(5): 343-50
Toke, E. R., Megyesi, M., Molnar, L., Tóth, J., Lorincz, O., Van Der Burg, S. H., Welters, M., Melief, C. J., Schönharting, W., Urban, S., Roehnisch, T., Heine, U., Somogyi, E., Csiszovszki, Z., Pántya, K., Páles, P., Miklós, I., Lori, F. & Lisziewicz, J., 2019. Prediction the clinical outcomes of cancer patients after peptide vaccination. Journal of Clinical Oncology, 37, e14295.
Trevor R. F. Smith, Ami Patel, Stephanie Ramos, Dustin Elwood, Xizhou Zhu, Jian Yan, Maria Yang, Neethu Chokkalingam, Ebony N. Gary, Katherine Schultheis, Patrick Pezzoli, Jewell Walters, Susanne N. Walker, Elizabeth Parzych, Emma L. Reuschel, Arthur Doan, Nicholas Tursi, Miguel Vasquez, Jihae Choi, Igor Maricic, Mamadou A. Bah, Yuanhan Wu, Dinah Amante, Daniel Park, Yaya Dia, Ali Raza Ali, Faraz I. Zaidi, Alison Generotti, Kevin Y. Kim, Timothy A. Herring, Sophia Reeder, Ami Shah Brown, Makan Khoshnejad, Nianshuang Wang, Jacqueline Chu, Daniel Wrapp, Jason S McLellan, Kar Muthumani, J Joseph Kim, Jean Boyer, Daniel W. Kulp, Laurent M. P. F. Humeau, David B. Weiner, Kate E. Broderick. Rapid development of a synthetic DNA vaccine for COVID-19. under review 10.21203/rs.3.rs-16261/v1
Valmori, D., Souleimanian, N. E., Tosello, V., Bhardwaj, N., Adams, S., O'neill, D., Pavlick, A., Escalon, J. B., Cruz, C. M., Angiulli, A., Angiulli, F., Mears, G., Vogel, S. M., Pan, L., Jungbluth, A. A., Hoffmann, E. W., Venhaus, R., Ritter, G., Old, L. J. & Ayyoub, M., 2007. Vaccination with NY-ESO-1 protein and CpG in Montanide induces integrated antibody/Th1 responses and CD8 T cells through cross-priming. Proc Natl Acad Sci USA, 104, 8947-52.
Wada, H., Isobe, M., Kakimi, K., Mizote, Y., Eikawa, S., Sato, E., Takigawa, N., Kiura, K., Tsuji, K., Iwatsuki, K., Yamasaki, M., Miyata, H., Matsushita, H., Udono, H., Seto, Y., Yamada, K., Nishikawa, H., Pan, L., Venhaus, R., Oka, M., Doki, Y. & Nakayama, E., 2014. Vaccination with NY-ESO-1 overlapping peptides mixed with Picibanil OK-432 and montanide ISA-51 in patients with cancers expressing the NY-ESO-1 antigen. J Immunother, 37, 84-92.
Van den Mooter G, Weuts I, De Ridder T, Blaton N. Evaluation of Inutec SP1 as a new carrier in the formulation of solid dispersions for poorly soluble drugs. Int J Pharm. 2006 Jun. 19; 316(1-2):1-6.
Yuan, J., Adamow, M., Ginsberg, B. A., Rasalan, T. S., Ritter, E., Gallardo, H. F., Xu, Y., Pogoriler, E., Terzulli, S. L., Kuk, D., Panageas, K. S., Ritter, G., Sznol, M., Halaban, R., Jungbluth, A. A., Allison, J. P., Old, LI., Wolchok, J. D. & Gnjatic, S., 2011. Integrated NY-ESO-1 antibody and CD8+ T-cell responses correlate with clinical benefit in advanced melanoma patients treated with ipilimumab. Proc Natl Acad Sci USA, 108, 16723-8.

1. A panel of two or more polypeptides of up to 50 amino acids in length, wherein each polypeptide comprises a different amino acid sequence selected from SEQ ID NOs: 1 to 17.

2. The panel of polypeptides of claim 1, wherein each polypeptide consists of a fragment of a Coronaviridae protein.

3. The panel of polypeptides of claim 2, wherein each polypeptide consists of a fragment of a SARS-CoV-2 protein.

4. The panel of polypeptides of any one of claims 1 to 3, wherein each polypeptide comprises an amino acid sequence selected from SEQ ID NOs: 1 to 17 that is a fragment of a different Coronaviridae or SARS-CoV-2 protein.

5. The panel of polypeptides of any one of claims 1 to 4, wherein the panel of polypeptides includes at least one sequence from at least two, three or all four of the following groups:
(a) SEQ ID NOs: 1 to 11;
(b) SEQ ID NOs: 12 to 15;
(c) SEQ ID NO: 16; and
(d) SEQ ID NO: 17.

6. The panel of polypeptides of claim 5, wherein the panel comprises ten polypeptides comprising or consisting of the amino acid sequences of SEQ ID NOs: 2, 5, 7, 9, 12, 13, 14, 15, 16, and 17.

7. The panel of polypeptides of claim 5, wherein the panel comprises nine polypeptides comprising or consisting of the amino acid sequences of SEQ ID NOs: 2, 5, 9, 12, 13, 14, 15, 16, and 17.

8. A pharmaceutical composition or kit having the panel of polypeptides according to claim any one of claims 1 to 7 as active ingredients.

9. The pharmaceutical composition or kit of claim 8, comprising polynucleic acid, ribonucleic acid, or one or more vectors or cells that together encode each of the polypeptides.

10. The pharmaceutical composition or kit of claim 9, comprising one or more polynucleotides or polyribonucleotides comprising at least two sequences selected from SEQ ID NOs: 234 to 251 or 252 to 268.

11. A method of vaccination, providing immunotherapy or inducing immune responses in a subject, the method comprising administering to the subject the panel of polypeptides of any one of claims 1 to 7 or the pharmaceutical composition of any one of claims 8 to 10.

12. The method of claim 11 that is a method of treating a Coronaviridae infection or a disease or condition associated with a Coronaviridae infection in the subject.

13. The method of claim 12 that is a method of treating a SARS-CoV-2 infection or COVID-19 disease in the subject.

14. The method of claim 11 that is a method of preventing a Coronaviridae infection or the development of a disease or condition associated with Coronaviridae infection in the subject.

15. The method of claim 14 that is a method of preventing a SARS-CoV-2 infection or development of COVID-19 disease in the subject.

16. The method of any one of claims 11 to 15, wherein one or more of the polypeptides comprises a fragment of a Coronaviridae protein that is a CD8+ T cell epitope predicted to be restricted to at least two HLA class I alleles of the subject.

17. The method of claim 16, wherein one or more of the polypeptides comprises a fragment of a Coronaviridae protein that is a CD4+ T cell epitope predicted to be restricted to at least two HLA class II alleles of the subject.

18. The method of any one of claims 11 to 17, wherein one or more of the polypeptides comprises a linear B cell epitope.
